(19) United States
(12) Patent Application Publication
(10) Pub. No.: US 2014/0288077 A1
(43) Pub. Date: Sep. 25, 2014

(54) SUBSTITUTED 4-PHENOXYPHENOL ANALOGS AS MODULATORS OF PROLIFERATING CELL NUCLEAR ANTIGEN ACTIVITY

(71) Applicants: Naoaki Fujii, Germantown, TN (US); Marcelo Actis, Bartlett, TN (US); Chandanamali Punchihewa, Cordova, TN (US); Michele Connelly, Memphis, TN (US); Sean Wu, Cleveland, OH (US)

(72) Inventors: Naoaki Fujii, Germantown, TN (US); Marcelo Actis, Bartlett, TN (US); Chandanamali Punchihewa, Cordova, TN (US); Michele Connelly, Memphis, TN (US); Sean Wu, Cleveland, OH (US)

(73) Assignee: ST. JUDE CHILDREN'S RESEARCH HOSPITAL, Memphis, TN (US)

(21) Appl. No.: 14/130,885

(22) PCT Filed: Jul. 5, 2012
(86) PCT No.: PCT/US2012/045624

§ 371 Date: Jun. 13, 2014

Related U.S. Application Data
(60) Provisional application No. 61/504,620, filed on Jul. 5, 2011.
(60) Provisional application No. 61/564,114, filed on Nov. 28, 2011.

(51) Int. Cl. C07C 235/46; C07F 15/00; C07D 241/08; C07D 295/16; C07C 275/24; C07C 311/04

(52) U.S. Cl. CPC C07C 235/46; C07C 275/24; C07C 311/04; C07D 241/08; C07D 295/16; C07F 15/0093
USPC 514/237.5; 564/156; 514/616; 564/47; 514/595; 564/99; 514/605; 564/171; 514/622; 544/384; 514/255.02; 544/176; 556/137; 514/492; 546/245; 514/330

(57) ABSTRACT

In one aspect, the invention relates to substituted 4-phenoxyphenol analogs, derivatives thereof, and related compounds, which are useful as inhibitors of proliferating cell nuclear antigen (PCNA); synthetic methods for making the compounds; pharmaceutical compositions comprising the compounds; and methods of treating hyperproliferative disorders associated with PCNA using the compounds and compositions. This abstract is intended as a scanning tool for purposes of searching in the particular art and is not intended to be limiting of the present invention.

### CROSS-REFERENCE TO RELATED APPLICATIONS

This application claims the benefit of U.S. Application Nos. 61/504,620, filed Jul. 5, 2011, and U.S. 61/564,114, filed Nov. 28, 2011, both of which are hereby incorporated by reference in their entirety.

### BACKGROUND

The estimated number of new cancer cases for 2011 in the United States alone is about 1.5 million, with number of deaths from cancer in the same period estimated to be nearly 600,000, according to data from the American Cancer Society. Despite decades of effort directed to the development of new therapeutic agents to treat cancer, including intense effort beginning with the “War on Cancer” with the National Cancer Act of 1971 in the United States, cancer remains a major cause of death.

A major category of therapeutic agents used in the treatment of cancer act by damaging DNA. Damaged DNA can inhibit the ability of cells to replicate and divide. Furthermore, damaged DNA can disrupt the viability of the cell to maintain normal cellular function interfering with the ability of the cell to properly express gene function via transcription of a gene to RNA. Thus, it the maintenance of genomic intergrity is critical to both normal and cancer cell function. Maintenance of genomic integrity within the cell requires a co-ordination between cell-cycle regulated DNA replication, and DNA repair. In the presence of damaged DNA, proliferating cells must cease DNA replication, so that lesions do not become fixed, and repair all damage before replication can recommence. Therefore, the co-ordination of these two processes is critical to avoid mutation and genomic instability. One protein known to be involved in both in DNA replication and in nucleotide excision repair is proliferating cell nuclear antigen (PCNA).

PCNA is member of the sliding clamp family of proteins which are functionally conserved from bacteria to higher eukaryotes, and whose main function is to provide replicative polymerases with the high processivity needed for duplication of the genome (e.g. see review by Moldovan, et al. Cell (2007) 129, 665-679). In live S-phase cells, PCNA tagged with green fluorescent protein (GFP) forms distinct foci representing sites of replication (Kisielewska, et al., Biol. Cell (2005) 97, 221-229). It can therefore be used as an S-phase marker. PCNA is an essential auxiliary protein for the processes of both DNA replication and repair. It stimulates the activity of DNA polymerase δ (pol δ) and increases its processivity (Prelich, G. et al., Nature (1987) 326, 517-20) by acting as a clamp platform that slides along the DNA template (Krishna, et al., Cell (1994) 79, 1233-1243). Apart from pol δ, PCNA associates with a host of other proteins, either involved directly in DNA replication and repair, or in the regulation of these processes (Warbrick, E. Bioessays (2000) 22, 997-1006). The presence of a common PCNA-binding motif in such proteins suggests that regulation may depend largely on PCNA partner proteins competing with one another for access to PCNA.

Numerous proteins involved in cellular processes such as DNA repair, chromatin assembly, epigenetic and chromatin remodelling, sister-chromatid cohesion, cell cycle control and survival are localised in so-called replication factories which contain more than a dozen replication forks. Many of these proteins interact with PCNA through the conserved PCNA interacting peptide sequence called the PIP-box (QxxL/I/MxxF/DF/Y), wherein x can be any amino acid (Moldovan, et al. Cell (2007) 129, 665-679). An alternative PCNA binding motif called the KAx box was identified using a peptide display library, but this motif has not been verified to be important for PCNA interactions in vivo (Moldovan, et al. Cell (2007) 129, 665-679).

Deregulation of PCNA expression is a hallmark of many proliferative diseases and in the clinic PCNA serves as a general proliferative marker, especially in the prognosis of tumour development (Paunesku, T. et al., Int. J. Radiat. Biol. (2001) 77, 1007-21). In fact PCNA expression levels are directly related to the malignancy of various tumours and antisense oligonucleotide-mediated suppression of PCNA expression was demonstrated to selectively inhibit gastric cancer cell proliferation in vitro and in vivo (Sakakura, C. et al., Br. J. Cancer (1994) 70, 1060-6). The fact that PCNA is required absolutely for cell proliferation indicates that pharmacological modulation of PCNA function should not be able to be circumvented by compensatory pathways. Furthermore, the ablation of PCNA expression or function in cells under proliferative stimuli appears to constitute an apoptotic trigger (Paunesku, T. et al., Int. J. Radiat. Biol. (2001) 77, 1007-21), suggesting that effective elimination of hyper-proliferative cells should be possible in a therapeutic setting.

Although successful treatment of cancer is of great importance to medical community, agents that target PCNA have use beyond this therapeutic area. In general, interruption of PCNA function can interfere with cell replication in a number diseases characterized as hyperproliferative or proliferative disorders. Examples of such disorders include psoriatic arthritis, rheumatoid arthritis, gastric hyperproliferative disorders such as inflammatory bowel disease, skin disorders including psoriasis, Reiter's syndrome, pityriasis rubra pilaris, and hyperproliferative variants of disorders of keratinization. Antisense strategies targeting PCNA mRNA have also shown promise in models of other proliferative diseases, including glomerular nephritis (Maeshima, et al. J. Amer. Soc. Nephrol. (1996) 7, 2219-29 (1996), restenosis (Speir, E. & Epstein, S. E. Circulation (1992) 86, 538-47) and rheumatoid arthritis (Morita, Y. et al. Arthritis And Rheumatism (1997) 40, 1292-7). Furthermore, the ablation of PCNA expression or function in cells under proliferative stimuli appears to constitute an apoptotic trigger (Paunesku, T. et al., Int. J. Radiat. Biol. (2001) 77, 1007-21), suggesting that effective elimination of hyperproliferative cells should be possible in a therapeutic setting.

Collectively these facts indicate that PCNA may represent an attractive target for intervention in proliferative disease. Despite advances in the understanding and physiology of PCNA, there is still a scarcity of compounds that are both potent, efficacious, and selective inhibitors of the activity of PCNA and also effective in the treatment of hyperproliferative disorders associated with PCNA and diseases in which the PCNA is involved. These needs and other needs are satisfied by the present invention.

### SUMMARY

In accordance with the purpose(s) of the invention, as embodied and broadly described herein, the invention, in one aspect, relates to compounds useful as inhibitors of PCNA activity, methods of making same, pharmaceutical compositions comprising same, and methods of treating hyperproliferative disorders using same.

Disclosed are compounds having a structure represented by a formula:

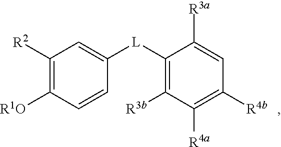

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein one of R^{4a} and R^{4b} is selected from hydrogen, halo, C1-C3 alkyl, C1-C3 haloalkyl, and C1-C3 polyhaloalkyl, and the other is selected from:

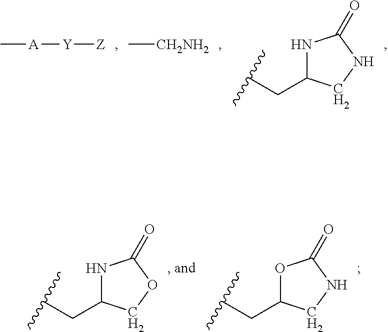

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

Disclosed are compounds having a structure represented by a formula:

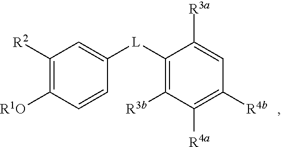

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹¹ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

Disclosed are compounds having a structure represented by a formula:

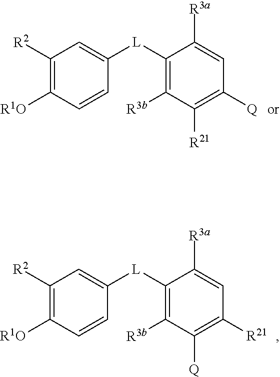

wherein L is selected from O, CH₂, CHOH, and C═O; wherein Q is selected from a structure represented by a formula:

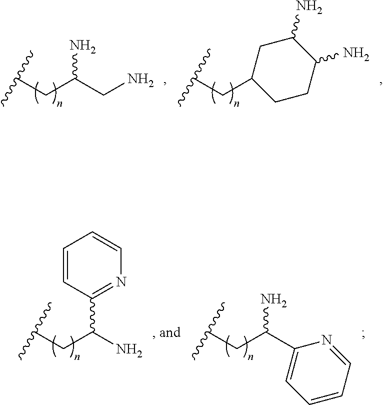

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

Also disclosed are methods of synthesis for making the disclosed compounds.

Also disclosed are pharmaceutical compositions comprising a therapeutically effective amount of a disclosed compound and a pharmaceutically acceptable carrier.

Also disclosed are methods for manufacturing a medicament comprising combining at least one disclosed compound or at least one disclosed product with a pharmaceutically acceptable carrier or diluent.

Also disclosed are uses of a disclosed compound or a disclosed product in the manufacture of a medicament for the treatment of a hyperproliferative disorder.

Also disclosed are methods of using the disclosed compounds: treatment of a disorder associated with PCNA dysfunction in a mammal, modulation of PCNA activity in a mammal, treatment of a proliferative disorder in a mammal, inhibiting cell growth in a mammal, cytostatic therapy in a mammal, and modulation of PCNA activity in cells. For example, the methods can comprise administering an effective amount of at least one compound having a structure represented by a formula:

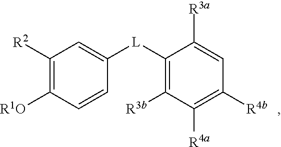

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In a further aspect, the methods can comprise administering an effective amount of at least one compound having a structure represented by a formula:

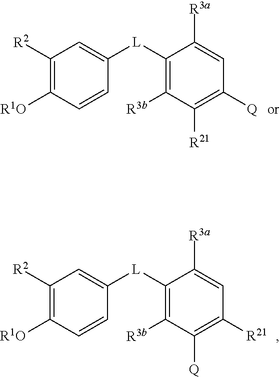

wherein L is selected from O, CH₂, CHOH, and C═O; wherein Q is selected from a structure represented by a formula:

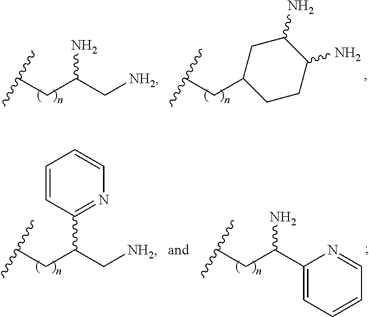

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

Also disclosed are kits comprising at least one compound having a structure represented by a formula:

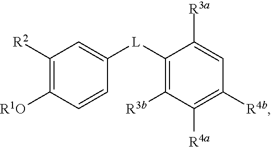

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof, and one or more of: (a) at least one agent known to decrease PCNA activity; (b) at least one agent known to increase PCNA activity; (c) at least one agent known to treat a proliferative disorder; or (d) instructions for treating a disorder associated with PCNA dysfunction.

In a further aspect, kits are disclosed comprising at least one compound having a structure represented by a formula:

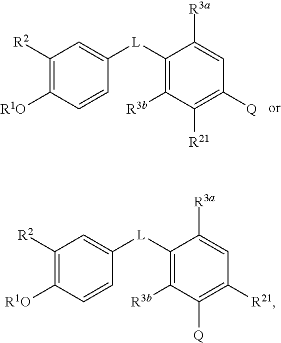

wherein L is selected from O, CH₂, CHOH, and C═O; wherein Q is selected from a structure represented by a formula:

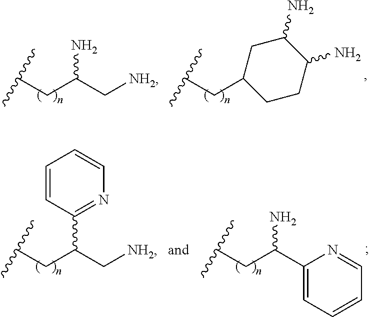

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a model of chemotherapeutic or radiation therapy sensitizing effect of PCNA inhibitors.

FIG. 2 shows representative crystal structure data on the binding of a compound to PCNA.

FIG. 3 shows aspects of structure-activity relationships of representative disclosed compounds.

FIG. 4 shows representative data for a disclosed compound in a fluorescent polarization assay and a TR (thyroid hormone receptor) transcription activation assay.

FIG. 5 shows representative data for effect of representative disclosed compounds in an assay of binding of PCNA and p21.

FIG. 6 shows representative data on the effect of a representative disclosed compound on co-immunoprecipitation of proteins with PCNA.

FIG. 7 shows representative data on the dose-response effect of a representative disclosed compound on cisplatin-induced DNA double-strand breads in HeLa cells.

FIG. 8 shows representative data on the effect of a representative disclosed compound on translesion DNA synthesis in an in vitro assay.

FIG. 9 shows representative data on the chemosensitizing effect of a representative disclosed compound on the potency of cisplatin in inhibiting the growth of HeLa cells.

FIG. 10 shows representative mass spectrometry data for (S)-4-(4-(2-amino-3-hydroxypropyl)-2,6-diiodophenoxy)phenol.

FIG. 11 shows representative ¹H NMR data for (S)-4-(4-(2-amino-3-hydroxypropyl)-2,6-diiodophenoxy)phenol.

FIG. 12 shows representative ¹H NMR data for 3-(4-(4-hydroxyphenoxy)-3,5-dimethylphenyl)propanoic acid.

FIG. 13 shows representative mass spectrometry data for dichloro((S)-4-(4-(2,3-diaminopropyl)-2,6-diiodophenoxy)phenol)platinum(II).

FIG. 14 shows representative ¹H NMR data for dichloro((S)-4-(4-(2,3-diaminopropyl)-2,6-diiodophenoxy)phenol)platinum(II).

FIG. 15 shows representative data obtained in a fluorescent polarization assay after 5 min incubation with (S)-4-(4-(2,3-diaminopropyl)-2,6-diiodophenoxy)phenol and dichloro((S)-4-(4-(2,3-diaminopropyl)-2,6-diiodophenoxy)phenol)platinum(II) as indicated.

FIG. 16 shows representative data obtained in a fluorescent polarization assay after 1 hr incubation with (S)-4-(4-(2,3-diaminopropyl)-2,6-diiodophenoxy)phenol and dichloro((S)-4-(4-(2,3-diaminopropyl)-2,6-diiodophenoxy)phenol)platinum(II) as indicated.

FIG. 17 shows representative data obtained in a fluorescent polarization assay after 2 hr incubation with (S)-4-(4-(2,3-diaminopropyl)-2,6-diiodophenoxy)phenol and dichloro((S)-4-(4-(2,3-diaminopropyl)-2,6-diiodophenoxy)phenol)platinum(II) as indicated.

FIG. 18 shows representative data obtained in a fluorescent polarization assay after 16 hr incubation with (S)-4-(4-(2,3-diaminopropyl)-2,6-diiodophenoxy)phenol and dichloro((S)-4-(4-(2,3-diaminopropyl)-2,6-diiodophenoxy)phenol)platinum(II) as indicated.

FIG. 19 shows representative data on the effect of representative disclosed compounds on DNA replication and chemosensitivity to cisplatin.

FIG. 20 shows representative data on the effect of representative disclosed compounds in a translesion DNA synthesis assay using cisplatin-damaged DNA.

FIG. 21 shows representative data on the effect of representative compounds on U2OS cell viability in the presence of various concentrations of cisplatin.

FIG. 22 shows representative data on the effect of representative compounds on inhibition of PCNA protein-protein interactions.

FIG. 23 shows representative data on the co-crystal structure of a representative PCNA-T3 complex.

FIG. 24 shows representative data on the effect of a representative compound on inhibition PCNA-DNA polymerase interaction on chromatin.

FIG. 25 shows representative data on the functional response of cells upon treatment with a representative compound.

FIG. 26 shows representative data on the induction of DNA replication stress by a representative compound.

FIG. 27 shows representative data on the inhibition of translesion DNA synthesis (“TLS”) by a representative compound.

FIG. 28 shows representative on the increase of cisplatin-induced DNA damage by a representative compound.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the term “PCNA inhibitor” and “PCNA antagonist” can be used interchangeably, and both refer to any exogenously administered compound or agent that directly or indirectly inhibits the activity or function of PCNA.

As used herein, the term “PCNA” and “proliferating cell nuclear antigen” can be used interchangeably, and refer to a protein encoded by a gene designated in human as the PCNA gene, which is located human chromosome 20 and described by Entrez Gene cytogenetic band: 20pter-p12; Ensembl cytogenetic band: 20p12.3; and, HGNC cytogenetic band: 20pter-p12. The term PCNA refers to a human protein that has 261 amino acids and has a molecular weight of about 28769 Da. The term is inclusive of splice isoforms, transcript variants or other mRNA variants, and their protein translation products, including the two currently known gene transcripts of 1344 and 1359 nucleotides. The term is also inclusive of that protein referred to by such alternative designations as: MGC83672, cyclin, and DNA polymerase delta auxiliary protein, as used by those skilled in the art to that protein encoded by human gene PCNA. The term is also inclusive of the non-human orthologs or homologs thereof.

As used herein, the term “PL peptide” and “Pogo-ligase peptide” can be used interchangeably, and refer to a peptide as previously described by Kontopidis, et al. PNAS (2005) 102, 1871-1876. The PL peptide has the sequence represented by the amino acid sequence SAVLQKKITDYFHPKK.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for inhibition of PCNA prior to the administering step. In some aspects of the disclosed method, the subject has been diagnosed with a need for inhibition of PCNA prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a hyperproliferative disorder” means having been subjected to a physical examination by a person of skill, for example, a physician, and found to have a condition that can be diagnosed or treated by a compound or composition that can inhibit the activity of PCNA. As a further example, “diagnosed with a need for treatment of a hyperproliferative disorder” refers to having been subjected to a physical examination by a person of skill, for example, a physician, and found to have a condition characterized by a cell proliferation dysfunction. Such a diagnosis can be in reference to a disorder, such as a neurodegenerative disease, and the like, as discussed herein. For example, the term “diagnosed with a need for inhibition of PCNA activity” refers to having been subjected to a physical examination by a person of skill, for example, a physician, and found to have a condition that can be diagnosed or treated by inhibition of PCNA activity. For example, “diagnosed with a need for treatment with a cytostatic agent” means having been subjected to a physical examination by a person of skill, for example, a physician, and found to have a condition that can be diagnosed or treated by inhibition of cell growth activity. The cell growth can be inhibited by inhibition of PCNA activity. For example, “diagnosed with a need for treatment of one or more hyperproliferative disorders associated with PCNA dysfunction” means having been subjected to a physical examination by a person of skill, for example, a physician, and found to have one or more hyperproliferative disorders associated with one or more PCNA dysfunctions.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to a PCNA dysfunction) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target PCNA protein, or other biological entity together in such a manner that the compound can affect the activity of the target (e.g., PCNA, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration of a substance (e.g., a compound or a drug) that is required for 50% agonism or activation of a biological process, or component of a process, including a protein, subunit, organelle, ribonucleoprotein, etc. Generally, EC₅₀ refers to the refers to the half maximal (50%) concentration of agonist or activator that provokes a response halfway between the baseline and maximum response. In a yet further aspect, the response is in vitro, wherein the in vitro assay system can be a cell-free or cell-based assay. For example, the response can be measured using fluorescent polarization to determine the effect of an agent on the binding of PCNA to a protein or peptide. Alternatively, the response can be measured using surface plasmon resonance to assess the effect of an agent on the binding of PCNA to a protein or peptide. Other in vitro assay systems can determine the effect of an agent on in vitro translesion DNA synthesis. Cell-based systems can assess the response in terms of cell-cycle arrest using FACS analysis or other cell-based analysis. Alternatively, the response can be termed in terms of the effect of an agent on cell growth of cells grown in an in vitro cell culture system.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. Generally, IC₅₀ refers to the refers to the concentration of antagonist or inhibitor where the response (or binding) is reduced by half. In a yet further aspect, the response is in vitro, wherein the in vitro assay system can be a cell-free or cell-based assay. For example, the response can be measured using fluorescent polarization to determine the effect of an agent on the binding of PCNA to a protein or peptide. Alternatively, the response can be measured using surface plasmon resonance to assess the effect of an agent on the binding of PCNA to a protein or peptide. Other in vitro assay systems can determine the effect of an agent on in vitro translesion DNA synthesis. Cell-based systems can assess the response in terms of cell-cycle arrest using FACS analysis or other cell-based analysis. Alternatively, the response can be termed in terms of the effect of an agent on cell growth of cells grown in an in vitro cell culture system.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula C(O)OH.

The term “ester” as used herein is represented by the formula —OC(O)A¹ or C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes azetidine, dioxane, furan, imidazole, isothiazole, isoxazole, morpholine, oxazole, oxazole, including, 1,2,3-oxadiazole, 1,2,5-oxadiazole and 1,3,4-oxadiazole, piperazine, piperidine, pyrazine, pyrazole, pyridazine, pyridine, pyrimidine, pyrrole, pyrrolidine, tetrahydrofuran, tetrahydropyran, tetrazine, including 1,2,4,5-tetrazine, tetrazole, including 1,2,3,4-tetrazole and 1,2,4,5-tetrazole, thiadiazole, including, 1,2,3-thiadiazole, 1,2,5-thiadiazole, and 1,3,4-thiadiazole, thiazole, thiophene, triazine, including 1,3,5-triazine and 1,2,4-triazine, triazole, including, 1,2,3-triazole, 1,3,4-triazole, and the like.

The term “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄ N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{o}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; (CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘}S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched)alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*², ═NNHC(O)R*, ═NNHC(O)OR*, ═NNHS(O)₂R*, ═NR*, ═NOR*, —O(C(R^{*}₂))₂₋₃O—, or —S(C(R^{*}₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, brosylate, and halides.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

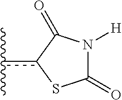

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

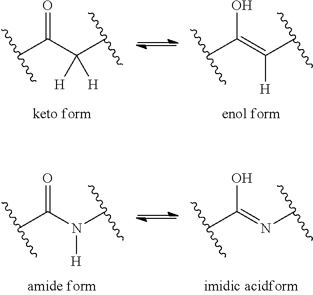 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

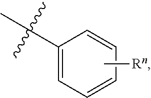

which is understood to be equivalent to a formula:

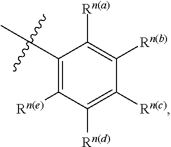

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, the invention relates to compounds useful as modulators of PCNA activity. In a further aspect, the disclosed compounds and products of the disclosed methods of making are modulators of PCNA activity. More specifically, in one aspect, the present invention relates to compounds that bind to a PCNA protein and negatively modulate PCNA activity. The compounds can, in one aspect, selectivity for modulation of PCNA activity compared to other proteins. In a still further aspect, the compounds exhibit selectivity for inhibition of PCNA activity compared to the TR (thyroid hormone receptor).

In one aspect, the disclosed compounds and products of disclosed methods of making exhibit inhibition of PCNA activity. In a further aspect, the disclosed compounds and products of disclosed methods of making exhibit inhibition of PCNA activity in a fluorescent polarization assay that measures binding of PCNA to the Pogo-Ligase (PL) peptide. In a still further aspect, the disclosed compounds and products of the disclosed methods of making bind to the PCNA protein. In a yet further aspect, the disclosed compounds and products of disclosed methods of making inhibit cell-growth. In an even further aspect, the disclosed compounds and products of disclosed methods of making inhibit in vitro cell growth. In a still further aspect, the disclosed compounds and products of disclosed methods of making inhibit growth in a cancer cell-line. In an even further aspect, the cancer cell-line is the HeLa cell-line.

In one aspect, the compounds of the invention are useful in the treatment oif a hyperproliferative disorder. In a further aspect, the hyperproliferative disorder is associated with a PCNA dysfunction and other diseases in which a PCNA protein is involved, as further described herein. In a still further aspect, the disclosed compounds and products of the disclosed methods of making are useful in the treatment of a cancer. In a yet further aspect, the compounds are useful in enhancing the effect of other chemotherapeutic agents used in the treatment of cancer. In an even further aspect, the compounds are useful in enhancing the effect of radiation therapy as used in the treatment of cancer

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspects, the invention relates to a compound having a structure represented by a formula:

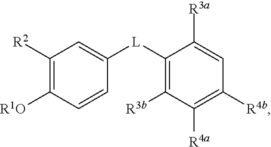

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein one of R^{4a} and R^{4b} is selected from hydrogen, halo, C1-C3 alkyl, C1-C3 haloalkyl, and C1-C3 polyhaloalkyl, and the other is selected from:

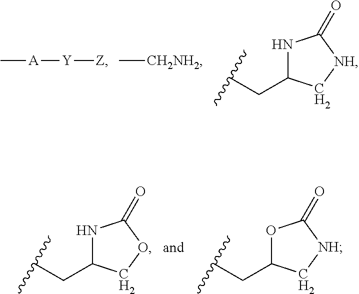

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In a further aspect, R² is selected from hydrogen, halo, (C═O)NR⁵SO₂R⁶, and (C═O)NR⁵R⁶; one of R^{4a} and R^{4b} is selected from hydrogen, halo, C1-C3 alkyl, C1-C3 haloalkyl, and C1-C3 polyhaloalkyl, and the other is -A-Y—Z; Z is selected from (C═O)NR⁹R¹⁰, (C═O)NHSO₂R⁹, CH₂OH, CH₂NH₂, CH₂NH(C═O)R⁹, and CH₂NHSO₂R⁹; and R⁹ is selected from C1-C6 alkyl, C3-C6 cycloalkyl, C1-C6 heterocycloalkyl, aryl, heteroaryl, and is substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl.

In various aspects, the invention relates to a compound having a structure represented by a formula:

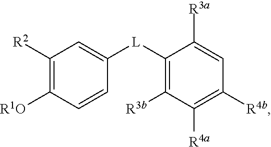

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In a further aspect, L is O, and both of R^{3a} and R^{3b} are halogen (e.g., iodo).

In a further aspect, the compound has a structure represented by a formula selected from:

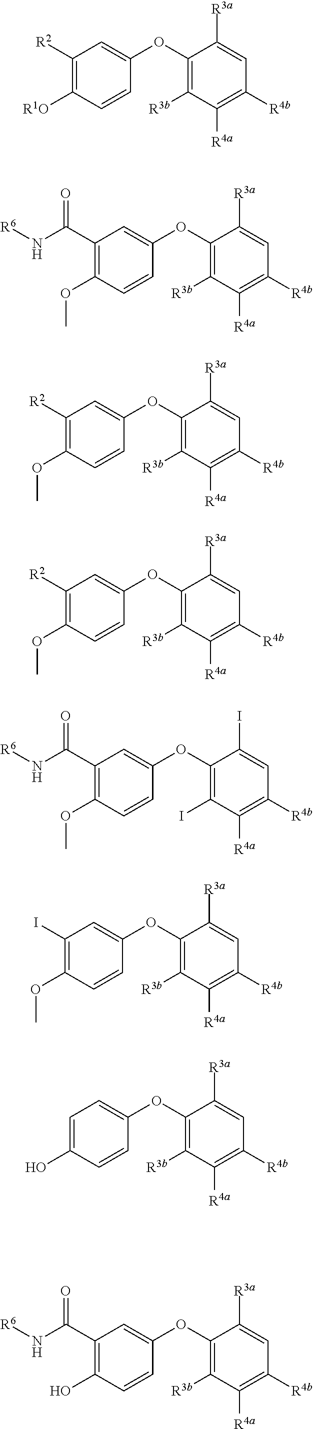

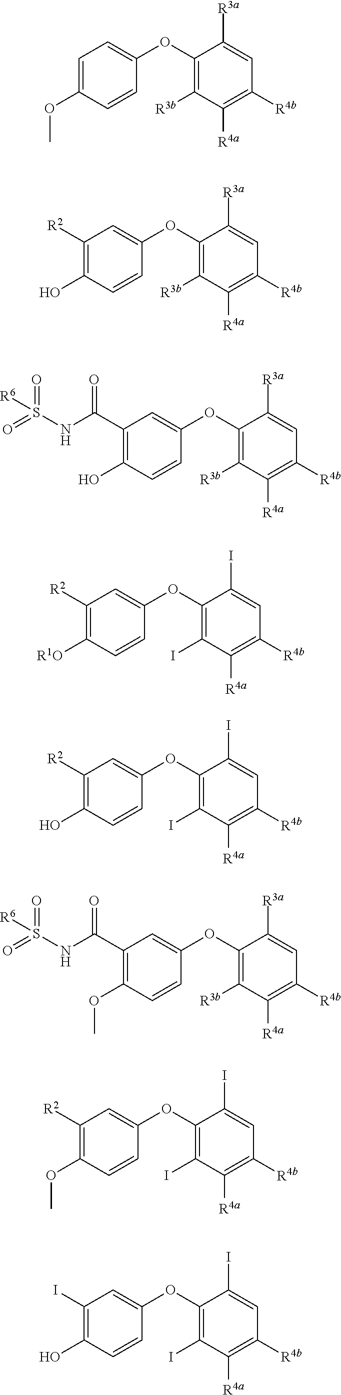

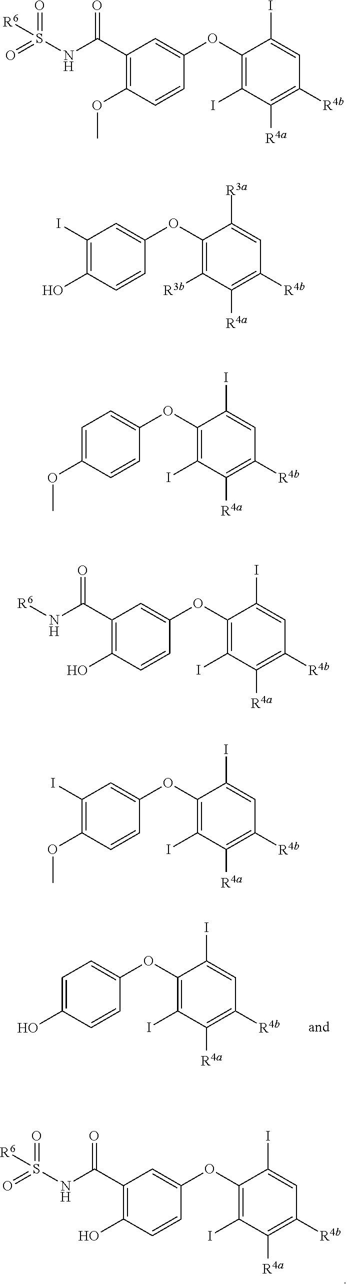

In various further aspects, the invention relates to a compound having a structure represented by a formula:

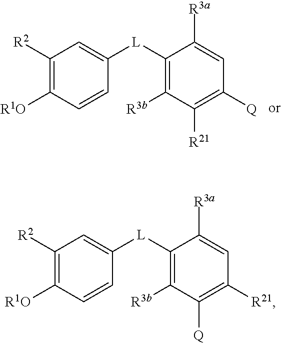

wherein L is selected from O, CH₂, CHOH, and C═O; wherein Q is selected from a structure represented by a formula:

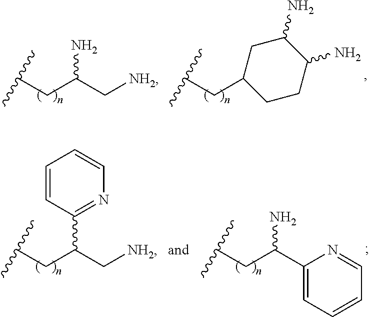

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound further comprises Pt(Z)₂; wherein each Z is selected from a halo; and wherein Q is selected from a structure represented by a formula:

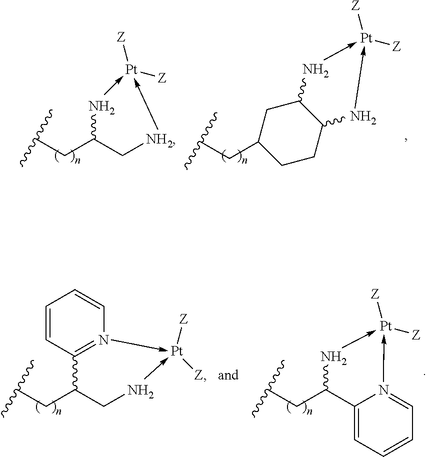

In a further aspect, the compound further comprises platinum (II) oxalate; and wherein Q is selected from a structure represented by a formula:

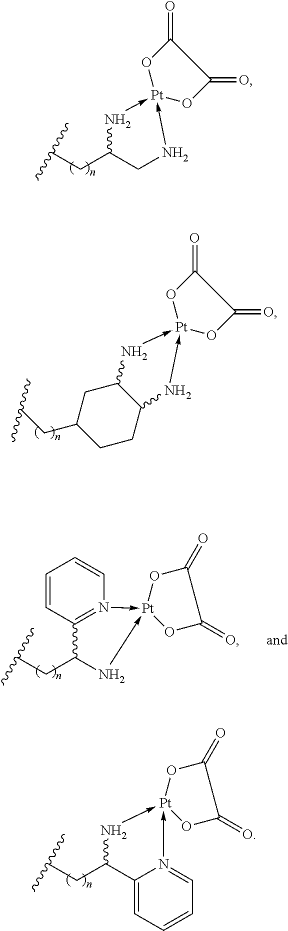

In a further aspect, the compound further comprises platinum (II) 1,1-cyclobutanedicarboxylate; and wherein Q is selected from a structure represented by a formula:

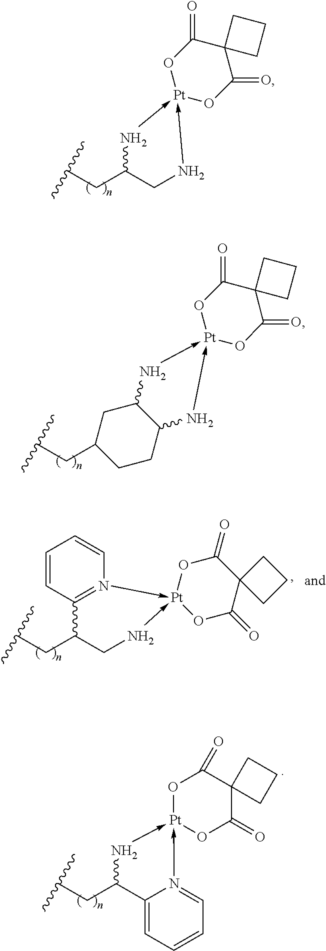

In a further aspect, the compound has a structure represented by a formula selected from:

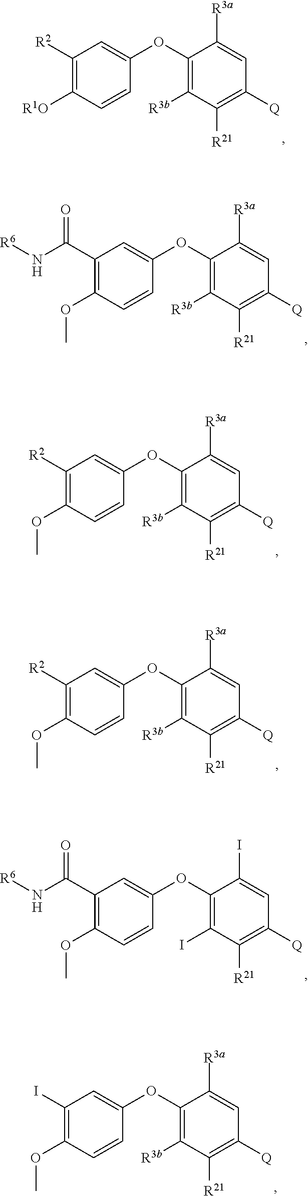

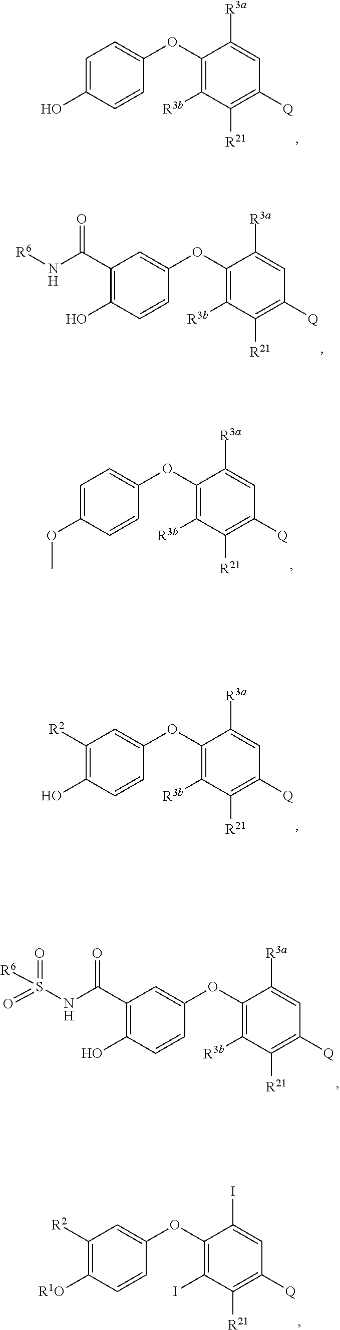

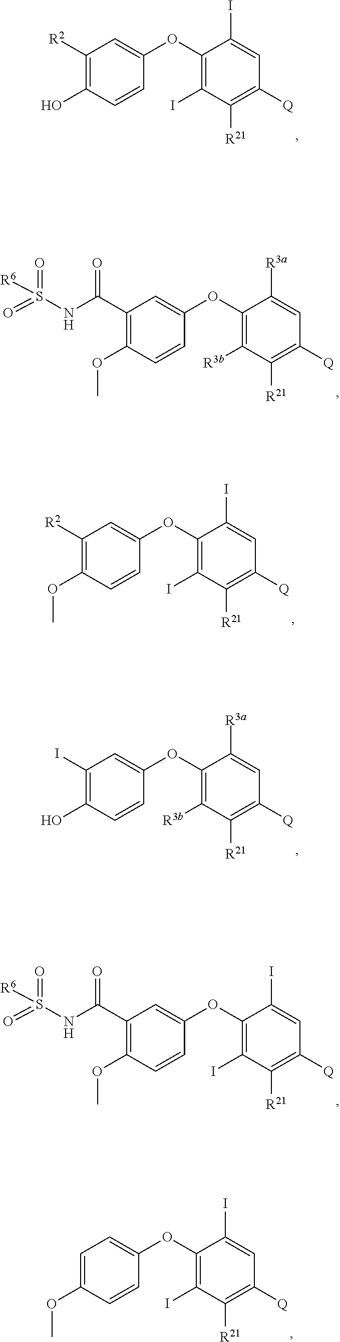

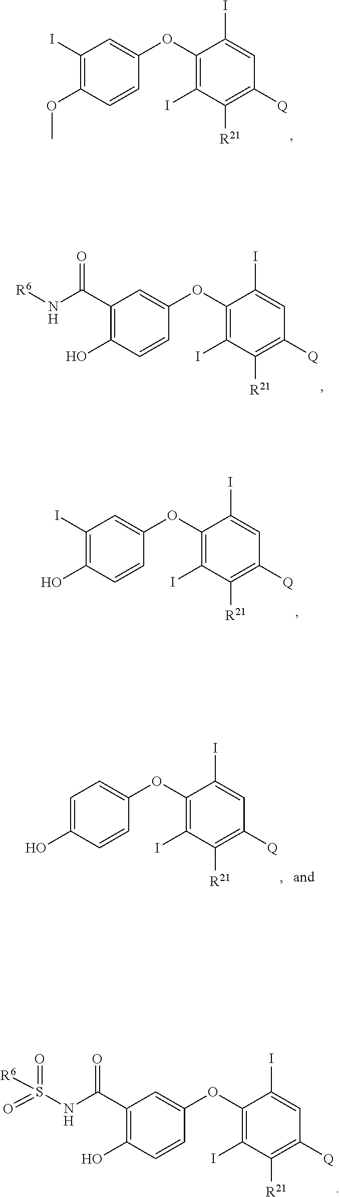

In a further aspect, the compound has a structure represented by a formula selected from:

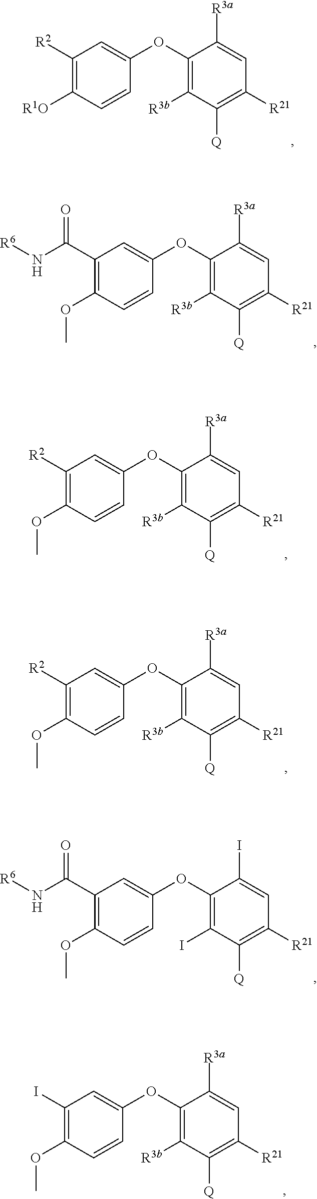

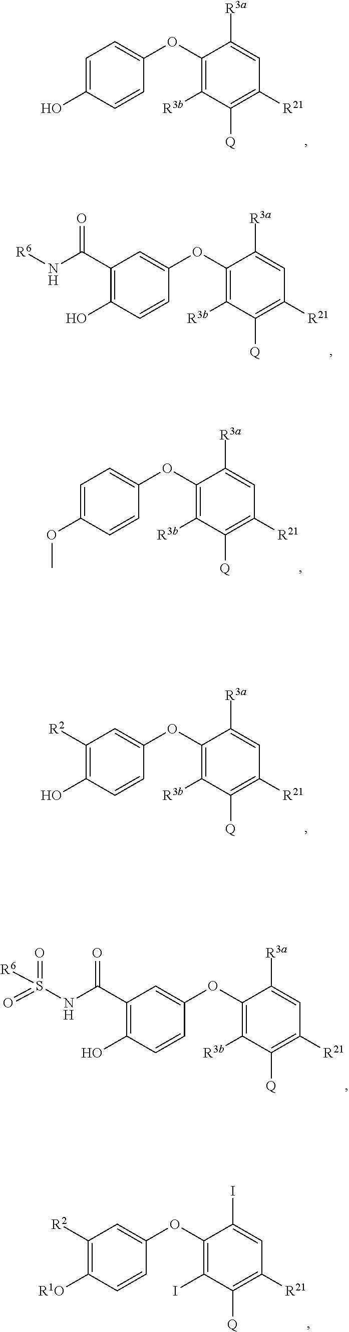

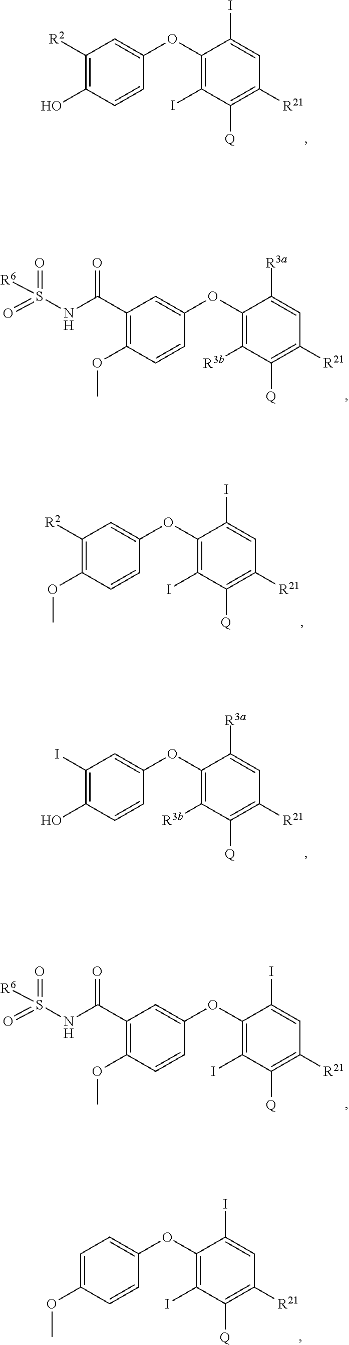

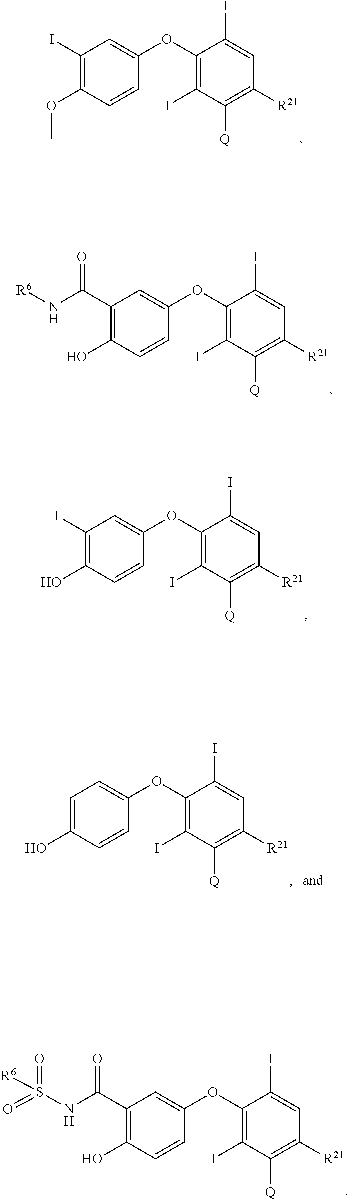

In a further aspect, the compound has a structure represented by a formula selected from:

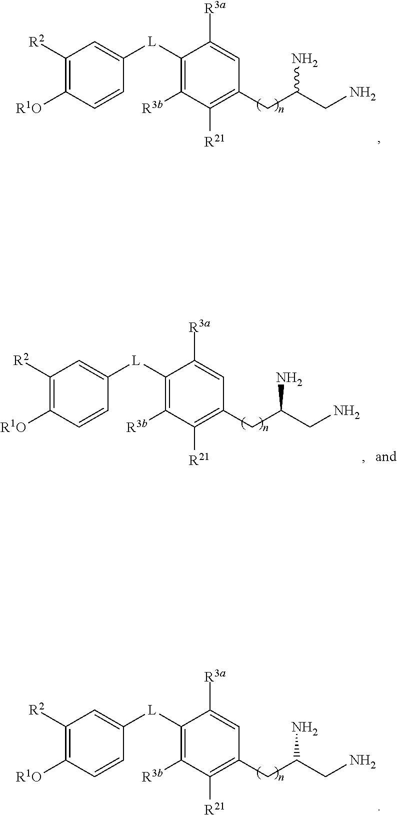

In a further aspect, the compound has a structure represented by a formula selected from:

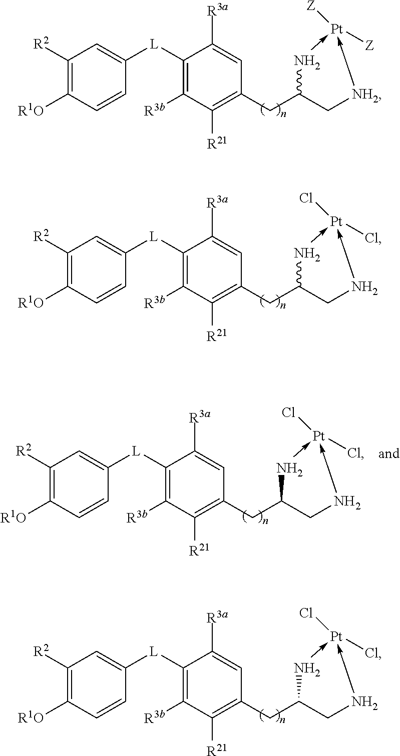

In a further aspect, the compound has a structure represented by a formula selected from:

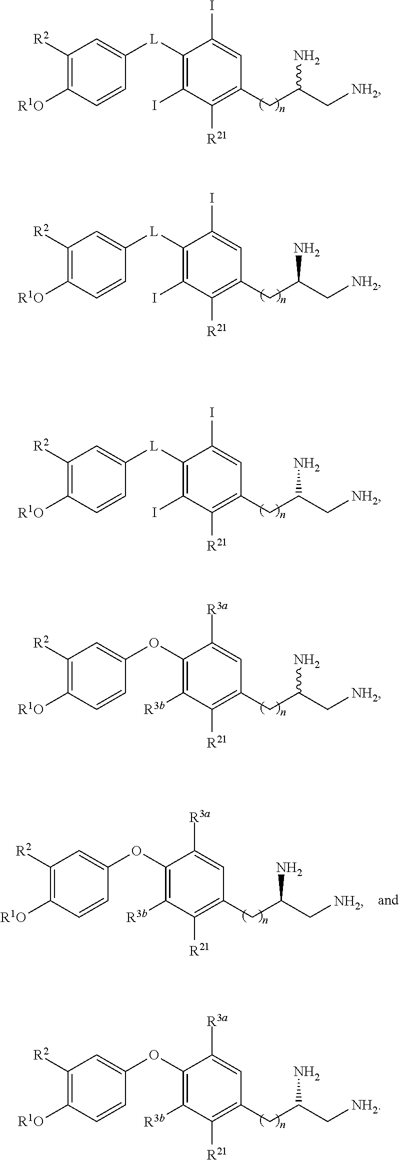

In a further aspect, the compound has a structure represented by a formula selected from:

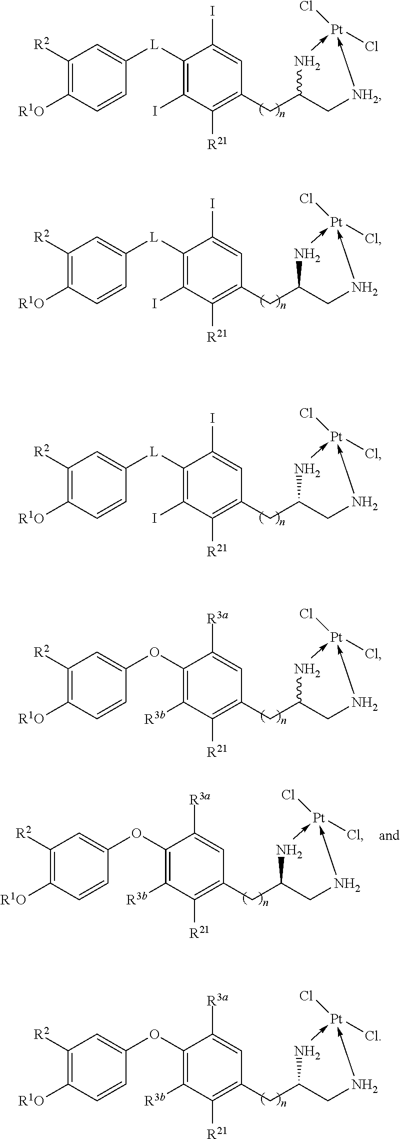

In a further aspect, the compound has a structure represented by a formula selected from:

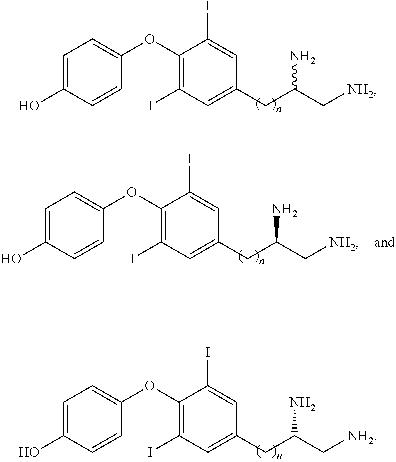

In a further aspect, the compound has a structure represented by a formula selected from:

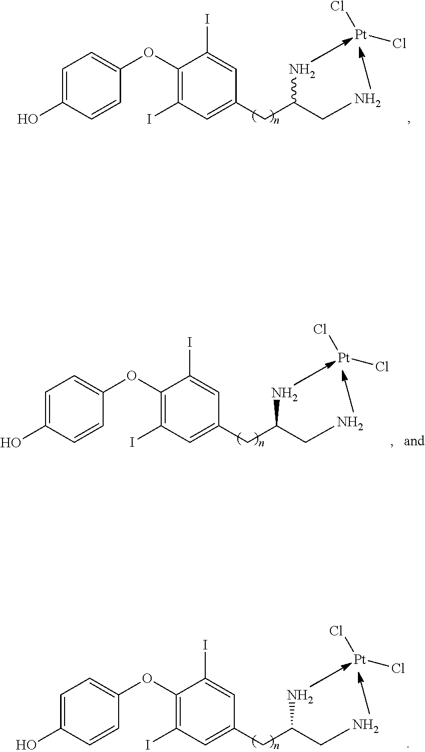

In a further aspect, the compound has a structure represented by a formula selected from:

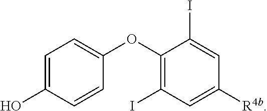

In a further aspect, the compound has a structure represented by a formula selected from:

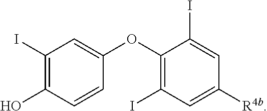

In a further aspect, the compound has a structure represented by a formula selected from:

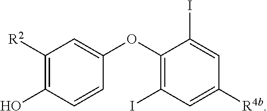

a. L Groups

In one aspect, L is selected from O, CH₂, CHOH, and C═O. In a further aspect, L is O. In a further aspect, L is selected from CH₂, CHOH, and C═O. In a further aspect, L is CH₂. In a further aspect, L is CHOH. In a further aspect, L is C═O.

b. R¹ Groups

In one aspect, R¹ is selected from hydrogen and C1-C3 alkyl. For example, R¹ can be selected from hydrogen and methyl. In a further aspect, R¹ is hydrogen. In a further aspect, R¹ is C1-C3 alkyl, for example, methyl, ethyl, n-propyl, i-propyl, or cyclopropyl.

c. R²Groups

In one aspect, R² is selected from hydrogen, halo, (C═O)NR⁵SO₂R⁶, and (C═O)NR⁵R⁶. In a further aspect, R² is selected from halo, (C═O)NR⁵SO₂R⁶, and (C═O)NR⁵R⁶. In a further aspect, R² is hydrogen. In a further aspect, R² is halo. In a further aspect, R² is selected from chloro, bromo, and iodo. In a further aspect, R² is iodo.

In a further aspect, R² is (C═O)NHR⁶. In a further aspect, R² is (C═O)NR⁵SO₂R⁶. In a further aspect, R² is selected from hydrogen, iodo, (C═O)NR⁵SO₂R⁶, and (C═O)NHR⁶.

d. R³Groups

In one aspect, R^{3a} is selected from hydrogen, halo, cyano, C1-C3 alkyl, C1-C3 haloalkyl, and C1-C3 polyhaloalkyl. In a further aspect, R^{3a} is selected from halo, cyano, C1-C3 alkyl, C1-C3 haloalkyl, and C1-C3 polyhaloalkyl. In a further aspect, R^{3a} is selected from halo and cyano. In a further aspect, R^{3a} is selected from C1-C3 alkyl, C1-C3 haloalkyl, and C1-C3 polyhaloalkyl. In a further aspect, R^{3a} is hydrogen. In a further aspect, R^{3a} is cyano. In a further aspect, R^{3a} is halo, for example, chloro, bromo, or iodo. In a further aspect, R^{3a} is C1-C3 alkyl, for example, methyl, ethyl, n-propyl, i-propyl, or cyclopropyl. In a further aspect, R^{3a} is C1-C3 haloalkyl, for example, iodomethyl, iodoethyl, or iodopropyl. In a further aspect, R^{3a} is C1-C3 polyhaloalkyl, for example trifluoromethyl.

In one aspect, R^{3b} is selected from hydrogen, halo, cyano, C1-C3 alkyl, C1-C3 haloalkyl, and C1-C3 polyhaloalkyl. In a further aspect, R^{3b} is selected from halo, cyano, C1-C3 alkyl, C1-C3 haloalkyl, and C1-C3 polyhaloalkyl. In a further aspect, R^{3b} is selected from halo and cyano. In a further aspect, R^{3b} is selected from C1-C3 alkyl, C1-C3 haloalkyl, and C1-C3 polyhaloalkyl. In a further aspect, R^{3b} is hydrogen. In a further aspect, R^{3b} is cyano. In a further aspect, R^{3b} is halo, for example, chloro, bromo, or iodo. In a further aspect, R^{3b} is C1-C3 alkyl, for example, methyl, ethyl, n-propyl, i-propyl, or cyclopropyl. In a further aspect, R^{3b} is C1-C3 haloalkyl, for example, iodomethyl, iodoethyl, or iodopropyl. In a further aspect, R^{3b} is C1-C3 polyhaloalkyl, for example trifluoromethyl.

In a further aspect, R^{3a} is selected from halo, cyano, methyl, and CF₃, and R^{3b} is hydrogen. In a further aspect, R^{3a} is hydrogen, and R^{3b} is selected from halo, cyano, methyl, and CF₃. In a further aspect, each of R^{3a} and R^{3b} is independently selected from halo, cyano, methyl, and CF₃. In a further aspect, R^{3a} is selected from chloro, bromo, iodo, cyano, methyl, and CF₃, and R^{3b} is hydrogen. In a further aspect, R^{3a} is hydrogen, and R^{3b} is selected from chloro, bromo, iodo, cyano, methyl, and CF₃. In a further aspect, each of R^{3a} and R^{3b} is independently selected from chloro, bromo, iodo, cyano, methyl, and CF₃. In a further aspect, R^{3a} is iodo and R^{3b} is hydrogen. In a further aspect, R^{3a} is hydrogen and R^{3b} is iodo. In a further aspect, each of R^{3a} and R^{3b} is iodo.

In one aspect, R^{3a} and R^{3b} are not both hydrogen. That is, R^{3a} can be hydrogen, while R^{3b} is non-hydrogen, or R^{3b} can be hydrogen, while R^{3a} is non-hydrogen.

e. R⁴Groups

In one aspect, one of R^{4a} and R^{4b} is selected from hydrogen, halo, C1-C3 alkyl, C1-C3 haloalkyl, and C1-C3 polyhaloalkyl, and the other is -A-Y—Z. That is, R^{4a} is selected from hydrogen, halo, C1-C3 alkyl, C1-C3 haloalkyl, and C1-C3 polyhaloalkyl, while R^{4b} is -A-Y—Z, or R^{4b} is selected from hydrogen, halo, C1-C3 alkyl, C1-C3 haloalkyl, and C1-C3 polyhaloalkyl, while R^{4a} is -A-Y—Z.

In a further aspect, R^{4a} is selected from hydrogen, halo, C1-C3 alkyl, C1-C3 haloalkyl, and C1-C3 polyhaloalkyl. In a further aspect, R^{4a} is selected from halo, C1-C3 alkyl, C1-C3 haloalkyl, and C1-C3 polyhaloalkyl. In a further aspect, R^{4a} is selected from C1-C3 alkyl, C1-C3 haloalkyl, and C1-C3 polyhaloalkyl. In a further aspect, R^{4a} is hydrogen. In a further aspect, R^{4a} is halo, for example, chloro, bromo, or iodo. In a further aspect, R^{4a} is C1-C3 alkyl, for example, methyl, ethyl, n-propyl, i-propyl, or cyclopropyl. In a further aspect, R^{4a} is C1-C3 haloalkyl, for example, iodomethyl, iodoethyl, or iodopropyl. In a further aspect, R^{4a} is C1-C3 polyhaloalkyl, for example trifluoromethyl. In a further aspect, R^{4a} is -A-Y—Z.

In a further aspect, R^{4b} is selected from hydrogen, halo, C1-C3 alkyl, C1-C3 haloalkyl, and C1-C3 polyhaloalkyl. In a further aspect, R^{4b} is selected from halo, C1-C3 alkyl, C1-C3 haloalkyl, and C1-C3 polyhaloalkyl. In a further aspect, R^{4b} is selected from C1-C3 alkyl, C1-C3 haloalkyl, and C1-C3 polyhaloalkyl. In a further aspect, R^{4b} is hydrogen. In a further aspect, R^{4b} is halo, for example, chloro, bromo, or iodo. In a further aspect, R^{4b} is C1-C3 alkyl, for example, methyl, ethyl, n-propyl, i-propyl, or cyclopropyl. In a further aspect, R^{4b} is C1-C3 haloalkyl, for example, iodomethyl, iodoethyl, or iodopropyl. In a further aspect, R^{4b} is C1-C3 polyhaloalkyl, for example trifluoromethyl. In a further aspect, R^{4b} is -A-Y—Z.

f. R⁵ Groups

In one aspect, R⁵ is selected from hydrogen and C1-C3 alkyl. For example, R⁵ can be selected from hydrogen and methyl. In a further aspect, R⁵ is hydrogen. In a further aspect, R⁵ is C1-C3 alkyl, for example, methyl, ethyl, n-propyl, i-propyl, or cyclopropyl.

g. R⁶ Groups

In one aspect, R⁶ is optionally substituted and is selected from C1-C6 alkyl, C3-C6 cycloalkyl, C1-C6 heterocycloalkyl, monocyclic aryl (i.e., phenyl), and monocyclic heteroaryl. R⁶ can be substituted with 0-5 groups independently selected from halo, hydroxyl, cyano, amino, C1-C3 alkyl, C1-C3 alkoxy, C1-C3 haloalkyl, and C1-C3 polyhaloalkyl.

In a further aspect, R⁶ is C1-C6 alkyl, for example, C1-C4 alkyl or C1-C2 alkyl. In a further aspect, R⁶ can be selected from one or more of methyl, ethyl, n-propyl, i-propyl, cyclopropyl, n-butyl, i-butyl, s-butyl, t-butyl, cyclobutyl, pentyl, cyclopentyl, hexyl, or cyclohexyl.

In a further aspect, R⁶ is C1-C6 heterocycloalkyl, for example, C1-C4 heterocycloalkyl or C1-C2 heterocycloalkyl. In a further aspect, heterocycloalkyl is a cycloalkyl wherein 1, 2, or 3 carbon atoms have been replaced with a heteroatom selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane.

In a further aspect, R⁶ is phenyl. In a further aspect, R⁶ is monocyclic heteroaryl, for example, oxazolyl, isoxazolyl, pyrazolyl, furanyl, pyranyl, imidazolyl, thiophenyl, pyridinyl, pyridazinyl, pyrimidinyl, pyrazinyl, triazinyl, or tetrazinyl. In a further aspect, heteroaryl can be 2-pyridinyl, 3-pyridinyl, or 4-pyridinyl. In one aspect, heteroaryl can be C1-4, C1-6, or C1-8.

If optionally substituted, R⁶ can bear, for example, 0, 0-1, 0-2, 0-3, 0-4, or 0-5 groups. If substituted, there can be, for example, 1, 2, 3, 4, 5, 1-2, 1-3, 2-3, 1-4, 2-4, 3-4, 1-5, 2-5, 3-5, or 4-5 groups. Suitable groups include, for example, halogen (e.g, fluoro, chloro, bromo, or iodo), hydroxyl, cyano, amino, alkylamino, dialkylamino, C1-C3 alkyl (e.g., methyl, ethyl, n-propyl, or i-propyl), C1-C3 alkoxy (e.g., methoxyl, ethoxyl, n-propoxyl, or i-propoxyl), and C1-C3 haloalkyl (e.g., fluoromethyl or chloropropyl), C1-C3 polyhaloalkyl (e.g., trifluoromethyl or perfluoroethyl).

h. A Groups

In one aspect, A is optionally present, and when present is selected from O and CH₂. In a further aspect, A is present. In a further aspect, A is absent, and -A-Y—Z is equivalent to —Y—Z. In a further aspect, A is O. In a further aspect, A is CH₂.

i. Y Groups

In one aspect, Y is selected from CH₂, CH₂CH₂, CHNH₂, CHNH(C═O)R⁸, CHNH(C═O)OR⁸, CHNH(C═O)NHR⁸, and CHNHSO₂R⁸. In a further aspect, Y is selected from CH₂ and CH₂CH₂. In a further aspect, Y is selected from CHNH₂, CHNH(C═O)R⁸, CHNH(C═O)OR⁸, CHNH(C═O)NHR⁸, and CHNHSO₂R⁸.

j. Z Groups

In one aspect, Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹. In a further aspect, Z is selected from (C═O)NR⁹R¹⁰, (C═O)NHSO₂R⁹, CH₂OH, CH₂NH₂, CH₂NH(C═O)R⁹, and CH₂NHSO₂R⁹.

k. R⁸ Groups

In one aspect, R⁸ is optionally substituted and selected from C1-C6 alkyl, C3-C6 cycloalkyl, C1-C6 heterocycloalkyl, aryl, and heteroaryl. R⁸ can be substituted with 0-5 groups independently selected from halo, hydroxyl, cyano, amino, C1-C3 alkyl, C1-C3 alkoxy, C1-C3 haloalkyl, and C1-C3 polyhaloalkyl.

In a further aspect, R⁸ is C1-C6 alkyl, for example, C1-C4 alkyl or C1-C2 alkyl. In a further aspect, R⁸ can be selected from one or more of methyl, ethyl, n-propyl, i-propyl, cyclopropyl, n-butyl, i-butyl, s-butyl, t-butyl, cyclobutyl, pentyl, cyclopentyl, hexyl, or cyclohexyl.

In a further aspect, R⁸ is C1-C6 heterocycloalkyl, for example, C1-C4 heterocycloalkyl or C1-C2 heterocycloalkyl. In a further aspect, heterocycloalkyl is a cycloalkyl wherein 1, 2, or 3 carbon atoms have been replaced with a heteroatom selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane.

In a further aspect, R⁸ is aryl, which can be monocyclic or bicyclic. For example, an aryl group can be phenyl or naphthyl. In one aspect, the group is phenyl. In a further aspect, R⁸ is heteroaryl, which can be monocyclic or bicyclic. For example, a heteroaryl group can be oxazolyl, isoxazolyl, pyrazolyl, furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, or benzotriazolyl. In a further aspect, heteroaryl can be 2-pyridinyl, 3-pyridinyl, or 4-pyridinyl. In one aspect, heteroaryl can be C1-4, C1-6, or C1-8.

If optionally substituted, R⁸ can bear, for example, 0, 0-1, 0-2, 0-3, 0-4, or 0-5 groups. If substituted, there can be, for example, 1, 2, 3, 4, 5, 1-2, 1-3, 2-3, 1-4, 2-4, 3-4, 1-5, 2-5, 3-5, or 4-5 groups. Suitable groups include, for example, halogen (e.g, fluoro, chloro, bromo, or iodo), hydroxyl, cyano, amino, alkylamino, dialkylamino, C1-C3 alkyl (e.g., methyl, ethyl, n-propyl, or i-propyl), C1-C3 alkoxy (e.g., methoxyl, ethoxyl, n-propoxyl, or i-propoxyl), and C1-C3 haloalkyl (e.g., fluoromethyl or chloropropyl), C1-C3 polyhaloalkyl (e.g., trifluoromethyl or perfluoroethyl).

l. R⁹ Groups

In one aspect, R⁹ is optionally substituted and selected from C1-C6 alkyl, C3-C6 cycloalkyl, C1-C6 heterocycloalkyl, aryl, and heteroaryl. R⁹ can be substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl.

In a further aspect, R⁹ is C1-C6 alkyl, for example, C1-C4 alkyl or C1-C2 alkyl. In a further aspect, R⁹ can be selected from one or more of methyl, ethyl, n-propyl, i-propyl, cyclopropyl, n-butyl, i-butyl, s-butyl, t-butyl, cyclobutyl, pentyl, cyclopentyl, hexyl, or cyclohexyl.

In a further aspect, R⁹ is C3-C6 cycloalkyl, for example, C3-C4 cycloalkyl or C3-C5 cycloalkyl. In various aspects, cycloalkyl can be cyclopropyl, cyclobutyl, cyclopentyl, cyclohexyl, or bicyclo[3.1.0]hexyl. In a further aspect, cycloalkyl can be cycloalkenyl selected from cyclobutenyl, cyclopentenyl, cyclopentadienyl, cyclohexenyl, and cyclohexadienyl.

In a further aspect, R⁹ is C1-C6 heterocycloalkyl, for example, C1-C4 heterocycloalkyl or C1-C2 heterocycloalkyl. In a further aspect, heterocycloalkyl is a cycloalkyl wherein 1, 2, or 3 carbon atoms have been replaced with a heteroatom selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane.

In a further aspect, R⁹ is aryl, which can be monocyclic or bicyclic. For example, an aryl group can be phenyl or naphthyl. In one aspect, the group is phenyl. In a further aspect, R⁹ is heteroaryl, which can be monocyclic or bicyclic. For example, a heteroaryl group can be oxazolyl, isoxazolyl, pyrazolyl, furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, or benzotriazolyl. In a further aspect, heteroaryl can be 2-pyridinyl, 3-pyridinyl, or 4-pyridinyl. In one aspect, heteroaryl can be C1-4, C1-6, or C1-8.

If optionally substituted, R⁹ can bear, for example, 0, 0-1, 0-2, 0-3, 0-4, or 0-5 groups. If substituted, there can be, for example, 1, 2, 3, 4, 5, 1-2, 1-3, 2-3, 1-4, 2-4, 3-4, 1-5, 2-5, 3-5, or 4-5 groups. Suitable groups include, for example, halogen (e.g, fluoro, chloro, bromo, or iodo), hydroxyl, cyano, amino, alkylamino, dialkylamino, SO₂R¹¹, C1-C3 alkyl (e.g., methyl, ethyl, n-propyl, or i-propyl), C1-C3 alkoxy (e.g., methoxyl, ethoxyl, n-propoxyl, or i-propoxyl), and C1-C3 haloalkyl (e.g., fluoromethyl or chloropropyl), C1-C3 polyhaloalkyl (e.g., trifluoromethyl or perfluoroethyl).

M. R¹⁰ Groups

In one aspect, R¹⁰ is selected from hydrogen and C1-C6 alkyl. In a further aspect, R¹⁰ is hydrogen. In a further aspect, R¹⁰ is C1-C6 alkyl, for example, C1-C4 alkyl or C1-C2 alkyl. In a further aspect, R¹⁰ is selected from one or more of methyl, ethyl, n-propyl, i-propyl, cyclopropyl, n-butyl, i-butyl, s-butyl, t-butyl, cyclobutyl, pentyl, cyclopentyl, hexyl, or cyclohexyl.

Alternatively, R⁹ and R¹⁰ can be optionally covalently bonded and, together with the intermediate carbons, comprise a 3- to 7-membered heterocycloalkyl substituted with 0-5 groups independently selected from hydroxyl, amino, alkylamino, dialkylamino, (C═O), SO₂R¹¹, C1-C3 alkyl (e.g., methyl, ethyl, n-propyl, or i-propyl), C1-C3 alkoxy (e.g., methoxyl, ethoxyl, n-propoxyl, or i-propoxyl), C1-C3 haloalkyl (e.g., fluoromethyl or chloropropyl), and C1-C3 polyhaloalkyl (e.g., trifluoromethyl or perfluoroethyl). The substituted 3- to 7-membered heterocycloalkyl can bear, for example, 0, 0-1, 0-2, 0-3, 0-4, or 0-5 groups. If substituted, there can be, for example, 1, 2, 3, 4, 5, 1-2, 1-3, 2-3, 1-4, 2-4, 3-4, 1-5, 2-5, 3-5, or 4-5 groups.

In various aspects, the 3- to 7-membered heterocycloalkyl can have 3-7, 3-6, 3-5, 3-4, 4-7, 5-7, 6-7, 4-6, 5, or 6 members. In various further aspects, the 3- to 7-membered heterocycloalkyl is a cycloalkyl wherein 1, 2, or 3 carbon atoms have been replaced with a heteroatom selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane.

In various further aspects, the 3- to 7-membered heterocycloalkyl can be seleced from among:

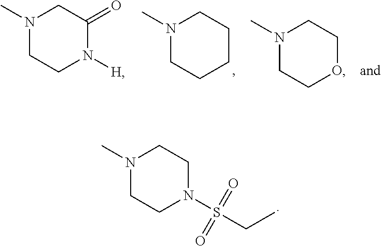

n. R¹¹ GROUPS

In one aspect, R¹¹ is selected from hydrogen and C1-C6 alkyl. In a further aspect, R¹¹ is hydrogen. In a further aspect, R¹¹ is C1-C6 alkyl, for example, C1-C4 alkyl or C1-C2 alkyl. In a further aspect, R¹¹ is selected from one or more of methyl, ethyl, n-propyl, i-propyl, cyclopropyl, n-butyl, i-butyl, s-butyl, t-butyl, cyclobutyl, pentyl, cyclopentyl, hexyl, or cyclohexyl.

o. Q Groups

In one aspect, Q is selected from a structure represented by a formula:

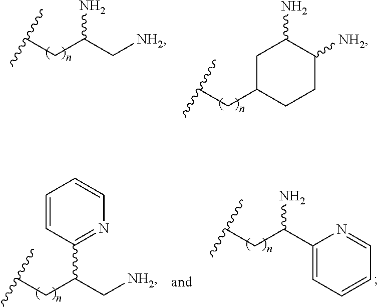

and wherein n is an integer selected from 0, 1, 2, 3, 4, 5, and 6.

In a further aspect, n is an integer selected from 0, 1, 2, 3, 4, and 5. In a still further aspect, n is an integer selected from 0, 1, 2, 3, and 4. In a yet further aspect, n is an integer selected from 0, 1, 2, and 3. In an even further aspect, n is an integer selected from 0, 1, and 2. In a still further aspect, n is an integer selected from 0 and 1. In an even further aspect n is an integer selected from 0, 1, 2, 3, 4, and 6 In yet further aspect, n is an integer selected from 0, 1, 2, 3, 5, and 6. In an even further aspect, n is an integer selected from 0, 1, 2, 4, 5, and 6. In a still further aspect, n is an integer selected from 0, 1, 3, 4, 5, and 6. In a yet further aspect, n is an integer selected from 0, 2, 3, 4, 5, and 6. In an even further aspect, n is an integer selected from 1, 2, 3, 4, 5, and 6. In a still further aspect, n is an integer with a value of 0. In a yet further aspect, n is an integer with a value of 1. In an even further aspect, n is an integer with a value of 2. In a still further aspect, n is an integer with a value of 3. In a yet further aspect, n is an integer with a value of 4. In an even further aspect, n is an integer with a value of 5. In a still further aspect, n is an integer with a value of 6.

In various further aspects, Q is selected from a structure represented by a formula:

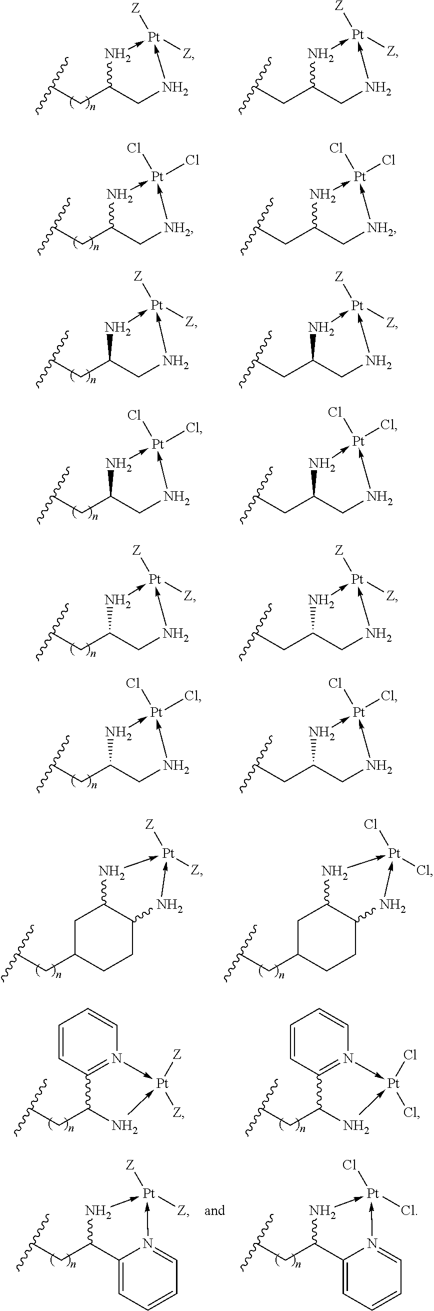

In various further aspects, Q is selected from a structure represented by a formula:

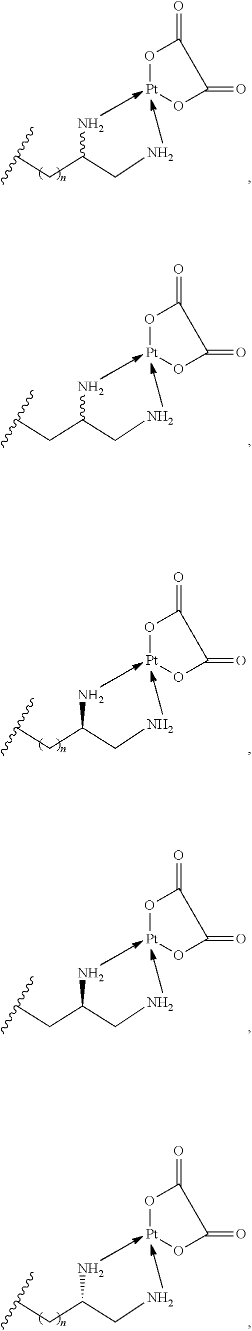

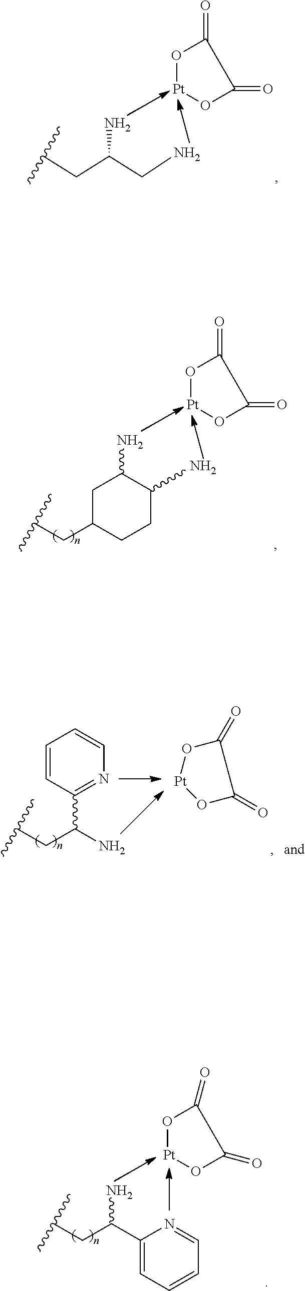

In various further aspects, Q is selected from a structure represented by a formula:

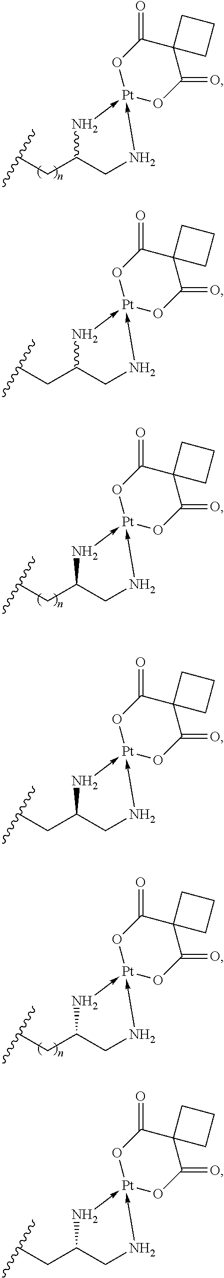

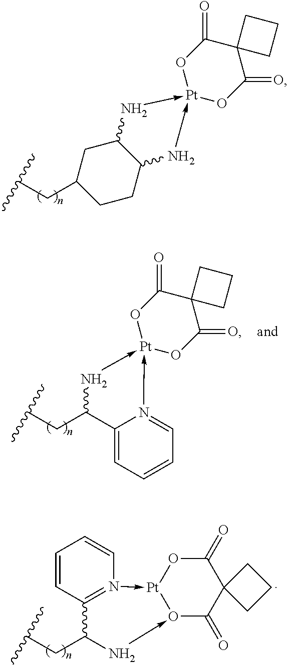

p. R²¹ Groups

In one aspect, R²¹ is selected from hydrogen, halo, C1-C3 alkyl, C1-C3 haloalkyl, and C1-C3 polyhaloalkyl.

In a further aspect, R²¹ is selected from hydrogen, halo, C1-C3 alkyl, C1-C3 haloalkyl, and C1-C3 polyhaloalkyl. In a further aspect, R²¹ is selected from halo, C1-C3 alkyl, C1-C3 haloalkyl, and C1-C3 polyhaloalkyl. In a further aspect, R²¹ is selected from C1-C3 alkyl, C1-C3 haloalkyl, and C1-C3 polyhaloalkyl. In a further aspect, R²¹ is hydrogen. In a further aspect, R²¹ is halo, for example, chloro, bromo, or iodo. In a further aspect, R²¹ is C1-C3 alkyl, for example, methyl, ethyl, n-propyl, i-propyl, or cyclopropyl. In a further aspect, R²¹ is C1-C3 haloalkyl, for example, iodomethyl, iodoethyl, or iodopropyl. In a further aspect, R²¹ is C1-C3 polyhaloalkyl, for example trifluoromethyl.

q. R²² Groups

In one aspect, R²² is selected from hydrogen and C1-C3 alkyl. For example, R²² can be selected from hydrogen and methyl. In a further aspect, R²² is hydrogen. In a further aspect, R²² is C1-C3 alkyl, for example, methyl, ethyl, n-propyl, i-propyl, or cyclopropyl.

r. R²³ Groups

In one aspect, R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl.

In a further aspect, R²³ is C1-C6 alkyl, for example, C1-C4 alkyl or C1-C2 alkyl. In a further aspect, R²³ can be selected from one or more of methyl, ethyl, n-propyl, i-propyl, cyclopropyl, n-butyl, i-butyl, s-butyl, t-butyl, cyclobutyl, pentyl, cyclopentyl, hexyl, or cyclohexyl.

In a further aspect, R²³ is C1-C6 heterocycloalkyl, for example, C1-C4 heterocycloalkyl or C1-C2 heterocycloalkyl. In a further aspect, heterocycloalkyl is a cycloalkyl wherein 1, 2, or 3 carbon atoms have been replaced with a heteroatom selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane.

In a further aspect, R²³ is phenyl. In a further aspect, R²³ is monocyclic heteroaryl, for example, oxazolyl, isoxazolyl, pyrazolyl, furanyl, pyranyl, imidazolyl, thiophenyl, pyridinyl, pyridazinyl, pyrimidinyl, pyrazinyl, triazinyl, or tetrazinyl. In a further aspect, heteroaryl can be 2-pyridinyl, 3-pyridinyl, or 4-pyridinyl. In one aspect, heteroaryl can be C1-4, C1-6, or C1-8

If optionally substituted, R²³ can bear, for example, 0, 0-1, 0-2, 0-3, 0-4, or 0-5 groups. If substituted, there can be, for example, 1, 2, 3, 4, 5, 1-2, 1-3, 2-3, 1-4, 2-4, 3-4, 1-5, 2-5, 3-5, or 4-5 groups. Suitable groups include, for example, halogen (e.g, fluoro, chloro, bromo, or iodo), hydroxyl, cyano, amino, alkylamino, dialkylamino, C1-C3 alkyl (e.g., methyl, ethyl, n-propyl, or i-propyl), C1-C3 alkoxy (e.g., methoxyl, ethoxyl, n-propoxyl, or i-propoxyl), and C1-C3 haloalkyl (e.g., fluoromethyl or chloropropyl), C1-C3 polyhaloalkyl (e.g., trifluoromethyl or perfluoroethyl).

s. Z Groups

In one aspect, each Z is selected from a halogen. In a further aspect, Z is chloro.

t. Halogen

In one aspect, halogen is fluoro, chloro, bromo or iodo. In a still further aspect, halogen is fluoro, chloro, or bromo. In a yet further aspect, halogen is fluoro or chloro. In a further aspect, halogen is fluoro. In an even further aspect, halogen is chloro or bromo. In an even further aspect, halogen is chloro. In a yet further aspect, halogen is iodo. In a still further aspect, halogen is bromo. In an even further aspect, halogen is chloro, bromo and iodo. In a yet further aspect, halogen is bromo and iodo. In a still further aspect, halogen is chloro and iodo.

It is also contemplated that pseudohalogens (e.g. triflate, mesylate, brosylate, etc.) can be used as leaving groups in place of halogens in certain aspects.

2. Example Compounds

In one aspect, a compound can be present as:

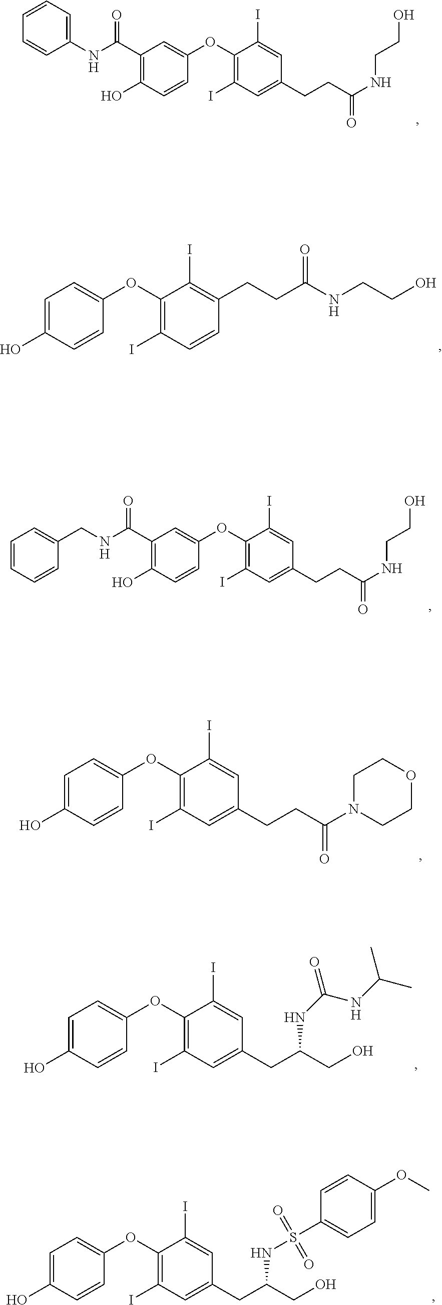 [SMILES: O=C(CCc1cc(I)c(Oc2ccc(O)c(C(=O)Nc3ccccc3)c2)c(I)c1)NCCO ; O=C(CCc1ccc(I)c(Oc2ccc(O)cc2)c1I)NCCO ; O=C(CCc1cc(I)c(Oc2ccc(O)c(C(=O)NCc3ccccc3)c2)c(I)c1)NCCO ; O=C(CCc1cc(I)c(Oc2ccc(O)cc2)c(I)c1)N1CCOCC1 ; CC(C)NC(=O)N[C@H](CO)Cc1cc(I)c(Oc2ccc(O)cc2)c(I)c1 ; COc1ccc(S(=O)(=O)N[C@H](CO)Cc2cc(I)c(Oc3ccc(O)cc3)c(I)c2)cc1]

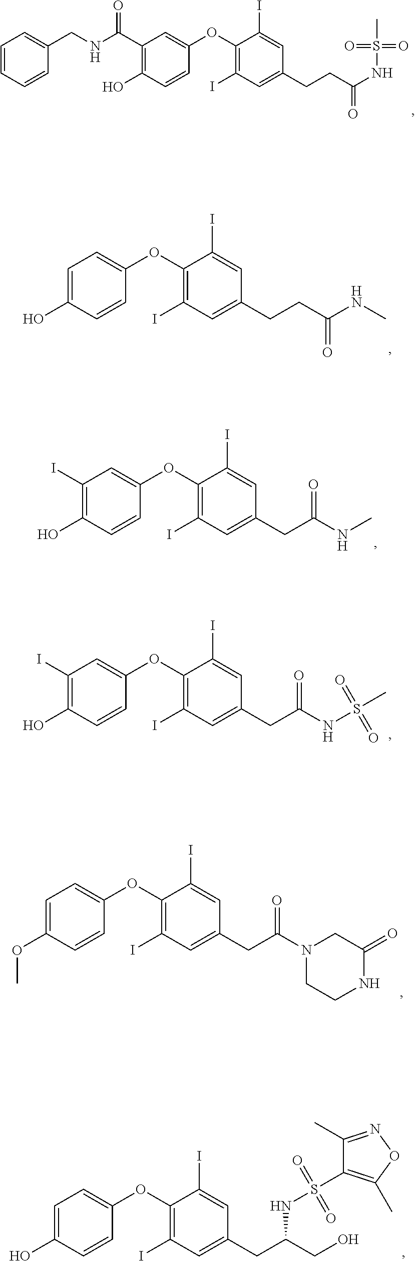 [SMILES: CS(=O)(=O)NC(=O)CCc1cc(I)c(Oc2ccc(O)c(C(=O)NCc3ccccc3)c2)c(I)c1 ; CNC(=O)CCc1cc(I)c(Oc2ccc(O)cc2)c(I)c1 ; CNC(=O)Cc1cc(I)c(Oc2ccc(O)c(I)c2)c(I)c1 ; CS(=O)(=O)NC(=O)Cc1cc(I)c(Oc2ccc(O)c(I)c2)c(I)c1 ; COc1ccc(Oc2c(I)cc(CC(=O)N3CCNC(=O)C3)cc2I)cc1 ; Cc1noc(C)c1S(=O)(=O)N[C@H](CO)Cc1cc(I)c(Oc2ccc(O)cc2)c(I)c1]

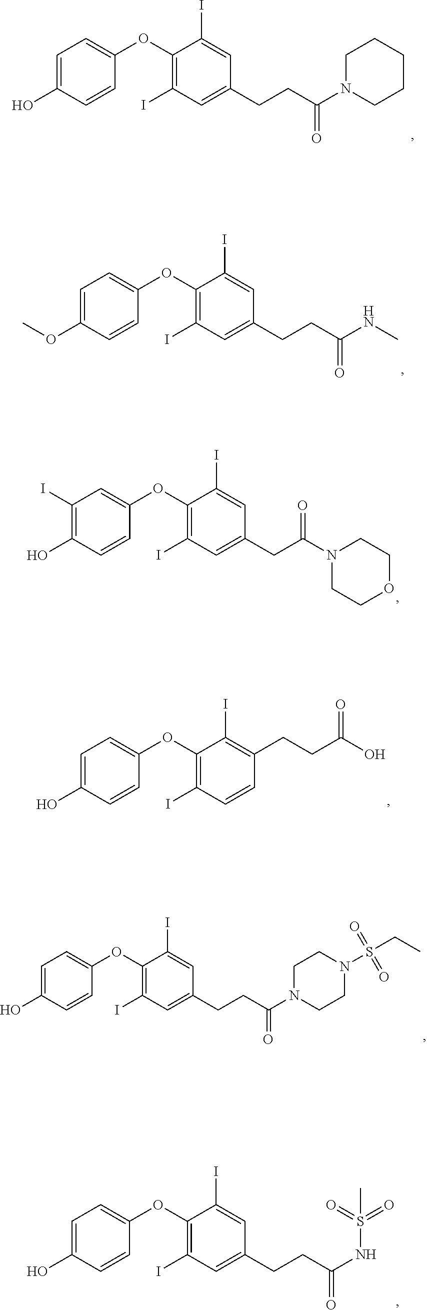

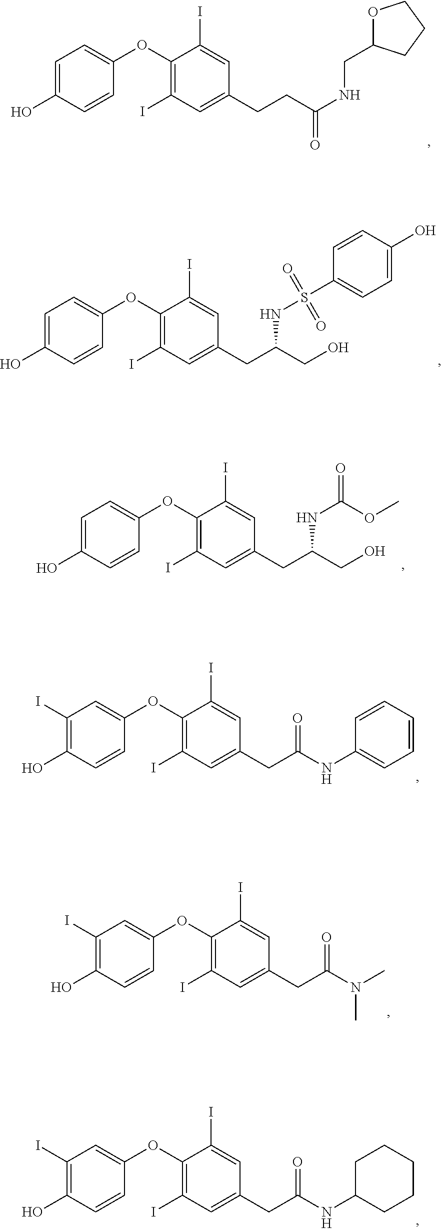 [SMILES: O=C(CCc1cc(I)c(Oc2ccc(O)cc2)c(I)c1)NCC1CCCO1 ; O=S(=O)(N[C@H](CO)Cc1cc(I)c(Oc2ccc(O)cc2)c(I)c1)c1ccc(O)cc1 ; COC(=O)N[C@H](CO)Cc1cc(I)c(Oc2ccc(O)cc2)c(I)c1 ; O=C(Cc1cc(I)c(Oc2ccc(O)c(I)c2)c(I)c1)Nc1ccccc1 ; CN(C)C(=O)Cc1cc(I)c(Oc2ccc(O)c(I)c2)c(I)c1 ; O=C(Cc1cc(I)c(Oc2ccc(O)c(I)c2)c(I)c1)NC1CCCCC1]

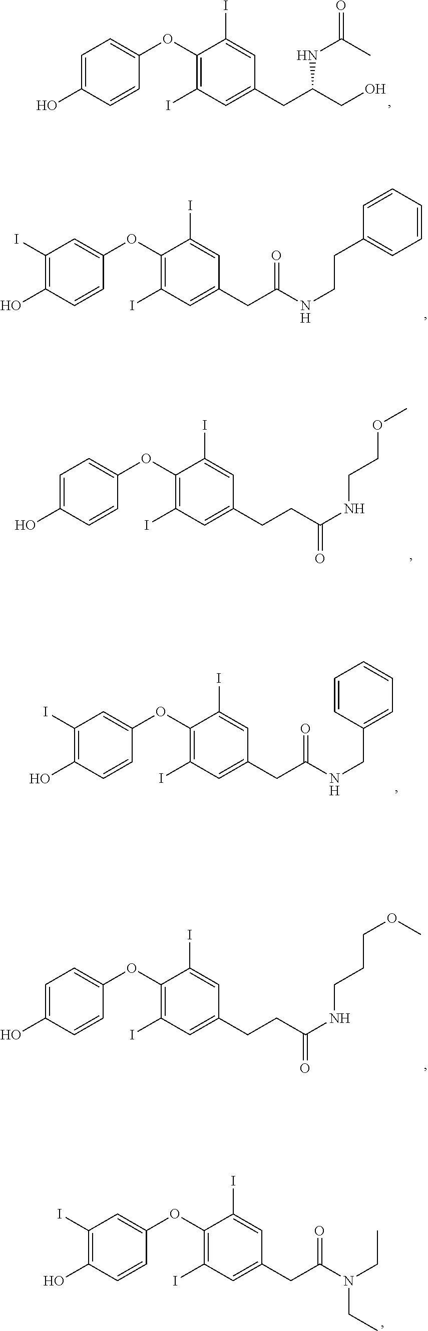 [SMILES: CC(=O)N[C@H](CO)Cc1cc(I)c(Oc2ccc(O)cc2)c(I)c1 ; O=C(Cc1cc(I)c(Oc2ccc(O)c(I)c2)c(I)c1)NCCc1ccccc1 ; COCCNC(=O)CCc1cc(I)c(Oc2ccc(O)cc2)c(I)c1 ; O=C(Cc1cc(I)c(Oc2ccc(O)c(I)c2)c(I)c1)NCc1ccccc1 ; COCCCNC(=O)CCc1cc(I)c(Oc2ccc(O)cc2)c(I)c1 ; CCN(CC)C(=O)Cc1cc(I)c(Oc2ccc(O)c(I)c2)c(I)c1]

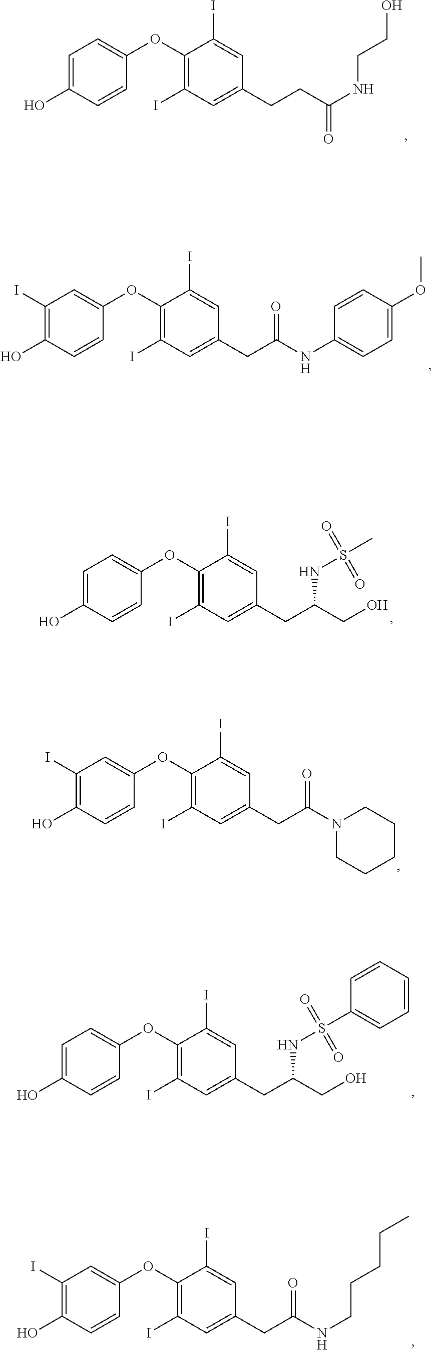 [SMILES: O=C(CCc1cc(I)c(Oc2ccc(O)cc2)c(I)c1)NCCO ; COc1ccc(NC(=O)Cc2cc(I)c(Oc3ccc(O)c(I)c3)c(I)c2)cc1 ; CS(=O)(=O)N[C@H](CO)Cc1cc(I)c(Oc2ccc(O)cc2)c(I)c1 ; O=C(Cc1cc(I)c(Oc2ccc(O)c(I)c2)c(I)c1)N1CCCCC1 ; O=S(=O)(N[C@H](CO)Cc1cc(I)c(Oc2ccc(O)cc2)c(I)c1)c1ccccc1 ; CCCCCNC(=O)Cc1cc(I)c(Oc2ccc(O)c(I)c2)c(I)c1]

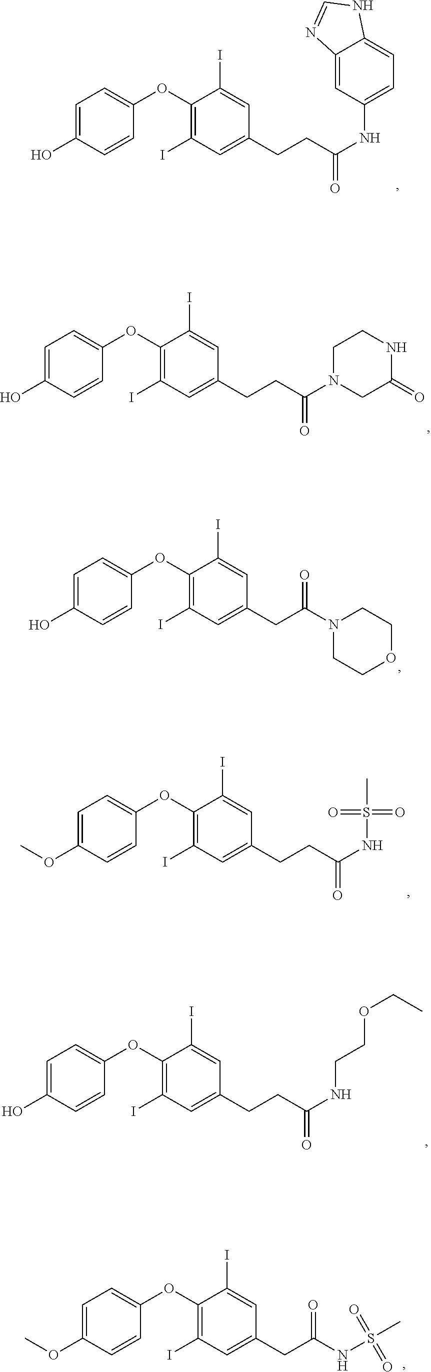

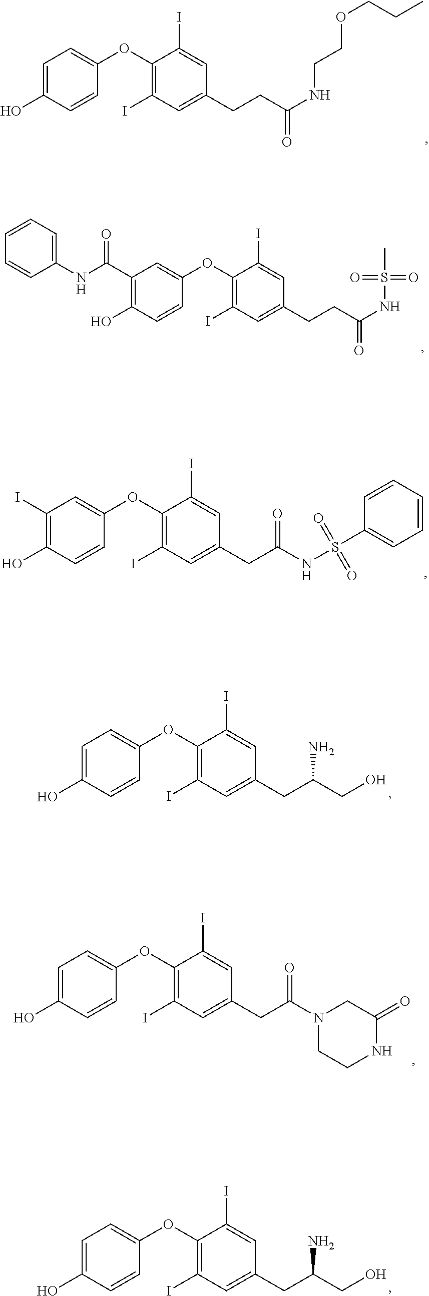 [SMILES: CCCOCCNC(=O)CCc1cc(I)c(Oc2ccc(O)cc2)c(I)c1 ; CS(=O)(=O)NC(=O)CCc1cc(I)c(Oc2ccc(O)c(C(=O)Nc3ccccc3)c2)c(I)c1 ; O=C(Cc1cc(I)c(Oc2ccc(O)c(I)c2)c(I)c1)NS(=O)(=O)c1ccccc1 ; N[C@H](CO)Cc1cc(I)c(Oc2ccc(O)cc2)c(I)c1 ; O=C1CN(C(=O)Cc2cc(I)c(Oc3ccc(O)cc3)c(I)c2)CCN1 ; N[C@@H](CO)Cc1cc(I)c(Oc2ccc(O)cc2)c(I)c1]

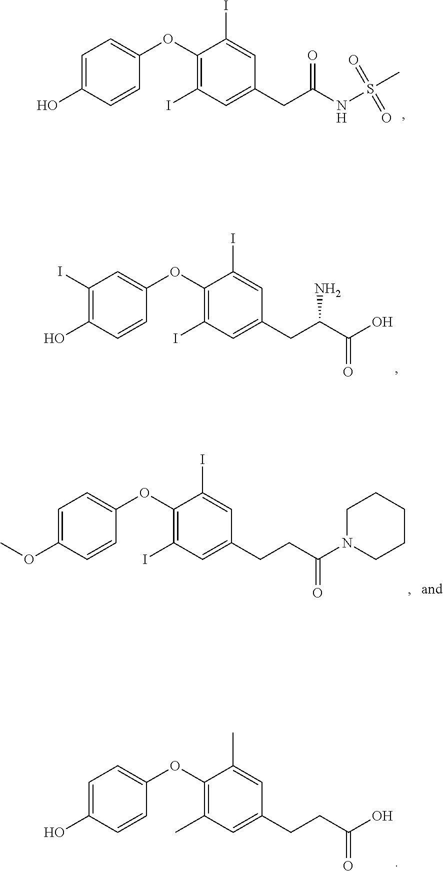

In one aspect, a compound can be present as:

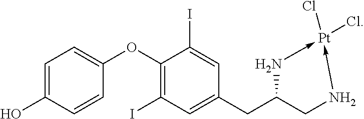

In one aspect, a compound can be present as:

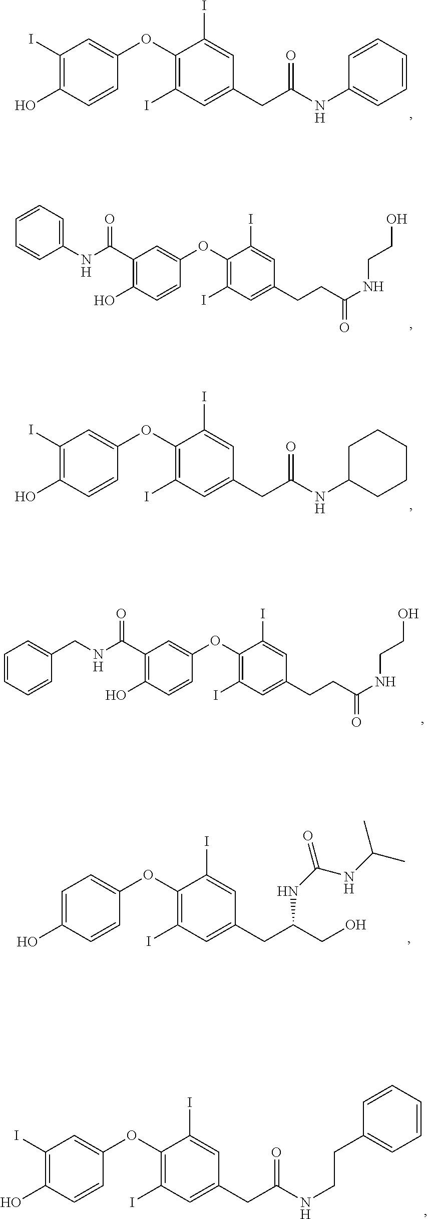 [SMILES: O=C(Cc1cc(I)c(Oc2ccc(O)c(I)c2)c(I)c1)Nc1ccccc1 ; O=C(CCc1cc(I)c(Oc2ccc(O)c(C(=O)Nc3ccccc3)c2)c(I)c1)NCCO ; O=C(Cc1cc(I)c(Oc2ccc(O)c(I)c2)c(I)c1)NC1CCCCC1 ; O=C(CCc1cc(I)c(Oc2ccc(O)c(C(=O)NCc3ccccc3)c2)c(I)c1)NCCO ; CC(C)NC(=O)N[C@H](CO)Cc1cc(I)c(Oc2ccc(O)cc2)c(I)c1 ; O=C(Cc1cc(I)c(Oc2ccc(O)c(I)c2)c(I)c1)NCCc1ccccc1]

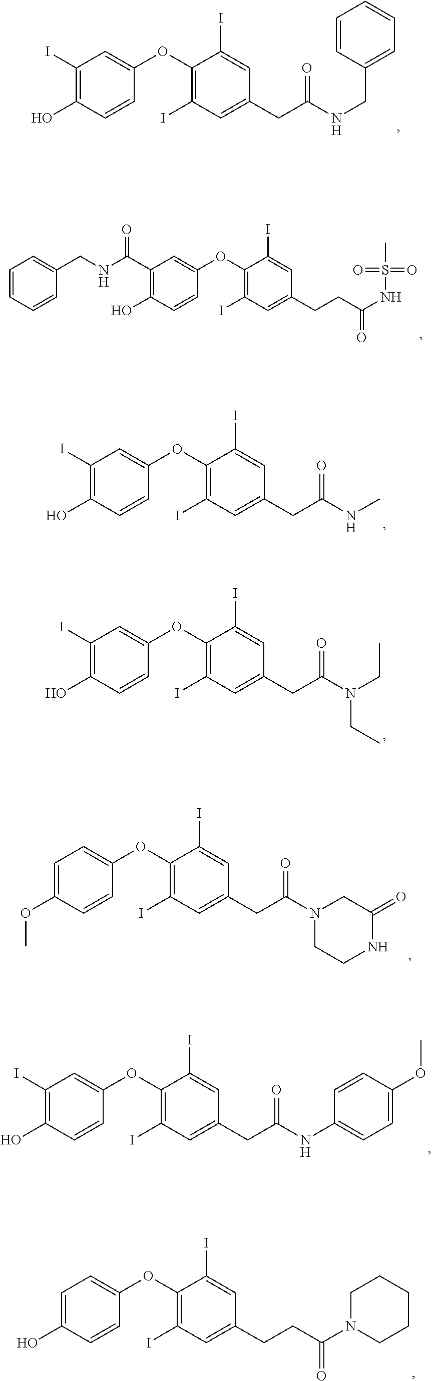

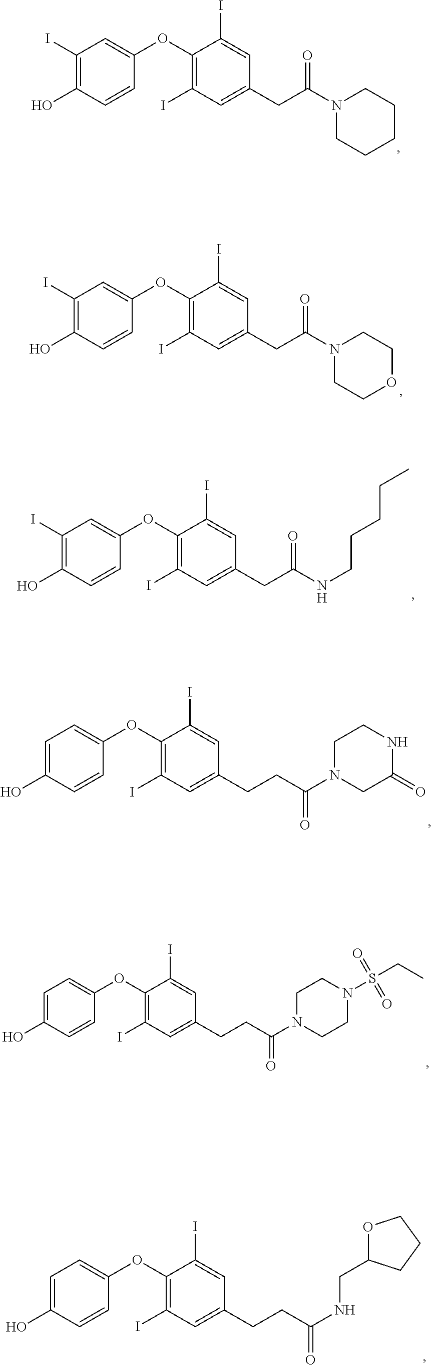 [SMILES: O=C(Cc1cc(I)c(Oc2ccc(O)c(I)c2)c(I)c1)N1CCCCC1 ; O=C(Cc1cc(I)c(Oc2ccc(O)c(I)c2)c(I)c1)N1CCOCC1 ; CCCCCNC(=O)Cc1cc(I)c(Oc2ccc(O)c(I)c2)c(I)c1 ; O=C1CN(C(=O)CCc2cc(I)c(Oc3ccc(O)cc3)c(I)c2)CCN1 ; CCS(=O)(=O)N1CCN(C(=O)CCc2cc(I)c(Oc3ccc(O)cc3)c(I)c2)CC1 ; O=C(CCc1cc(I)c(Oc2ccc(O)cc2)c(I)c1)NCC1CCCO1]

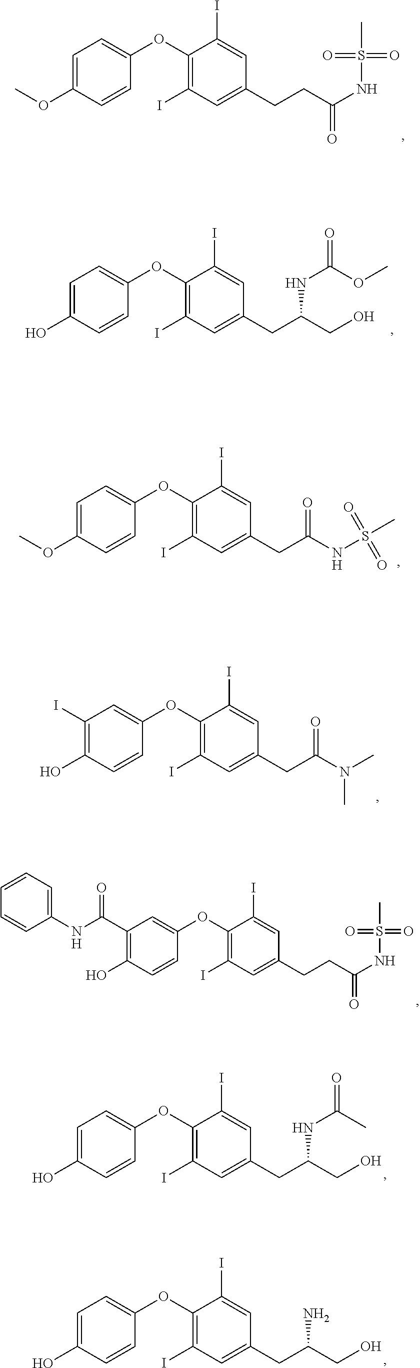

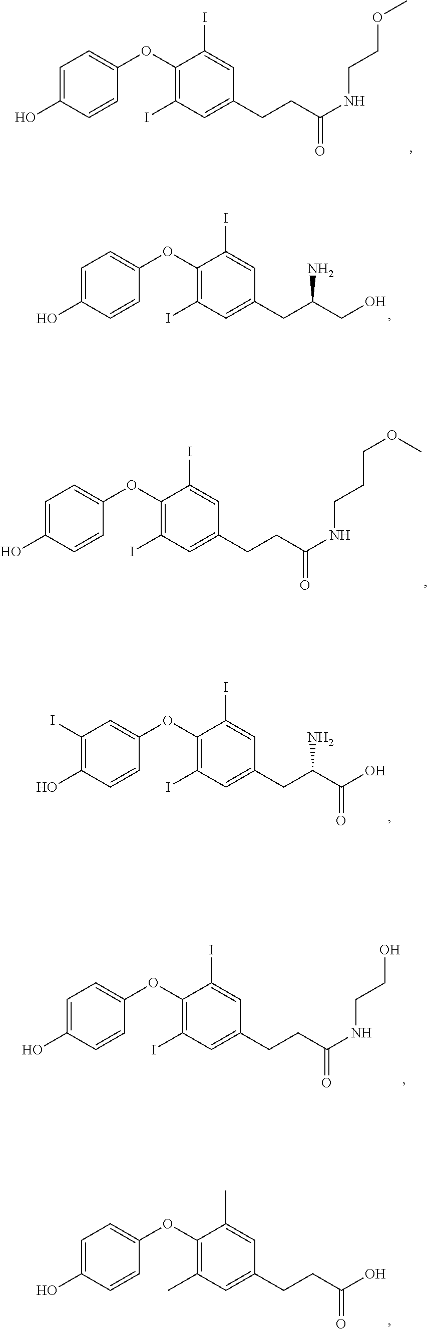 [SMILES: COCCNC(=O)CCc1cc(I)c(Oc2ccc(O)cc2)c(I)c1 ; N[C@@H](CO)Cc1cc(I)c(Oc2ccc(O)cc2)c(I)c1 ; COCCCNC(=O)CCc1cc(I)c(Oc2ccc(O)cc2)c(I)c1 ; N[C@@H](Cc1cc(I)c(Oc2ccc(O)c(I)c2)c(I)c1)C(=O)O ; O=C(CCc1cc(I)c(Oc2ccc(O)cc2)c(I)c1)NCCO ; Cc1cc(CCC(=O)O)cc(C)c1Oc1ccc(O)cc1]

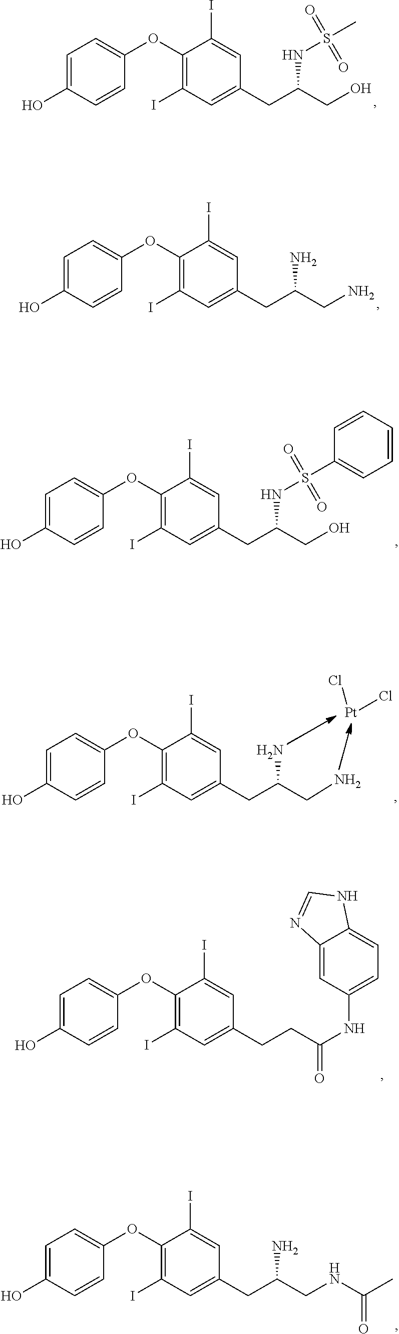

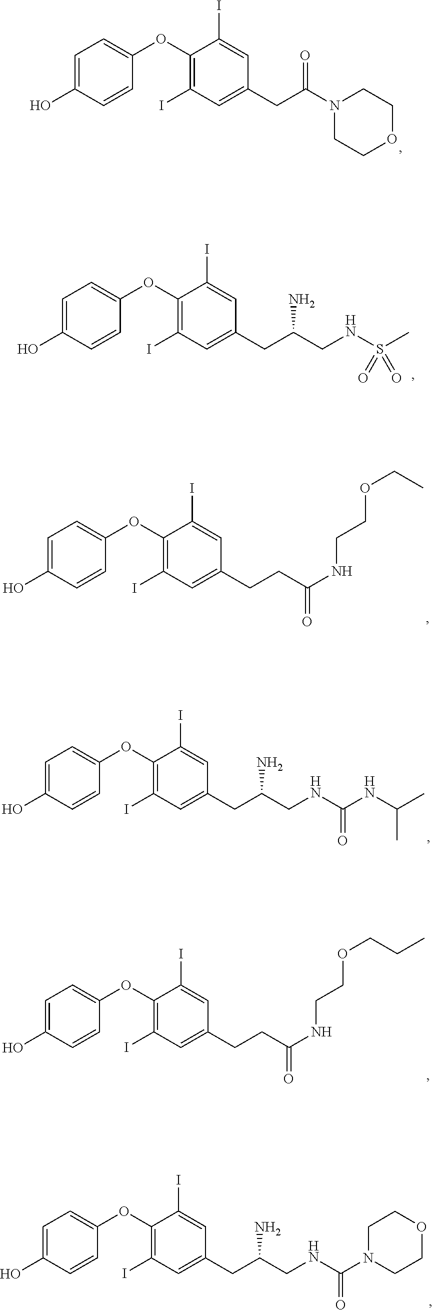 [SMILES: O=C(Cc1cc(I)c(Oc2ccc(O)cc2)c(I)c1)N1CCOCC1 ; CS(=O)(=O)NC[C@@H](N)Cc1cc(I)c(Oc2ccc(O)cc2)c(I)c1 ; CCOCCNC(=O)CCc1cc(I)c(Oc2ccc(O)cc2)c(I)c1 ; CC(C)NC(=O)NC[C@@H](N)Cc1cc(I)c(Oc2ccc(O)cc2)c(I)c1 ; CCCOCCNC(=O)CCc1cc(I)c(Oc2ccc(O)cc2)c(I)c1 ; N[C@H](CNC(=O)N1CCOCC1)Cc1cc(I)c(Oc2ccc(O)cc2)c(I)c1]

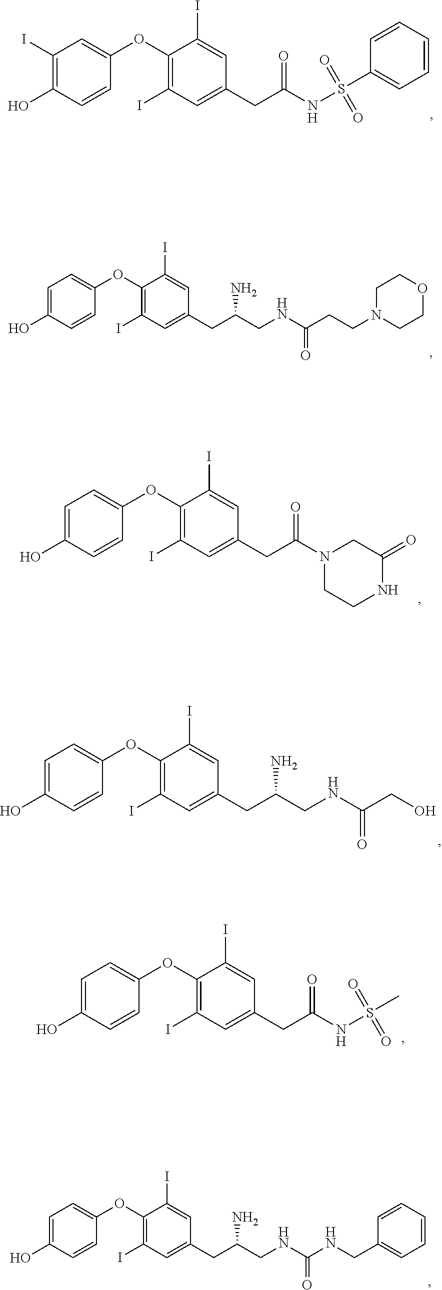 [SMILES: O=C(Cc1cc(I)c(Oc2ccc(O)c(I)c2)c(I)c1)NS(=O)(=O)c1ccccc1 ; N[C@H](CNC(=O)CCN1CCOCC1)Cc1cc(I)c(Oc2ccc(O)cc2)c(I)c1 ; O=C1CN(C(=O)Cc2cc(I)c(Oc3ccc(O)cc3)c(I)c2)CCN1 ; N[C@H](CNC(=O)CO)Cc1cc(I)c(Oc2ccc(O)cc2)c(I)c1 ; CS(=O)(=O)NC(=O)Cc1cc(I)c(Oc2ccc(O)cc2)c(I)c1 ; N[C@H](CNC(=O)NCc1ccccc1)Cc1cc(I)c(Oc2ccc(O)cc2)c(I)c1]

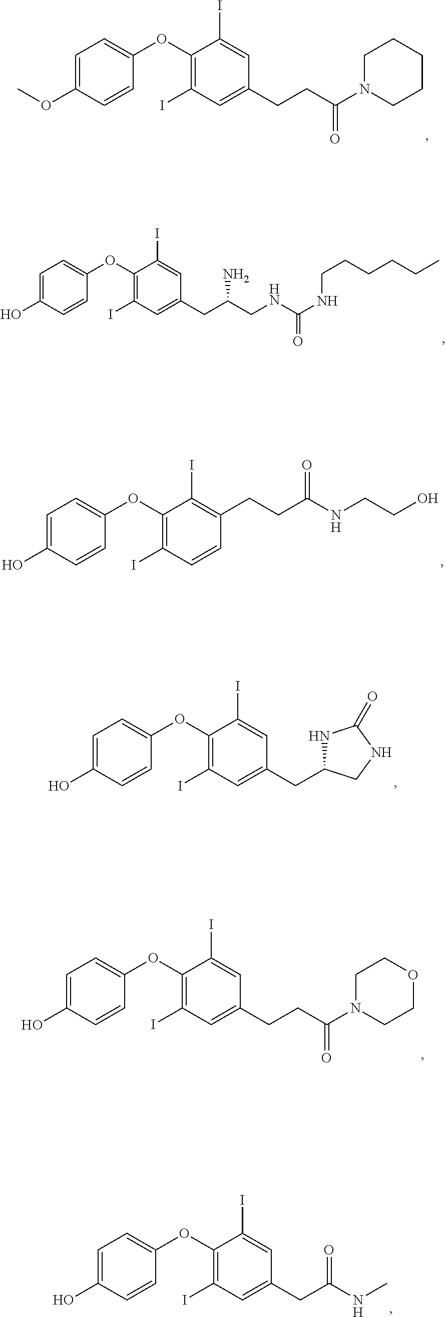 [SMILES: COc1ccc(Oc2c(I)cc(CCC(=O)N3CCCCC3)cc2I)cc1 ; CCCCCCNC(=O)NC[C@@H](N)Cc1cc(I)c(Oc2ccc(O)cc2)c(I)c1 ; O=C(CCc1ccc(I)c(Oc2ccc(O)cc2)c1I)NCCO ; O=C1NC[C@H](Cc2cc(I)c(Oc3ccc(O)cc3)c(I)c2)N1 ; O=C(CCc1cc(I)c(Oc2ccc(O)cc2)c(I)c1)N1CCOCC1 ; CNC(=O)Cc1cc(I)c(Oc2ccc(O)cc2)c(I)c1]

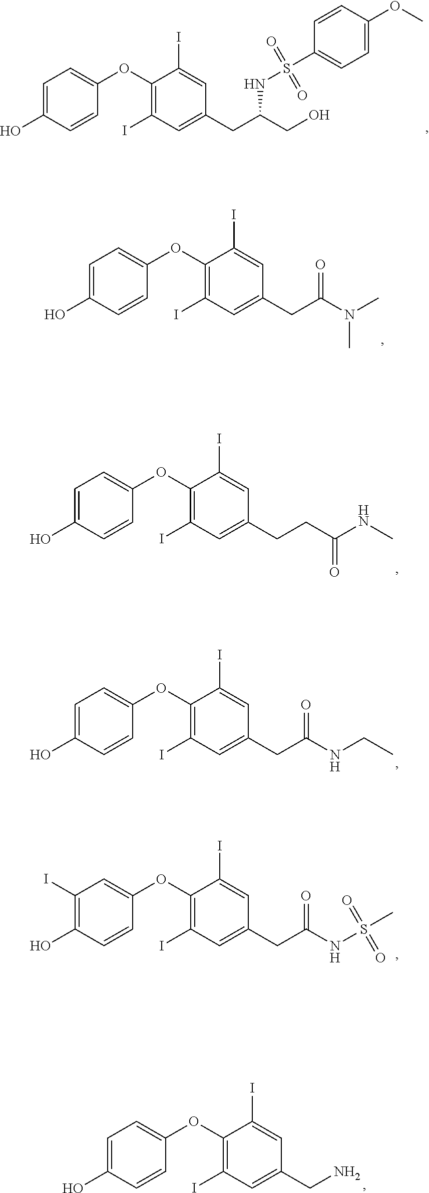 [SMILES: COc1ccc(S(=O)(=O)N[C@H](CO)Cc2cc(I)c(Oc3ccc(O)cc3)c(I)c2)cc1 ; CN(C)C(=O)Cc1cc(I)c(Oc2ccc(O)cc2)c(I)c1 ; CNC(=O)CCc1cc(I)c(Oc2ccc(O)cc2)c(I)c1 ; CCNC(=O)Cc1cc(I)c(Oc2ccc(O)cc2)c(I)c1 ; CS(=O)(=O)NC(=O)Cc1cc(I)c(Oc2ccc(O)c(I)c2)c(I)c1 ; NCc1cc(I)c(Oc2ccc(O)cc2)c(I)c1]

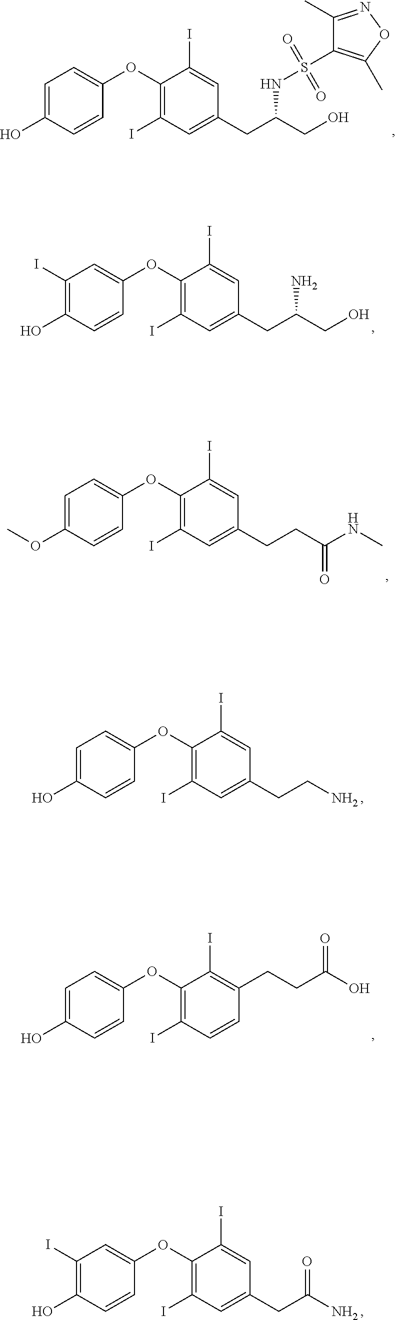

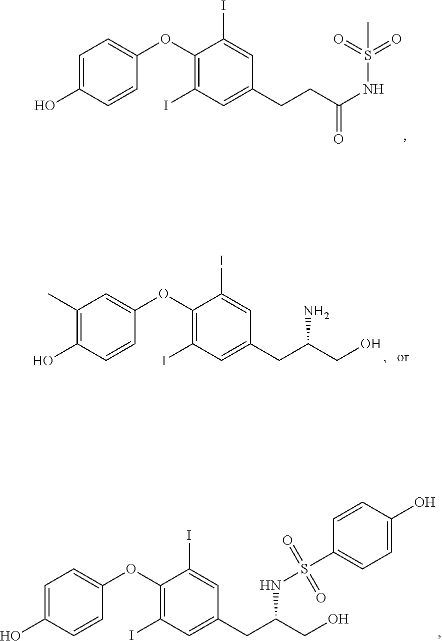 [SMILES: CS(=O)(=O)NC(=O)CCc1cc(I)c(Oc2ccc(O)cc2)c(I)c1 ; Cc1cc(Oc2c(I)cc(C[C@H](N)CO)cc2I)ccc1O ; O=S(=O)(N[C@H](CO)Cc1cc(I)c(Oc2ccc(O)cc2)c(I)c1)c1ccc(O)cc1]

or a subgroup thereof.

In one aspect, a compound can be present as:

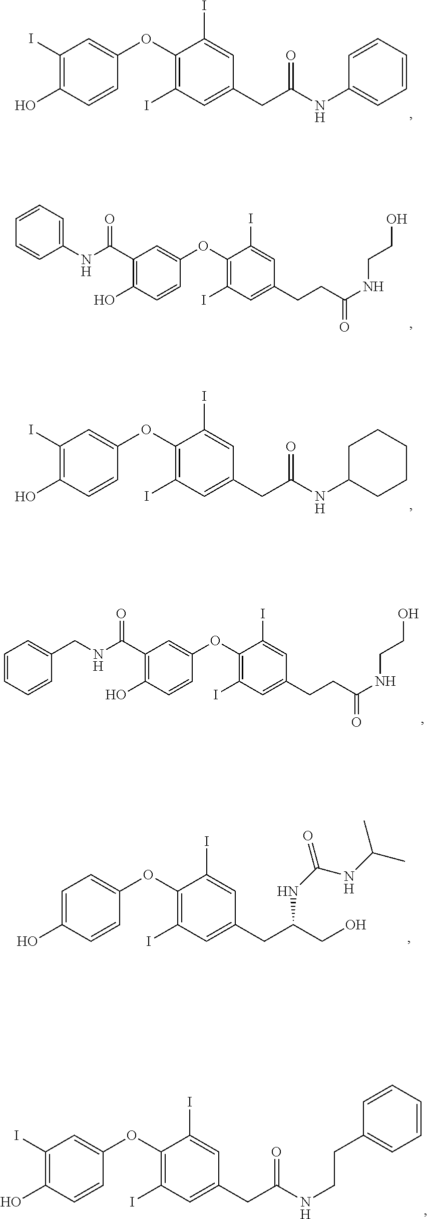 [SMILES: O=C(Cc1cc(I)c(Oc2ccc(O)c(I)c2)c(I)c1)Nc1ccccc1 ; O=C(CCc1cc(I)c(Oc2ccc(O)c(C(=O)Nc3ccccc3)c2)c(I)c1)NCCO ; O=C(Cc1cc(I)c(Oc2ccc(O)c(I)c2)c(I)c1)NC1CCCCC1 ; O=C(CCc1cc(I)c(Oc2ccc(O)c(C(=O)NCc3ccccc3)c2)c(I)c1)NCCO ; CC(C)NC(=O)N[C@H](CO)Cc1cc(I)c(Oc2ccc(O)cc2)c(I)c1 ; O=C(Cc1cc(I)c(Oc2ccc(O)c(I)c2)c(I)c1)NCCc1ccccc1]

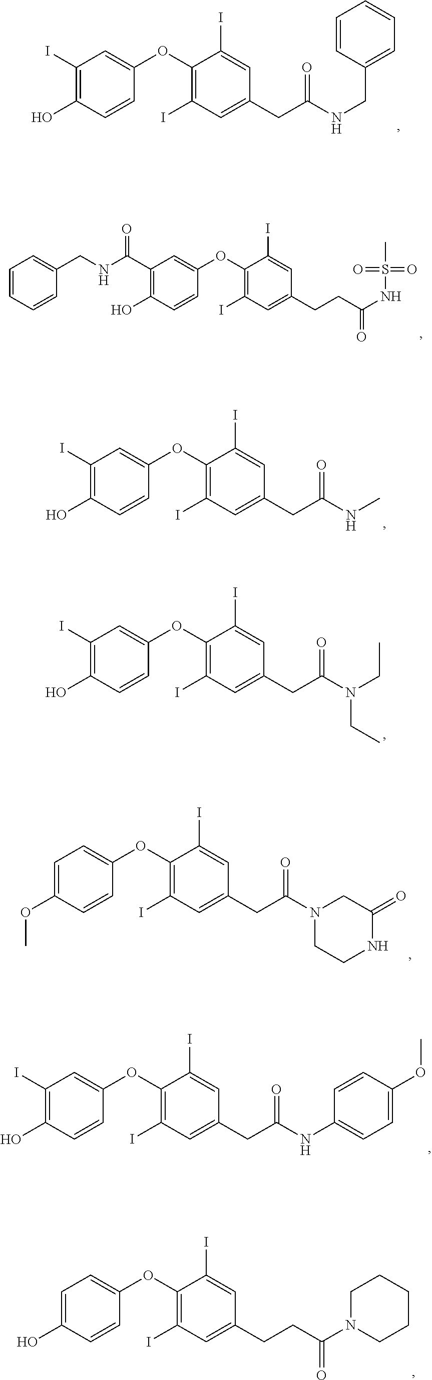

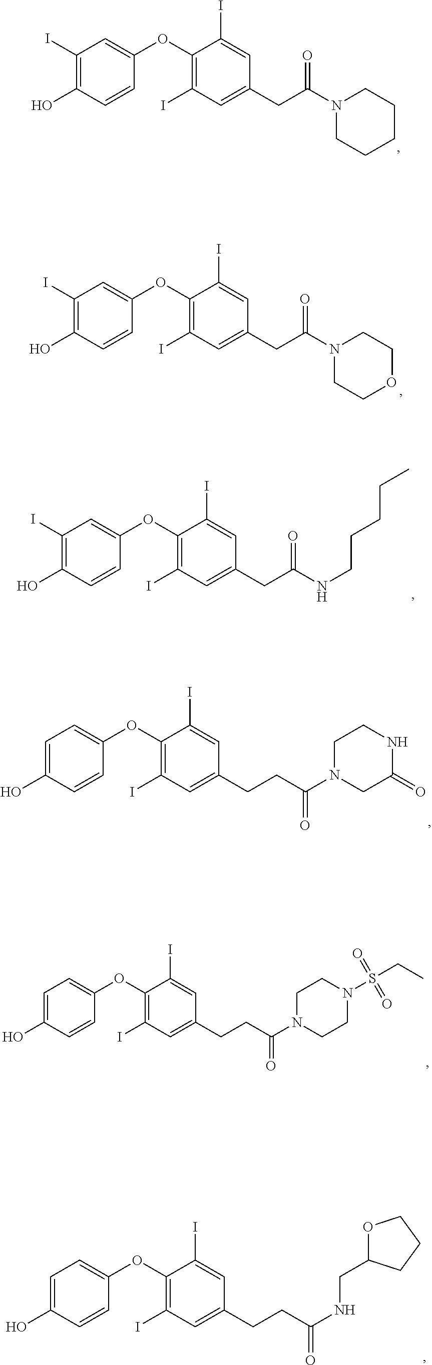 [SMILES: O=C(Cc1cc(I)c(Oc2ccc(O)c(I)c2)c(I)c1)N1CCCCC1 ; O=C(Cc1cc(I)c(Oc2ccc(O)c(I)c2)c(I)c1)N1CCOCC1 ; CCCCCNC(=O)Cc1cc(I)c(Oc2ccc(O)c(I)c2)c(I)c1 ; O=C1CN(C(=O)CCc2cc(I)c(Oc3ccc(O)cc3)c(I)c2)CCN1 ; CCS(=O)(=O)N1CCN(C(=O)CCc2cc(I)c(Oc3ccc(O)cc3)c(I)c2)CC1 ; O=C(CCc1cc(I)c(Oc2ccc(O)cc2)c(I)c1)NCC1CCCO1]

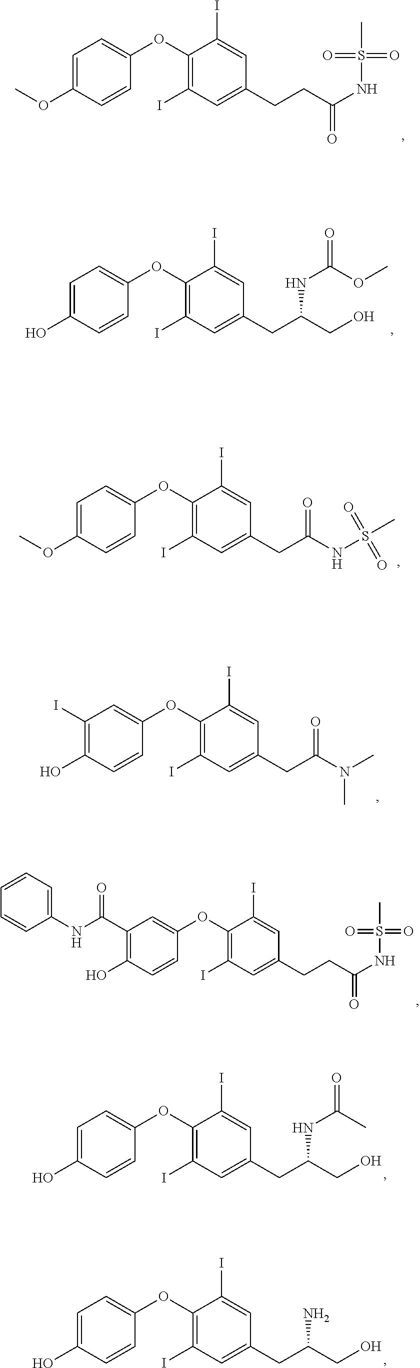

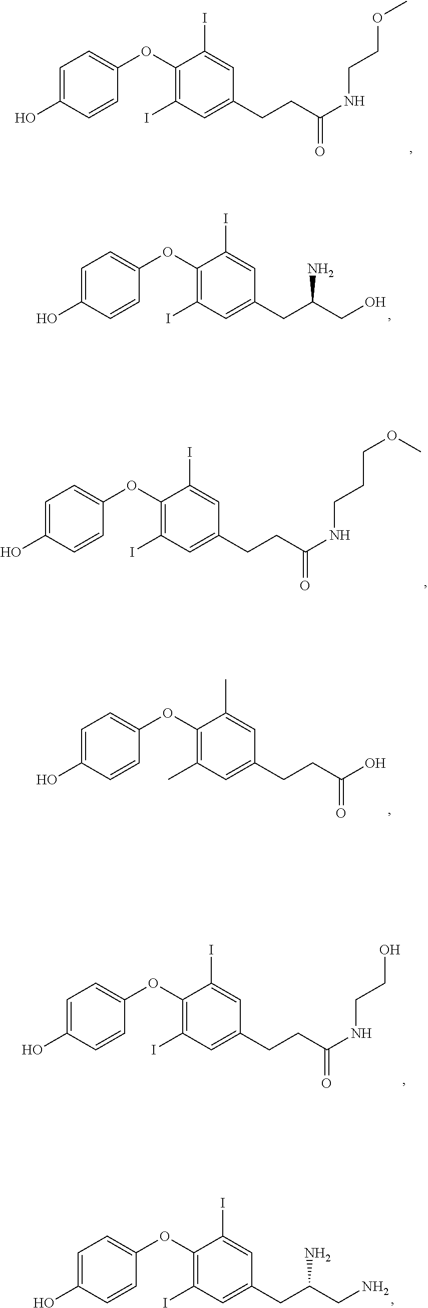 [SMILES: COCCNC(=O)CCc1cc(I)c(Oc2ccc(O)cc2)c(I)c1 ; N[C@@H](CO)Cc1cc(I)c(Oc2ccc(O)cc2)c(I)c1 ; COCCCNC(=O)CCc1cc(I)c(Oc2ccc(O)cc2)c(I)c1 ; Cc1cc(CCC(=O)O)cc(C)c1Oc1ccc(O)cc1 ; O=C(CCc1cc(I)c(Oc2ccc(O)cc2)c(I)c1)NCCO ; NC[C@@H](N)Cc1cc(I)c(Oc2ccc(O)cc2)c(I)c1]

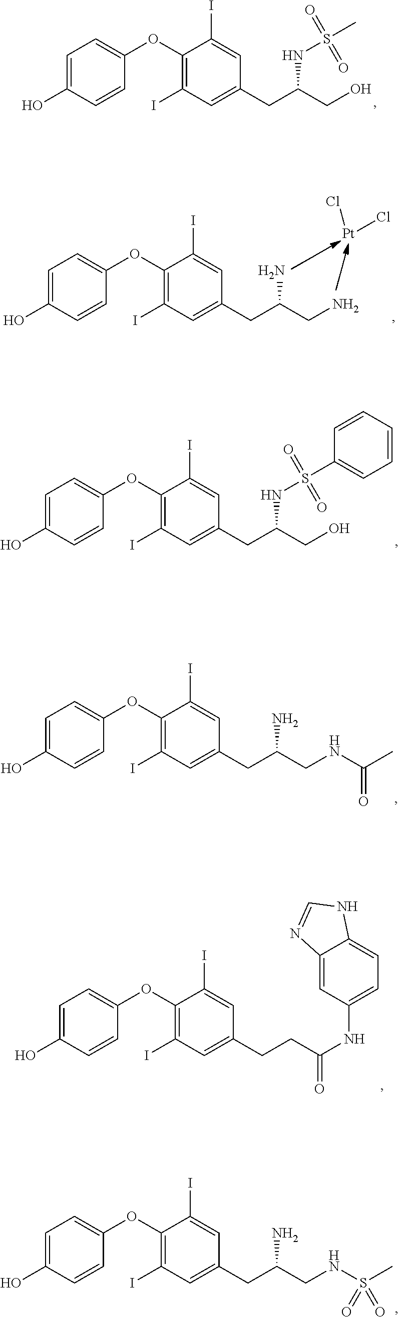

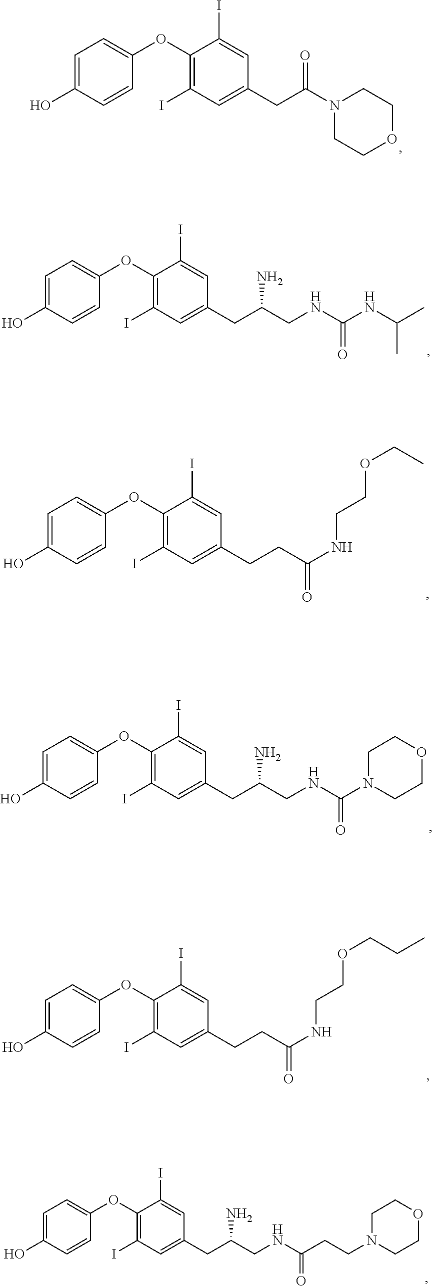 [SMILES: O=C(Cc1cc(I)c(Oc2ccc(O)cc2)c(I)c1)N1CCOCC1 ; CC(C)NC(=O)NC[C@@H](N)Cc1cc(I)c(Oc2ccc(O)cc2)c(I)c1 ; CCOCCNC(=O)CCc1cc(I)c(Oc2ccc(O)cc2)c(I)c1 ; N[C@H](CNC(=O)N1CCOCC1)Cc1cc(I)c(Oc2ccc(O)cc2)c(I)c1 ; CCCOCCNC(=O)CCc1cc(I)c(Oc2ccc(O)cc2)c(I)c1 ; N[C@H](CNC(=O)CCN1CCOCC1)Cc1cc(I)c(Oc2ccc(O)cc2)c(I)c1]

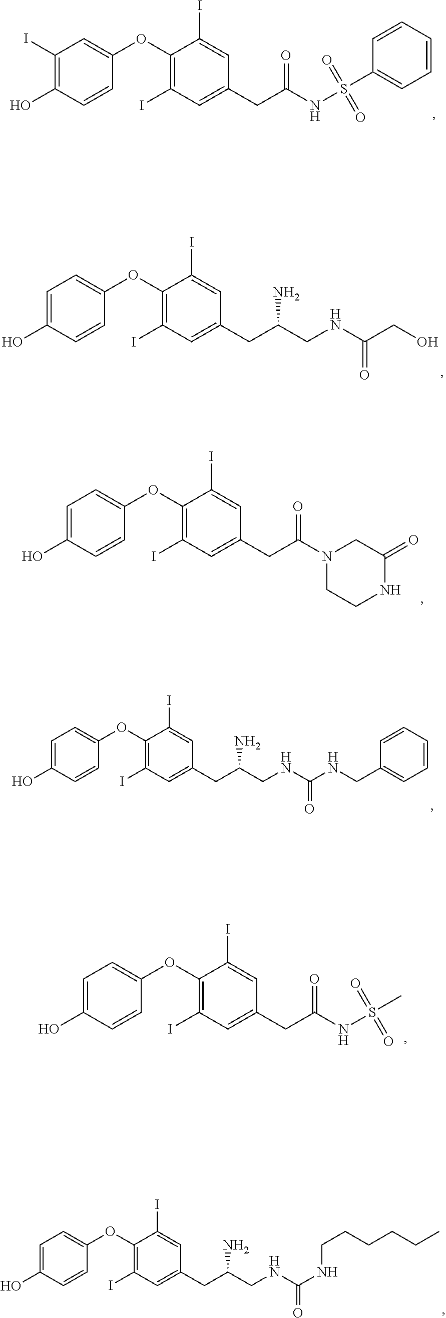 [SMILES: O=C(Cc1cc(I)c(Oc2ccc(O)c(I)c2)c(I)c1)NS(=O)(=O)c1ccccc1 ; N[C@H](CNC(=O)CO)Cc1cc(I)c(Oc2ccc(O)cc2)c(I)c1 ; O=C1CN(C(=O)Cc2cc(I)c(Oc3ccc(O)cc3)c(I)c2)CCN1 ; N[C@H](CNC(=O)NCc1ccccc1)Cc1cc(I)c(Oc2ccc(O)cc2)c(I)c1 ; CS(=O)(=O)NC(=O)Cc1cc(I)c(Oc2ccc(O)cc2)c(I)c1 ; CCCCCCNC(=O)NC[C@@H](N)Cc1cc(I)c(Oc2ccc(O)cc2)c(I)c1]

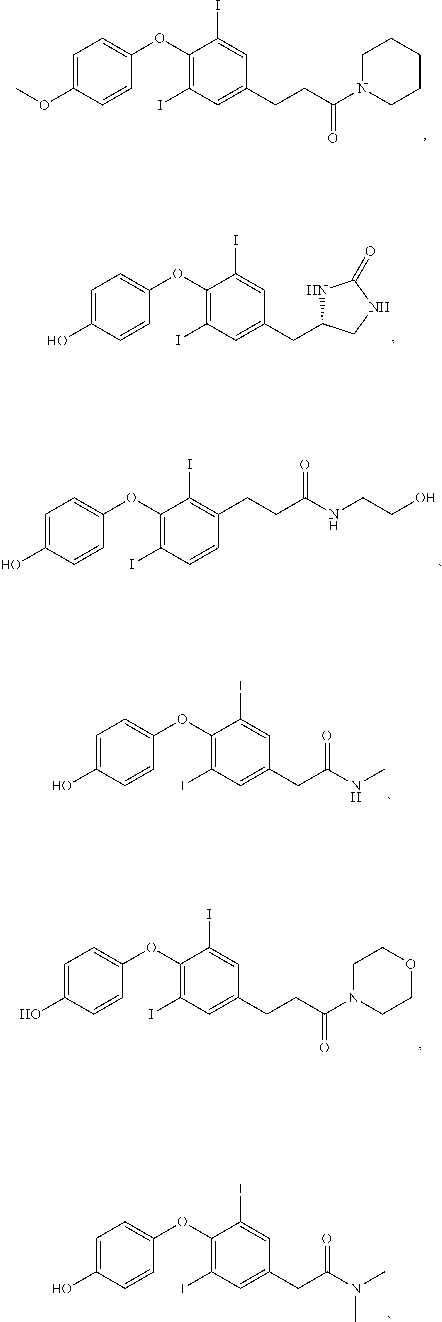 [SMILES: COc1ccc(Oc2c(I)cc(CCC(=O)N3CCCCC3)cc2I)cc1 ; O=C1NC[C@H](Cc2cc(I)c(Oc3ccc(O)cc3)c(I)c2)N1 ; O=C(CCc1ccc(I)c(Oc2ccc(O)cc2)c1I)NCCO ; CNC(=O)Cc1cc(I)c(Oc2ccc(O)cc2)c(I)c1 ; O=C(CCc1cc(I)c(Oc2ccc(O)cc2)c(I)c1)N1CCOCC1 ; CN(C)C(=O)Cc1cc(I)c(Oc2ccc(O)cc2)c(I)c1]

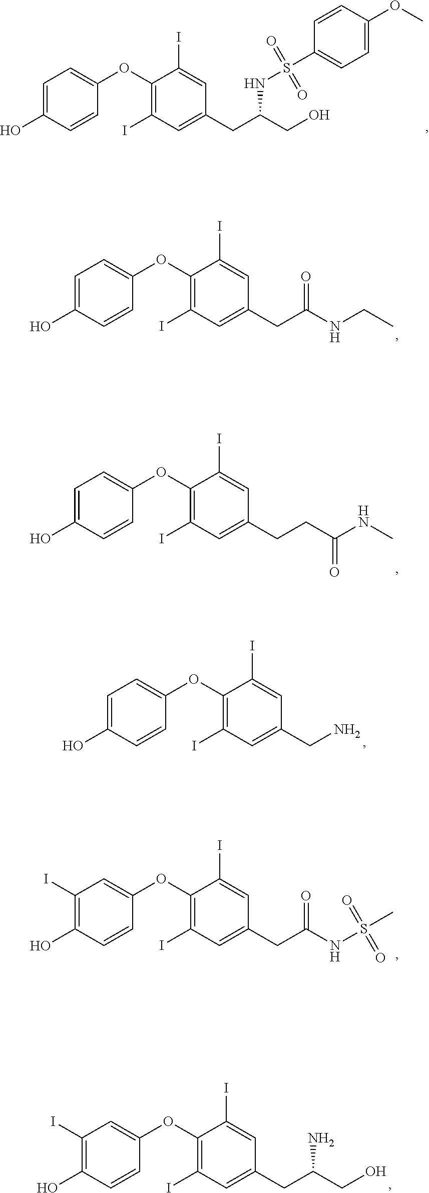 [SMILES: COc1ccc(S(=O)(=O)N[C@H](CO)Cc2cc(I)c(Oc3ccc(O)cc3)c(I)c2)cc1 ; CCNC(=O)Cc1cc(I)c(Oc2ccc(O)cc2)c(I)c1 ; CNC(=O)CCc1cc(I)c(Oc2ccc(O)cc2)c(I)c1 ; NCc1cc(I)c(Oc2ccc(O)cc2)c(I)c1 ; CS(=O)(=O)NC(=O)Cc1cc(I)c(Oc2ccc(O)c(I)c2)c(I)c1 ; N[C@H](CO)Cc1cc(I)c(Oc2ccc(O)c(I)c2)c(I)c1]

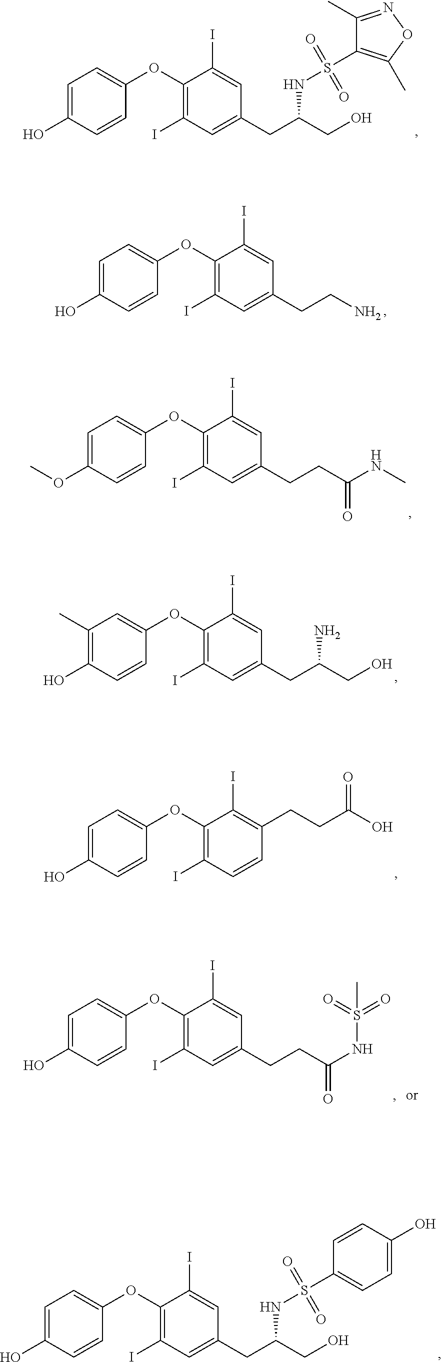

or a subgroup thereof.

In one aspect, a compound can be present as:

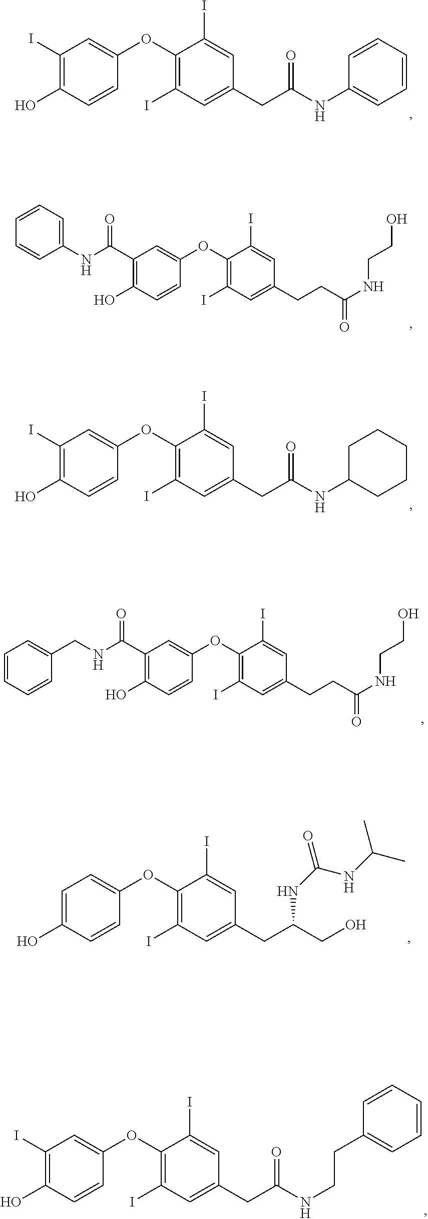 [SMILES: O=C(Cc1cc(I)c(Oc2ccc(O)c(I)c2)c(I)c1)Nc1ccccc1 ; O=C(CCc1cc(I)c(Oc2ccc(O)c(C(=O)Nc3ccccc3)c2)c(I)c1)NCCO ; O=C(Cc1cc(I)c(Oc2ccc(O)c(I)c2)c(I)c1)NC1CCCCC1 ; O=C(CCc1cc(I)c(Oc2ccc(O)c(C(=O)NCc3ccccc3)c2)c(I)c1)NCCO ; CC(C)NC(=O)N[C@H](CO)Cc1cc(I)c(Oc2ccc(O)cc2)c(I)c1 ; O=C(Cc1cc(I)c(Oc2ccc(O)c(I)c2)c(I)c1)NCCc1ccccc1]

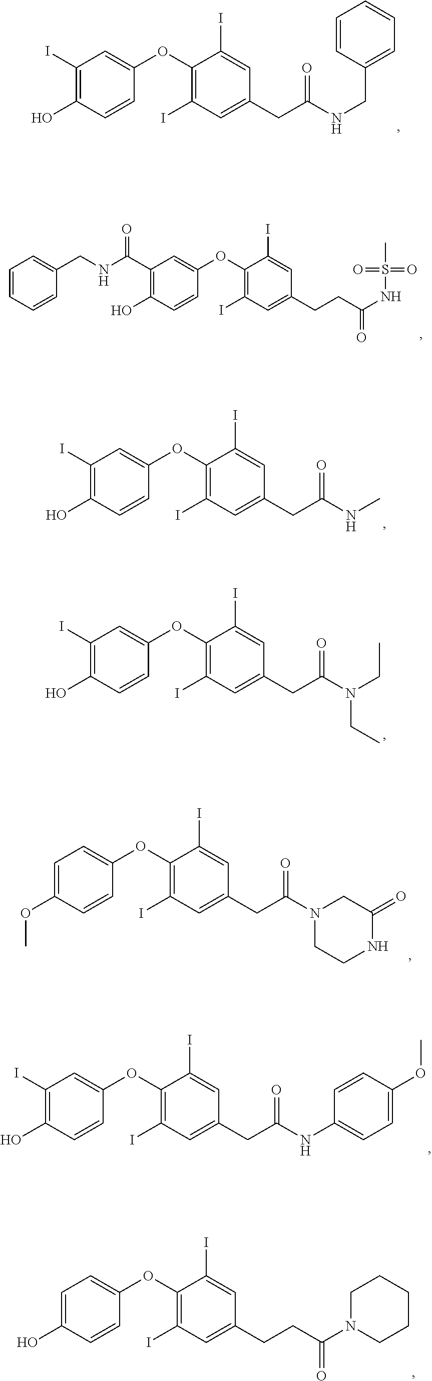

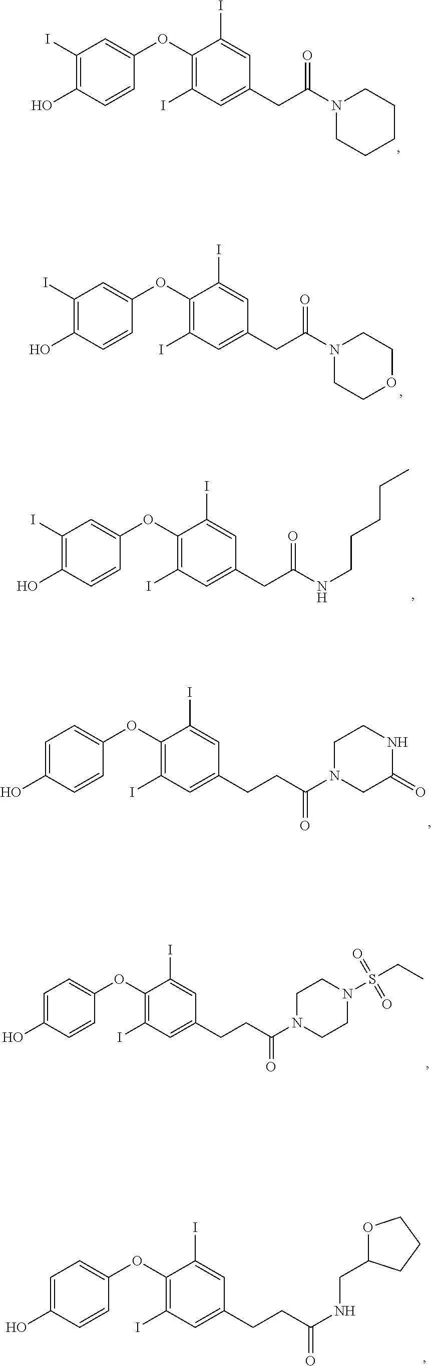 [SMILES: O=C(Cc1cc(I)c(Oc2ccc(O)c(I)c2)c(I)c1)N1CCCCC1 ; O=C(Cc1cc(I)c(Oc2ccc(O)c(I)c2)c(I)c1)N1CCOCC1 ; CCCCCNC(=O)Cc1cc(I)c(Oc2ccc(O)c(I)c2)c(I)c1 ; O=C1CN(C(=O)CCc2cc(I)c(Oc3ccc(O)cc3)c(I)c2)CCN1 ; CCS(=O)(=O)N1CCN(C(=O)CCc2cc(I)c(Oc3ccc(O)cc3)c(I)c2)CC1 ; O=C(CCc1cc(I)c(Oc2ccc(O)cc2)c(I)c1)NCC1CCCO1]

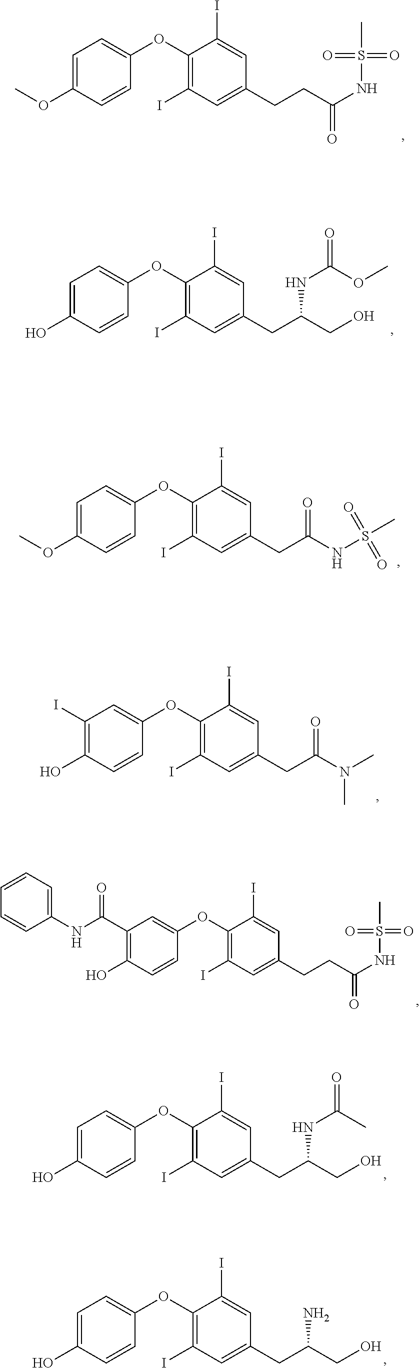

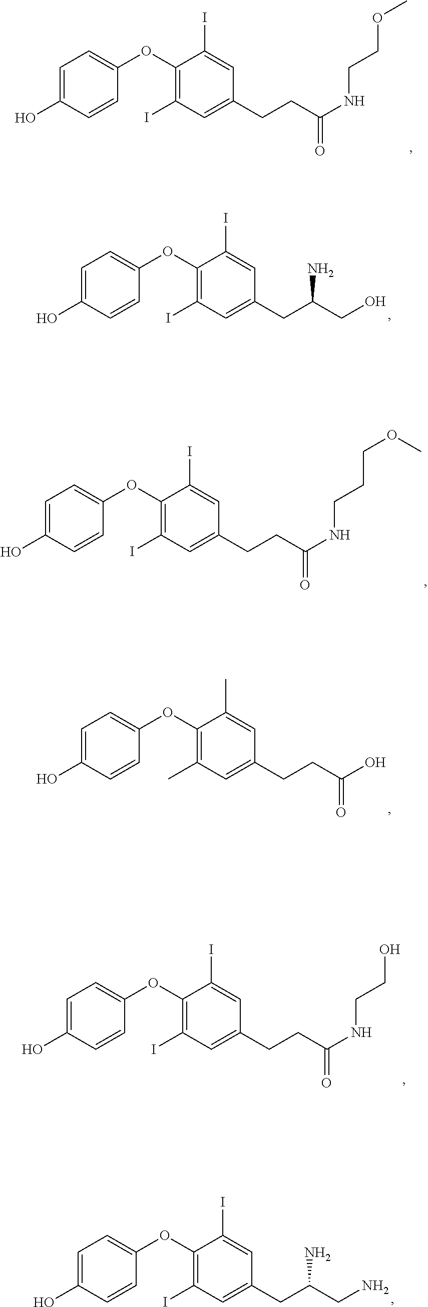 [SMILES: COCCNC(=O)CCc1cc(I)c(Oc2ccc(O)cc2)c(I)c1 ; N[C@@H](CO)Cc1cc(I)c(Oc2ccc(O)cc2)c(I)c1 ; COCCCNC(=O)CCc1cc(I)c(Oc2ccc(O)cc2)c(I)c1 ; Cc1cc(CCC(=O)O)cc(C)c1Oc1ccc(O)cc1 ; O=C(CCc1cc(I)c(Oc2ccc(O)cc2)c(I)c1)NCCO ; NC[C@@H](N)Cc1cc(I)c(Oc2ccc(O)cc2)c(I)c1]

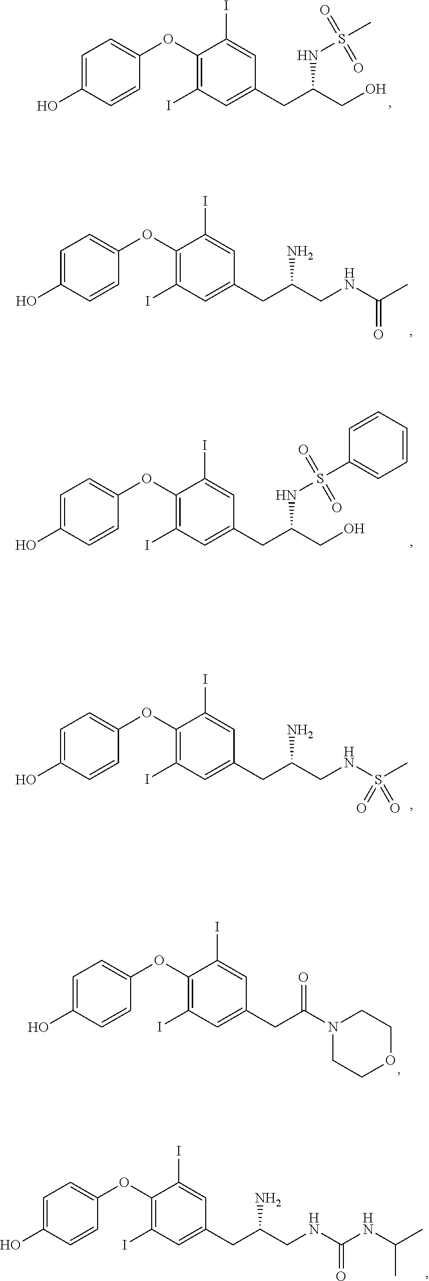 [SMILES: CS(=O)(=O)N[C@H](CO)Cc1cc(I)c(Oc2ccc(O)cc2)c(I)c1 ; CC(=O)NC[C@@H](N)Cc1cc(I)c(Oc2ccc(O)cc2)c(I)c1 ; O=S(=O)(N[C@H](CO)Cc1cc(I)c(Oc2ccc(O)cc2)c(I)c1)c1ccccc1 ; CS(=O)(=O)NC[C@@H](N)Cc1cc(I)c(Oc2ccc(O)cc2)c(I)c1 ; O=C(Cc1cc(I)c(Oc2ccc(O)cc2)c(I)c1)N1CCOCC1 ; CC(C)NC(=O)NC[C@@H](N)Cc1cc(I)c(Oc2ccc(O)cc2)c(I)c1]

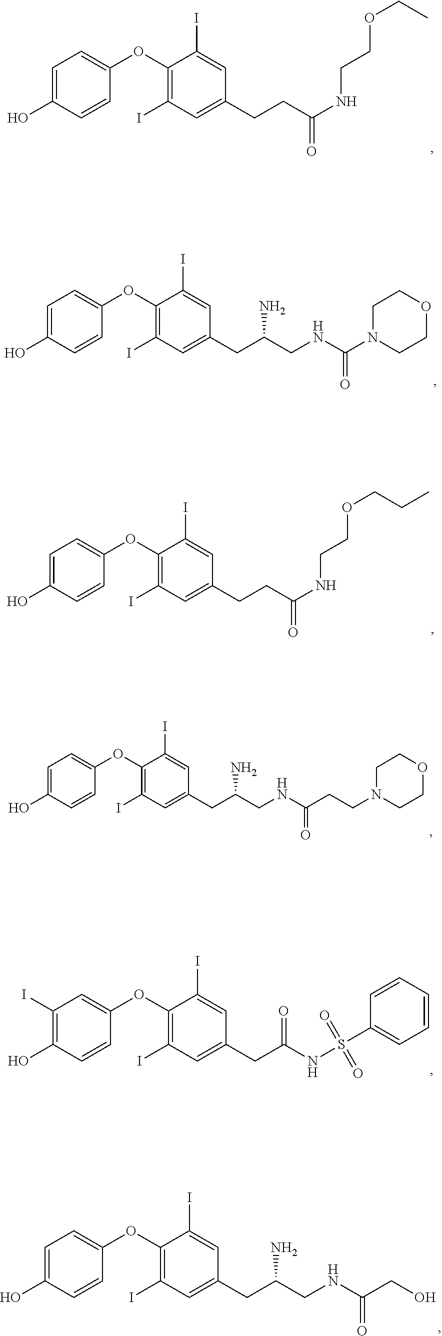 [SMILES: CCOCCNC(=O)CCc1cc(I)c(Oc2ccc(O)cc2)c(I)c1 ; N[C@H](CNC(=O)N1CCOCC1)Cc1cc(I)c(Oc2ccc(O)cc2)c(I)c1 ; CCCOCCNC(=O)CCc1cc(I)c(Oc2ccc(O)cc2)c(I)c1 ; N[C@H](CNC(=O)CCN1CCOCC1)Cc1cc(I)c(Oc2ccc(O)cc2)c(I)c1 ; O=C(Cc1cc(I)c(Oc2ccc(O)c(I)c2)c(I)c1)NS(=O)(=O)c1ccccc1 ; N[C@H](CNC(=O)CO)Cc1cc(I)c(Oc2ccc(O)cc2)c(I)c1]

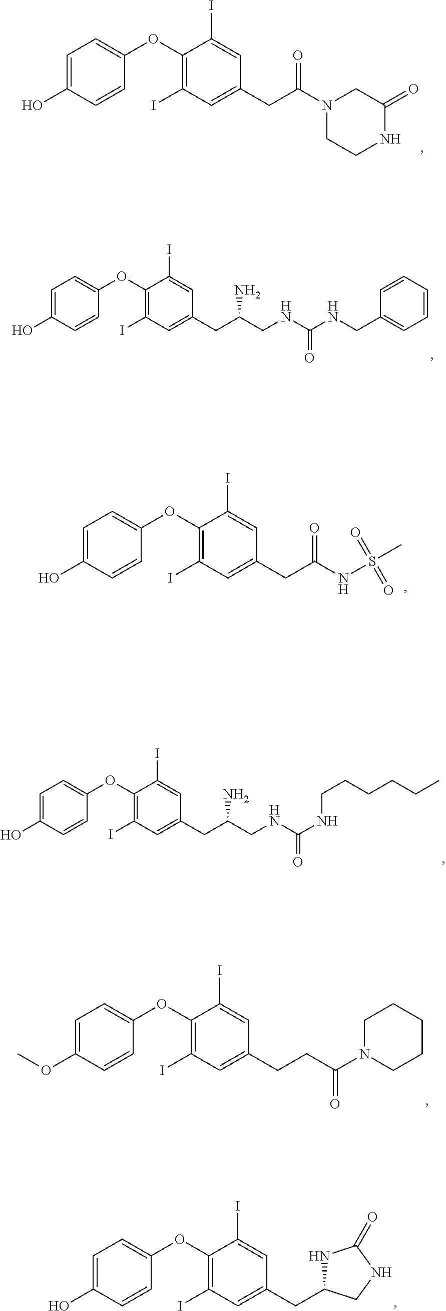

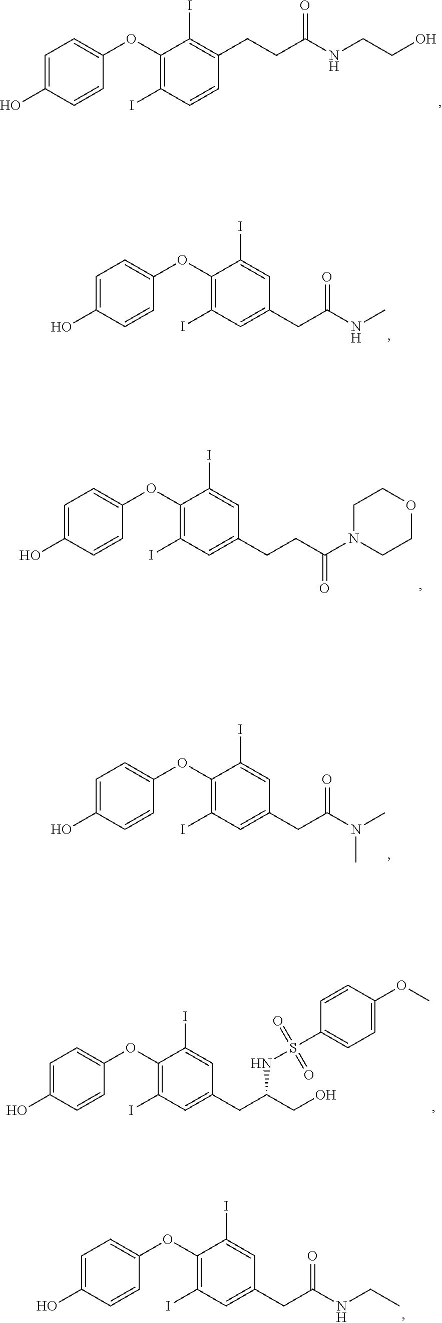 [SMILES: O=C(CCc1ccc(I)c(Oc2ccc(O)cc2)c1I)NCCO ; CNC(=O)Cc1cc(I)c(Oc2ccc(O)cc2)c(I)c1 ; O=C(CCc1cc(I)c(Oc2ccc(O)cc2)c(I)c1)N1CCOCC1 ; CN(C)C(=O)Cc1cc(I)c(Oc2ccc(O)cc2)c(I)c1 ; COc1ccc(S(=O)(=O)N[C@H](CO)Cc2cc(I)c(Oc3ccc(O)cc3)c(I)c2)cc1 ; CCNC(=O)Cc1cc(I)c(Oc2ccc(O)cc2)c(I)c1]

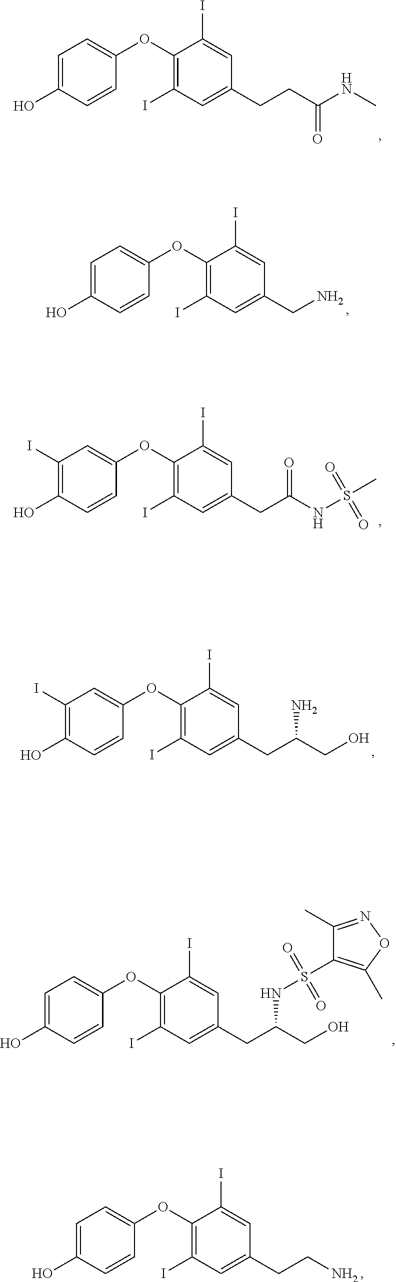 [SMILES: CNC(=O)CCc1cc(I)c(Oc2ccc(O)cc2)c(I)c1 ; NCc1cc(I)c(Oc2ccc(O)cc2)c(I)c1 ; CS(=O)(=O)NC(=O)Cc1cc(I)c(Oc2ccc(O)c(I)c2)c(I)c1 ; N[C@H](CO)Cc1cc(I)c(Oc2ccc(O)c(I)c2)c(I)c1 ; Cc1noc(C)c1S(=O)(=O)N[C@H](CO)Cc1cc(I)c(Oc2ccc(O)cc2)c(I)c1 ; NCCc1cc(I)c(Oc2ccc(O)cc2)c(I)c1]

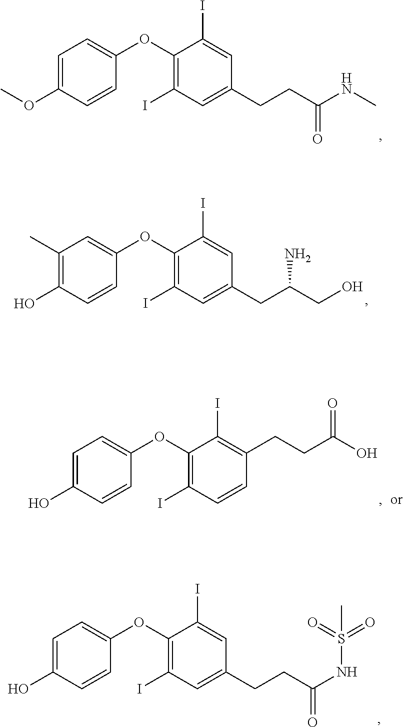

or a subgroup thereof.

In one aspect, a compound can be present as:

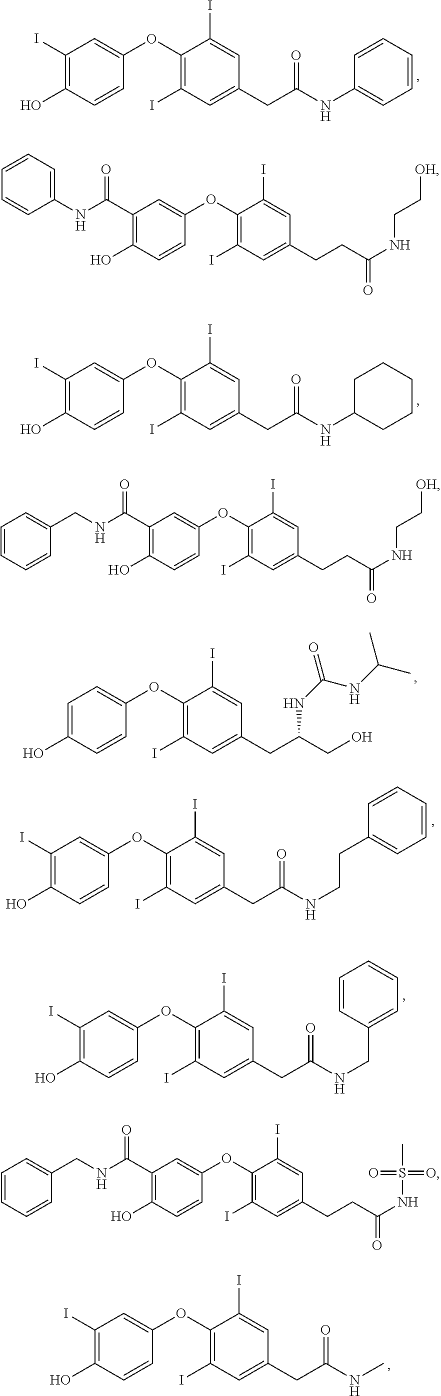

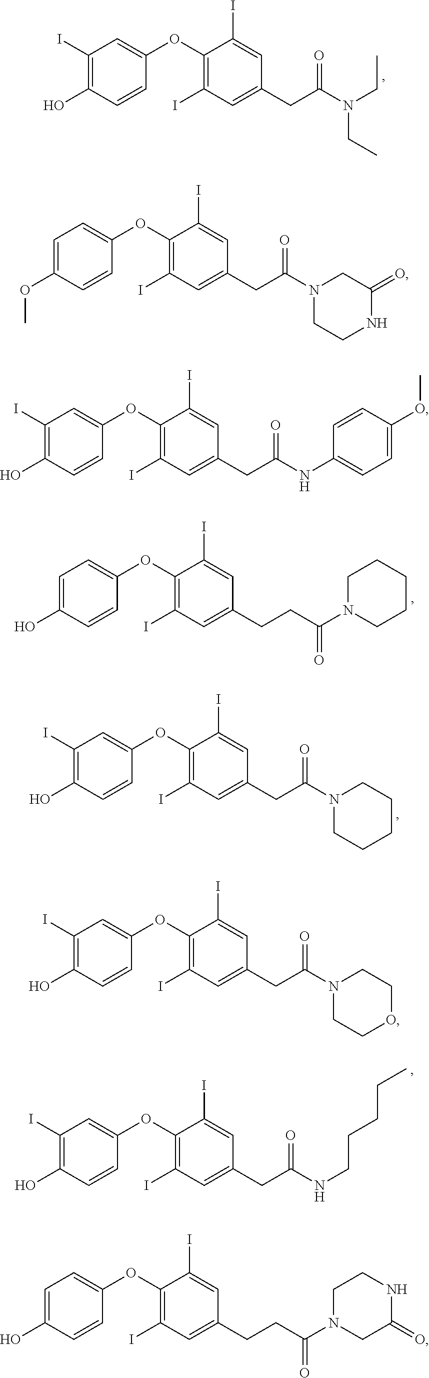

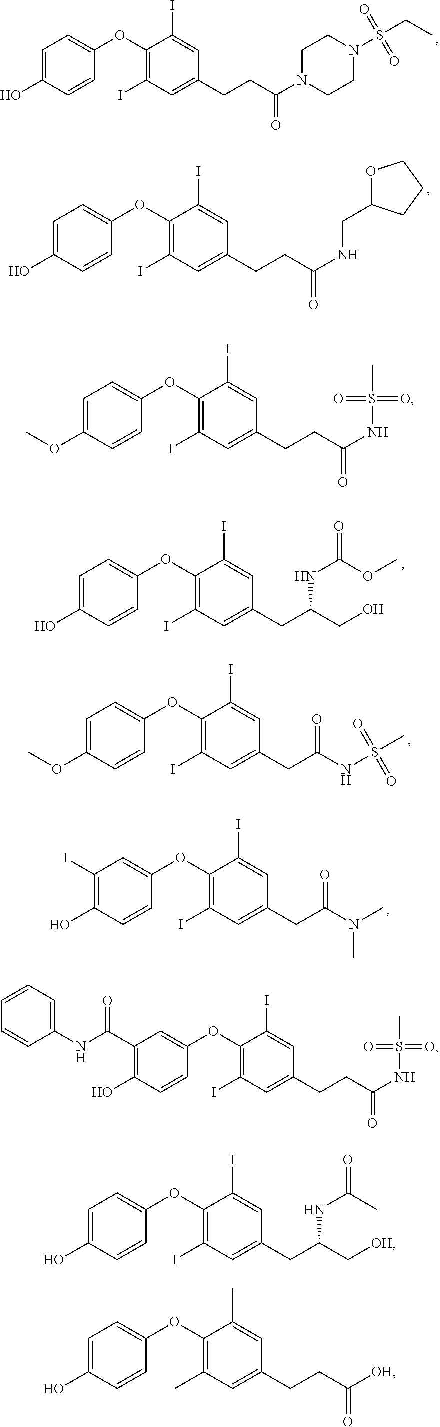

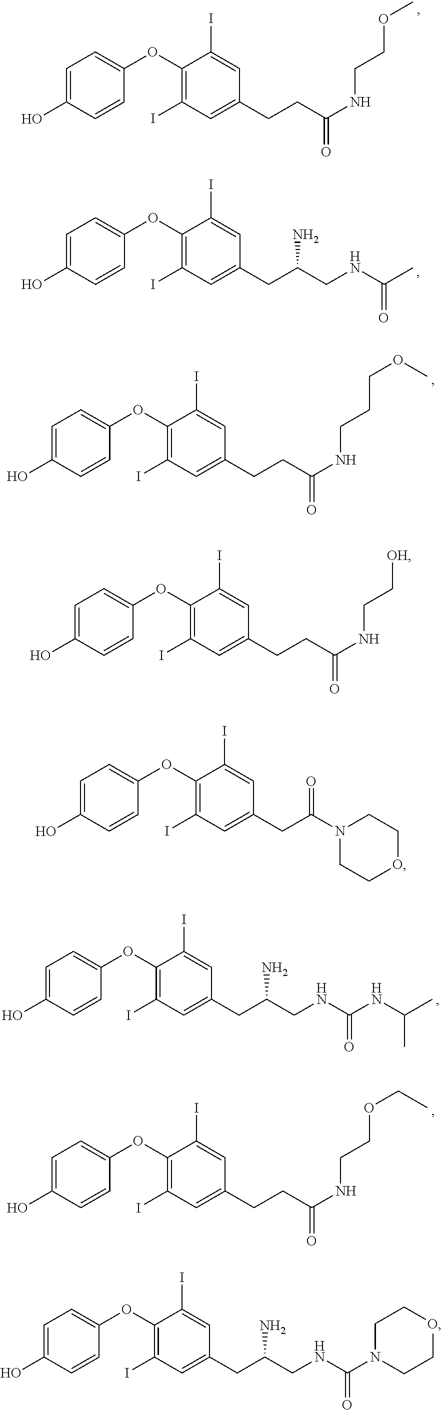

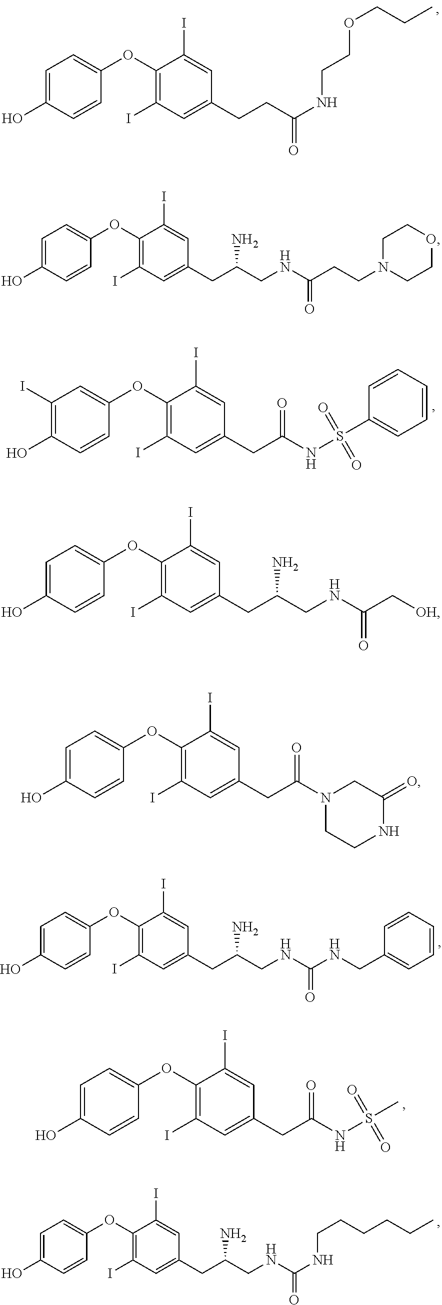

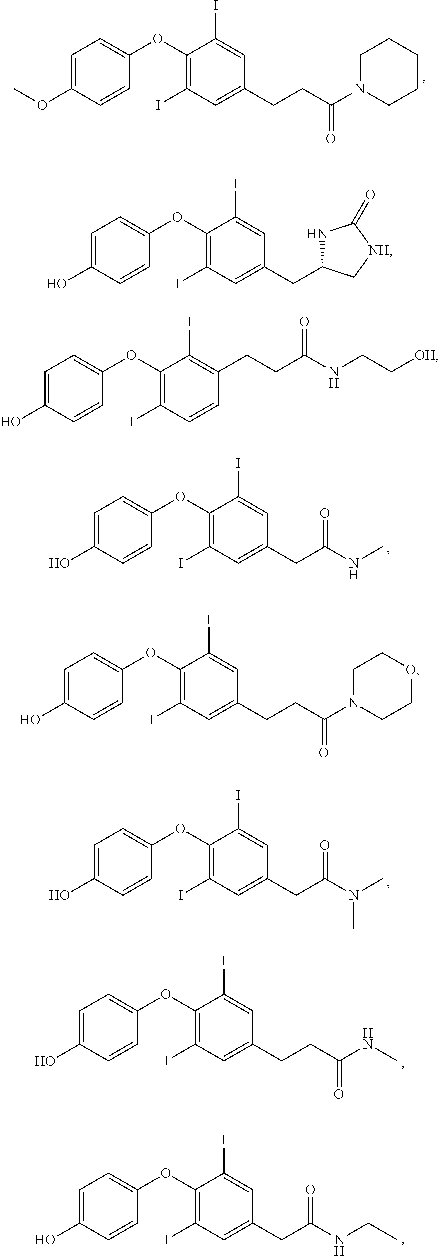

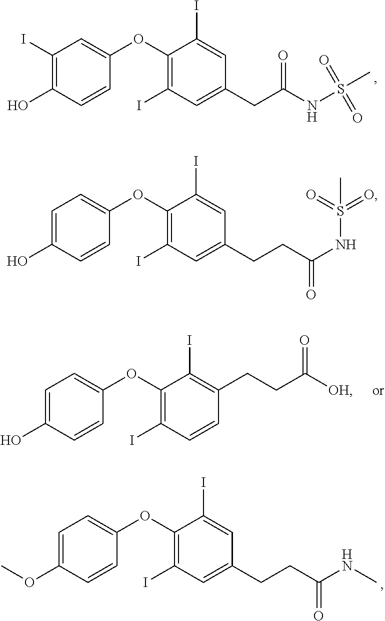

or a subgroup thereof.

In one aspect, a compound can be present as:

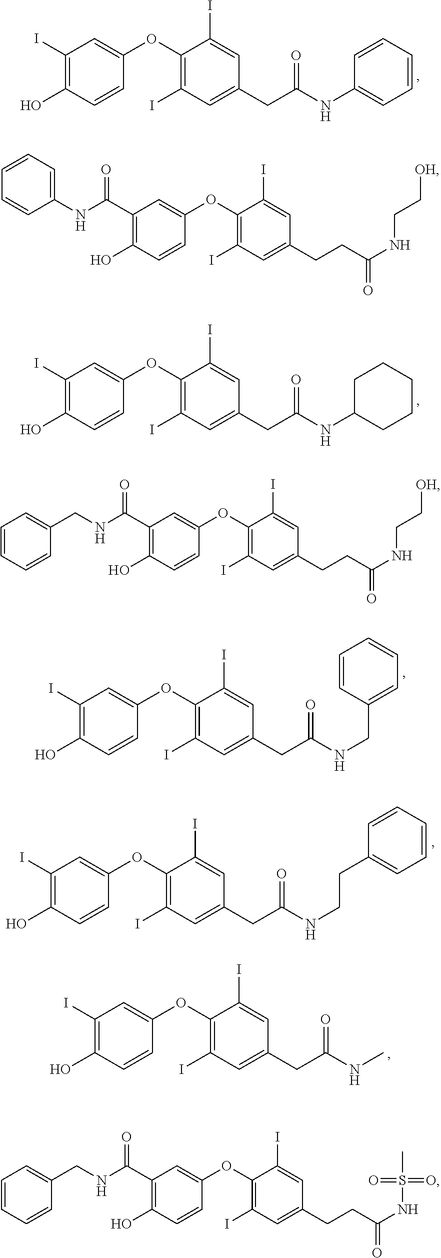

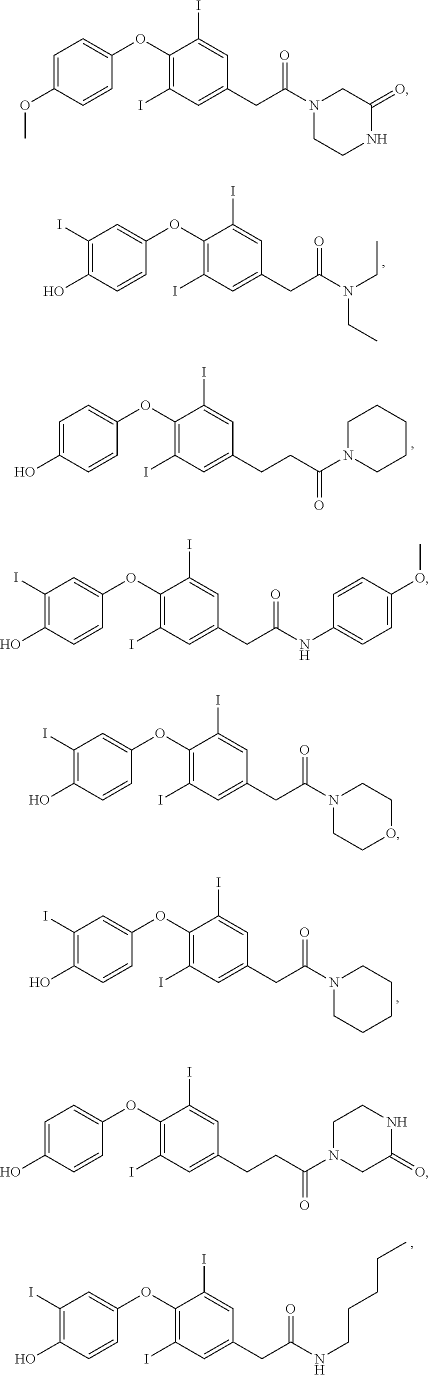

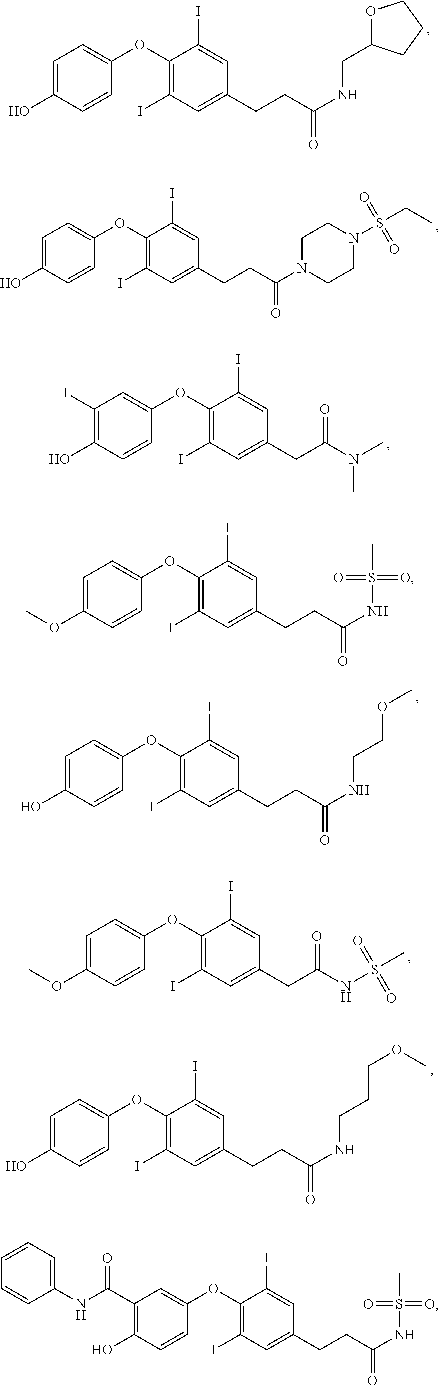

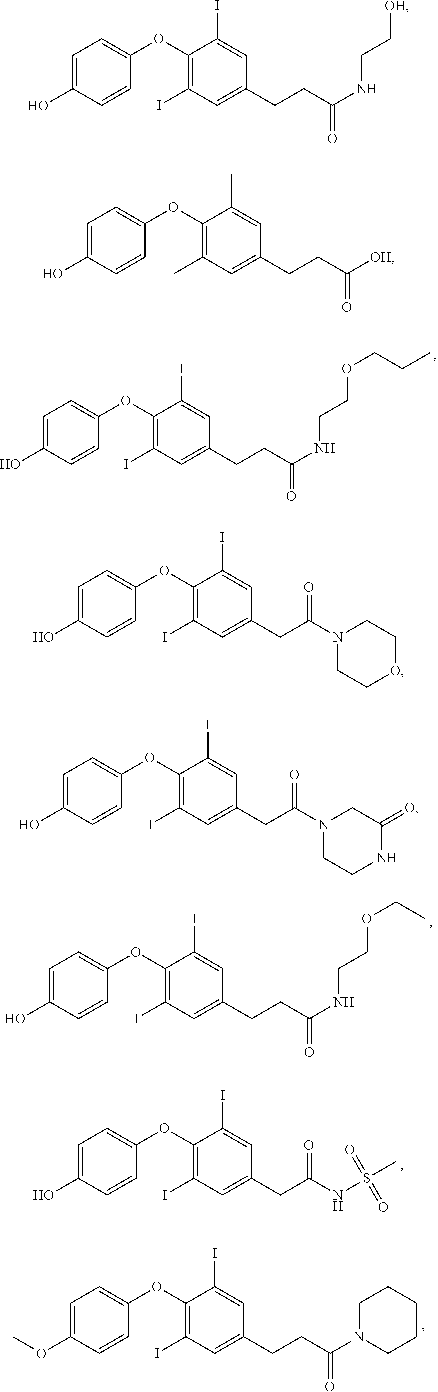

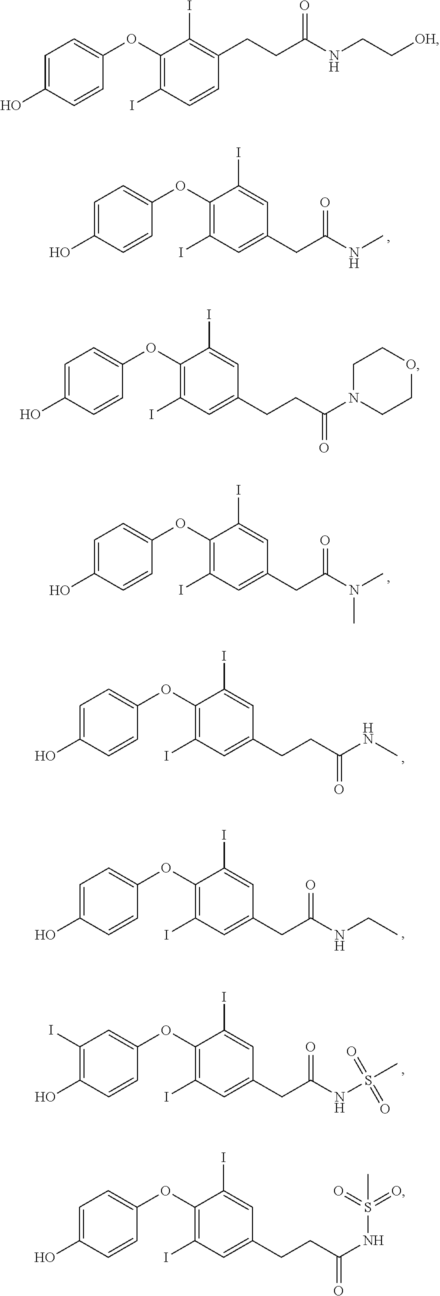

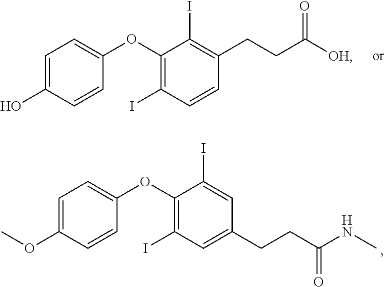

or a subgroup thereof.

In one aspect, a compound can be present as:

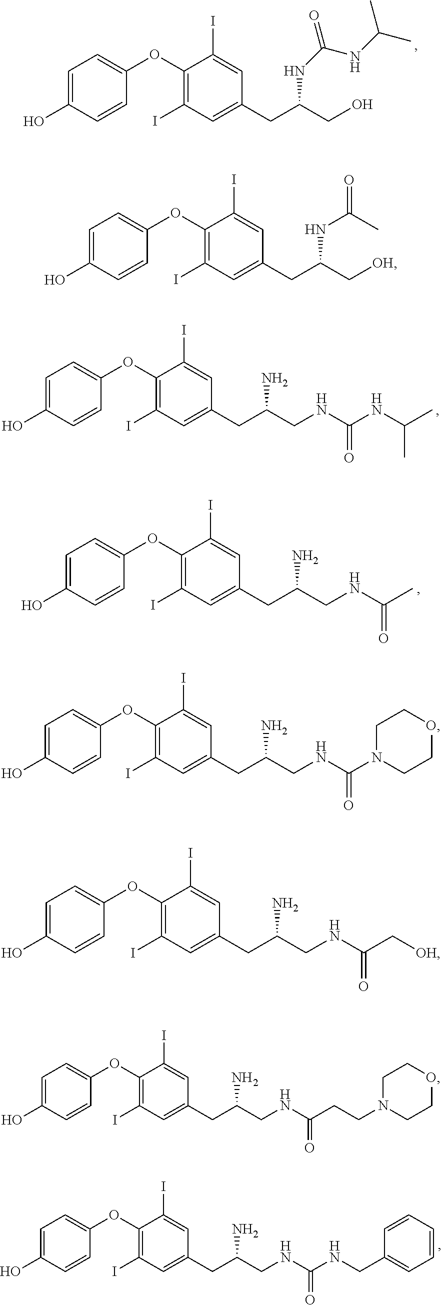

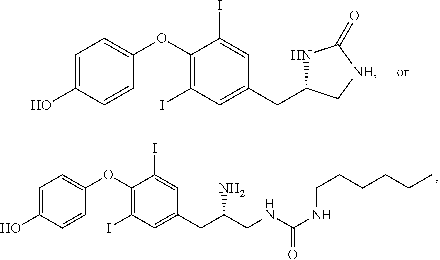 [SMILES: O=C1NC[C@H](Cc2cc(I)c(Oc3ccc(O)cc3)c(I)c2)N1 ; CCCCCCNC(=O)NC[C@@H](N)Cc1cc(I)c(Oc2ccc(O)cc2)c(I)c1]

or a subgroup thereof.

In one aspect, a compound can be present as:

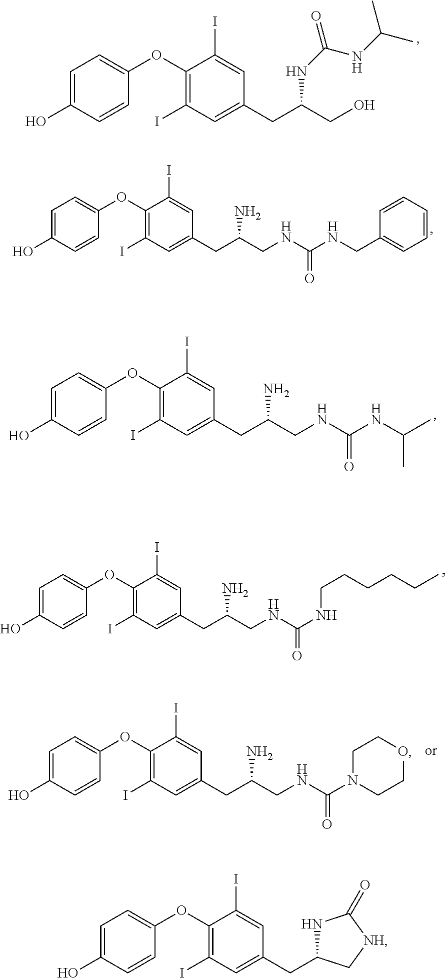 [SMILES: CC(C)NC(=O)N[C@H](CO)Cc1cc(I)c(Oc2ccc(O)cc2)c(I)c1 ; N[C@H](CNC(=O)NCc1ccccc1)Cc1cc(I)c(Oc2ccc(O)cc2)c(I)c1 ; CC(C)NC(=O)NC[C@@H](N)Cc1cc(I)c(Oc2ccc(O)cc2)c(I)c1 ; CCCCCCNC(=O)NC[C@@H](N)Cc1cc(I)c(Oc2ccc(O)cc2)c(I)c1 ; N[C@H](CNC(=O)N1CCOCC1)Cc1cc(I)c(Oc2ccc(O)cc2)c(I)c1 ; O=C1NC[C@H](Cc2cc(I)c(Oc3ccc(O)cc3)c(I)c2)N1]

or a subgroup thereof.

In one aspect, a compound can be present as:

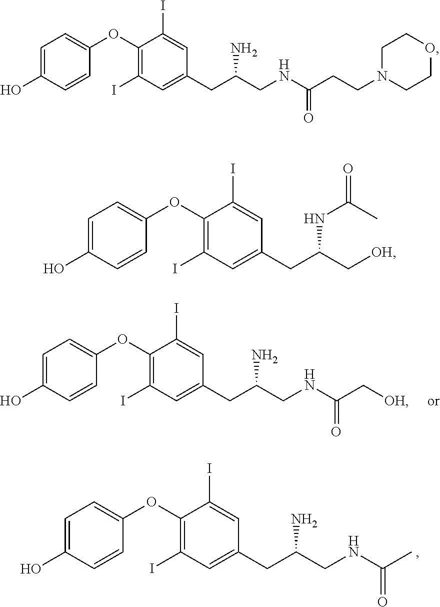

or a subgroup thereof.

In one aspect, a compound can be present as:

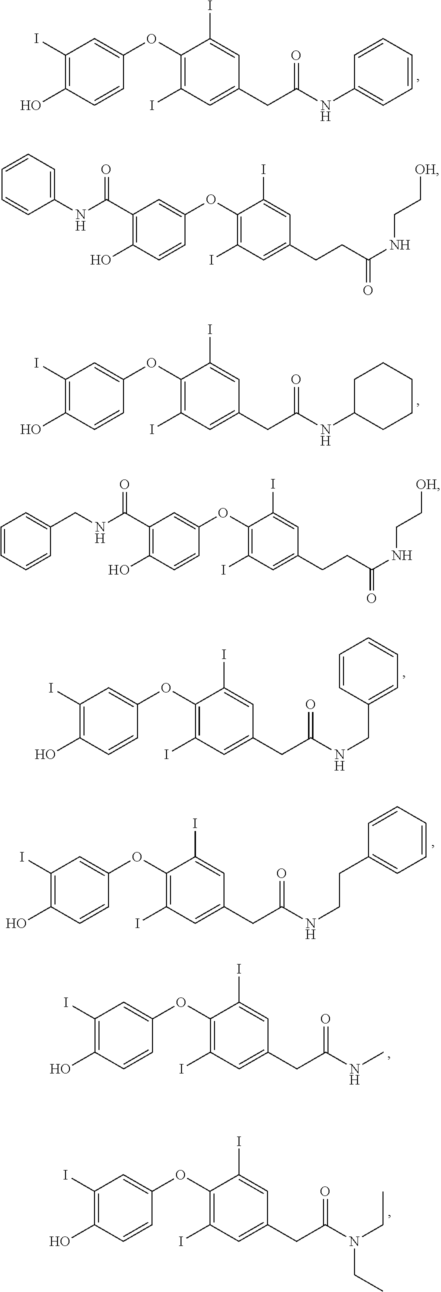

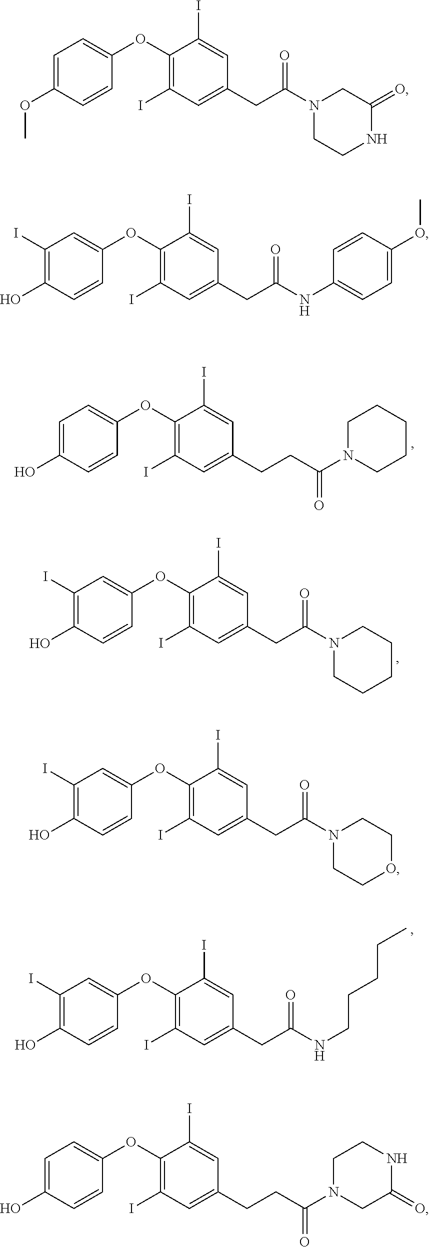

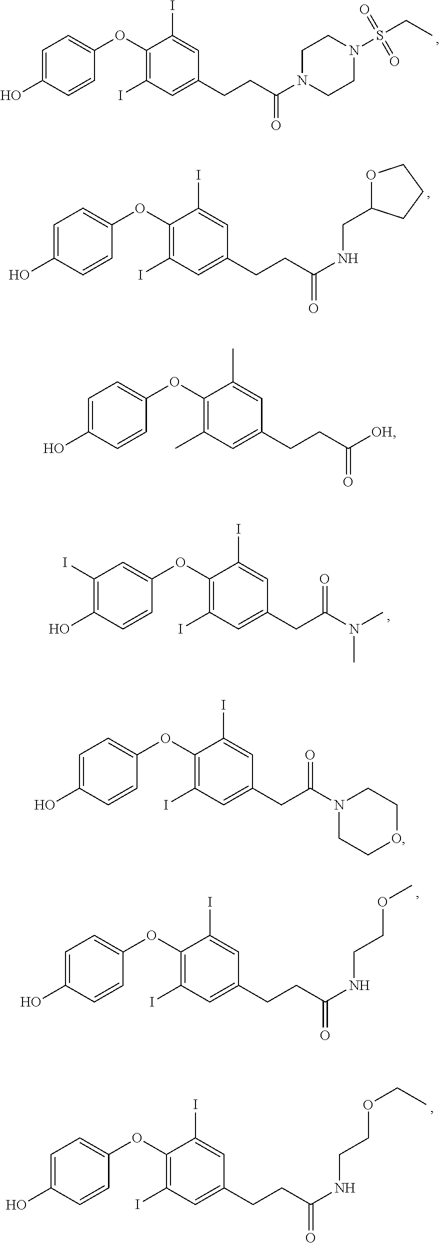

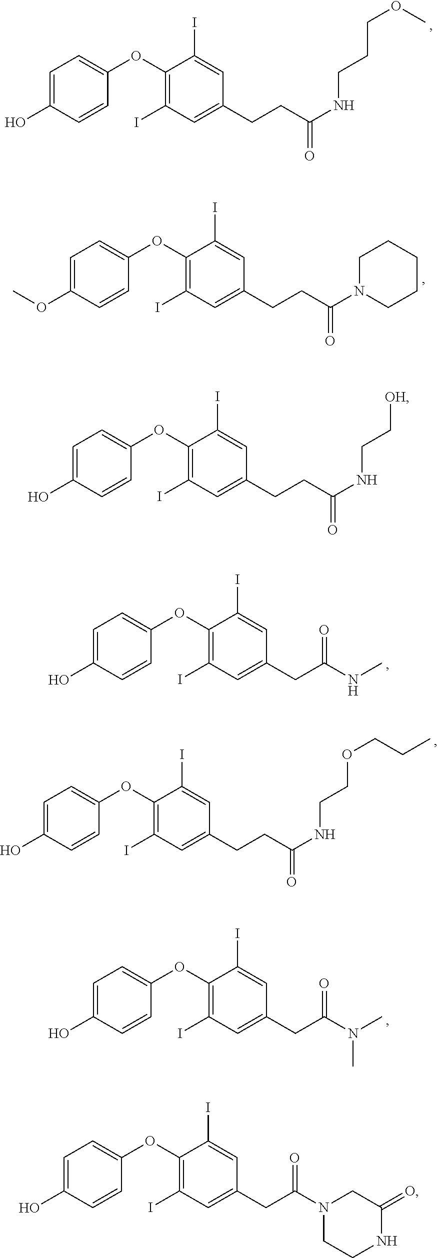

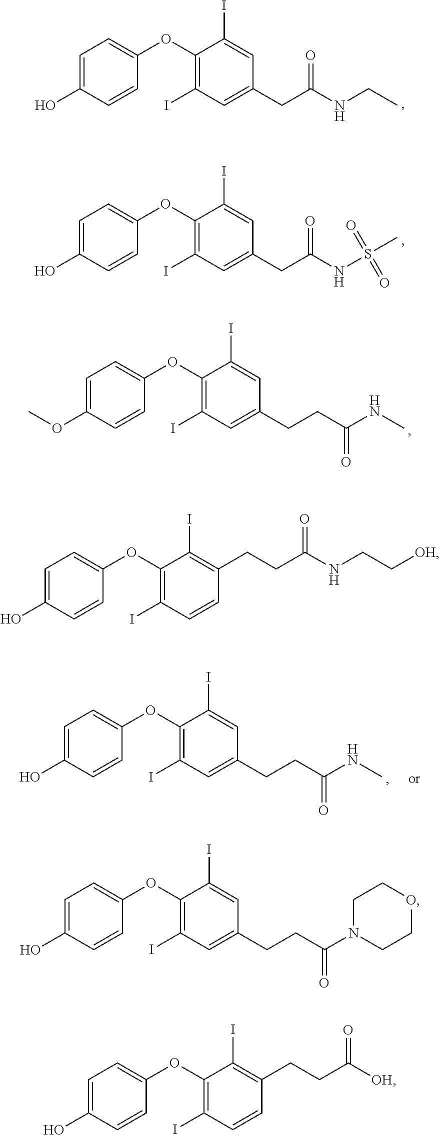

or a subgroup thereof.

In one aspect, a compound can be present as:

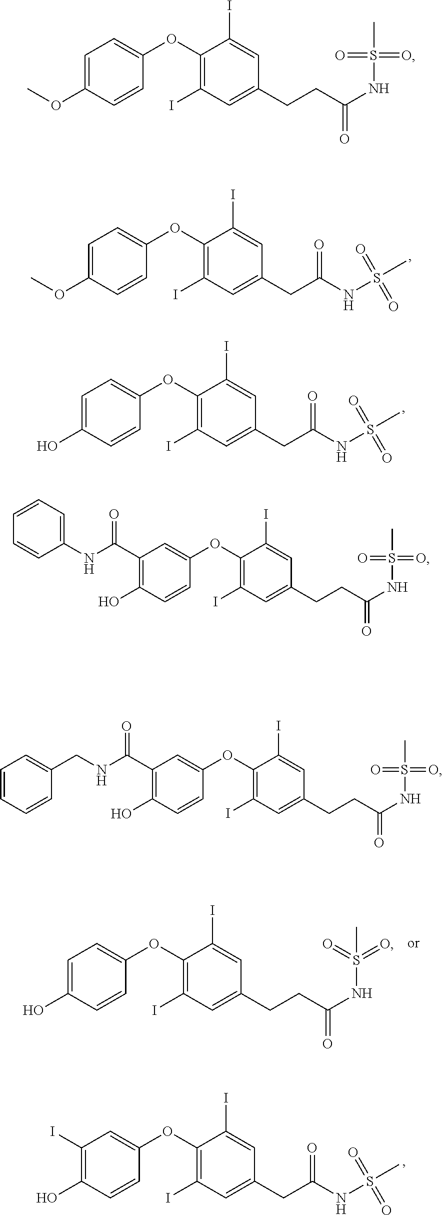

or a subgroup thereof.

In one aspect, a compound can be present as:

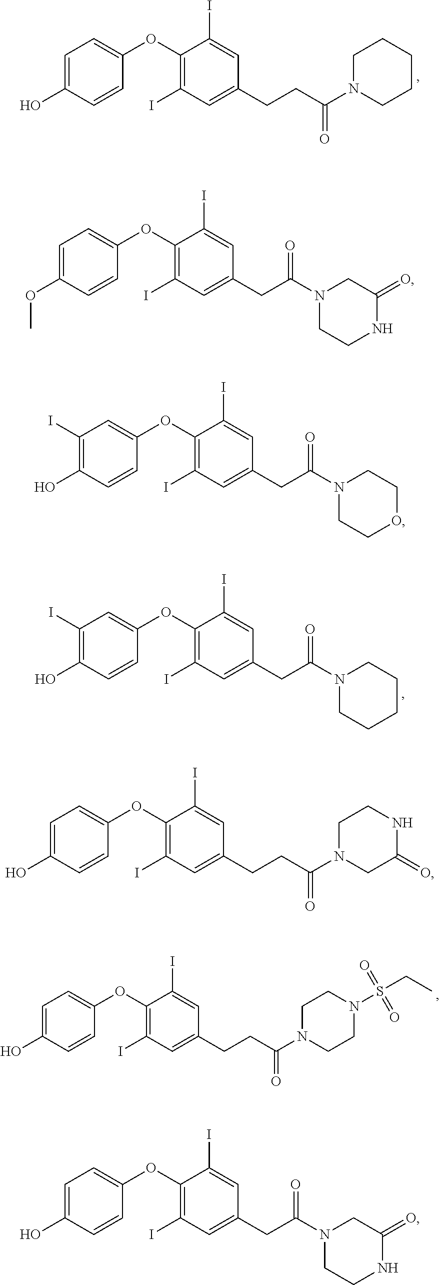

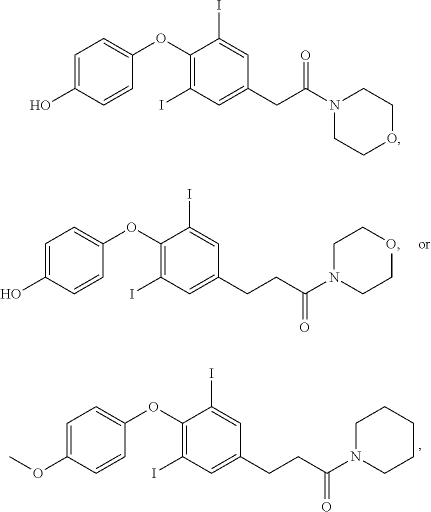 [SMILES: O=C(Cc1cc(I)c(Oc2ccc(O)cc2)c(I)c1)N1CCOCC1 ; O=C(CCc1cc(I)c(Oc2ccc(O)cc2)c(I)c1)N1CCOCC1 ; COc1ccc(Oc2c(I)cc(CCC(=O)N3CCCCC3)cc2I)cc1]

or a subgroup thereof.

In one aspect, a compound can be present as:

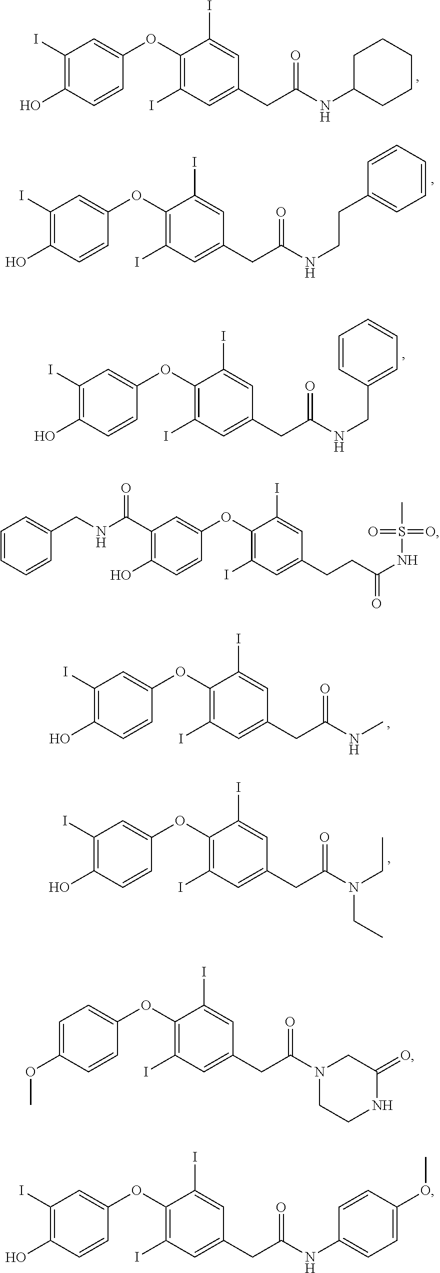

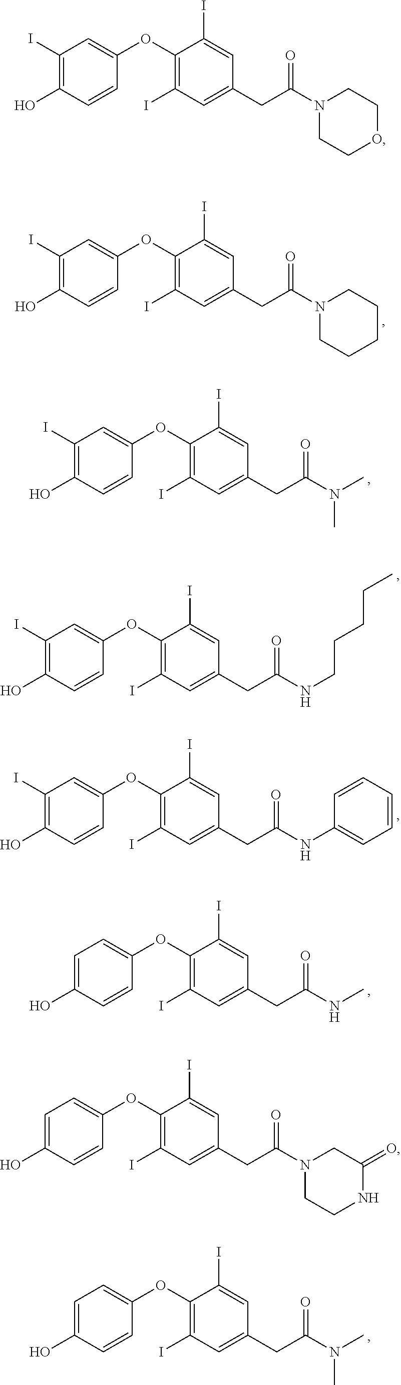

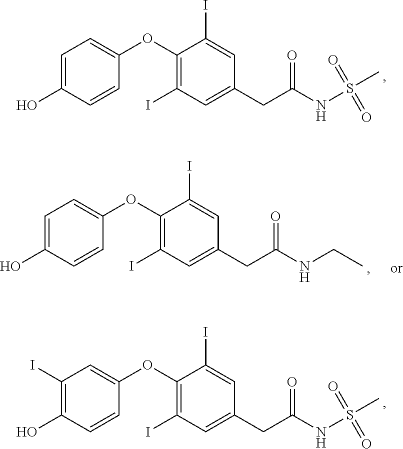 [SMILES: CS(=O)(=O)NC(=O)Cc1cc(I)c(Oc2ccc(O)cc2)c(I)c1 ; CCNC(=O)Cc1cc(I)c(Oc2ccc(O)cc2)c(I)c1 ; CS(=O)(=O)NC(=O)Cc1cc(I)c(Oc2ccc(O)c(I)c2)c(I)c1]

or a subgroup thereof.

In one aspect, a compound can be present as:

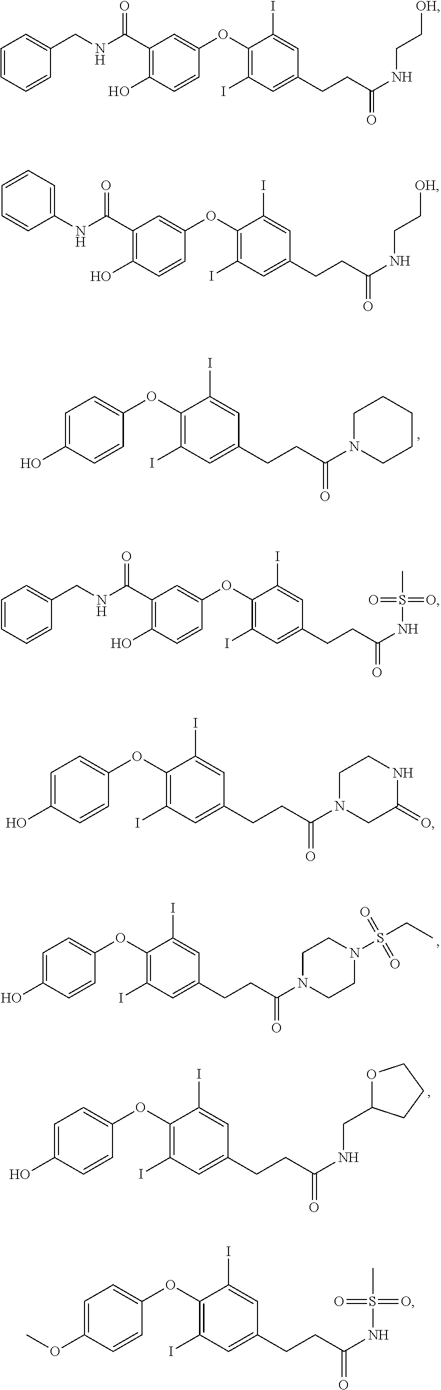

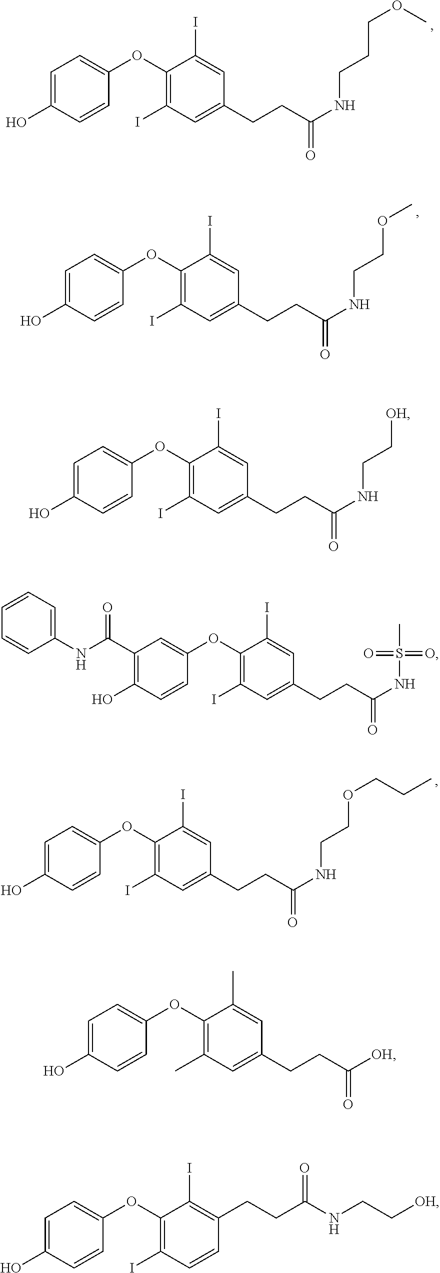

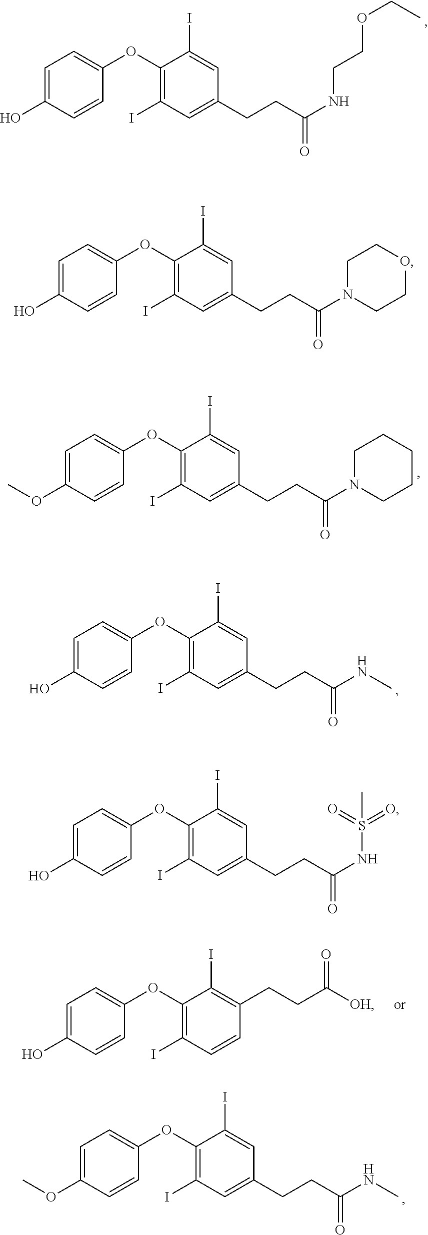

or a subgroup thereof.

In one aspect, a compound can be present as:

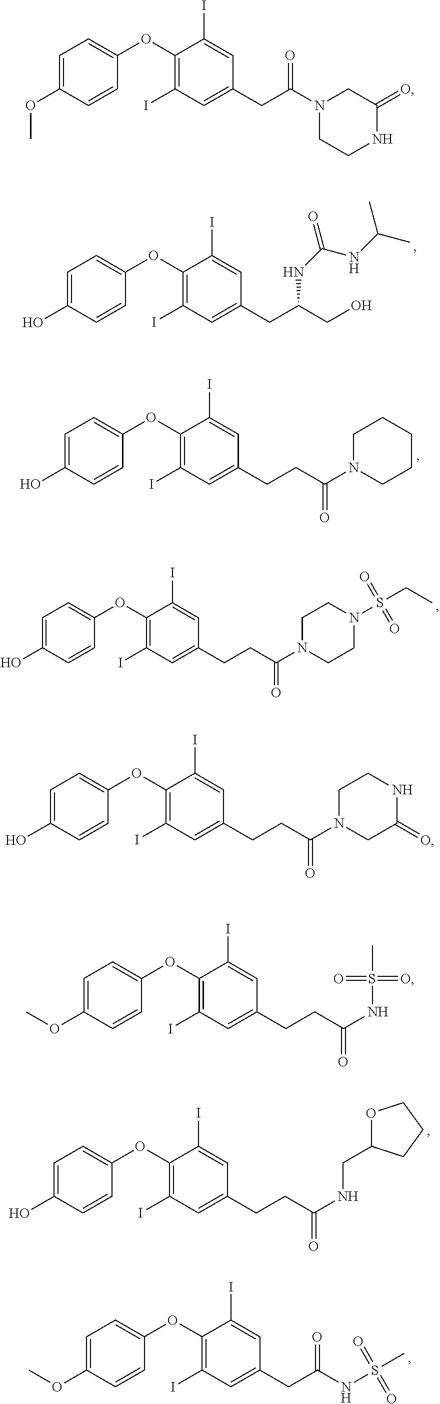

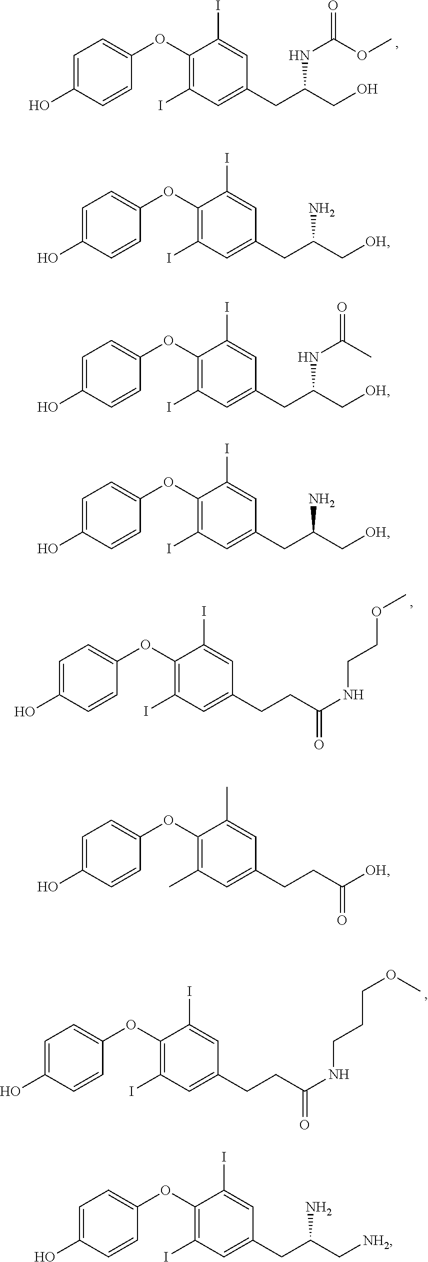

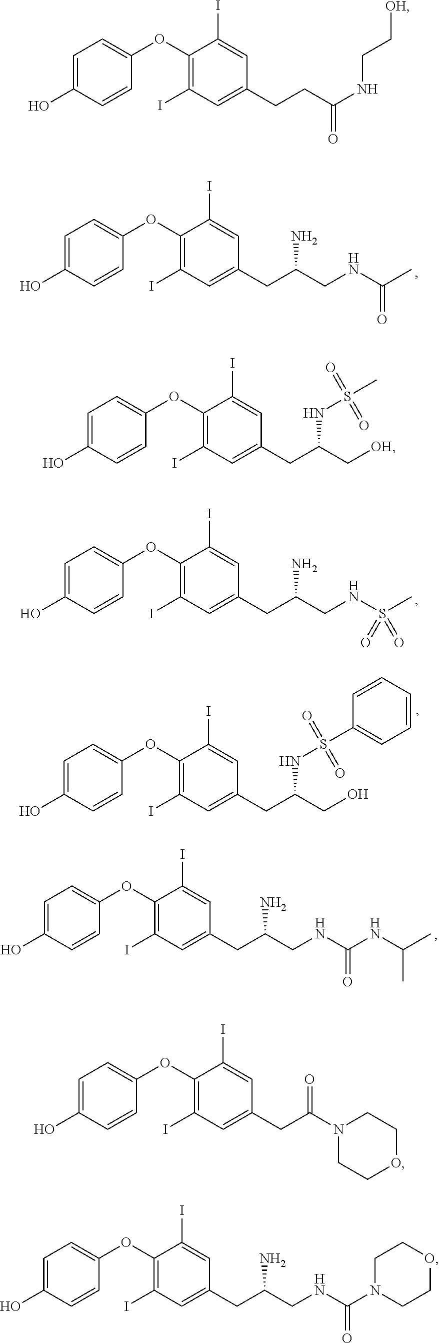

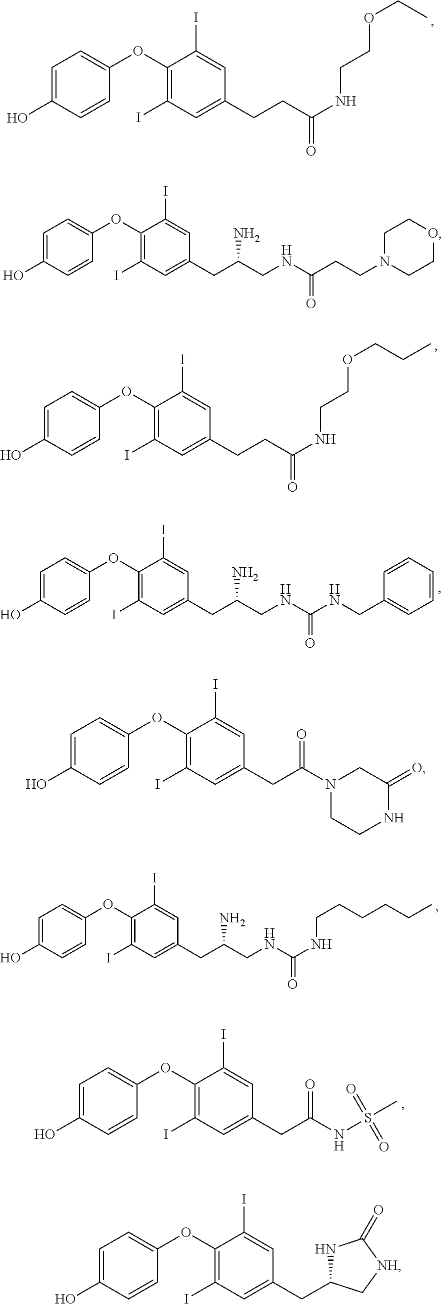

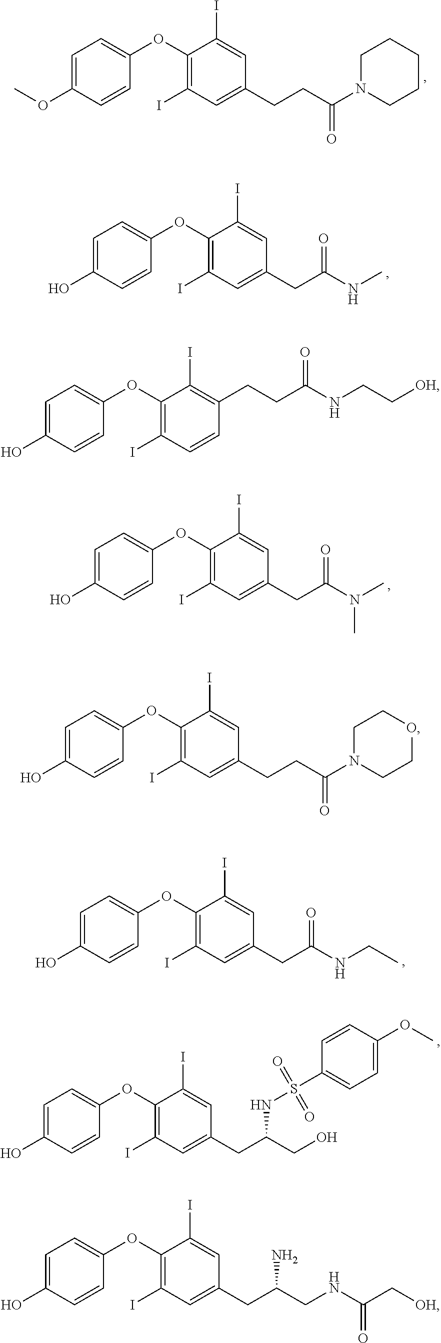

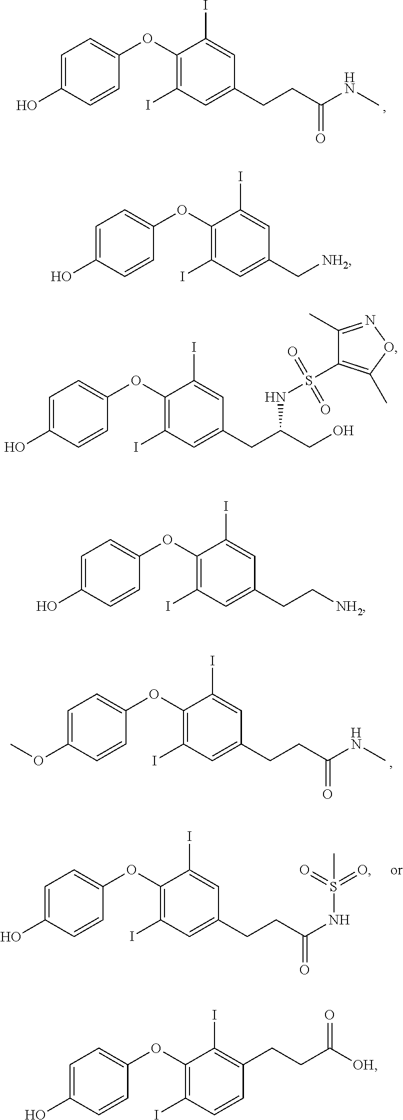

or a subgroup thereof.

In one aspect, a compound can be present as:

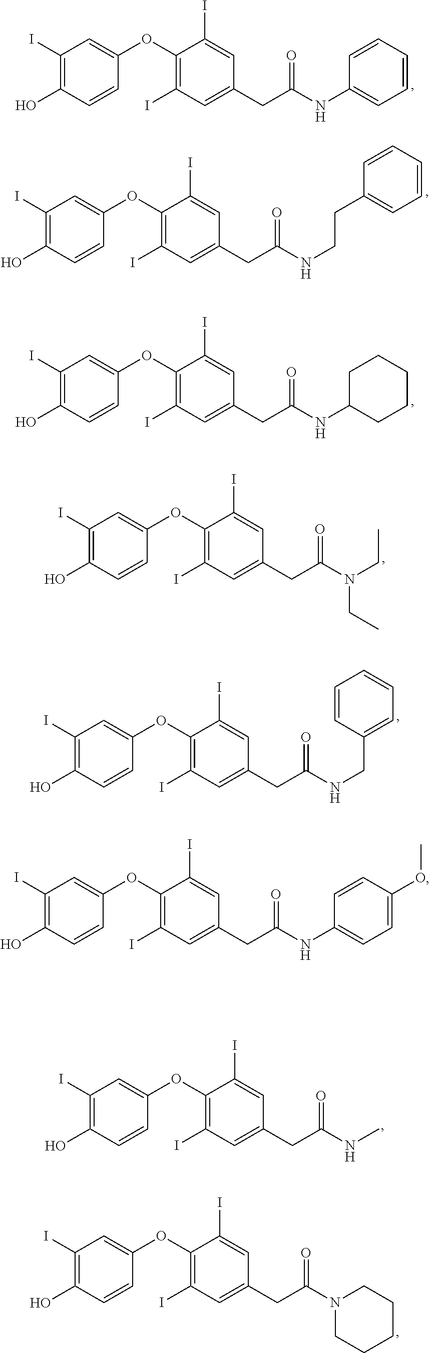

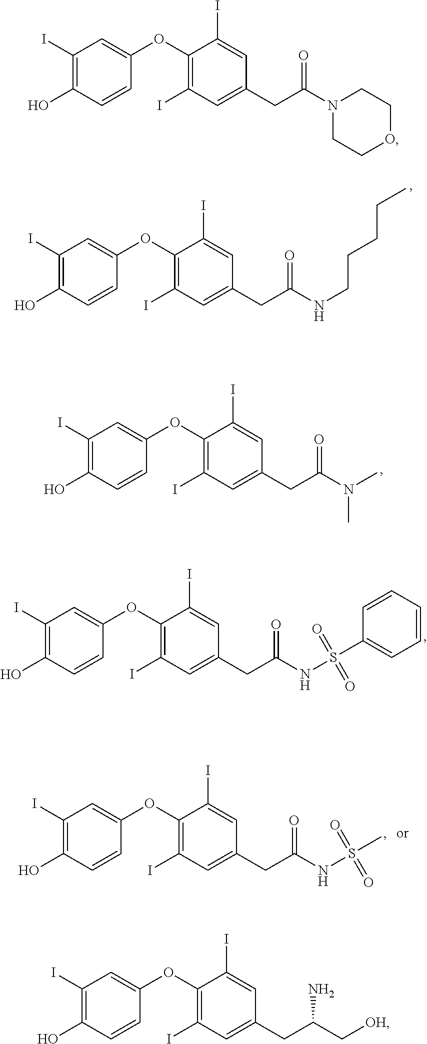 [SMILES: O=C(Cc1cc(I)c(Oc2ccc(O)c(I)c2)c(I)c1)N1CCOCC1 ; CCCCCNC(=O)Cc1cc(I)c(Oc2ccc(O)c(I)c2)c(I)c1 ; CN(C)C(=O)Cc1cc(I)c(Oc2ccc(O)c(I)c2)c(I)c1 ; O=C(Cc1cc(I)c(Oc2ccc(O)c(I)c2)c(I)c1)NS(=O)(=O)c1ccccc1 ; CS(=O)(=O)NC(=O)Cc1cc(I)c(Oc2ccc(O)c(I)c2)c(I)c1 ; N[C@H](CO)Cc1cc(I)c(Oc2ccc(O)c(I)c2)c(I)c1]

or a subgroup thereof.

In one aspect, a compound can be present as:

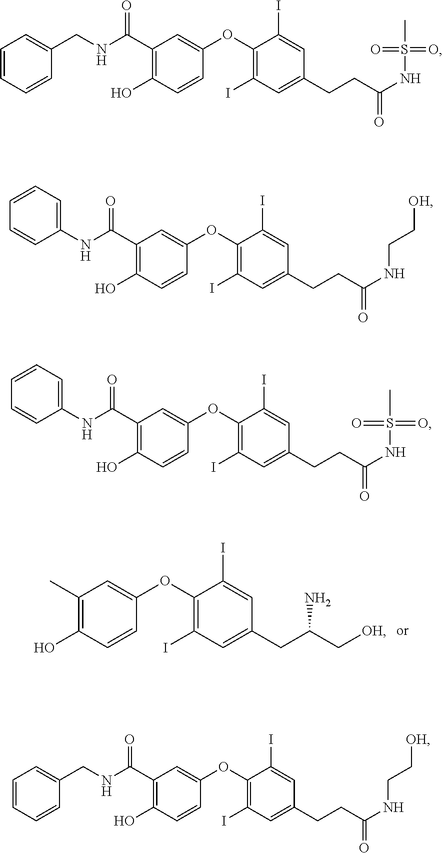 [SMILES: CS(=O)(=O)NC(=O)CCc1cc(I)c(Oc2ccc(O)c(C(=O)NCc3ccccc3)c2)c(I)c1 ; O=C(CCc1cc(I)c(Oc2ccc(O)c(C(=O)Nc3ccccc3)c2)c(I)c1)NCCO ; CS(=O)(=O)NC(=O)CCc1cc(I)c(Oc2ccc(O)c(C(=O)Nc3ccccc3)c2)c(I)c1 ; Cc1cc(Oc2c(I)cc(C[C@H](N)CO)cc2I)ccc1O ; O=C(CCc1cc(I)c(Oc2ccc(O)c(C(=O)NCc3ccccc3)c2)c(I)c1)NCCO]

or a subgroup thereof.

In one aspect, a compound can be present as:

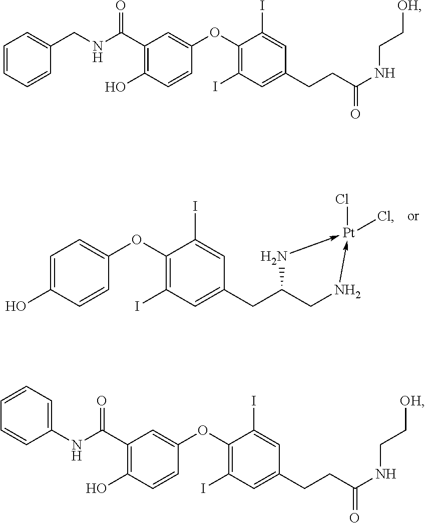

or a subgroup thereof.

In one aspect, a compound can be present as:

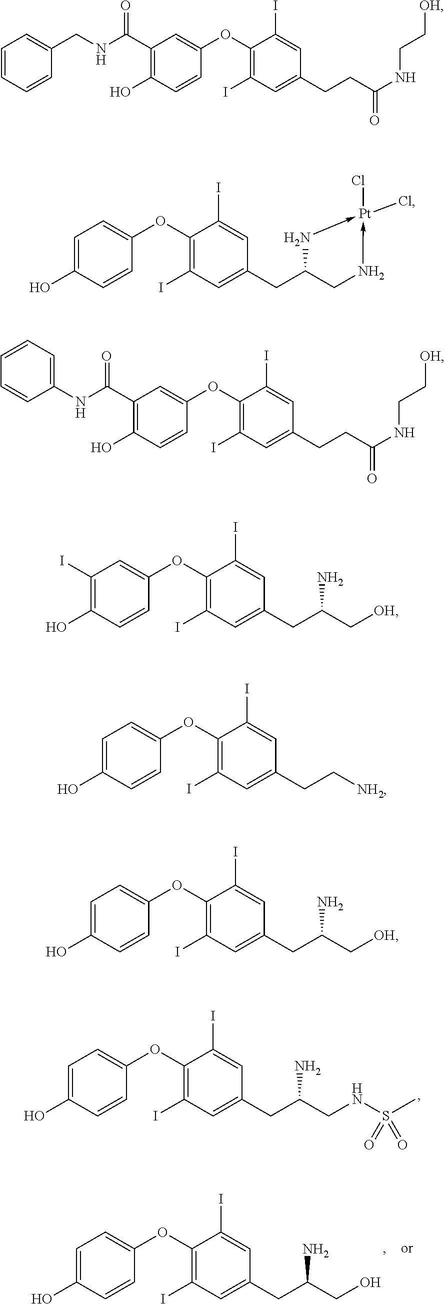

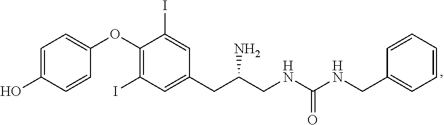 [SMILES: N[C@H](CNC(=O)NCc1ccccc1)Cc1cc(I)c(Oc2ccc(O)cc2)c(I)c1]

or a subgroup thereof.

In one aspect, a compound can be present as:

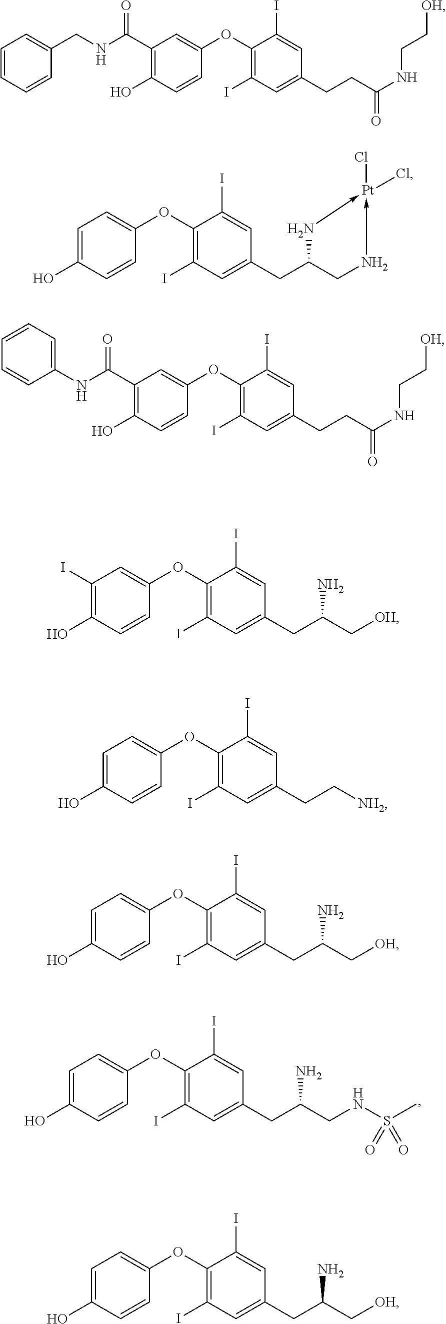

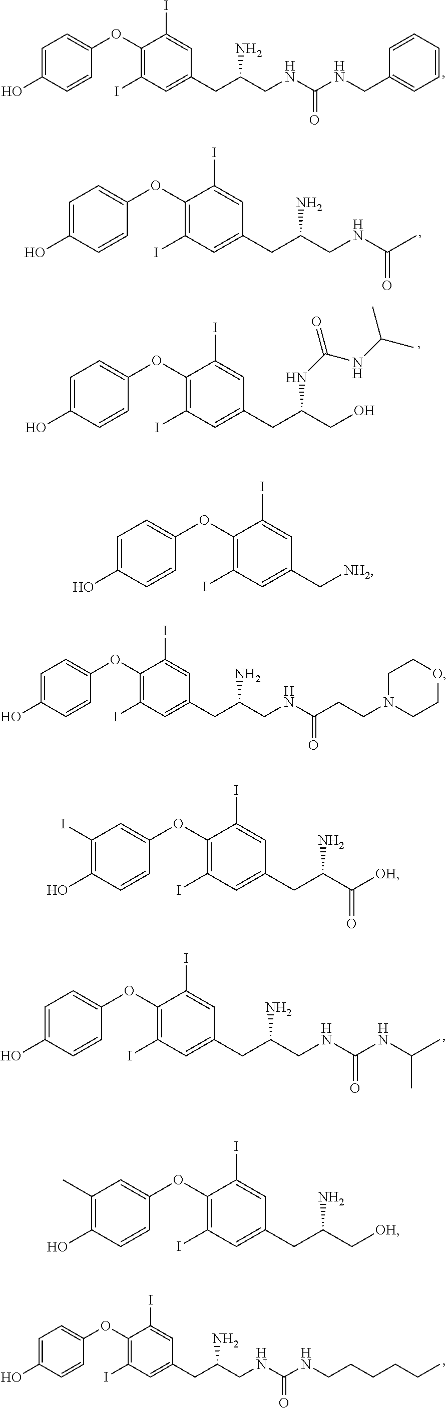

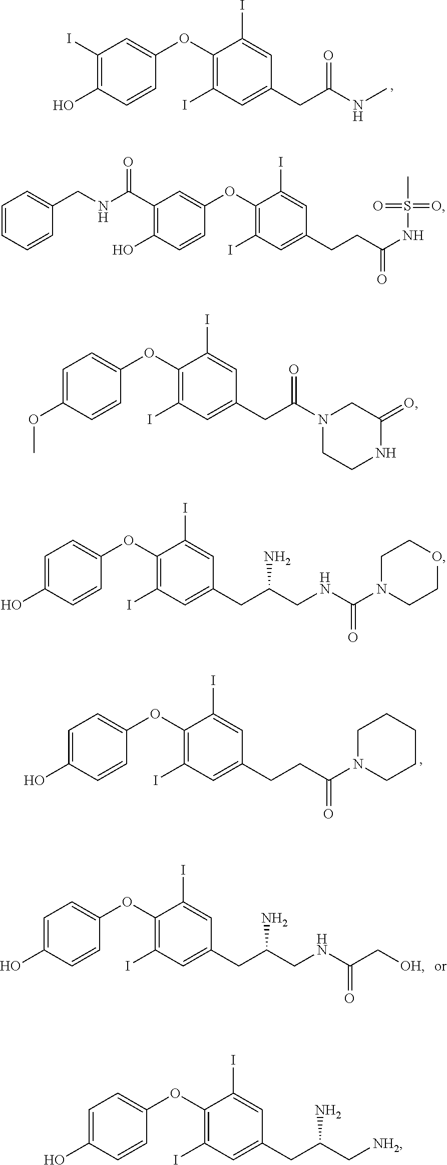

or a subgroup thereof.

In one aspect, a compound can be present as:

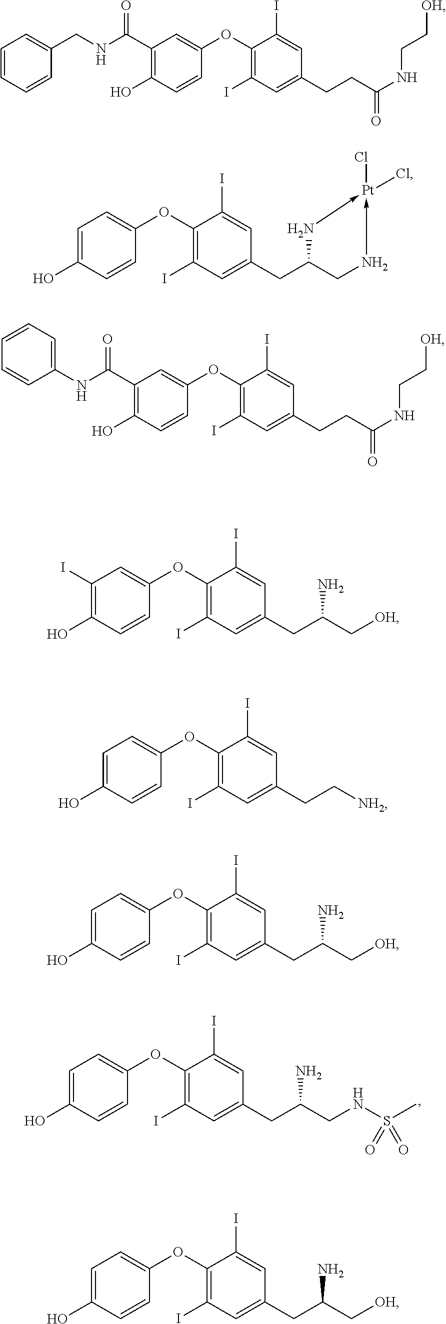

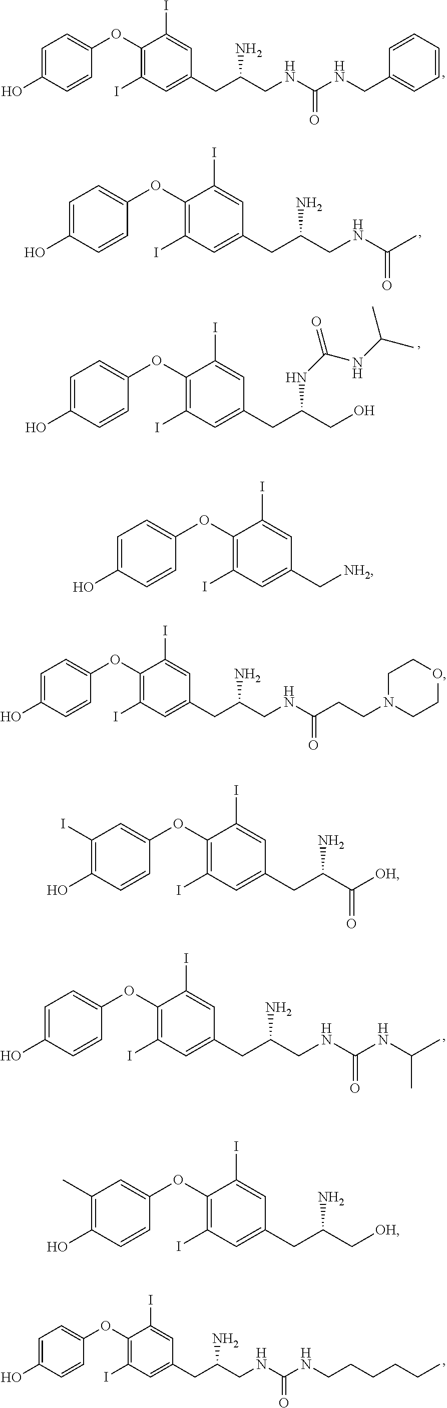

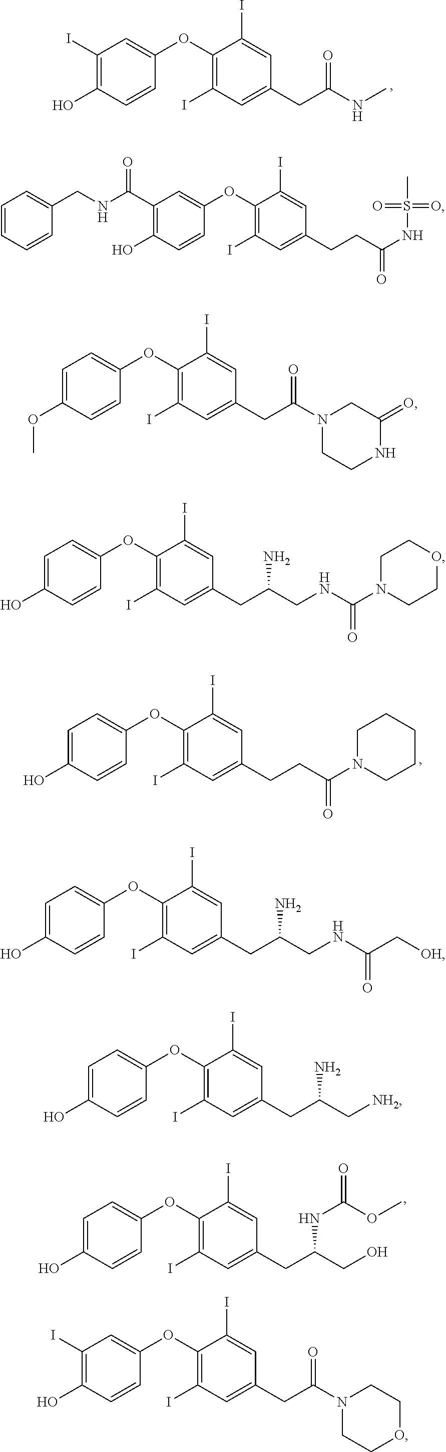

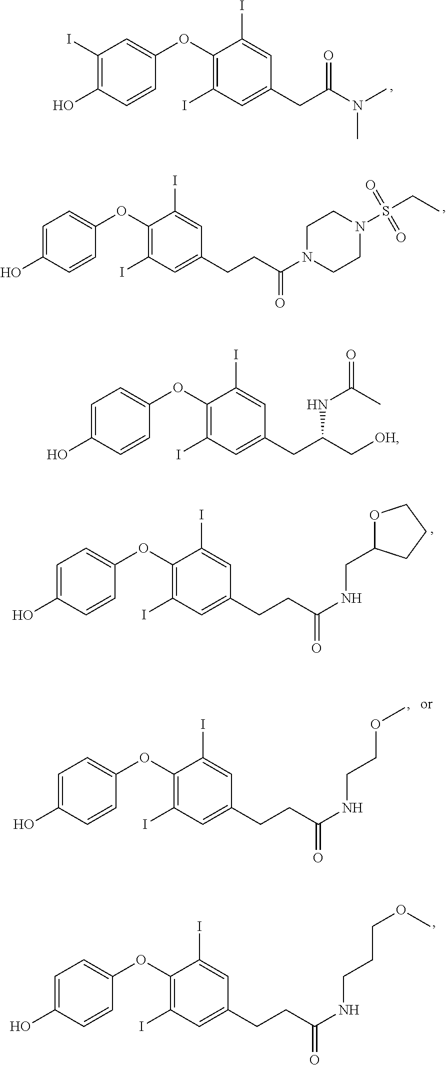 [SMILES: CN(C)C(=O)Cc1cc(I)c(Oc2ccc(O)c(I)c2)c(I)c1 ; CCS(=O)(=O)N1CCN(C(=O)CCc2cc(I)c(Oc3ccc(O)cc3)c(I)c2)CC1 ; CC(=O)N[C@H](CO)Cc1cc(I)c(Oc2ccc(O)cc2)c(I)c1 ; O=C(CCc1cc(I)c(Oc2ccc(O)cc2)c(I)c1)NCC1CCCO1 ; COCCNC(=O)CCc1cc(I)c(Oc2ccc(O)cc2)c(I)c1 ; COCCCNC(=O)CCc1cc(I)c(Oc2ccc(O)cc2)c(I)c1]

or a subgroup thereof.

In one aspect, a compound can be present as:

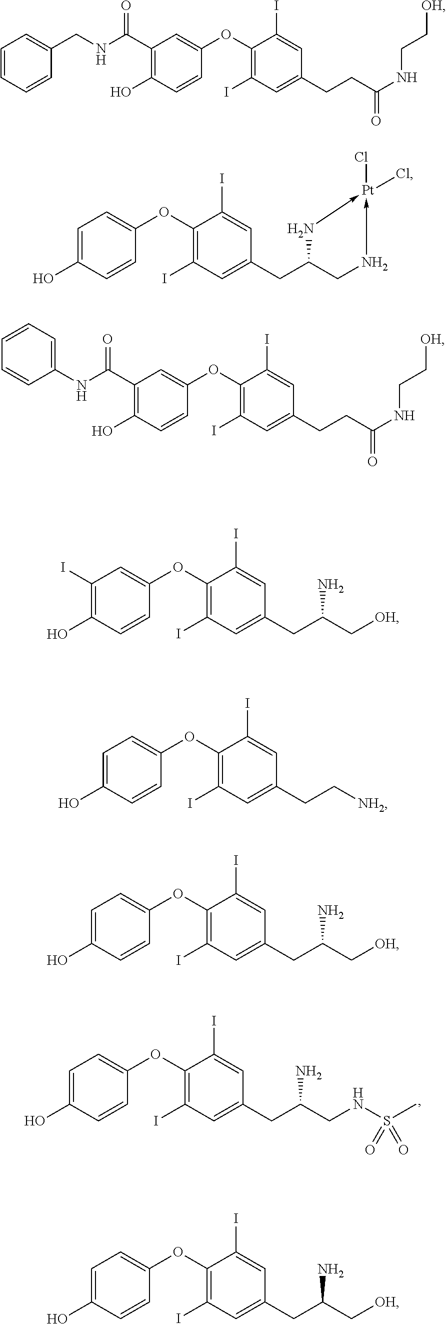

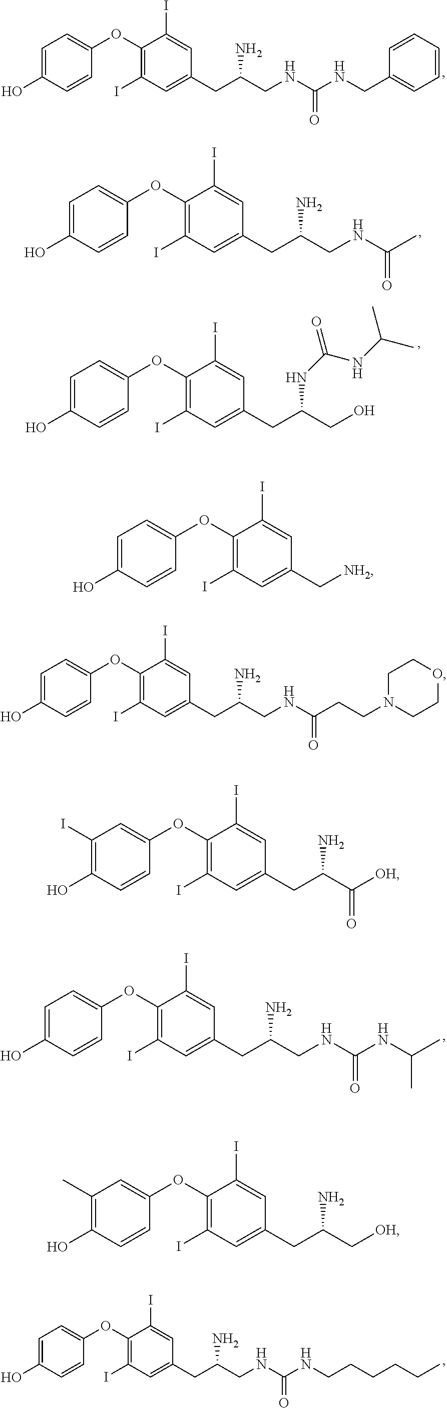

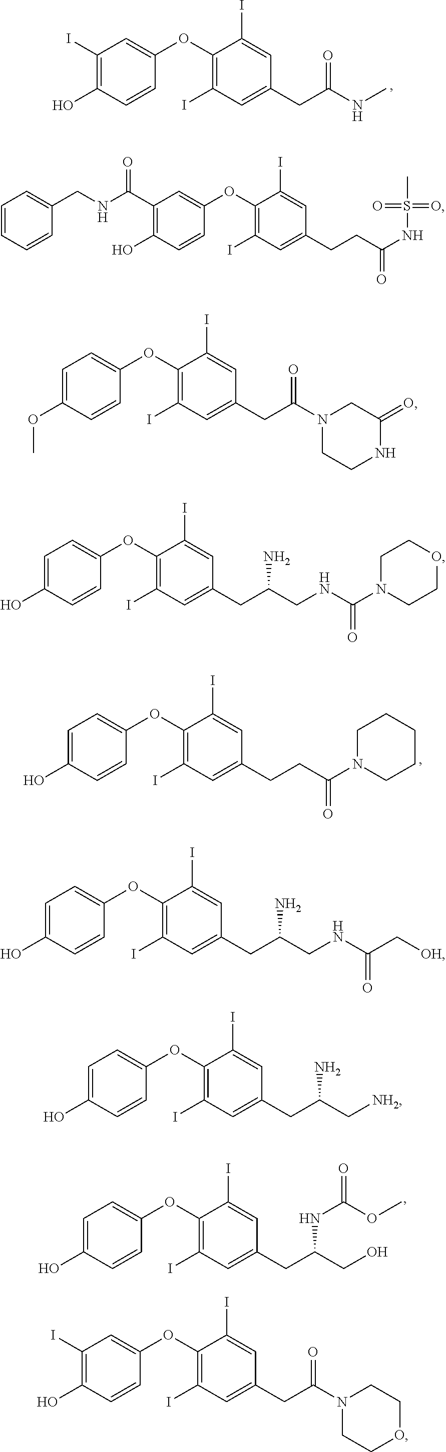

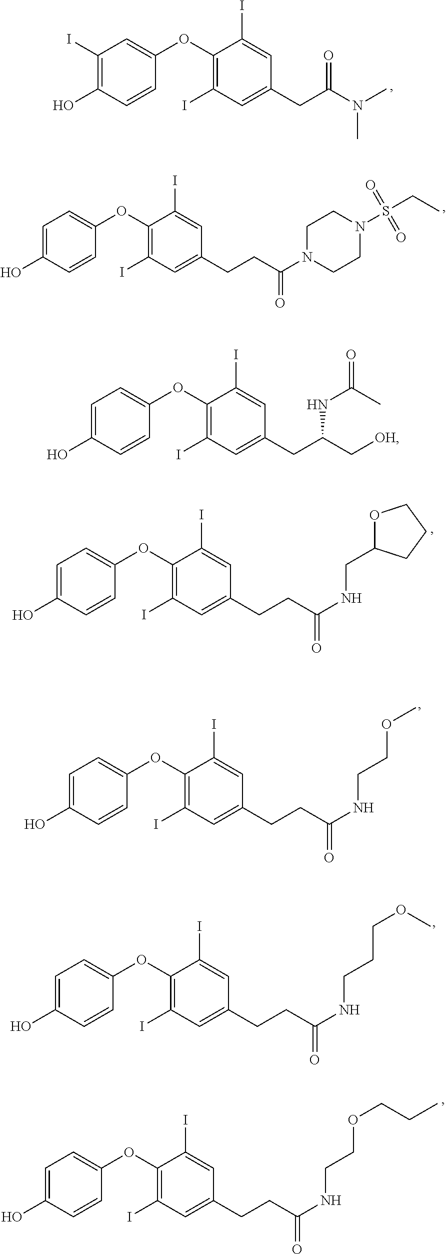

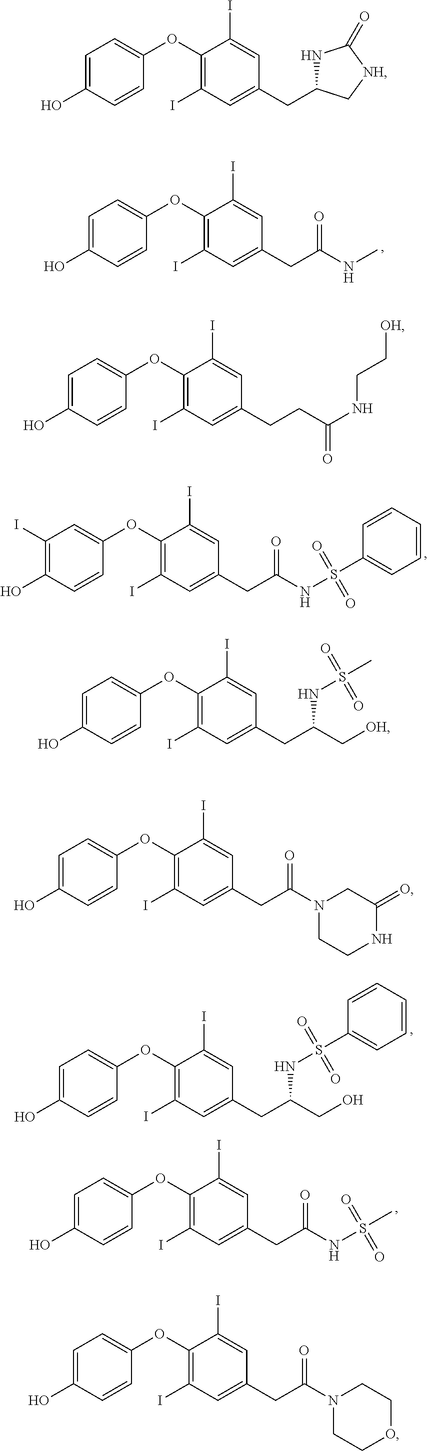

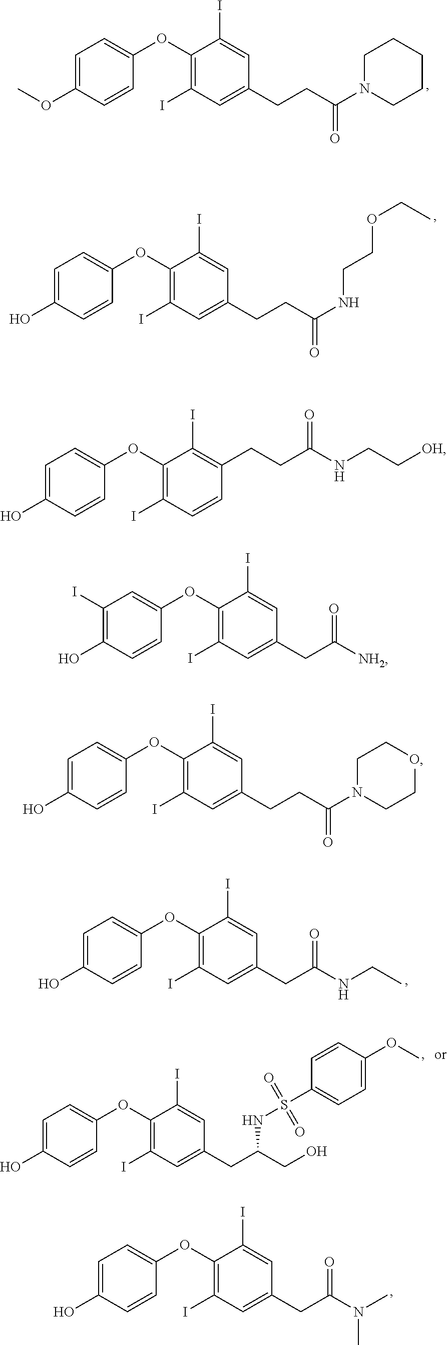

or a subgroup thereof.

In one aspect, a compound can be present as:

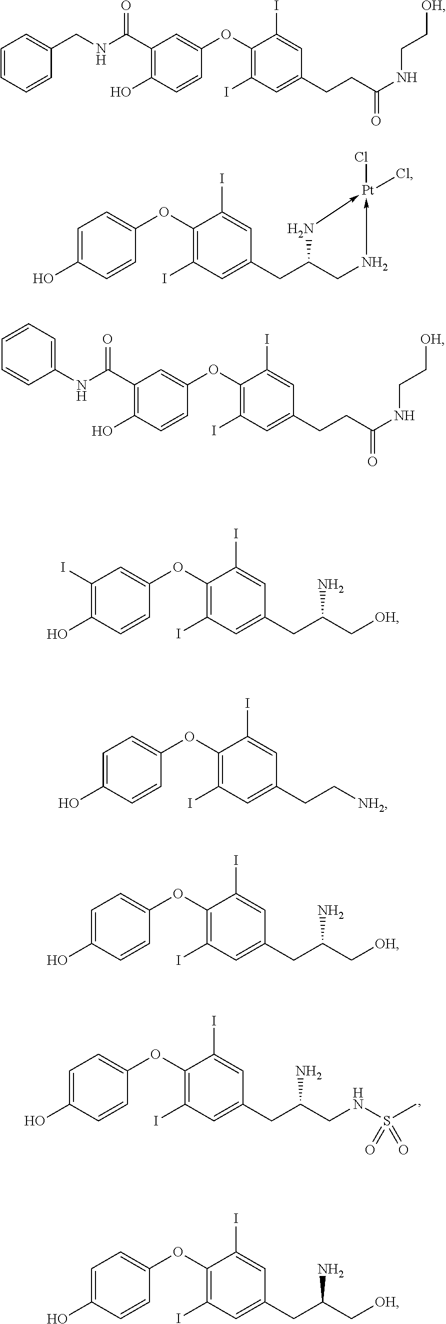

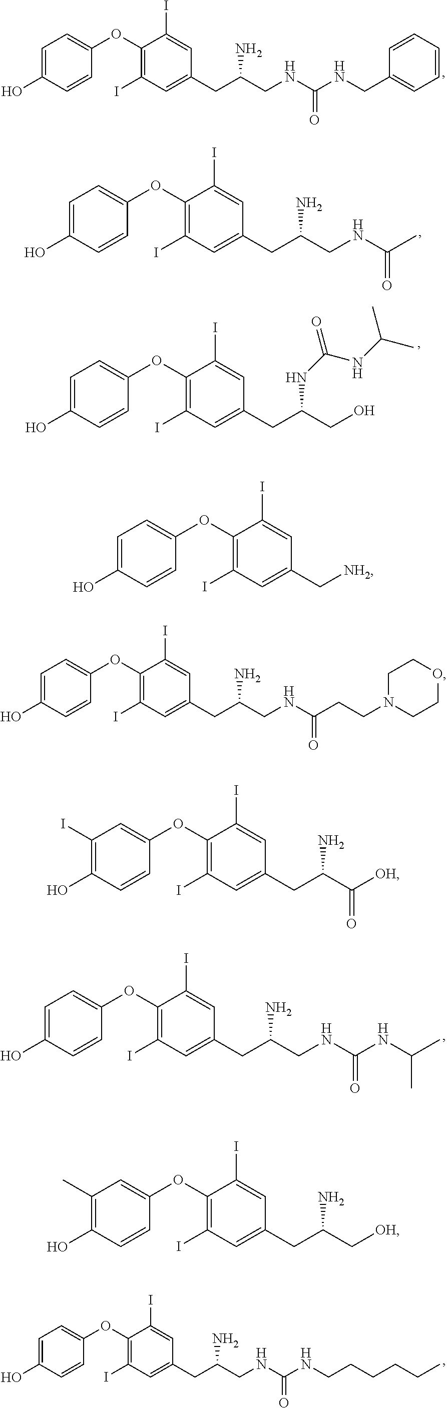

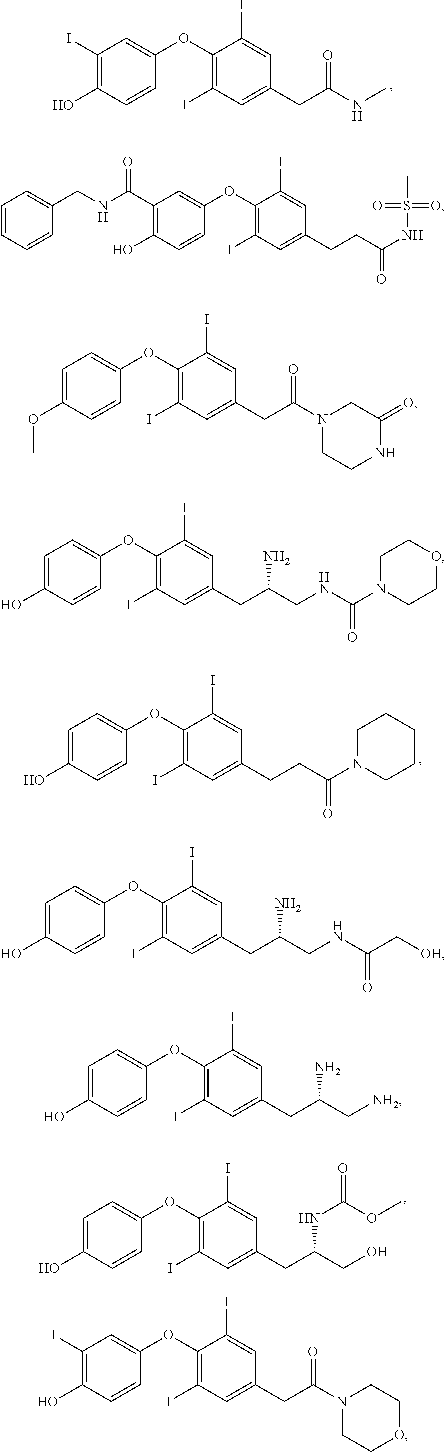

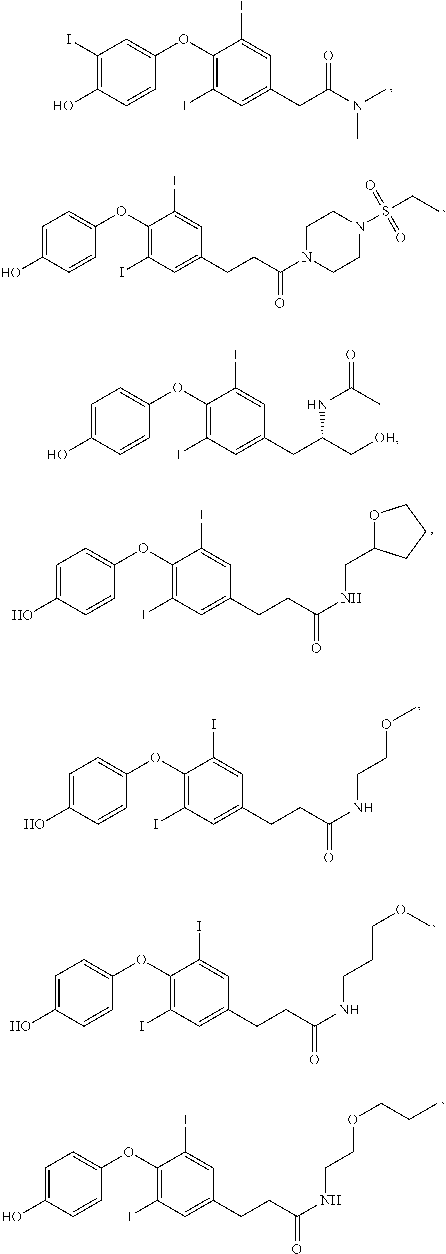

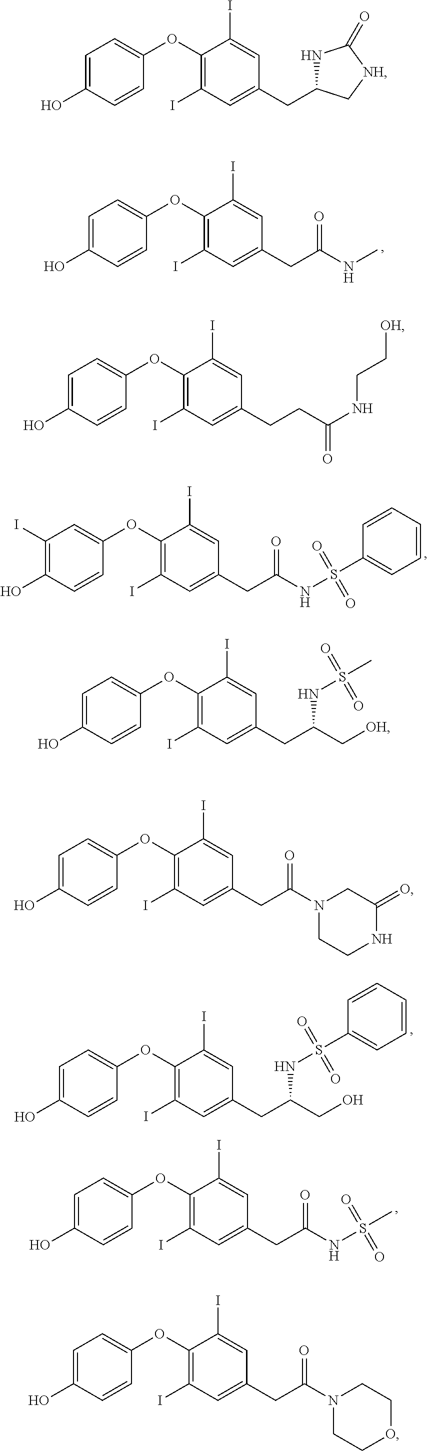

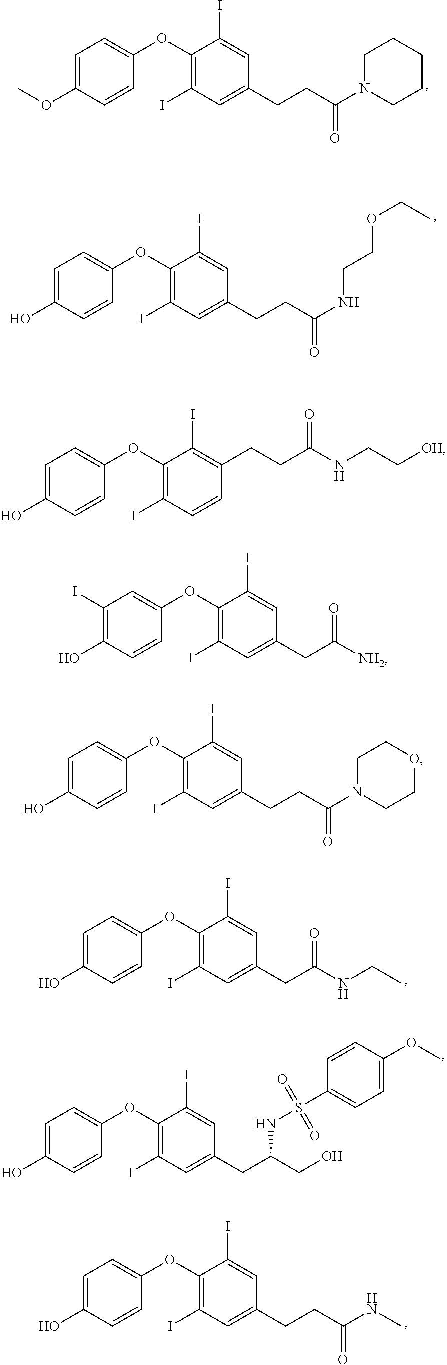

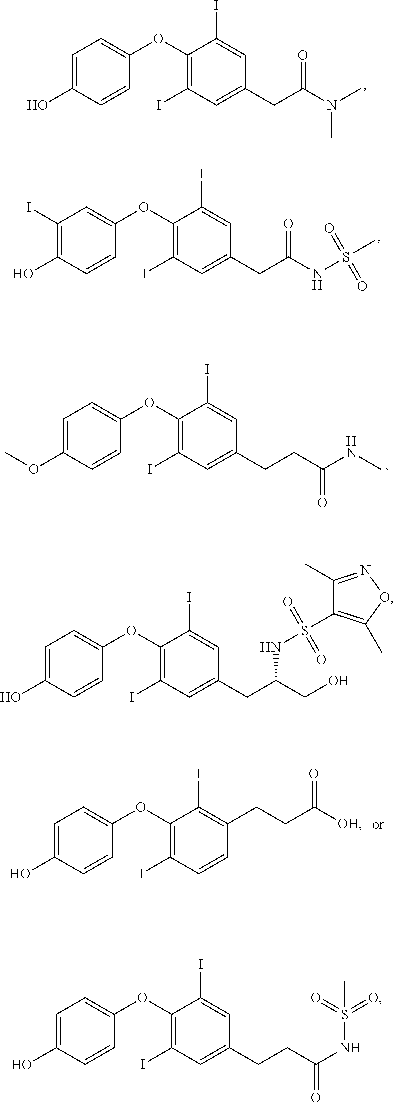

or a subgroup thereof.

In one aspect, a compound can be present as:

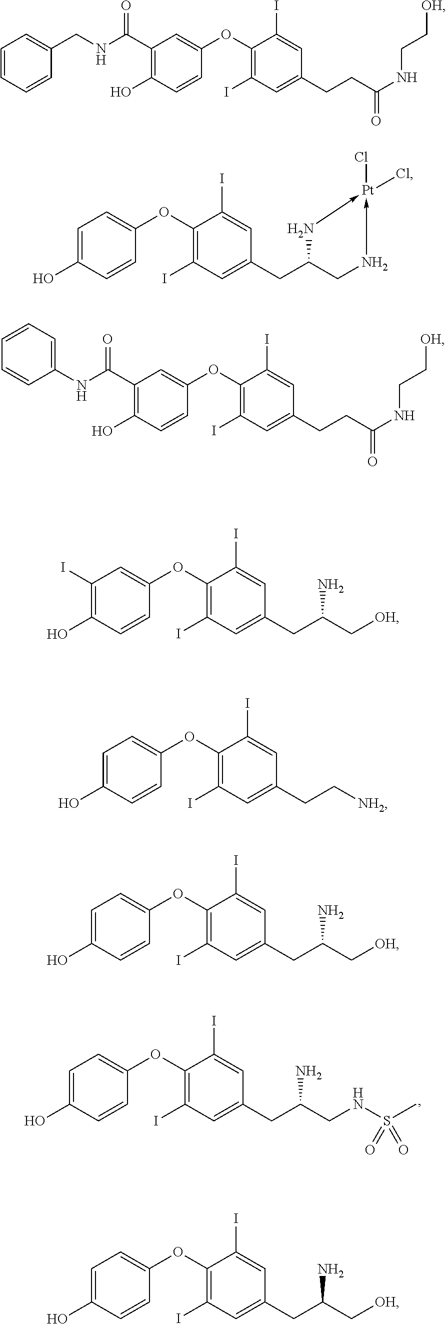

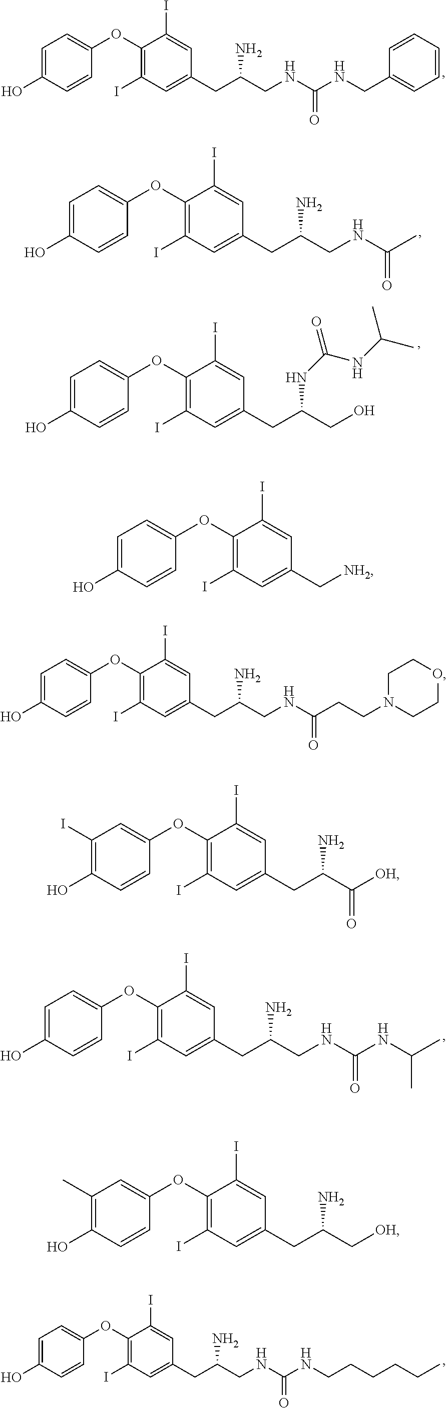

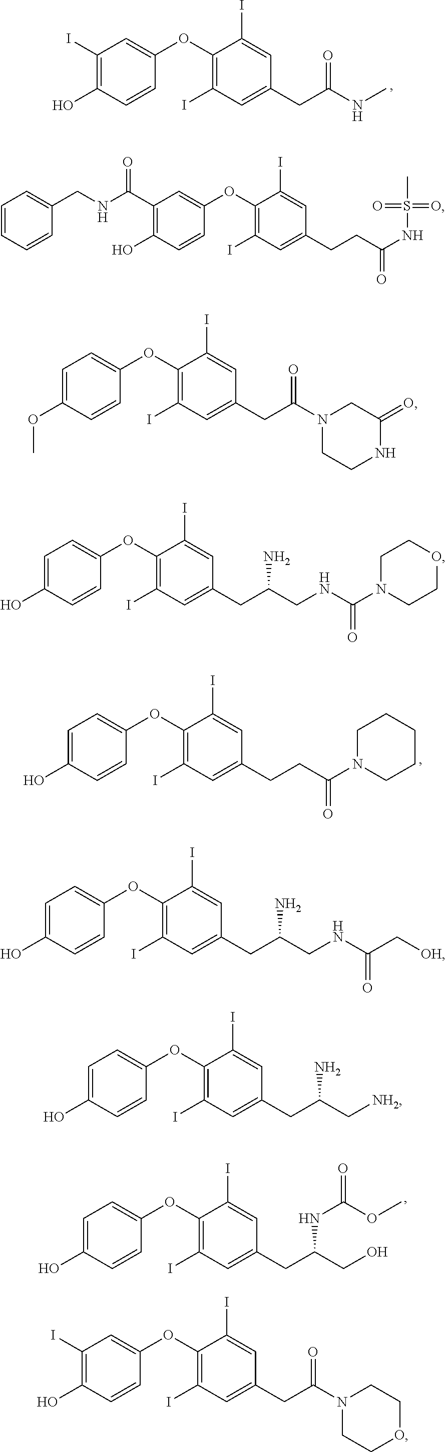

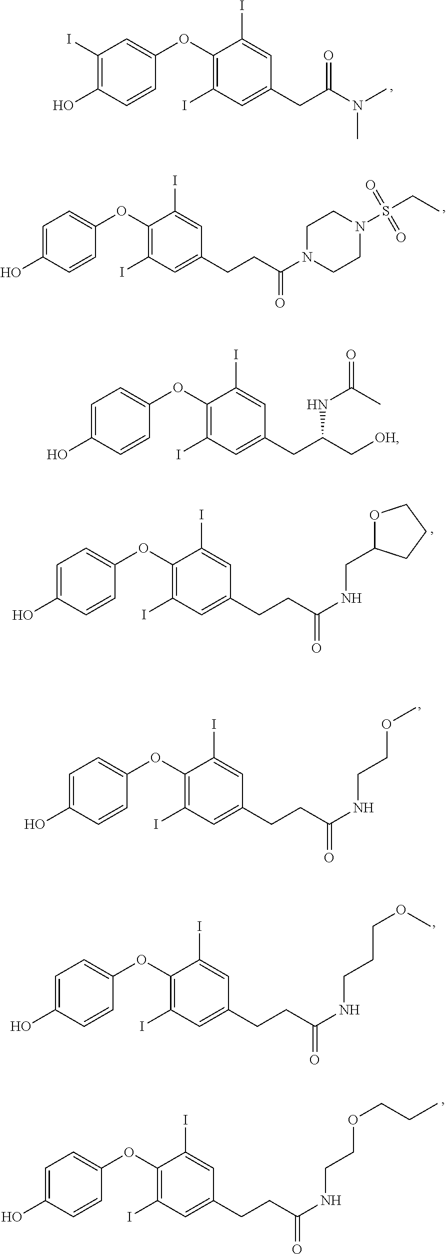

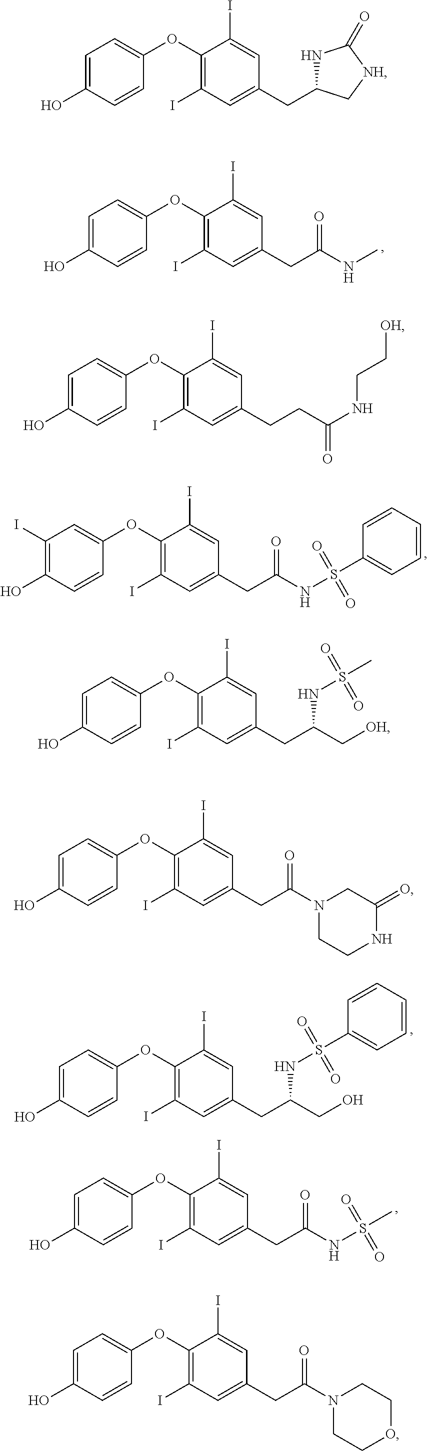

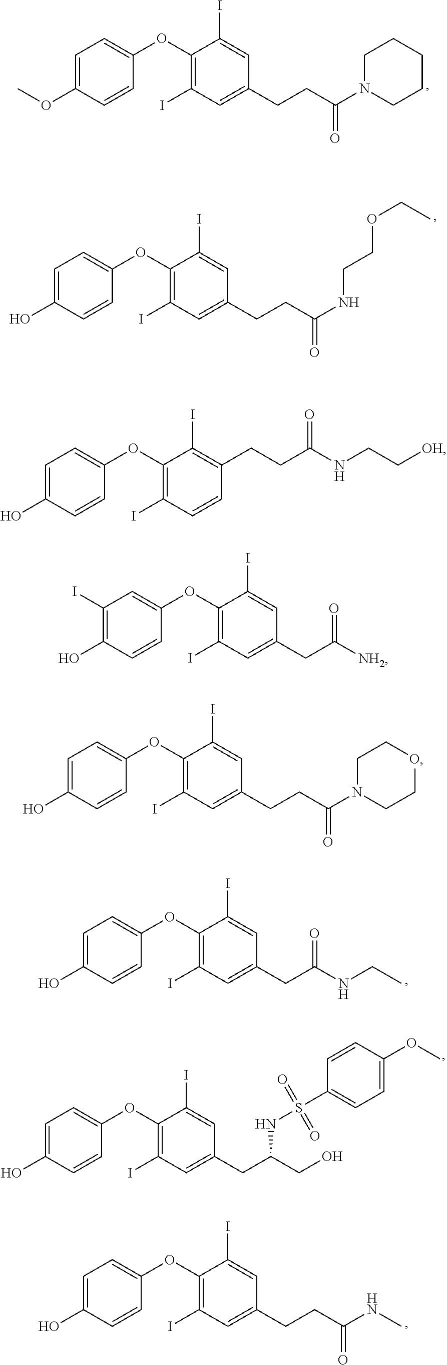

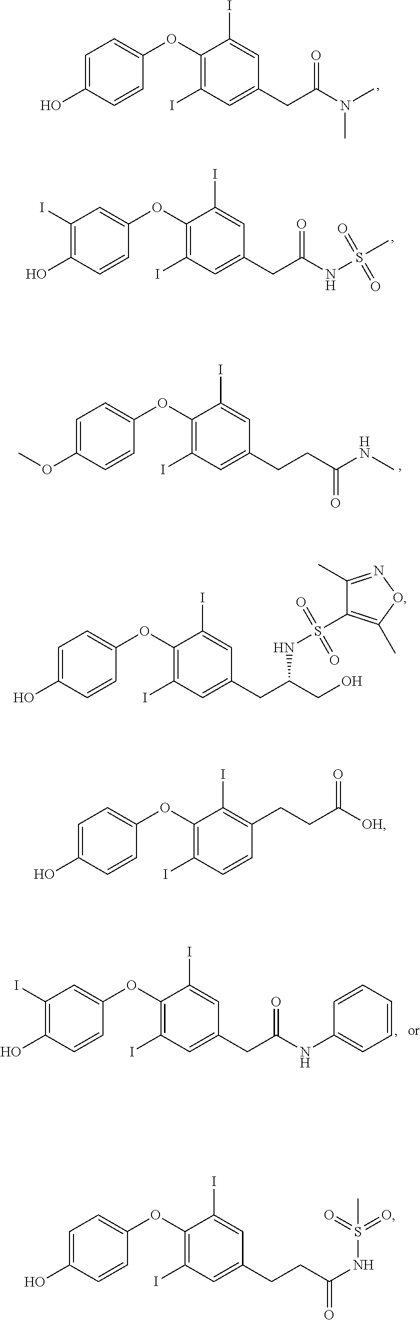

or a subgroup thereof.

In one aspect, a compound can be present as:

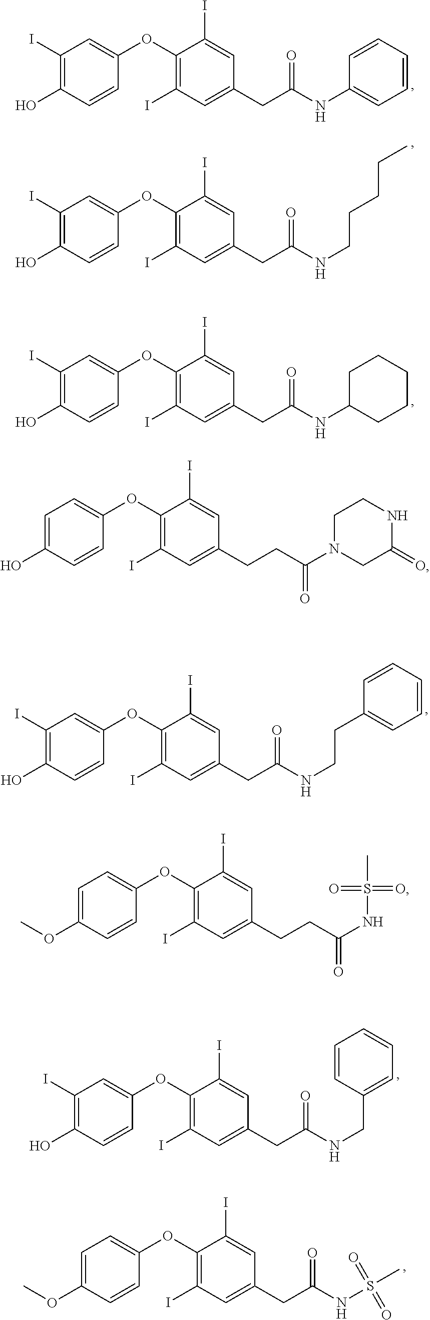

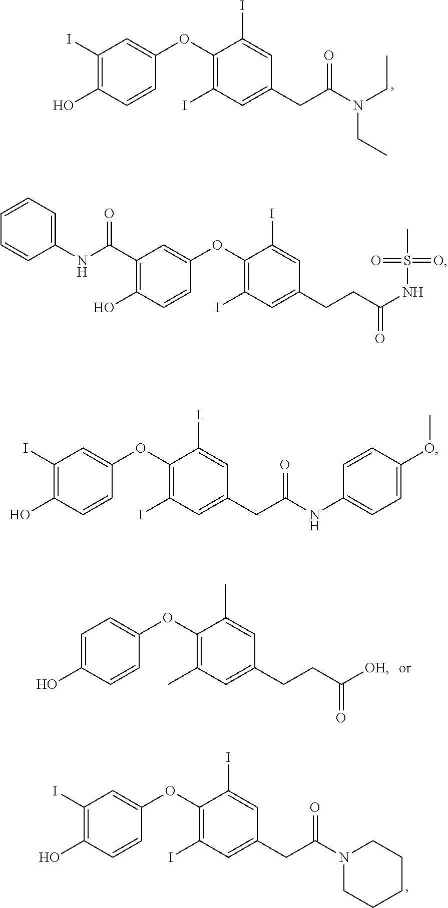 [SMILES: CCN(CC)C(=O)Cc1cc(I)c(Oc2ccc(O)c(I)c2)c(I)c1 ; CS(=O)(=O)NC(=O)CCc1cc(I)c(Oc2ccc(O)c(C(=O)Nc3ccccc3)c2)c(I)c1 ; COc1ccc(NC(=O)Cc2cc(I)c(Oc3ccc(O)c(I)c3)c(I)c2)cc1 ; Cc1cc(CCC(=O)O)cc(C)c1Oc1ccc(O)cc1 ; O=C(Cc1cc(I)c(Oc2ccc(O)c(I)c2)c(I)c1)N1CCCCC1]

or a subgroup thereof.

It is understood that the disclosed compounds can be used in connection with the disclosed methods, compositions, kits, and uses.

The pharmaceutical acceptable derivatives of the compounds can include any suitable derivative, such as pharmaceutically acceptable salts as discussed below, isomers, radiolabeled analogs, tautomers, and the like.

In a further aspect, the disclosed compounds are inhibitors of PCNA protein activity.

3. Modulation of PCNA Activity

In one aspect, the disclosed compounds exhibit inhibition PCNA protein activity. In a yet further aspect, the disclosed compounds exhibit selective inhibition of PCNA protein activity compared to T3 hormone receptor. In a still further aspect, the disclosed compounds exhibit inhibition of PCNA interaction with proteins involved with DNA replication. In a yet further aspect, the disclosed compounds exhibit disruption of preformed or existing PCNA protein complexes. In a still further aspect, the disclosed compounds exhibit binding to the domain of PCNA that binds to PIP-box proteins. In an even further aspect, the disclosed compounds inhibit binding of PIP-box proteins to PCNA.\

Inhibition of PCNA activity can be determined by a variety of in vitro and in vivo methods known to one skilled in the art. For example, inhibition of PCNA protein activity can be determined using a primer extension assay using a damaged oligonucleotide template (“translesion DNA synthesis assay”). In one aspect, the disclosed compounds exhibit inhibition of PCNA protein activity with an IC₅₀ in a translesion DNA synthesis assay of less than about 100 μM, less than about 50 μM, less than about 10 μM, less than about 1 μM, less than about 500 nM, or of less than about 100 nM.

In one aspect, the disclosed compounds exhibit inhibition of Pogo-ligase (“PL”) peptide binding to PCNA. In a further aspect, the PL peptide has the sequence represented by the amino acid sequence SAVLQKKITDYFHPKK. In a yet further aspect, the PL peptide is linked to a fluorescent reporter molecule. In a still further aspect, the fluorescent reporter molecule is 5-carboxyfluorescein. In an even further aspect, the inhibition of binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 1 μM, less than about 500 nM, or of less than about 100 nM.

In one aspect, the disclosed compounds exhibit inhibition of p21 protein binding to PCNA. In it is understood that p21, as used herein, refers to the cyclin-dependent kinase inhibitor 1A, also referred to as Cip1. In a further aspect, the p21 is a full-length p21 protein. In a yet further aspect, binding of p21 to PCNA is determined using a pull-down competition assay. In a yet further aspect, the IC₅₀ for inhibition of p21 to PCNA is less than about 100 μM, less than about 50 μM, less than about 10 μM, less than about 1 μM, less than about 500 nM, or of less than about 100 nM.

In one aspect, the disclosed compounds are selective for PCNA. In a further aspect, the disclosed compounds exhibit preferential inhibition of PCNA activity compared to T3 hormone receptor. In still various aspects, the disclosed compounds show little apparent activity in an in vitro T3 hormone receptor transcription activation assay (“T3 transcription activation assay.” In various further aspects, the disclosed compounds have an EC₅₀ more than about 10 times the EC₅₀ of T3 in a T3 transcription activation assay. In a still further aspect, the disclosed compounds have an EC₅₀ more than about 50 times the EC₅₀ of T3 in a T3 transcription activation assay. In a yet further aspect, the disclosed compounds have an EC₅₀ more than about 100 times the EC₅₀ of T3 in a T3 transcription activation assay. In an even further aspect, the disclosed compounds have an EC₅₀ more than about 500 times the EC₅₀ of T3 in a T3 transcription activation assay. In a still further aspect, the disclosed compounds have an EC₅₀ more than about 1,000 times the EC₅₀ of T3 in a T3 transcription activation assay. In a yet further aspect, the disclosed compounds have an EC₅₀ more than about 5,000 times the EC₅₀ of T3 in a T3 transcription activation assay.

Alternatively, the inhibition of PCNA protein activity can be determined in a cell-based assay. There are a variety of cell-based assays that are suitable for determination of inhibition of PCNA protein activity known to one skilled in the art. In a yet further aspect, the activity of the disclosed compound is determined in a cell-line selected from a cell-line derived from breast cancer, prostate cancer, pancreatic cancer, lung cancer, and a gastrointestinal cancer. In a still further aspect, the activity of the disclosed compound is determined in the HeLa cell-line.

In one aspect, the disclosed compounds exhibit inhibition of cell growth. In a further aspect, the disclosed compounds exhibit inhibition of HeLa cell growth. There are various methods available to one skilled in the art to measure inhibition of cell growth using cultured cells. Such methods include measurement of incorporation of ³H-thymidine or measurement of cellular DNA synthesis using BrdU. For example, a compound can exhibit inhibition of cell growth with an IC₅₀ of less than about 500 μM, of less than about 250 μM, of less than about 100 μM, of less than about 50 μM, less than about 10 μM, less than about 1 μM, less than about 500 nM, or of less than about 100 nM.

In one aspect, the disclosed compounds exhibit cell cycle arrest. In a yet further aspect, cell cycle arrest is determined using flow cytometry. In a still further aspect, cell cycle arrest is determined using HeLa cells. In an even further aspect, the disclosed compounds exhibit cell cycle arrest at S-phase. In a yet further aspect, the IC₅₀ for inhibition of the cell cycle is less than about 100 μM, less than about 50 μM, less than about 10 μM, less than about 1 μM, less than about 500 nM, or of less than about 100 nM.

In one aspect, the disclosed compounds exhibit activity as sensitizers which enhance the action of chemotherapeutic agents. In a further aspect, the disclosed compounds decrease the IC₅₀ of a chemotherapeutic agent by about two-fold compared to the IC₅₀ of the chemotherapeutic agent in the absence of a disclosed compound. In a still further aspect, the disclosed compounds decrease the IC₅₀ of a chemotherapeutic agent by about five-fold, by about 10-fold, by about 50-fold, or by about 100-fold. In a yet further aspect, the chemotherapeutic agent is cisplatin. In an even further aspect, the effect of a disclosed compound on the activity of a chemotherapeutic agent is determined in an assay selected from a cell-viability assay, cell-growth inhibition assay, a cell apoptosis assay, and a cellular DNA synthesis assay. In a still further aspect, the assay is carried out us a cell-line. In a yet further aspect, the cell-line is the HeLa cell-line.

The in vivo efficacy for disclosed compounds can be measured in various animal models of hyperproliferative and proliferative disorders. The use of such models is would be known to one skilled in the art. For example, suitable rodent models, e.g. tumor xenograft models using nude mice, are frequently used to carry-out pre-clinical assessment of the effectiveness of novel agents for the treatment of cancer. Alternatively, there are established animal models of restenosis, a disorder that can be characterized as a proliferative disorder. A restenosis suitable model to assess the in vivo effectiveness of the disclosed compounds is the balloon injury model, which can be carried out in rodents, rabbits and pigs. For example, disclosed compounds can inhibit tumor growth in a subcutaneous tumor xenograft model at doses ranging from about 1 to about 200 mg/kg by i.v. administration, from about 10 to about 200 mg/kg by i.v. administration, from 1 to about 100 mg/kg by i.v. administration, from 1 to about 50 mg/kg by i.v. administration, from about 0.1 to about 100 mg/kg by i.v. administration, or from 0.1 to about 50 mg/kg by i.v. administration.

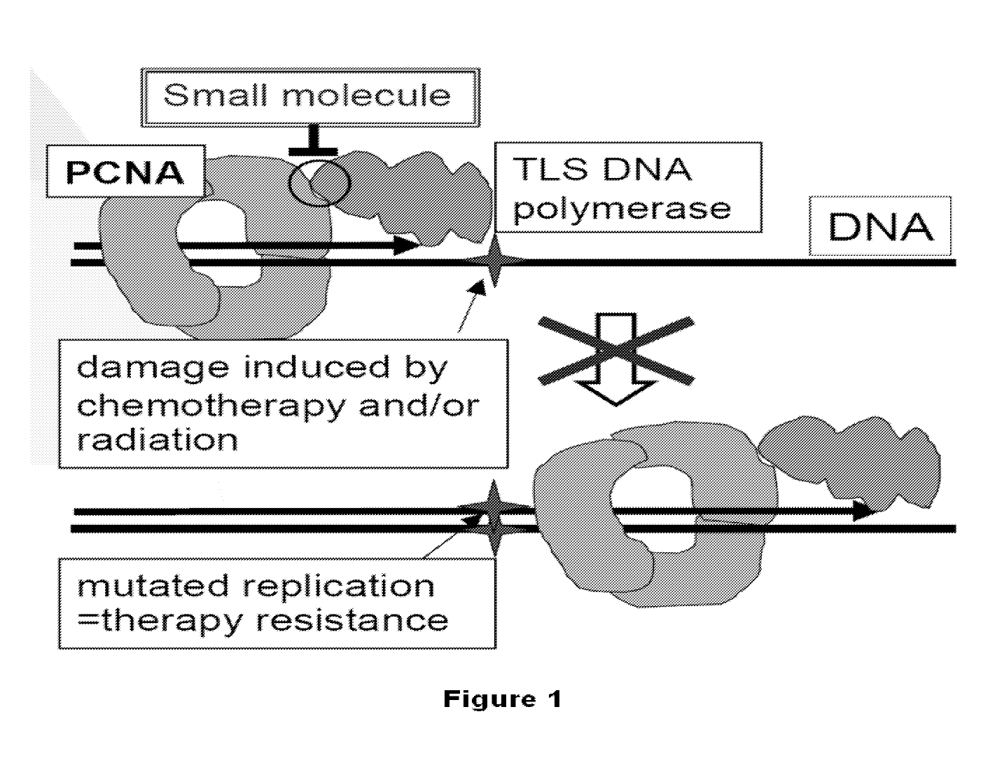

Without wishing to be bound by a particular theory, the disclosed compounds can increase the efficacy of chemotherapeutic agents by mechanism schematically shown in FIG. 1. Thus, in one aspect, the disclosed compounds can bind to PCNA a site or sites that interfere with the binding of the protein to a translesion DNA polymerase. In a further aspect, the translesion DNA polymerase is acting to repair cellular DNA damage caused by the chemotherapeutic agent. In the absence of a disclosed compound, the interaction of PCNA and the translesion DNA polymerase can allow DNA replication to occur. The occurrence of translesion DNA, which is requires PCNA, allows cells to replicate DNA even in the presence of DNA damage caused by a chemotherapeutic agent and escape cell death. Without wishing to be bound by a particular theory, the binding of a disclosed compound to PCNA results in the inability of a translesion DNA polymerase to carry-out synthesis through damage induced by a chemotherapeutic agent. In the context of treating a proliferative disorder such as a cancer, this effect can inhibit cell replication. In various aspects, the disruption of translesion DNA synthesis to occur can also suppress the ability of the cell to escape cell death and thus increase the sensitivity of a cell to a chemotherapeutic agent. In a further aspect, the disclosed compounds through inhibition of PCNA protein-protein actions such as those described above can also increase the efficacy of radiation therapy.

### C. PCNA PROTEIN

Proliferating cell nuclear antigen, or PCNA, is a non-histone nuclear protein and is an essential protein involved in DNA replication and repair. The protein has a basal isoelectric point of 4.57. The sequence of PCNA is well conserved between plants and animals, indicating a strong selective pressure for structure conservation, and suggesting that this type of DNA replication mechanism is conserved throughout eukaryotes (Suzuka I, et al., Eur. J. Biochem. (1991) 195, 571-5). PCNA stimulates the activity DNA polymerase δ, a DNA polymerase critical to leading strand DNA synthesis in eukaryotic cells. PCNA increases the processivity of DNA polymerase δ by acting as a clamp that slides along the DNA template and helps to keep DNA polymerase δ associated with the template strand. PCNA associates with a variety of proteins which are either directly or indirectly involved in DNA replication, DNA repair, or in the regulation of these critical cellular processes. The subcellular location for the protein is believed to be primarily in the nucleus, and generally the protein is believed to exist in the cell as a homotrimer. In response to DNA damage, this protein is ubiquitinated and is involved in the RAD6-dependent DNA repair pathway. Two transcript variants encoding the same protein have been found for this gene, and pseudogenes of this gene have been described on chromosome 4 and on the X chromosome.

PCNA has been shown to interact with various proteins involved in DNA replication and repair (e.g. see review by Moldovan, et al. Cell (2007) 129, 665-679). PCNA interactacts with the following gene products: with EXO1, POLH, POLK, DNMT1, ERCC5, FEN1, CDC6 and POLDIP2. PCNA also interacts with the gene product APEX2; this interaction is triggered by reactive oxygen species and increased by misincorporation of uracil in nuclear DNA. PNCA forms a ternary complex with the gene product DNTTIP2 and core histone. Other known or hypothesized interactions include the following following gene products: KCTD10 and PPP1R15A (based on similarity); POLD1, POLD3 and POLD4; BAZ1B, wherein the interaction is direct; HLTF and SHPRH; NUDT15, and interaction is disrupted in response to UV irradiation and acetylation; CDKN1A/p21(CIP1) and CDT1 via their PIP-box which also recruits the DCX(DTL) complex; and DDX11. PCNA forms nuclear foci representing sites of ongoing DNA replication and vary in morphology and number during S phase. It is, with APEX2, redistributed in discrete nuclear foci in presence of oxidative DNA damaging agents. In addition to these protein-protein interactions, PCNA has been shown to interact with Ku70, Werner syndrome ATP-dependent helicase, RFC2, RFC1, RFC4, RFC5, GADD45G, CDC25C, MUTYH, Flap structure-specific endonuclease 1, Cyclin O, CHTF18, Y box binding protein 1, Cyclin D1, Annexin A2, MSH6, DNMT1, HDAC1, KCTD13, XRCC1, Cyclin-dependent kinase 4, Ku80, HUS1, GADD45A, POLD2, ING1, POLH, KIAA0101, POLDIP2, EP300, MCL1, POLD3, [13][61] Cyclin-dependent kinase inhibitor 1C, POLL, Ubiquitin C and P21.

### D. METHODS OF MAKING THE COMPOUNDS

In one aspect, the invention relates to methods of making compounds useful as modulators of PCNA activity. In a further aspect, the products of the disclosed methods of making are modulators of PCNA activity. In a yet further aspect, the products of disclosed methods of making bind to a PCNA protein and negatively modulate PCNA activity. The products of disclosed methods, in one aspect, selectivity for modulation of PCNA activity compared to other proteins. In a still further aspect, the compounds exhibit selectivity for inhibition of PCNA activity compared to the TR (thyroid hormone receptor).

In one aspect, the invention relates to methods of making the disclosed compounds. The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, in addition to other standard manipulations known in the literature or to one skilled in the art. The following examples are provided so that the invention might be more fully understood, are illustrative only, and should not be construed as limiting.

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

1. Route I

In one aspect, substituted 4-phenoxyphenol analogs of the present invention can be prepared generically as shown below.

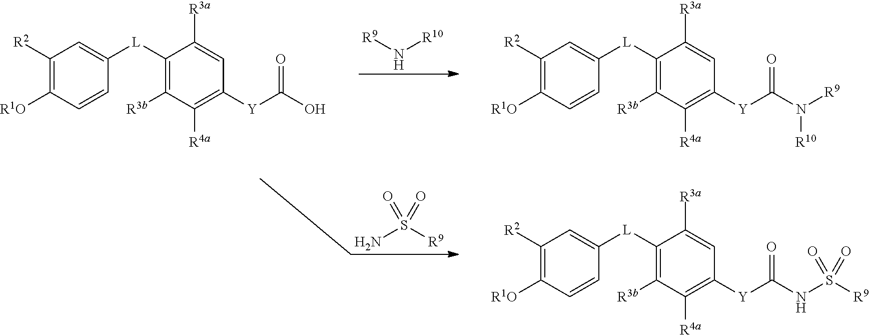

Compounds are represented in generic form, with substituents as noted in compound descriptions elsewhere herein. A more specific example is set forth below.

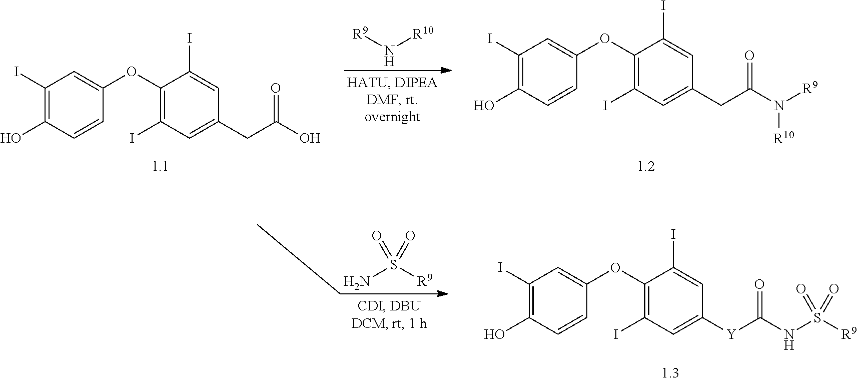

In one aspect, compounds of type 1.2 can be prepared by reaction of a carboxyl-functionalized compound with an amine, HNR⁹R¹⁰, thereby providing an amide. Such reaction can be effected with, e.g., the peptide coupling reagent 2-(1H-7-azabenzotriazol-1-yl)-1,1,3,3-tetramethyl uronium hexafluorophosphate methanaminium (HATU) in the presence of a suitable base, e.g., N,N-diisopropylethylamine (DIPEA). It is contemplated that activated carboxyl functionalities, e.g., acyl halides, can be alternatively employed.

In one aspect, compounds of type 1.3 can be prepared by reaction of a carboxyl-functionalized compound with a sulfonamide, HNSO₂R⁹, thereby providing, e.g., an N-(alkylsulfonyl)alkylamide. Such reaction can be effected with 1,1′-carbonyldiimidazole (CDI) in the presence of a suitable base, e.g., 1,8-diazabicyclo[5.4.0]undec-7-ene (DBU).

2. Route II

In one aspect, substituted 4-phenoxyphenol analogs analogs of the present invention can be prepared generically by one of the synthetic schemes as shown below.

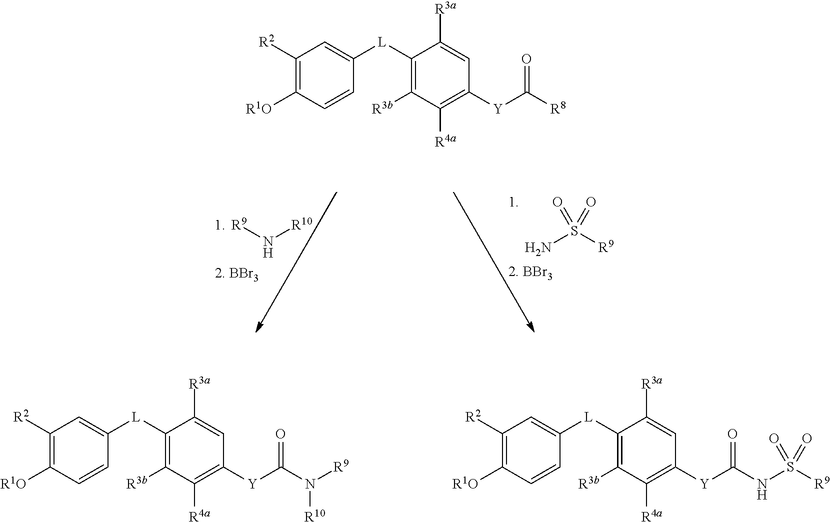

Compounds are represented in generic form, with substituents as noted in compound descriptions elsewhere herein. More specific examples are set forth below.

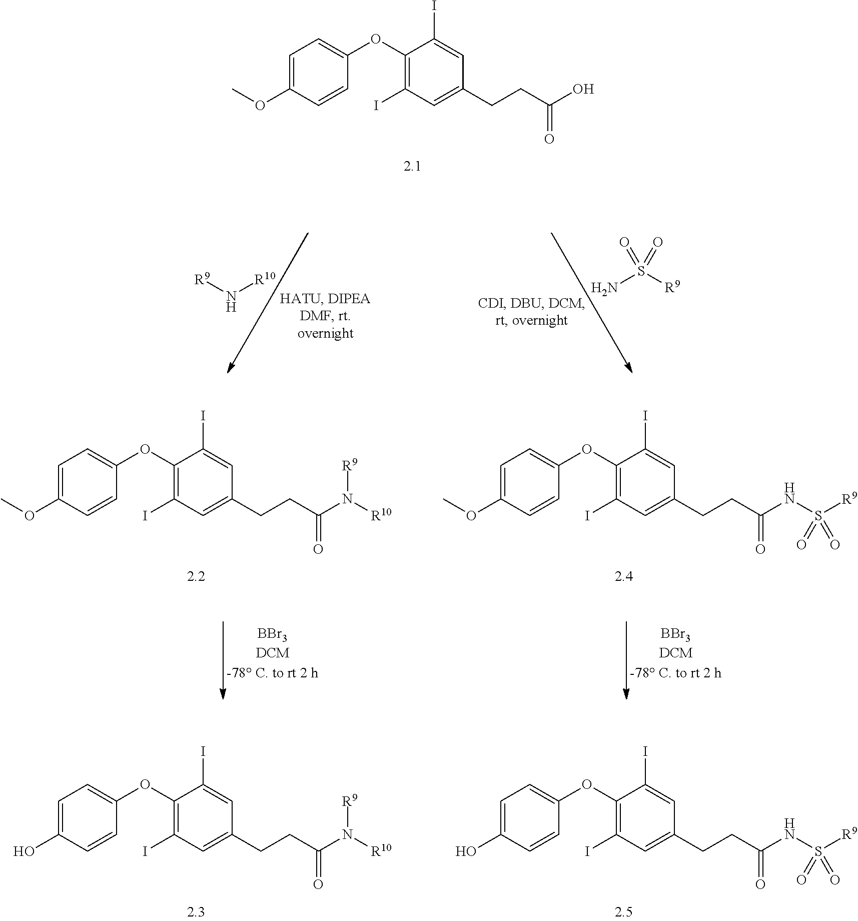

In one aspect, compounds of type 2.3 and 2.5 can be prepared by reaction of the corresponding ether analog (i.e. respectively, compound 2.1 and 2.4) to provide the phenolic analog as shown above. Such a reaction can be effected with, e.g. boron tribromide (BBr₃), in a suitable solvent, e.g. dichloromethane. It is contemplated that other methods of ether cleavage can be employed as appropriate. For example, 2-(diethylamino)ethanethiol (see Magano, J., et al., J. Org. Chem., (2006) 71, 7103-7105) or 1-n-butyl-3-methylimidazolium bromide ([bmim] [Br]; see Boovanahalli, S. K., et al. J. Org. Chem., (2004) 69, 3340-3344) can be used to effect ether cleavage as well.

In one aspect, compounds of type 2.3 can be prepared by reaction of a carboxyl-functionalized compound (I.e. compound 2.1) with an amine, HNR⁹R¹⁰, thereby providing an amide. Such reaction can be effected with, e.g., the peptide coupling reagent 2-(1H-7-azabenzotriazol-1-yl)-1,1,3,3-tetramethyl uronium hexafluorophosphate methanaminium (HATU) in the presence of a suitable base, e.g., N,N-diisopropylethylamine (DIPEA). It is contemplated that activated carboxyl functionalities, e.g., acyl halides, can be alternatively employed.

In one aspect, compounds of type 2.4 can be prepared by reaction of a carboxyl-functionalized compound with a sulfonamide, HNSO₂R⁹, thereby providing, e.g., an N-(alkylsulfonyl)alkylamide. Such reaction can be effected with 1,1′-carbonyldiimidazole (CDI) in the presence of a suitable base, e.g., 1,8-diazabicyclo[5.4.0]undec-7-ene (DBU).

3. Route III

In one aspect, substituted 4-phenoxyphenol analogs analogs of the present invention can be prepared generically as shown below.

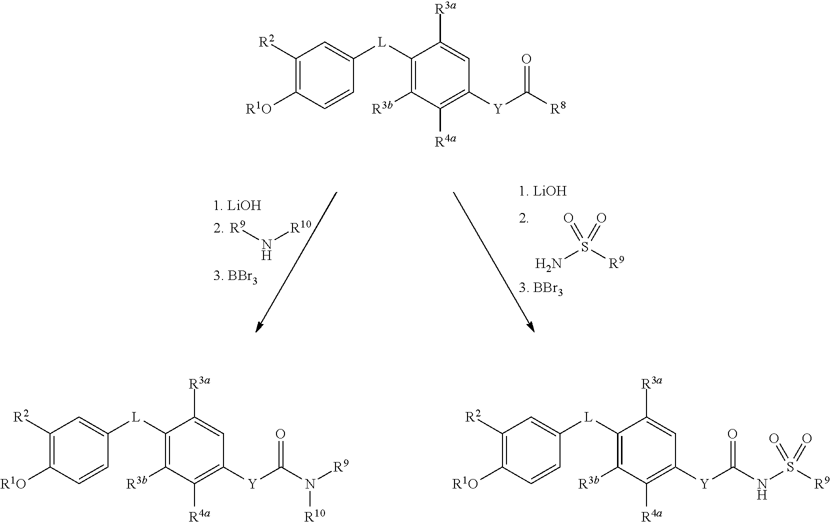

Compounds are represented in generic form, with substituents as noted in compound descriptions elsewhere herein. More specific example are set forth below.

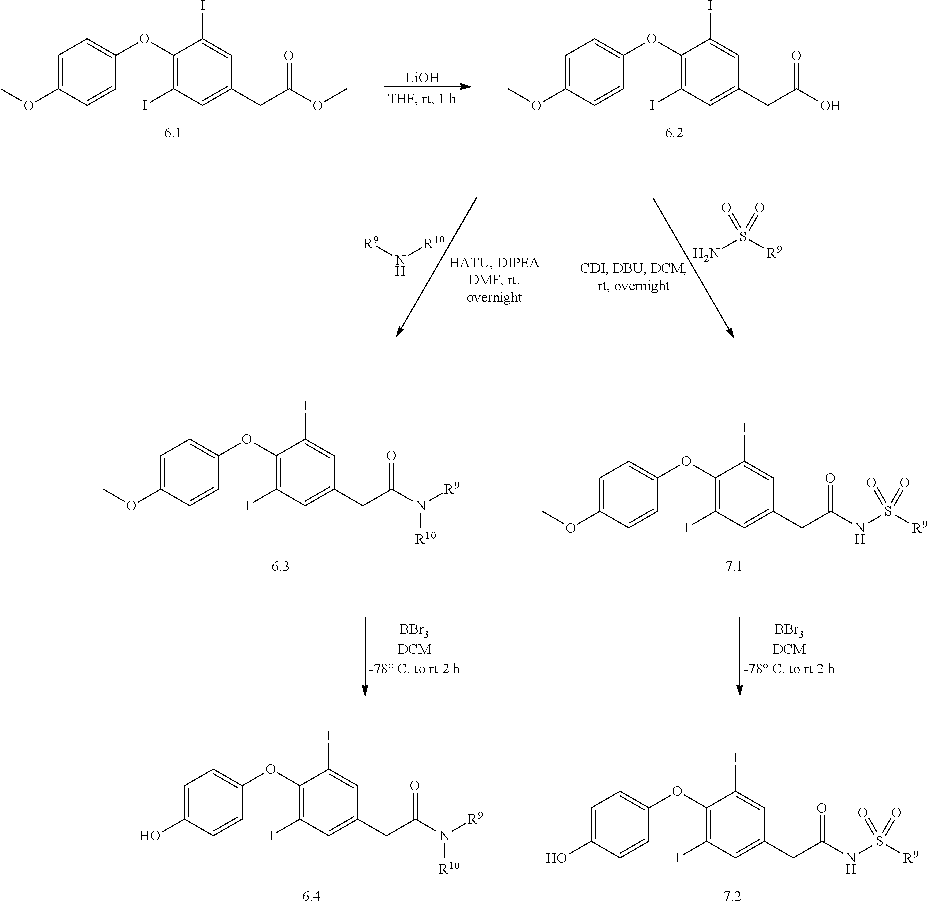

In one aspect, compounds of types 6.3, 6.4, 7.1 and 7.2 can be prepared from a compound of type 6.2 using methods as described above. A compound of type 6.2 can be prepared by reaction of a ester analog such as compound 6.1 in the presence of base. Such a reaction can be effected with a suitable base, e.g. LiOH, carried out in a suitable solvent, e.g. THF. Alternative approaches to prepare a suitable carboxylic acid of type 6.2 are available to one skill in the art.

4. Route Iv

In one aspect, substituted 4-phenoxyphenol analogs analogs of the present invention can be prepared generically as shown below.

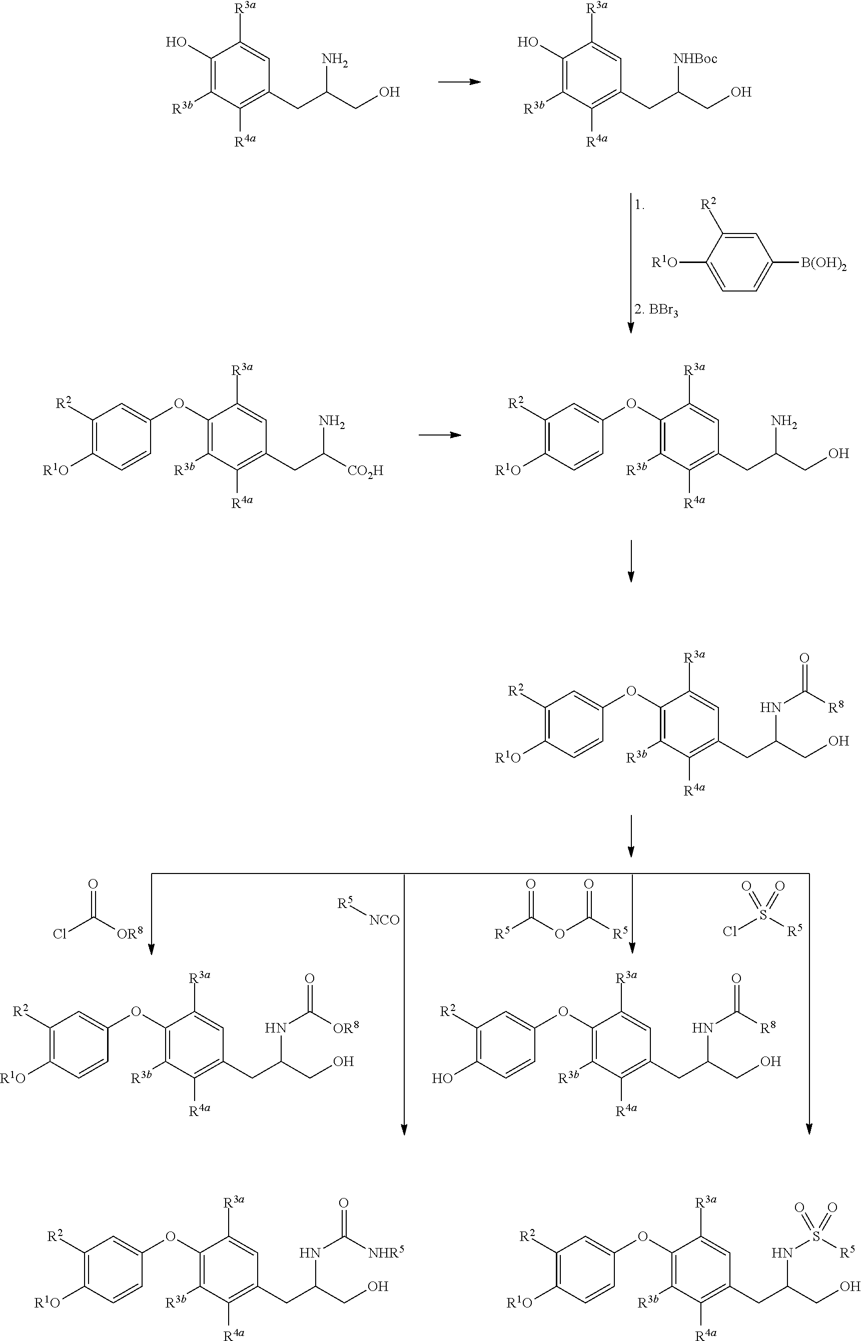

Compounds are represented in generic form, with substituents as noted in compound descriptions elsewhere herein. A more specific example is set forth below.

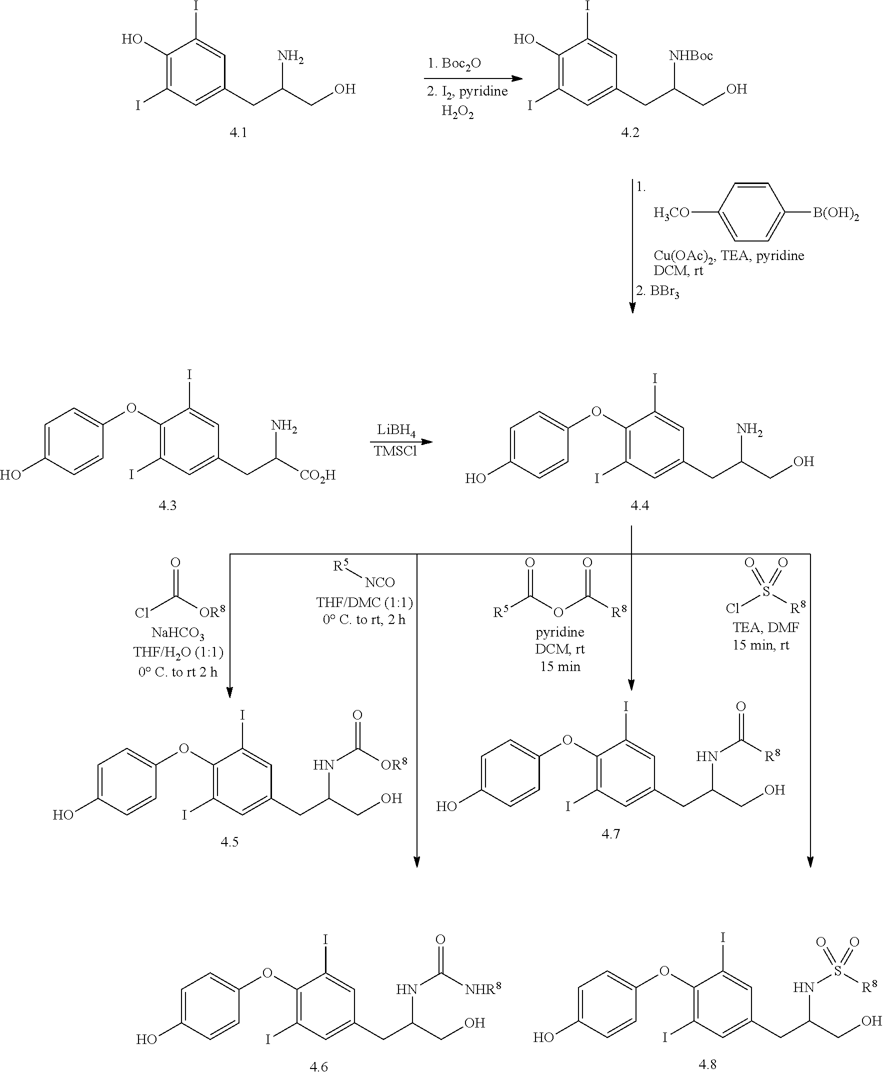

In one aspect, compounds of type 4.4 can be prepared by various methods, e.g. starting with a suitable carboxylic acid derivative such as compound 4.3 or beginning a suitable phenol such as compound 4.1. Beginning with compound 4.1, the amine group is protected with a suitable protecting group, e.g. Boc, using reaction conditions known to one skilled in the art, e.g. the conditions shown above, to provide a compound of type 4.2. Compound 4.2 is reacted with a suitable phenylboronic acid in the presence of Cu(OAc)₂ and TEA, followed by treatment with BBr₃ to provide a compound of type 4.4. This product can be utilized as a starting material for further reaction to yield compounds of type 4.5, 4.6, 4.7, and 4.8.

In one aspect, compounds of type 4.5 can be prepared by reaction of a suitable functionalized acyl halide, e.g. ClCO₂R⁸, with a compound of type 4.4 (prepared as described above), thereby providing the desired amide analog. Such a reaction can be effected under reaction conditions such as those shown above.

In one aspect, compounds of type 4.6 can be prepared by reaction of a suitable isocyanato derivative in a suitable solvent, e.g. dichloromethane, for a time sufficient, e.g. about 1-6 hours, at a suitable temperature, e.g. 0° C. with slow warming to room temperature, to achieve completion of the reaction.

In one aspect, compounds of type 4.7 can be prepared by reaction of a suitable anhydride derivative in a suitable solvent, e.g. THF/DMF, for a time sufficient, e.g. about 5-60 minutes, at a suitable temperature, e.g. room temperature, to achieve completion of the reaction.

5. Route V

In one aspect, substituted 4-phenoxyphenol analogs of the present invention can be prepared generically as shown below.

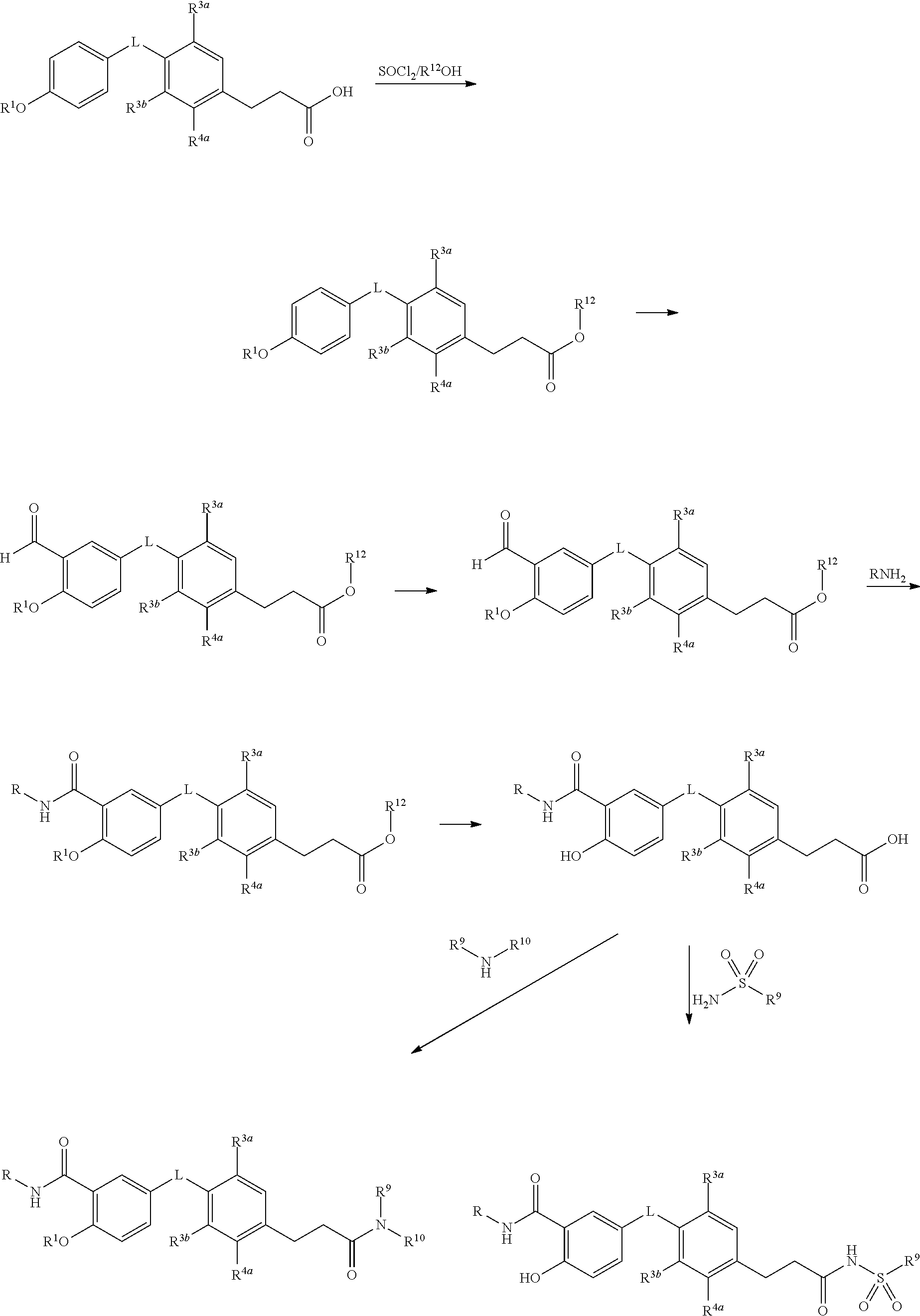

Compounds are represented in generic form, with substituents as noted in compound descriptions elsewhere herein. A more specific example is set forth below.

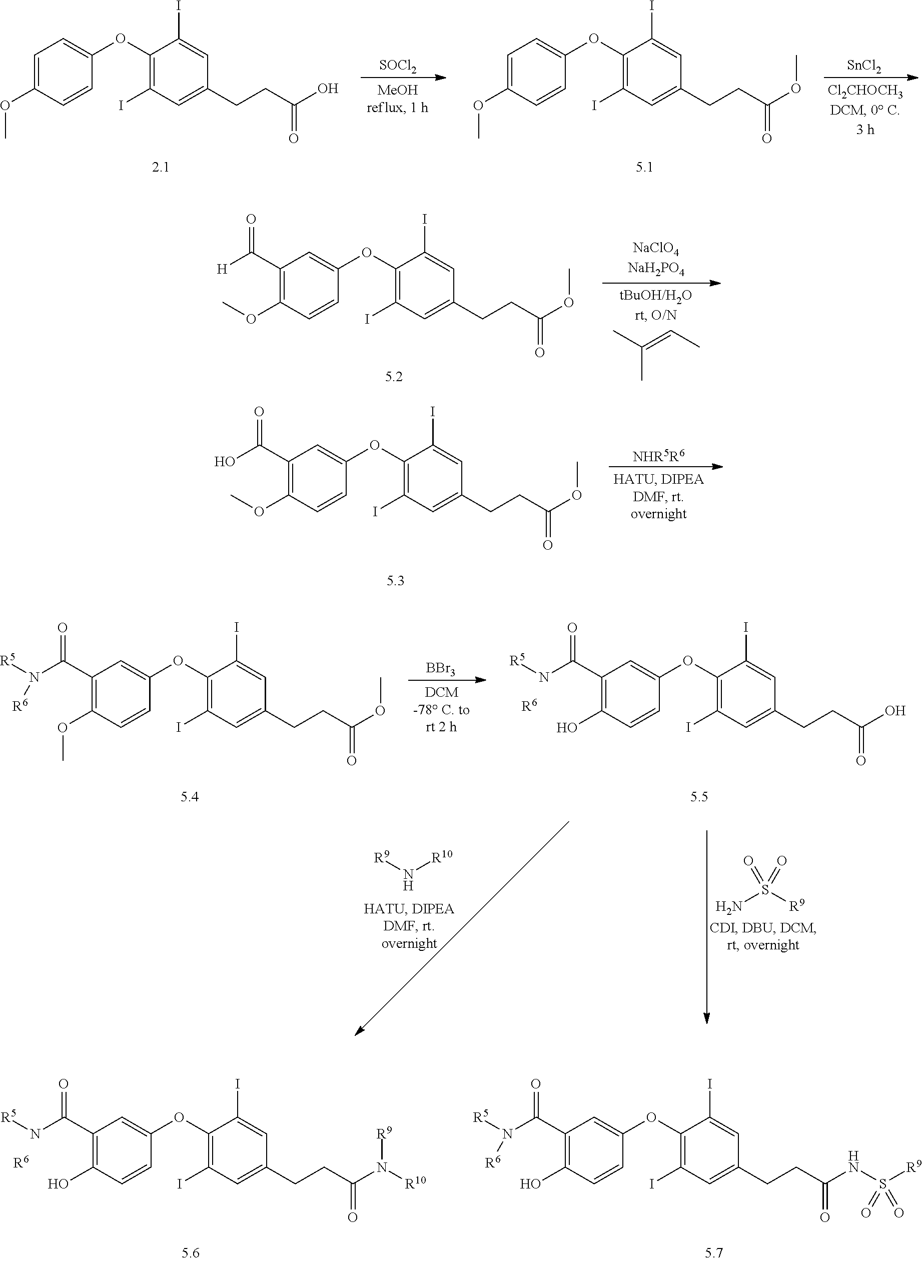

6. Route VI

In one aspect, substituted 4-phenoxyphenol analogs of the present invention can be prepared generically as shown below.

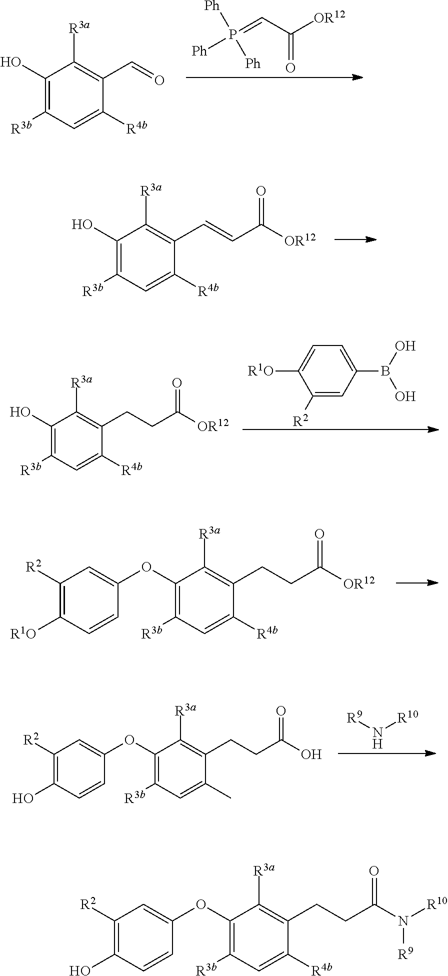

Compounds are represented in generic form, with substituents as noted in compound descriptions elsewhere herein. A more specific example is set forth below.

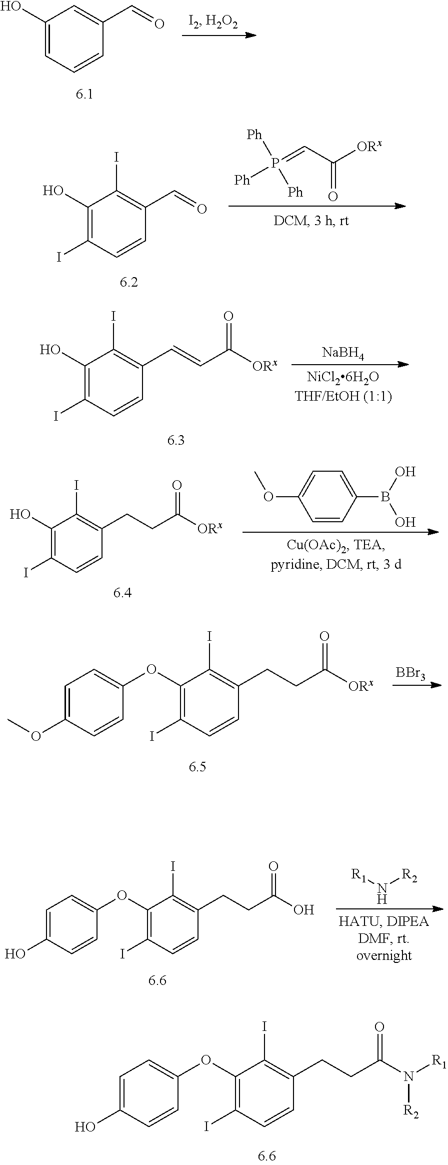

7. Route VII

In one aspect, substituted 4-phenoxyphenol analogs of the present invention can be prepared generically as shown below.

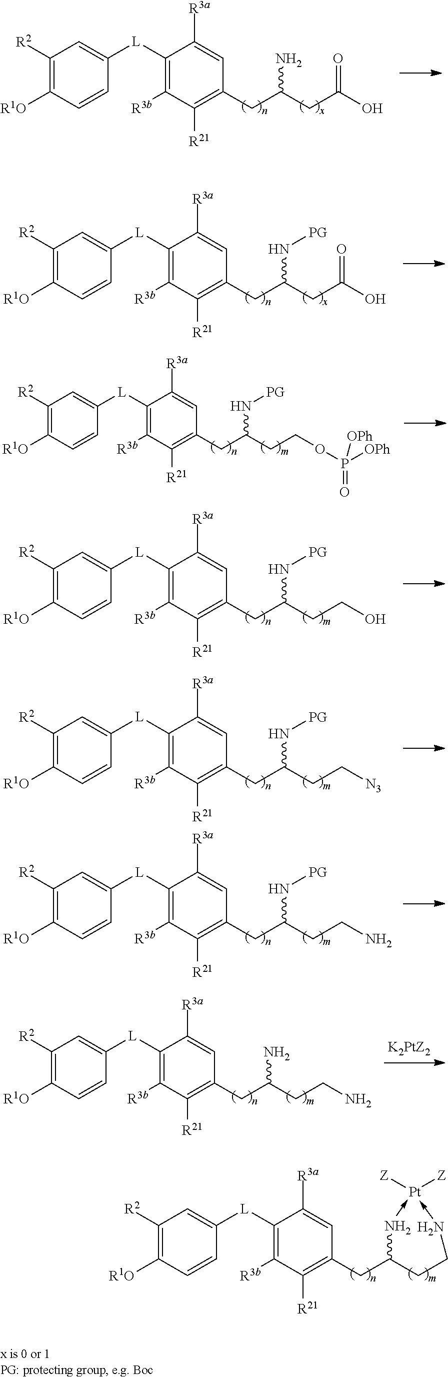

Compounds are represented in generic form, with substituents as noted in compound descriptions elsewhere herein. A more specific example is set forth below.

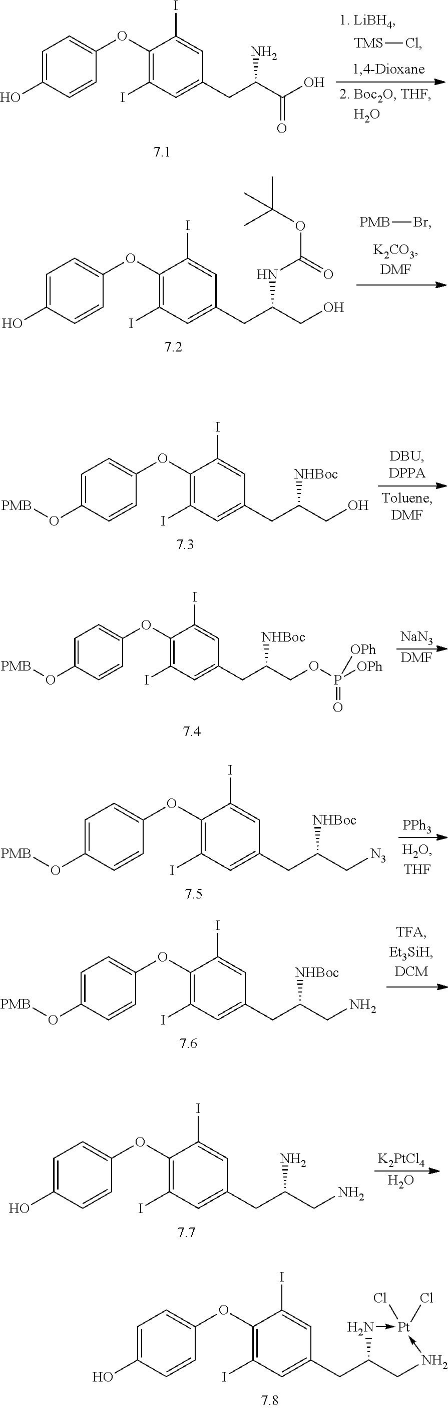

In one aspect, the compounds of type 7.8 can be prepared by various methods, e.g. the methods outlined in the synthesis scheme above. The synthesis begins with the appropriate substituted 4-phenoxyphenol analog with a alpha amino carboxylic acid moiety as shown in the example above. The amino group is protected with a suitable protecting group, e.g. a Boc group, using reaction conditions known to one skilled in the art, e.g. the conditions shown above, to provide a compound of type 7.2.

In one aspect, compounds of type 7.3 can be prepared by protection of the aryl hydroxyl moiety. Several methods are known to one skilled in the art, e.g. as shown above the p-methoxybenzyl ether is prepared from the corresponding p-methoxybenzyl halide such as p-methoxybenzyl bromide. Alternative approaches exist such as use of trichloroacetimidate of para-methoxybenzyl alcohol to produce a compound of type 7.3. As shown above, a compound of type 7.4 is prepared by conversion of the alkyl primary alcohol to the corresponding diphenyl phosphoryl ether using diphenylphosphoryl azide (DPPA), followed by conversion to the azide (compound of type 7.5) in the presence of sodium azide. Alternative approaches to convert the alkyl primary alcohol to the corresponding azide exist, and can be used as the circumstances warrant, e.g. variants of the Mitsonobu reaction utilizing hydrogen azide, triphenylphosphane, and diethyl azodicarboxylate (DEAD).

In one aspect, compounds of type 7.6 can be prepared by reduction of the azide of a compound of type 7.5. As shown above, one approach is using Staudinger reduction, i.e. reaction with triphenylphosphine followed by aqueous work-up to yield the desired amine. Alternatively, azides can be easily and chemoselectively reduced to the corresponding amines by reaction with dichloroindium hydride under very mild conditions (e.g. see L. Benati, G. Bencivenni, R. Leardini, D. Nanni, M. Minozzi, P. Spagnolo, R. Scialpi, G. Zanardi, Org. Lett., 2006, 8, 2499-2502). In the synthesis scheme above, the Boc and p-methoxybenzyl protecting groups are removed next using trifluoroacetic acid. The particular conditions used will be dictated by the choice of protecting groups, and as would be known to one skilled in the art.

In one aspect, compounds of type 7.6 are converted to the corresponding platinum (II) complex by reaction with an appropriate platinum (II) compound, e.g. K₂PtCl₄, as shown above. Compounds of type 7.6 can be easily converted to desired platinum complexes using methods known to one skilled in the art, e.g. replacement of chloro ligands with a bidentate oxalate moiety.

8. Route VIII

In one aspect, substituted 4-phenoxyphenol analogs of the present invention can be prepared generically as shown below.

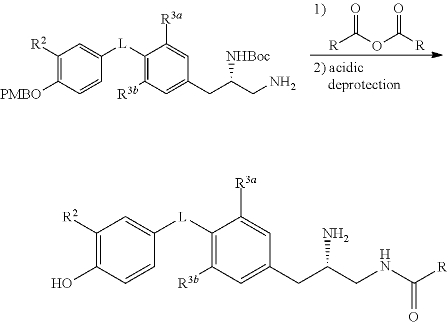

Compounds are represented in generic form, with substituents as noted in compound descriptions elsewhere herein. Generally, amidation of the terminal amine can be effected with an activated carboxyl functionality, e.g., an anhydride. Deprotection can then yield the desired compound. A more specific example is set forth below.

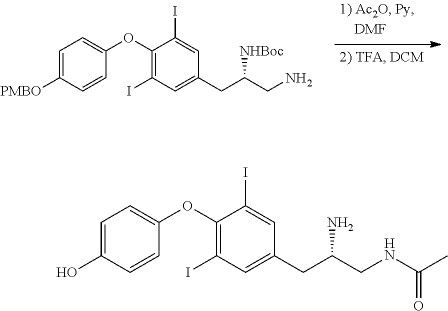

In one aspect, synthesis of the compounds begins with protection of the phenolic functionality and/or the secondary amino functionality, if needed. Suitable protecting groups include, but are not limited to, p-methoxybenzyl and n-butyloxycarbonyl. As an example, a doubly-protected (S)-4-(4-(2,3-diaminopropyl)-2,6-diiodophenoxy)phenol can be treated with acetic acid and pyridine in dimethylformamide to provide (S)—N-(2-amino-3-(4-(4-hydroxyphenoxy)-3,5-diiodophenyl)propyl)acetamide. Finally, protecting groups can be removed under suitable conditions, e.g., acidic deprotection with trifluoroacetic acid.

9. Route IX

In one aspect, substituted 4-phenoxyphenol analogs of the present invention can be prepared generically as shown below.

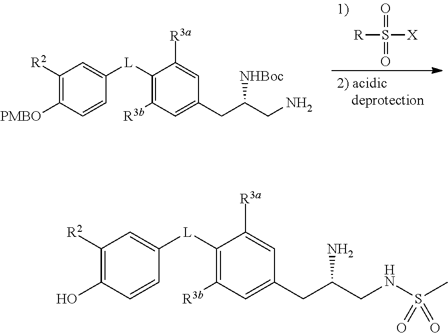

Compounds are represented in generic form, with substituents as noted in compound descriptions elsewhere herein. Generally, sulfonamidation of the terminal amine can be effected with an activated sulfonyl functionality, e.g., a sulfonyl halide. Deprotection can then yield the desired compound. A more specific example is set forth below.

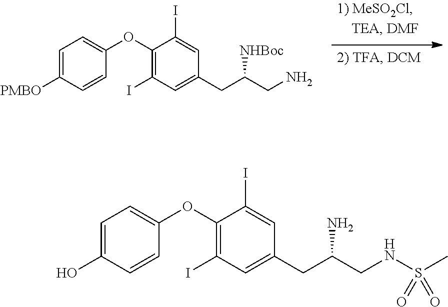

In one aspect, synthesis of the compounds begins with protection of the phenolic functionality and/or the secondary amino functionality, if needed. Suitable protecting groups include, but are not limited to, p-methoxybenzyl and n-butyloxycarbonyl. As an example, a doubly-protected (S)-4-(4-(2,3-diaminopropyl)-2,6-diiodo phenoxy)phenol can be treated with methylsulfonyl chloride and trieethylamine in dimethyl-formamide to provide (S)—N-(2-amino-3-(4-(4-hydroxyphenoxy)-3,5-diiodophenyl) propyl)methanesulfonamide. Finally, protecting groups can be removed under suitable conditions, e.g., acidic deprotection with trifluoroacetic acid.

10. Route X

In one aspect, substituted 4-phenoxyphenol analogs of the present invention can be prepared generically as shown below.

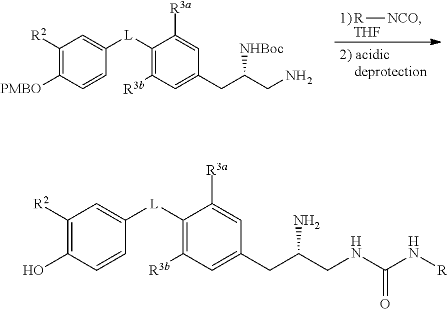

Compounds are represented in generic form, with substituents as noted in compound descriptions elsewhere herein. Generally, conversion of the terminal amine to a urea can be effected with an isocyanate. Deprotection can then yield the desired compound. A more specific example is set forth below.

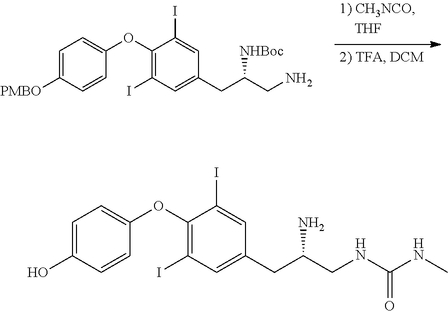

In one aspect, synthesis of the compounds begins with protection of the phenolic functionality and/or the secondary amino functionality, if needed. Suitable protecting groups include, but are not limited to, p-methoxybenzyl and n-butyloxycarbonyl. As an example, a doubly-protected (S)-4-(4-(2,3-diaminopropyl)-2,6-diiodophenoxy)phenol can be treated with methylisocyanate in tetrahydrofuran to provide (S)-1-(2-amino-3-(4-(4-hydroxyphenoxy)-3,5-diiodophenyl)propyl)-3-methylurea. Finally, protecting groups can be removed under suitable conditions, e.g., acidic deprotection with trifluoroacetic acid.

11. Route XI

In one aspect, substituted 4-phenoxyphenol analogs of the present invention can be prepared generically as shown below.

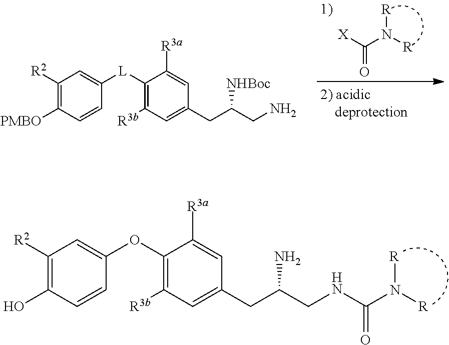

Compounds are represented in generic form, with substituents as noted in compound descriptions elsewhere herein. Generally, conversion of the terminal amine to a urea can be effected with an aminocarbonyl halide. Deprotection can then yield the desired compound. A more specific example is set forth below.

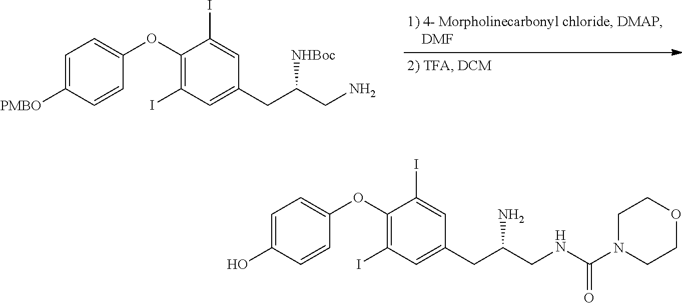

In one aspect, synthesis of the compounds begins with protection of the phenolic functionality and/or the secondary amino functionality, if needed. Suitable protecting groups include, but are not limited to, p-methoxybenzyl and n-butyloxycarbonyl. As an example, a doubly-protected (S)-4-(4-(2,3-diaminopropyl)-2,6-diiodophenoxy)phenol can be treated with 4-morpholinecarbonyl chloride and 4-dimethylaminopyridine in dimethylformamide to provide (S)—N-(2-amino-3-(4-(4-hydroxyphenoxy)-3,5-diiodophenyl)propyl)morpholine-4-carboxamide. Finally, protecting groups can be removed under suitable conditions, e.g., acidic deprotection with trifluoroacetic acid.

12. Route XII

In one aspect, substituted 4-phenoxyphenol analogs of the present invention can be prepared generically as shown below.

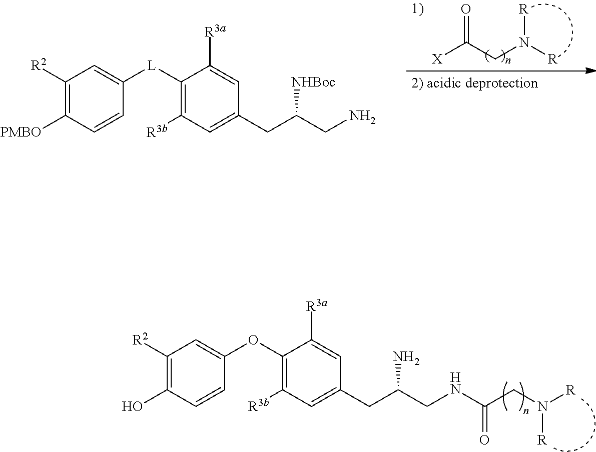

Compounds are represented in generic form, with substituents as noted in compound descriptions elsewhere herein. Generally, amidation of the terminal amine can be effected with an activated carboxyl functionality, e.g., an acyl halide. Deprotection can then yield the desired compound. A more specific example is set forth below.

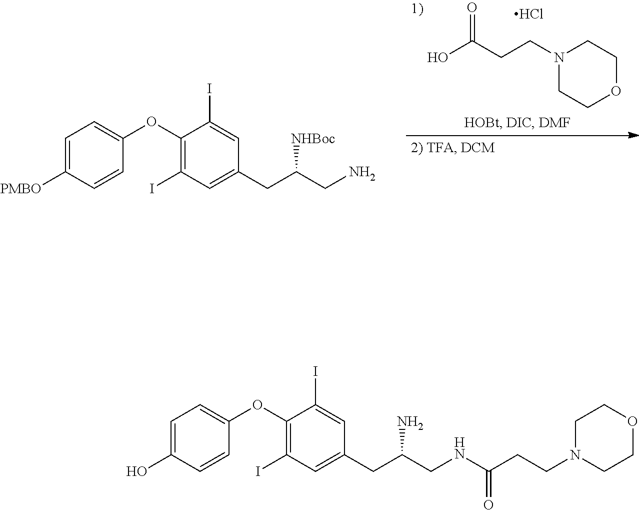

In one aspect, synthesis of the compounds begins with protection of the phenolic functionality and/or the secondary amino functionality, if needed. Suitable protecting groups include, but are not limited to, p-methoxybenzyl and n-butyloxycarbonyl. As an example, a doubly-protected (S)-4-(4-(2,3-diaminopropyl)-2,6-diiodophenoxy)phenol can be treated with 3-morpholinepropanoic acid hydrochloride and N-hydroxybenzotriazole and diisopropylcarbodiimide in dimethylformamide to provide (S)—N-(2-amino-3-(4-(4-hydroxyphenoxy)-3,5-diiodophenyl)propyl)-3-morpholinopropanamide. Finally, protecting groups can be removed under suitable conditions, e.g., acidic deprotection with trifluoroacetic acid.

13. Route XIII

In one aspect, substituted 4-phenoxyphenol analogs of the present invention can be prepared generically as shown below.

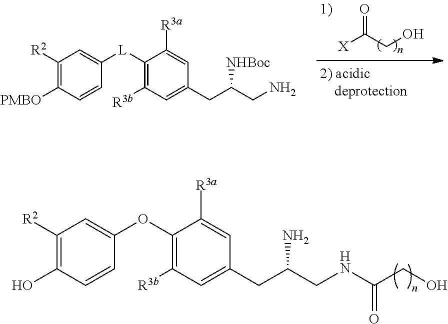

Compounds are represented in generic form, with substituents as noted in compound descriptions elsewhere herein. Generally, amidation of the terminal amine can be effected with an activated carboxyl functionality, e.g., an acyl halide. Deprotection can then yield the desired compound. A more specific example is set forth below.

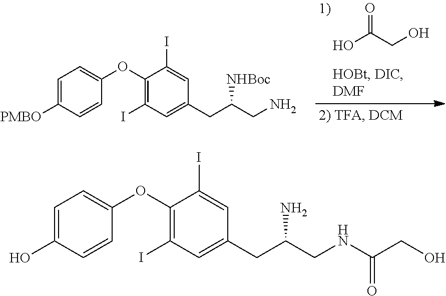

In one aspect, synthesis of the compounds begins with protection of the phenolic functionality and/or the secondary amino functionality, if needed. Suitable protecting groups include, but are not limited to, p-methoxybenzyl and n-butyloxycarbonyl. As an example, a doubly-protected (S)-4-(4-(2,3-diaminopropyl)-2,6-diiodophenoxy)phenol can be treated with 2-hydroxyacetic acid and N-hydroxybenzotriazole and diisopropylcarbodiimide in dimethylformamide to provide (S)—N-(2-amino-3-(4-(4-hydroxyphenoxy)-3,5-diiodophenyl)propyl)-2-hydroxyacetamide. Finally, protecting groups can be removed under suitable conditions, e.g., acidic deprotection with trifluoroacetic acid.

It is contemplated that each disclosed methods can further comprise additional steps, manipulations, and/or components. It is also contemplated that any one or more step, manipulation, and/or component can be optionally omitted from the invention. It is understood that a disclosed methods can be used to provide the disclosed compounds. It is also understood that the products of the disclosed methods can be employed in the disclosed methods of using.

### E. PHARMACEUTICAL COMPOSITIONS

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In one aspect, the invention relates to a pharmaceutical composition comprising a pharmaceutically acceptable carrier and an effective amount of a compound represented by a formula:

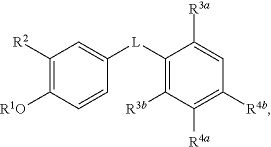

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein one of R^{4a} and R^{4b} is selected from hydrogen, halo, C1-C3 alkyl, C1-C3 haloalkyl, and C1-C3 polyhaloalkyl, and the other is selected from:

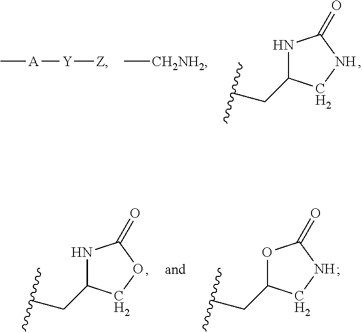

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a pharmaceutical composition comprising a pharmaceutically acceptable carrier and an effective amount of a compound represented by a formula:

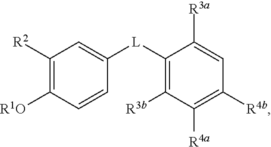

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a pharmaceutical composition comprising a pharmaceutically acceptable carrier and an effective amount of a compound represented by a formula:

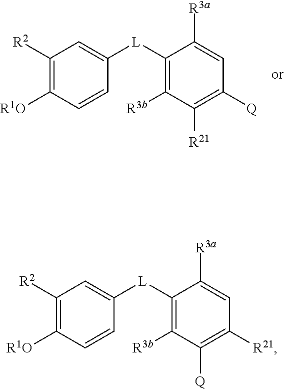

wherein L is selected from O, CH₂, CHOH, and C═O; wherein Q is selected from a structure represented by a formula:

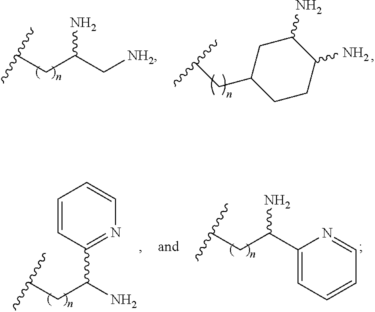

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound is any of the disclosed compounds or at least one product of the disclosed methods of making. In a yet further aspect, the pharmaceutical composition comprises one or more of any of the disclosed compounds or at least one product of the disclosed methods of making. In a still further aspect, an effective amount is a therapeutically effective amount.

In a further aspect, the pharmaceutical composition is administered to a mammal. In a yet further aspect, the mammal is a human. In a yet further aspect, the mammal has been diagnosed with a need for treatment of the disorder prior to the administering step. In a still further aspect, the method further comprises the step of identifying a mammal in need of treatment of the disorder.

In a further aspect, the compound inhibits PCNA protein activity. In an even further aspect, the inhibition of binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the compound that inhibits cell growth. In a still further aspect, the compound inhibits cell growth with an IC₅₀ of less than about 500 μM. In a yet further aspect, the compound inhibits cell growth with an IC₅₀ of less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, the pharmaceutical composition treats a PCNA dysfunction. In a still further aspect, the PCNA dysfunction is increased PCNA activity. In a still further aspect, the pharmaceutical composition treats a proliferative disorder. In a yet further aspect, the proliferative disorder is a hyperproliferative disorder.

In a further aspect, the pharmaceutical composition treats hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the pharmaceutical composition treats a hyperproliferative disorder that is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the pharmaceutical composition treats a cancer that is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the pharmaceutical composition treats a cancer that is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the pharmaceutical composition further comprises a a chemotherapeutic agent. In a yet further aspect, the chemotherapeutic agent is an alkylating agent. In a still further aspect, the chemotherapeutic agent is cisplatin. In an even further aspect, the chemotherapeutic agent is selected from actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require modulation of PCNA protein activity an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the from of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for modulating PCNA protein activity (e.g., treatment of one or more proliferative or hyperproliferative disorders associated with a PCNA dysfunction) in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### F. METHODS OF USING THE COMPOUNDS AND COMPOSITIONS

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

In one aspect, the subject compounds can be coadministered with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11IL-2Imatinib mesylate, Imidazole Carboxamide Interferon alfa, Interferon Alfa-2b (PEG Conjugate), Interleukin -2, Interleukin-11, Intron A® (interferon alfa-2b) Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin ®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone ®, Oxaliplatin, P, Paclitaxel, Paclitaxel Protein-bound, Pamidronate, Panitumumab, Panretin ®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a) Romiplostim, Rubex®, Rubidomycin hydrochloride, S, Sandostatin®, Sandostatin LAR ®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna ®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®.

In another aspect, the subject compounds can be administered in combination with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11IL-21matinib mesylate, Imidazole CarboxamideInterferon alfa, Interferon Alfa-2b (PEG Conjugate), Interleukin -2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred ®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin ®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone®, Oxaliplatin, P, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®

In another aspect, the subject compound can be used in combination with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11IL-21matinib mesylate, Imidazole CarboxamideInterferon alfa, Interferon Alfa-2b (PEG Conjugate), Interleukin -2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred ®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin ®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, 0, Octreotide, Octreotide acetate, Ofatumumab, Oncospar®, Oncovin®, Ontak®, Onxal™, Oprelvekin, Orapred®, Orasone®, Oxaliplatin, P, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR ®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®

The pharmaceutical compositions and methods of the present invention can further comprise other therapeutically active compounds as noted herein which are usually applied in the treatment of the above mentioned pathological conditions.

The pharmaceutical compositions and methods of the present invention can further comprise other therapeutically active compounds as noted herein which are usually applied in the treatment of the above mentioned pathological conditions.

1. Treatment Methods

The compounds disclosed herein are useful for treating, preventing, ameliorating, controlling or reducing the risk of a variety of hyperproliferative disorders associated with a PCNA protein activity dysfunction. In a yet further aspect, the hyperproliferative disorder is a cancer.

In one aspect, the invention relates to a method for the treatment of a disorder associated with a PCNA protein activity dysfunction in a mammal comprising the step of administering to the mammal at least one disclosed compound or at least one disclosed product in a dosage and amount effective to treat the disorder in the mammal. In a further aspect, the mammal is a human. In a further aspect, the mammal has been diagnosed with a need for treatment of the disorder prior to the administering step. In a further aspect, the method further comprises the step of identifying a mammal in need of treatment of the disorder.

It is understood that cancer refers to or describe the physiological condition in mammals that is typically characterized by hyperproliferation. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

Thus, provided is a method for treating or preventing a hyperproliferative disorder, comprising: administering to a subject at least one disclosed compound; at least one disclosed pharmaceutical composition; and/or at least one disclosed product in a dosage and amount effective to treat the disorder in the subject.

a. Treatment of a Disorder Associated PCNA Dysfunction

In one aspect, the invention relates to a method for the treatment of a disorder associated with PCNA dysfunction in a mammal comprising the step of administering to the mammal an effective amount of at least one compound having a structure represented by a formula:

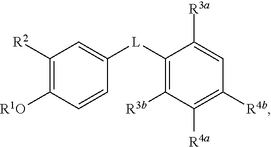

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is selected from:

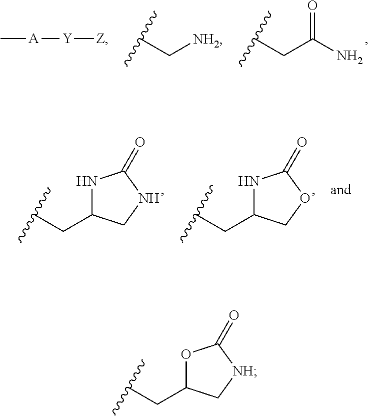

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for the treatment of a disorder associated with PCNA dysfunction in a mammal comprising the step of administering to the mammal an effective amount of at least one compound having a structure represented by a formula:

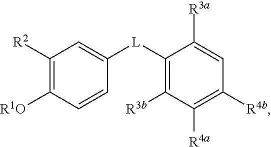

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for the treatment of a disorder associated with PCNA dysfunction in a mammal comprising the step of administering to the mammal an effective amount of at least one compound having a structure represented by a formula:

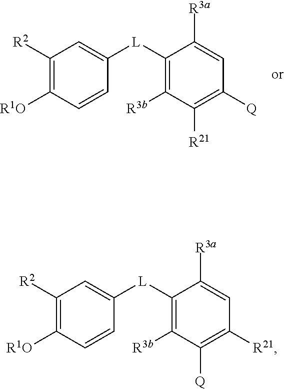

wherein L is selected from O, CH₂, CHOH, and C═O; wherein Q is selected from a structure represented by a formula:

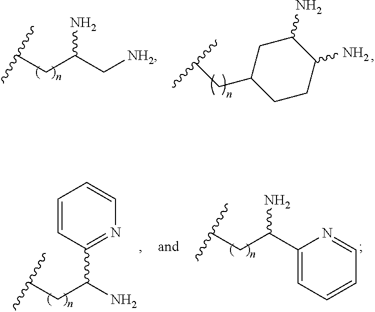

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound administered is any of the disclosed compounds or at least one product of the disclosed methods of making.

In a further aspect, the mammal is a human. In a yet further aspect, the mammal has been diagnosed with a need for treatment of the disorder prior to the administering step. In a still further aspect, the method further comprises the step of identifying a mammal in need of treatment of the disorder. In an even further aspect, an effective amount is a therapeutically effective amount.

In a further aspect, the compound inhibits PCNA protein activity. In an even further aspect, the compound inhibits binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the compound that inhibits cell growth. In a still further aspect, the compound inhibits cell growth with an IC₅₀ of less than about 500 μM, less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, the PCNA dysfunction is increased PCNA activity. In a still further aspect, the disorder is a proliferative disorder. In a yet further aspect, the proliferative disorder is a hyperproliferative disorder.

In a further aspect, the hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the hyperproliferative disorder is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the compound is co-administered with a chemotherapeutic agent. In a yet further aspect, the chemotherapeutic agent is an alkylating agent. In a still further aspect, the chemotherapeutic agent is cisplatin. In an even further aspect, the chemotherapeutic agent is selected from actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

b. Modulation of PCNA Activity

In one aspect, the invention relates to a method for modulation of PCNA activity in a mammal comprising the step of administering to the mammal an effective amount of at least one compound having a structure represented by formula:

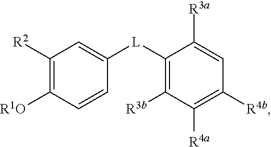

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is selected from:

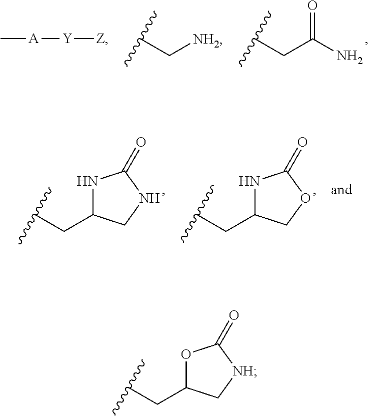

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for modulation of PCNA activity in a mammal comprising the step of administering to the mammal an effective amount of at least one compound having a structure represented by formula:

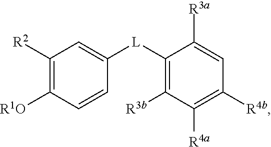

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for modulation of PCNA activity in a mammal comprising the step of administering to the mammal an effective amount of at least one compound having a structure represented by formula:

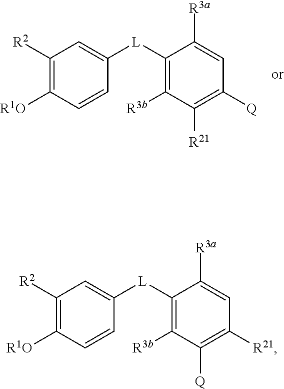

wherein L is selected from O, CH₂, CHOH, and C═O; wherein Q is selected from a structure represented by a formula:

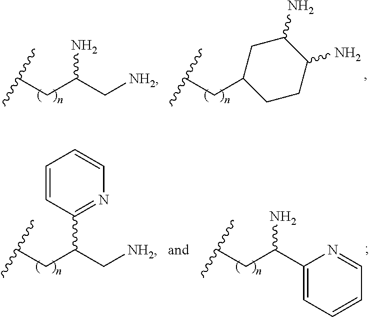

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound administered is any of the disclosed compounds or at least one product of the disclosed methods of making.

In a further aspect, the mammal is a human. In a yet further aspect, the mammal has been diagnosed with a need for treatment of the disorder prior to the administering step. In a still further aspect, the method further comprises the step of identifying a mammal in need of treatment of the disorder. In an even further aspect, an effective amount is a therapeutically effective amount.

In a further aspect, modulation of PCNA activity is inhibition of PCNA activity. In a still further aspect, modulation of PCNA activity is inhibition of PCNA protein-protein interactions with another protein. In yet further aspect, modulation of PCNA activity is antagonism of protein-protein interactions with another protein. In an even further aspect, modulation of PCNA activity is inhibition of binding of another protein to the PIP-box binding site on PCNA.

In a further aspect, the compound inhibits PCNA protein activity. In an even further aspect, the inhibition of binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the compound that inhibits cell growth. In a still further aspect, the compound inhibits cell growth with an IC₅₀ of less than about 500 μM, less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, the PCNA dysfunction is increased PCNA activity. In a still further aspect, the disorder is a proliferative disorder. In a yet further aspect, the proliferative disorder is a hyperproliferative disorder.

In a further aspect, the hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the hyperproliferative disorder is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the compound is co-administered with a chemotherapeutic agent. In a yet further aspect, the chemotherapeutic agent is an alkylating agent. In a still further aspect, the chemotherapeutic agent is cisplatin. In an even further aspect, the chemotherapeutic agent is selected from actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

c. Treatment of a Proliferative Disorder

In one aspect, the invention relates to a method for treatment of a proliferative disorder in a mammal comprising the step of administering to the mammal an effective amount of at least one compound having a structure represented by formula:

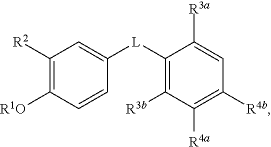

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is selected from:

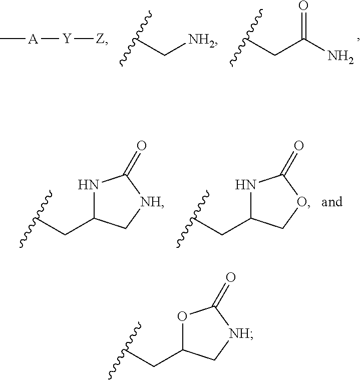

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for treatment of a proliferative disorder in a mammal comprising the step of administering to the mammal an effective amount of at least one compound having a structure represented by formula:

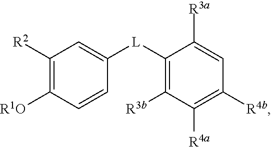

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for treatment of a proliferative disorder in a mammal comprising the step of administering to the mammal an effective amount of at least one compound having a structure represented by formula:

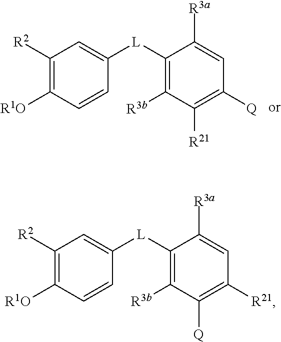

wherein L is selected from O, CH₂, CHOH, and C═O; wherein Q is selected from a structure represented by a formula:

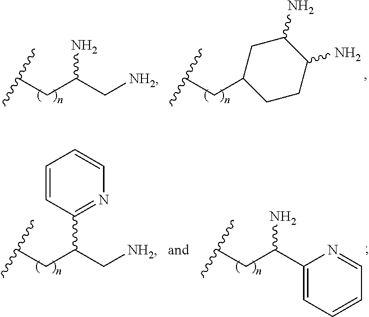

wherein n is an integer selected from 0, 1, 2, 3, 4, 5, and 6; wherein W is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound administered is any of the disclosed compounds or at least one product of the disclosed methods of making.

In a further aspect, the mammal is a human. In a yet further aspect, the mammal has been diagnosed with a need for treatment of the disorder prior to the administering step. In a still further aspect, the method further comprises the step of identifying a mammal in need of treatment of the disorder. In an even further aspect, an effective amount is a therapeutically effective amount.

In a further aspect, the compound inhibits PCNA protein activity. In an even further aspect, the inhibition of binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the compound that inhibits cell growth. In a still further aspect, the compound inhibits cell growth with an IC₅₀ of less than about 500 μM, less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, the proliferative disorder is associated with a PCNA dysfunction. In a yet further aspect, the PCNA dysfunction is increased PCNA activity. In a still further aspect, the proliferative disorder is a hyperproliferative disorder.

In a further aspect, the hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the hyperproliferative disorder is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the compound is co-administered with a chemotherapeutic agent. In a yet further aspect, the chemotherapeutic agent is an alkylating agent. In a still further aspect, the chemotherapeutic agent is cisplatin. In an even further aspect, the chemotherapeutic agent is selected from actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

d. Inhibition of Cell Growth

In one aspect, the invention relates to a method for inhibiting cell growth in a mammal comprising the step of administering to the mammal an effective amount of at least one compound having a structure represented by formula:

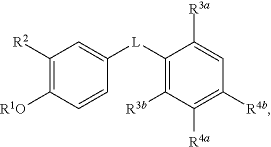

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is selected from:

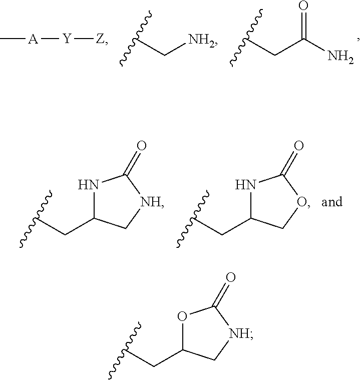

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for inhibiting cell growth in a mammal comprising the step of administering to the mammal an effective amount of at least one compound having a structure represented by formula:

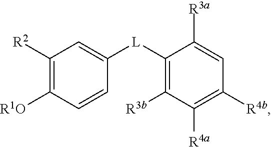

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for inhibiting cell growth in a mammal comprising the step of administering to the mammal an effective amount of at least one compound having a structure represented by formula:

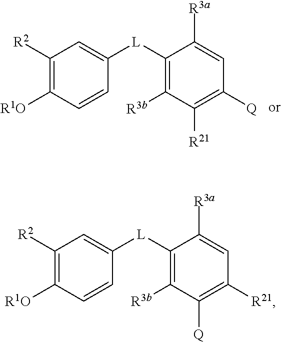

wherein L is selected from O, CH₂, CHOH, and C═O; wherein Q is selected from a structure represented by a formula:

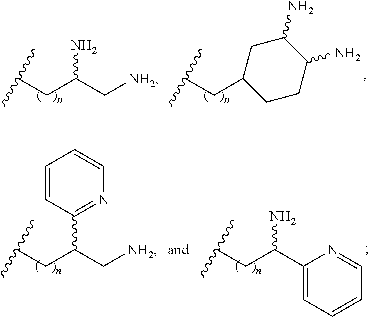

wherein n is an integer selected from 0, 1, 2, 3, 4, 5, and 6; wherein IV is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound administered is any of the disclosed compounds or at least one product of the disclosed methods of making.

In a further aspect, the mammal is a human. In a yet further aspect, the mammal has been diagnosed with a need for treatment of the disorder prior to the administering step. In a still further aspect, the method further comprises the step of identifying a mammal in need of treatment of the disorder. In an even further aspect, an effective amount is a therapeutically effective amount.

In a further aspect, the compound inhibits PCNA protein activity. In an even further aspect, the inhibition of binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the compound inhibits cell growth with an IC₅₀ of less than about 500 μM. In a yet further aspect, the compound inhibits cell growth with an IC₅₀ of less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, inhibiting cell growth treats a proliferative disorder. In a still further aspect, the proliferative disorder is associated with a PCNA dysfunction. In a yet further aspect, the PCNA dysfunction is increased PCNA activity. In a still further apect, the proliferative disorder is a hyperproliferative disorder.

In a further aspect, the hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the hyperproliferative disorder is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the compound is co-administered with a chemotherapeutic agent. In a yet further aspect, the chemotherapeutic agent is an alkylating agent. In a still further aspect, the chemotherapeutic agent is cisplatin. In an even further aspect, the chemotherapeutic agent is selected from actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

e. Cytostatic Therapy

In one aspect, the invention relates to a method for cytostatic therapy in a mammal comprising the step of administering to the mammal an effective amount of at least one compound having a structure represented by formula:

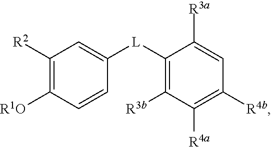

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is selected from:

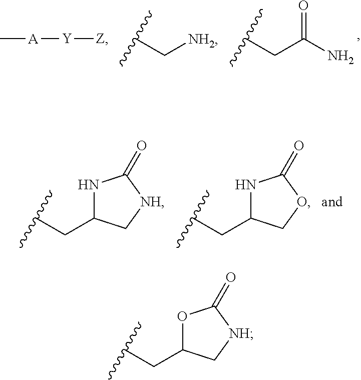

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹¹ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for cytostatic therapy in a mammal comprising the step of administering to the mammal an effective amount of at least one compound having a structure represented by formula:

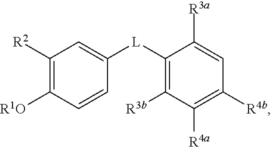

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for cytostatic therapy in a mammal comprising the step of administering to the mammal an effective amount of at least one compound having a structure represented by formula:

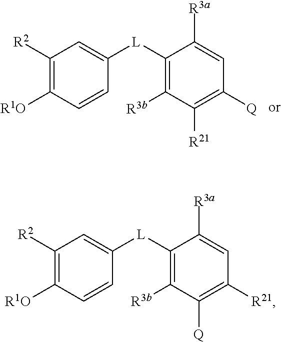

wherein L is selected from O, CH₂, CHOH, and C═O; wherein Q is selected from a structure represented by a formula:

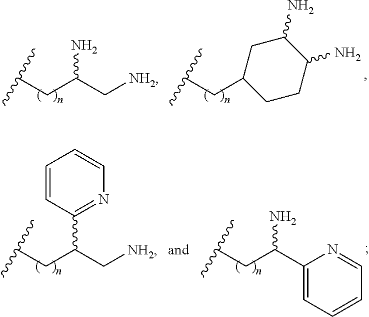

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound administered is any of the disclosed compounds or at least one product of the disclosed methods of making.

In a further aspect, the mammal is a human. In a yet further aspect, the mammal has been diagnosed with a need for treatment of the disorder prior to the administering step. In a still further aspect, the method further comprises the step of identifying a mammal in need of treatment of the disorder. In an even further aspect, an effective amount is a therapeutically effective amount.

In a further aspect, the compound inhibits PCNA protein activity. In an even further aspect, the inhibition of binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the compound that inhibits cell growth. In a still further aspect, the compound inhibits cell growth with an IC₅₀ of less than about 500 μM, less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, cytostatic therapy treats a proliferative disorder. In a still further aspect, the proliferative disorder is associated with a PCNA dysfunction. In a yet further aspect, the PCNA dysfunction is increased PCNA activity. In a still further apect, the proliferative disorder is a hyperproliferative disorder.

In a further aspect, the hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the hyperproliferative disorder is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the compound is co-administered with a chemotherapeutic agent. In a yet further aspect, the chemotherapeutic agent is an alkylating agent. In a still further aspect, the chemotherapeutic agent is cisplatin. In an even further aspect, the chemotherapeutic agent is selected from actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

f. Modulating PCNA Activity in Cells

In one aspect, the invention relates to a method for modulating PCNA activity in at least one cell, comprising the step of contacting the at least one cell with an effective amount of at least one disclosed compound or a product of a disclosed method of making.

Thus, in one aspect, the invention relates to a method for modulation of PCNA activity in at least one cell, comprising the step of contacting the at least one cell with an effective amount of at least one compound having a structure represented by a formula:

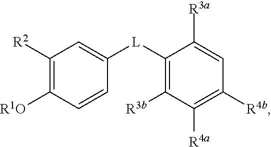

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is selected from:

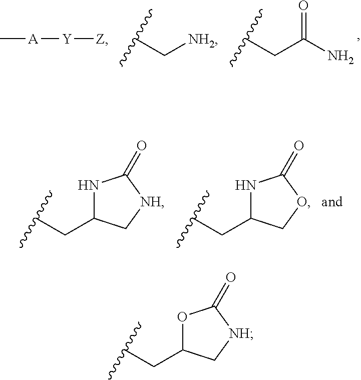

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for modulation of PCNA activity in at least one cell, comprising the step of contacting the at least one cell with an effective amount of at least one compound having a structure represented by a formula:

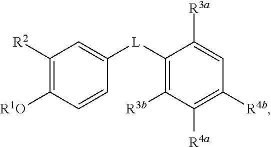

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a method for modulation of PCNA activity in at least one cell, comprising the step of contacting the at least one cell with an effective amount of at least one compound having a structure represented by a formula:

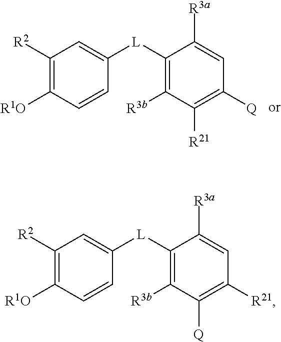

wherein L is selected from O, CH₂, CHOH, and C═O; wherein Q is selected from a structure represented by a formula:

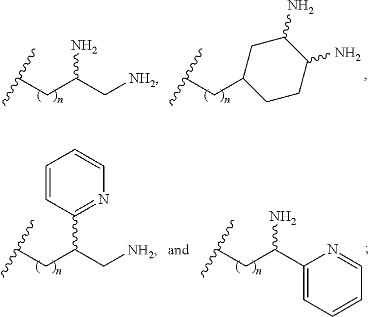

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound administered is any of the disclosed compounds or at least one product of the disclosed methods of making.

In a further aspect, the at least one cell is mammalian. In a still further aspect, the method further comprises administering to the mammal the compound in an amount sufficient to contact at least one cell in the mammal. In a yet further aspect, the cell has been isolated from a mammal prior to the contacting step. In an even further aspect, the cell has a PCNA dysfunction. In a still further aspect, one cell has increased PCNA activity.

In a further aspect, contacting is via administration to a mammal. In a further aspect, the mammal has been diagnosed with a need for modulating PCNA protein activity prior to the administering step. In a further aspect, the mammal has been diagnosed with a need for treatment of a disorder related to a PCNA protein activity dysfunction prior to the administering step.

In a further aspect, the at least one cell is in a human. In a yet further aspect, the mammal has been diagnosed with a need for treatment of the disorder prior to the contacting step. In a still further aspect, the method further comprises the step of identifying a mammal in need of treatment of the disorder.

In a further aspect, modulating is inhibition. In a still further aspect, the compound contacting the cell inhibits PCNA protein activity. In an even further aspect, the compound inhibits binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the compound that inhibits cell growth. In a still further aspect, the compound inhibits cell growth with an IC₅₀ of less than about 500 μM, less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, contacting the cell treats a disorder. In a still further aspect, the disorder is a proliferative disorder. In a yet further aspect, the proliferative disorder is a hyperproliferative disorder.

In a further aspect, the hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the hyperproliferative disorder is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the cell is co-contacted with a chemotherapeutic agent. In an even further aspect, co-contacting is via co-administration of a chemotherapeutic agent to a mammal. In a yet further aspect, the chemotherapeutic agent is an alkylating agent. In a still further aspect, the chemotherapeutic agent is cisplatin. In an even further aspect, the chemotherapeutic agent is selected from actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

2. Manufacture of a Medicament

In one aspect, the invention relates to a method for the manufacture of a medicament for treatment of a proliferative disorder activity in a mammal comprising combining a therapeutically effective amount of at least one disclosed compound or at least one product of a disclosed method with a pharmaceutically acceptable carrier or diluent.

3. Use of Compounds

In one aspect, the invention relates to the use of a compound having a structure represented by a formula:

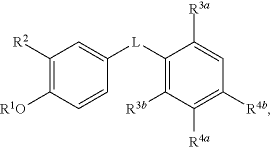

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is selected from:

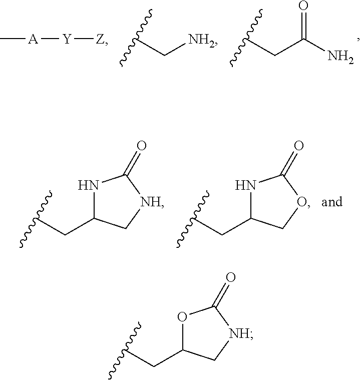

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C₁-C₃ haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C₁-C₃ alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to the use of a compound having a structure represented by a formula:

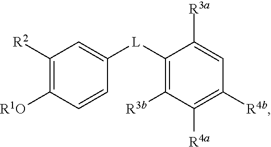

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to the use of a compound having a structure represented by a formula:

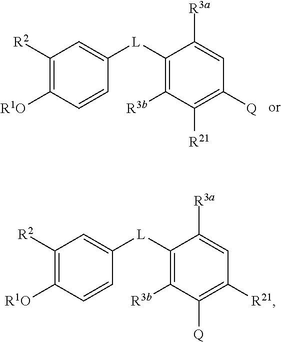

wherein L is selected from O, CH₂, CHOH, and C═O; wherein Q is selected from a structure represented by a formula:

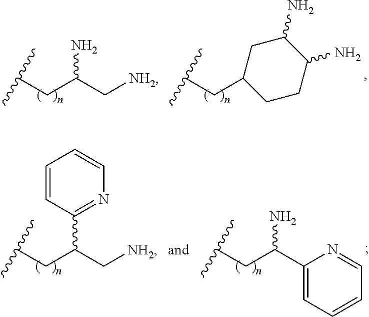

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof.

In a further aspect, the compound used is any of the disclosed compounds or at least one product of the disclosed methods of making.

In a further aspect, the use relates to inhibition of PCNA protein activity. In an even further aspect, the inhibition is of binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the use relates to inhibition of cell growth. In a still further aspect, the compound inhibits cell growth with an IC₅₀ of less than about 500 μM, less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, the use treats a PCNA dysfunction. In a still further aspect, the PCNA dysfunction is increased PCNA activity. In a still further aspect, the use treats a proliferative disorder. In a yet further aspect, the proliferative disorder is a hyperproliferative disorder.

In a further aspect, the use treats a hyperproliferative disorder selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the use treats a hyperproliferative disorder that is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the use treats a cancer that is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the use treats a cancer that is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the use further comprises use with a chemotherapeutic agent. In a yet further aspect, the chemotherapeutic agent is an alkylating agent. In a still further aspect, the chemotherapeutic agent is cisplatin. In an even further aspect, the chemotherapeutic agent is selected from actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

4. Kits

In one aspect, the invention relates to a kit comprising at least one compound having a structure represented by a formula:

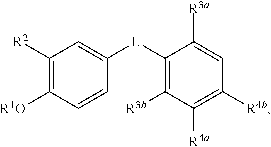

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is selected from:

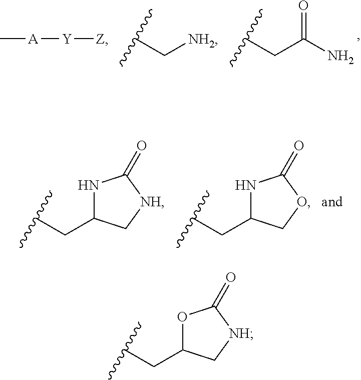

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C₁-C₃ haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C₁-C₃ alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof.

In various aspects, the invention relates to a kit comprising at least one compound having a structure represented by a formula:

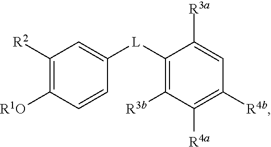

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof, and one or more of: (a) at least one agent known to decrease PCNA activity; (b) at least one agent known to increase PCNA activity; (c) at least one agent known to treat a proliferative disorder; or (d) instructions for treating a disorder associated with PCNA dysfunction.

In one aspect, the invention relates to a kit comprising at least one compound having a structure represented by a formula:

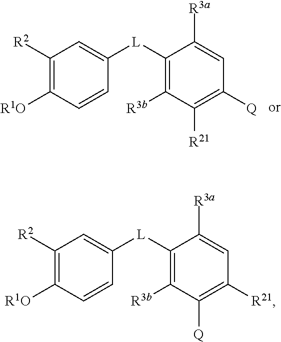

wherein L is selected from O, CH₂, CHOH, and C═O; wherein Q is selected from a structure represented by a formula:

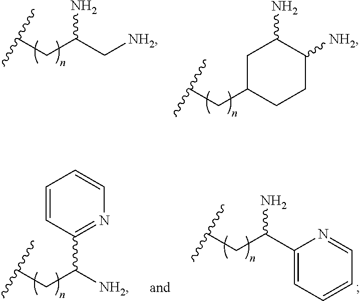

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof, and one or more of: (a) at least one agent known to decrease PCNA activity; (b) at least one agent known to increase PCNA activity; (c) at least one agent known to treat a proliferative disorder; or (d) instructions for treating a disorder associated with PCNA dysfunction.

In a further aspect, the kit comprises a disclosed compound or a product of a disclosed method.

In a further aspect, the at least one compound and the at least one agent are co-formulated. In a still further aspect, the at least one compound and the at least one agent are co-packaged.

In a further aspect, the at least one compound in the kit inhibits PCNA protein activity. In an even further aspect, the compound inhibits binding of PL peptide to PCNA. In a yet further aspect, the binding of PL peptide to PCNA is determined using a fluorescent polarization assay. In a yet further aspect, the IC₅₀ for inhibition of PL peptide binding to PCNA is less than about 100 μM, less than about 50 μM, less than about 10 μM, less than about 5 μM, less than about 1 μM, less than about 500 nM, or of less than about 100 nM.

In a further aspect, the at least one compound in the kit inhibits cell growth. In a still further aspect, the compound inhibits cell growth with an IC₅₀ of less than about 500 μM, less than about 250 μM, less than about 100 μM, less than about 50 μM, less than about 10 μM, less than about 1 μM, less than about 0.5 μM, or less than about 0.1 μM. In a still further aspect, the IC₅₀ for inhibition of cell growth is determined in a cell line derived from a cancer. In a yet further aspect, the IC₅₀ for inhibition of cell growth is determined in HeLa cells.

In a further aspect, the at least one compound in the kit treats a disorder is associated with a PCNA dysfunction. In a still further aspect, the PCNA dysfunction is increased PCNA activity.

In a further aspect, the at least one compound in the kit treats a proliferative disorder. In a yet further aspect, the proliferative disorder is a hyperproliferative disorder.

In a further aspect, the hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder. In a yet further aspect, the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the hyperproliferative disorder is a cancer. In a still further aspect, the cancer is a hematological cancer. In a yet further aspect, the cancer is selected from a cancer of the breast, lung, ovary, prostate, head, neck, and kidney. In a still further aspect, the cancer is selected from a cancer of the head, neck, pancreas, brain, ovary, kidney, prostate, breast, lung, colon, and liver. In an even further aspect, the cancer is selected from a cancer of the breast, ovary, prostate, head, neck, and kidney. In an even further aspect, the cancer is breast cancer. In a still further aspect, the cancer is pancreatic cancer. In a yet further aspect, the cancer is lung cancer. In an even further aspect, the lung cancer is non-small cell lung cancer. In a still further aspect, the lung cancer is small cell lung cancer.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the at least one agent known to treat a proliferative disorder is a hormone therapy agent. In a still further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the at least one agent known to treat proliferative disorder is a chemotherapeutic agent. In a yet further aspect, the chemotherapeutic agent is cisplatin. In a still further aspect, the chemotherapeutic agent is selected from one or more of actinomycin D, BCNU (carmustine), carboplatin, CCNU, campothecin (CPT), cantharidin, cisplatin, cyclophosphamide, cytarabine, dacarbazine, daunorubicin, docetaxel, doxorubicin, DTIC, epirubicin, etoposide, gefinitib, gemcitabine, ifosamide irinotecan, ionomycin, Melphalan, methotrexate, mitomycin C (MMC), mitozantronemercaptopurine, oxaliplatin, paclitaxel, PARP-1 inhibitor, taxotere, temozolomide, teniposide, topotecane, treosulfane vinorelbine, vincristine, vinblastine, 5-azacytidine, 5,6-dihydro-5-azacytidine and 5-fluorouracil.

In a further aspect, the chemotherapeutic agent is selected from one or more of the group consisting of an alkylating agent, an antimetabolite agent, an antineoplastic antibiotic agent, a mitotic inhibitor agent, a mTor inhibitor agent or other chemotherapeutic agent. In a still further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof. In an even further aspect, the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof. In a still further aspect, the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof. In a yet further aspect, the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof. In an even further aspect, the mTor inhibitor agent is selected from one or more of the group consisting of everolimus, siroliumus, and temsirolimus, or a pharmaceutically acceptable salt, hydrate, solvate, or polymorph thereof.

In a further aspect, the at least one compound and the at least one agent are co-packaged. In a still further aspect, the at least one agent that is co-packaged with the at least one compound is one of the agents described herein.

In a further aspect, the at least one compound and the at least one agent are co-formulated. In a still further aspect, the at least one agent that is co-formulated with the at least one compound is one of the agents described herein.

In a further aspect, the instructions further comprise providing the compound in connection surgery. In a still further aspect, the instructions provide that surgery is performed prior to the administering of at least one compound. In a yet further aspect, the instructions provide that surgery is performed after the administering of at least one compound. In an even further aspect, the instructions provide that the administering of at least one compound is to effect presurgical debulking of a tumor. In a yet further aspect, the instructions provide that surgery is performed at about the same time as the administering of at least one compound.

In a further aspect, the instructions further comprise providing the compound in connection with radiotherapy. In a yet further aspect, the instructions provide that radiotherapy is performed prior to the administering of at least one compound. In a still further aspect, the instructions provide that radiotherapy is performed after the step of the administering of at least one compound. In an even further aspect, the instructions provide that radiotherapy is performed at about the same time as the step of the administering of at least one compound. In a still further aspect, the instructions further comprise providing the compound in connection with at least one agent that is a chemotherapeutic agent.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

5. Non-Medical Uses

Also provided are the uses of the disclosed compounds and products as pharmacological tools in the development and standardization of in vitro and in vivo test systems for the evaluation of the effects of inhibitors of PCNA related activity in laboratory animals such as cats, dogs, rabbits, monkeys, rats and mice, as part of the search for new therapeutic agents of for new therapeutic agents targeting PCNA protein. Also provided are the uses of the disclosed compounds and products as pharmacological tools in the development and standardization of in vitro and in vivo test systems for the evaluation of the effects of inhibitors of PCNA protein related activity in laboratory animals such as cats, dogs, rabbits, monkeys, rats and mice, as part of the search for new therapeutic agents targeting PCNA protein.

### G. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following Examples. Starting materials and the requisite intermediates are in some cases commercially available, or can be prepared according to literature procedures or as illustrated herein.

The following exemplary compounds of the invention were synthesized. The Examples are provided herein to illustrate the invention, and should not be construed as limiting the invention in any way. The Examples are typically depicted in free base form, according to the IUPAC naming convention. However, some of the Examples were obtained or isolated in salt form.

### 1. Abbreviations

Boc, di-tert-butyl dicarbonate; CDI, 1,1′-carbonyldiimidazole; DBU, 1,8-diazabicyclo[5.4.0]undec-7-ene; DCM, dichloromethane; DIPEA, N,N-diisopropylethylamine; DMF, dimethylformamide; DPPA, diphenylphosphoryl azide; HATU, 2-(1H-7-azabenzo[d][1,2,3]triazol-1-yl)-1,1,3,3-tetramethyl-uronium hexafluorophosphate; PMB, paramethoxybenzyl; TEA, triethylamine; TFA, trifluoroacetic acid; THF, tetrahydrofuran; and TMS-Cl, trimethylsilyl chloride.

### 2. General Chemical Methods

All commercial reagents were used without further purification and the solvents were dried using the dry solvent system (Glass Contour Solvent Systems, SG Water USA). Reactions requiring the exclusion of air were carried out under an atmosphere of dry nitrogen in oven dried glassware. All reactions were monitored by thin-layer chromatography (TLC) carried out on EMD Chemicals silica gel 60-F 254 coated glass plates and visualized using UV light (254 nm). Analysis by LC-MS was performed by using an XBridge C18 column run at 1 mL/min, and using gradient mixtures of (A) water (0.05% TFA) and (B) methanol. Low resolution mass spectra (ESI) were collected on a Waters Micromass ZQ in positive-ion mode. Preparative TLC was performed on EMD Chemicals silica gel 60-F254 coated glass plates and visualized using UV light (254 nm). Flash-chromatography was performed on Biotage SP4 chromatography system using Biotage Flash+KP SIL pre-packed columns and the solvent mixture in brackets was used as eluent. Nuclear magnetic resonance (NMR) spectra were obtained on Bruker Avance II NMR spectrometer at 400 MHz for ¹H-NMR spectra. Chemical shifts (ppm) are reported relative to TMS or the solvent peak. Signals are designated as follows: s, singlet; d, doublet; dd, doublet of doublet; t, triplet; q, quadruplet; m, multiplet. Coupling constants (J) are shown in Hertz.

### 3. General Method (A) for Amide Coupling Using HATU

A solution of the carboxylic acid (0.016 mmol), HATU (0.040 mmol), and DIPEA (0.048 mmol), in DMF (200 μL), was allowed to stand for 30 min. The appropriate amine (0.019 mmol) was added to the reaction mixture and the solution was allowed to stand overnight. Water (3 mL) was added to the reaction mixture and extracted with ethyl acetate (3×3 mL). The combined organic layers were washed with saturated brine, dried over Na₂SO₄, filtered, and concentrated. The crude product was purified by either flash column chromatography (Biotage SP4, eluting with hexanes/ethyl acetate, or DCM/MeOH), or preparative TLC (eluting with DCM/MeOH, 9:1) to give the desired product.

### 4. General Method (B) for Sulfonamide Coupling Using CDI

CDI (0.035 mmol) was added to a solution of the carboxylic acid (0.032 mmol) in DCM (300 μL), at 0° C. After 5 min, the ice bath was removed and the reaction mixture was stirred at rt for 1 h. The appropriate sulfonamide (0.035 mmol) and DBU (0.064 mmol) were added to the reaction mixture and the solution stirred overnight. Aqueous 10% NaH₂PO₄ (3 mL) was added to the reaction mixture and extracted with DCM (3×3 mL). The combined organic layers were washed with saturated brine, dried over Na₂SO₄, filtered, and concentrated. The crude product was purified by either flash column chromatography (Biotage SP4, eluting with DCM/MeOH), or preparative TLC (eluting with DCM/MeOH, 9:1) to give the desired product.

### 5. General Method (C) for Demethylation Using BBR₃

A 1M in DCM solution of boron tribromide (0.210 mmol) was added dropwise to a solution of the aryl methyl ether (0.021 mmol) in DCM (300 μL), at −78° C. The solution was stirred for 5 min and then for 2 h at rt. The reaction mixture was added to water (3 mL), and extracted with ethyl acetate (3×3 mL). The combined organic layers were washed with saturated brine, dried over Na₂SO₄, filtered, and concentrated. The crude product was purified by either filtering through a pad of silica gel (eluting with DCM/MeOH, 9:1), flash column chromatography (Biotage SP4, eluting with DCM/MeOH), or by preparative TLC (eluting with DCM/MeOH, 9:1) to give the desired product.

### 6. General Method (D) for Sulfonamide Coupling Using Sulfonylchlorides and Tea

A solution of the amine (0.023 mmol), the appropriate sulfonylchloride (0.026 mmol), and TEA (0.026 mmol), in DMF (200 μL) was allowed to stand for 15 min. The reaction mixture was added to water (3 mL) and extracted with ethyl acetate (3×3 mL). The combined organic layers were washed with saturated brine, dried over Na₂SO₄, filtered, and concentrated. The crude product was purified by preparative TLC (eluting with DCM/MeOH, 9:1) to give the desired product.

### 7. Preparation of 2-(4-(4-Hydroxy-3-iodophenoxy)-3,5-diiodophenyl)-N-phenylacetamide (MLAE-029-11

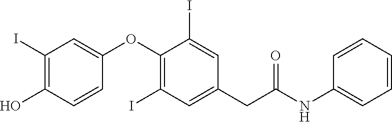 [SMILES: O=C(Cc1cc(I)c(Oc2ccc(O)c(I)c2)c(I)c1)Nc1ccccc1]

Starting with 2-(4-(4-hydroxy-3-iodophenoxy)-3,5-diiodophenyl)acetic acid, general method (A) was used to give the desired product (1.8 mg, 16% yield). MS (ESI) m/z, [M+H] calculated, 697.82; observed, 697.64.

### 8. Preparation of N-Cyclohexyl-2-(4-(4-hydroxy-3-iodophenoxy)-3,5-diiodophenyl)acetamide (MLAE-029-2)

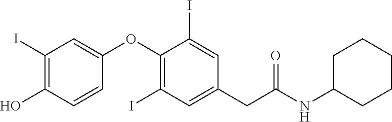 [SMILES: O=C(Cc1cc(I)c(Oc2ccc(O)c(I)c2)c(I)c1)NC1CCCCC1]

Starting with 2-(4-(4-hydroxy-3-iodophenoxy)-3,5-diiodophenyl)acetic acid, general method (A) was used to give the desired product (4.2 mg, 37% yield). MS (ESI) m/z, [M+H] calculated, 703.87; observed, 703.62.

### 9. Preparation of 2-(4-(4-hydroxy-3-iodophenoxy)-3,5-diiodophenyl)-N-phenethylacetamide (MLAE-029-3)

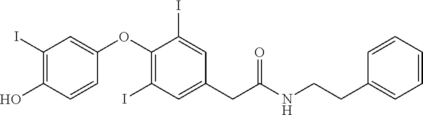 [SMILES: O=C(Cc1cc(I)c(Oc2ccc(O)c(I)c2)c(I)c1)NCCc1ccccc1]

Starting with 2-(4-(4-hydroxy-3-iodophenoxy)-3,5-diiodophenyl)acetic acid, general method (A) was used to give the desired product (3.7 mg, 32% yield). MS (ESI) m/z, [M+H] calculated, 725.85; observed, 725.61.

### 10. Preparation of N-Benzyl-2-(4-(4-hydroxy-3-iodophenoxy)-3,5-diiodophenyl)acetamide (MLAE-029-4)

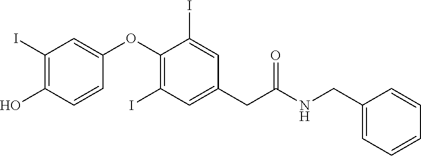 [SMILES: O=C(Cc1cc(I)c(Oc2ccc(O)c(I)c2)c(I)c1)NCc1ccccc1]

Starting with 2-(4-(4-hydroxy-3-iodophenoxy)-3,5-diiodophenyl)acetic acid, general method (A) was used to give the desired product (3.7 mg, 32% yield). MS (ESI) m/z, [M+H] calculated, 711.83; observed, 711.62.

### 11. Preparation of N,N-Diethyl-2-(4-(4-hydroxy-3-iodophenoxy)-3,5-diiodophenyl)acetamide (MLAE-029-6)

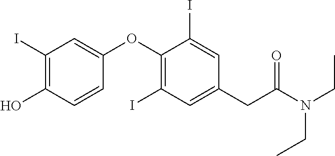 [SMILES: CCN(CC)C(=O)Cc1cc(I)c(Oc2ccc(O)c(I)c2)c(I)c1]

Starting with 2-(4-(4-hydroxy-3-iodophenoxy)-3,5-diiodophenyl)acetic acid, general method (A) was used to give the desired product (0.9 mg, 8% yield). MS (ESI) m/z, [M+H] calculated, 677.85; observed, 677.73.

### 12. Preparation of 2-(4-(4-Hydroxy-3-iodophenoxy)-3,5-diiodophenyl)-N-(4-methoxyphenyl)acetamide (MLAE-029-7)

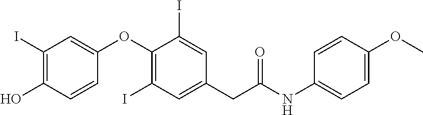 [SMILES: COc1ccc(NC(=O)Cc2cc(I)c(Oc3ccc(O)c(I)c3)c(I)c2)cc1]

Starting with 2-(4-(4-hydroxy-3-iodophenoxy)-3,5-diiodophenyl)acetic acid, general method (A) was used to give the desired product (0.9 mg, 8% yield). MS (ESI) m/z, [M+H] calculated, 727.83; observed, 727.76.

### 13. Preparation of 2-(4-(4-Hydroxy-3-iodophenoxy)-3,5-diiodophenyl)-1-(piperidin-1-yl)ethanone (MLAE-029-8)

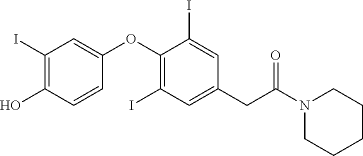 [SMILES: O=C(Cc1cc(I)c(Oc2ccc(O)c(I)c2)c(I)c1)N1CCCCC1]

Starting with 2-(4-(4-hydroxy-3-iodophenoxy)-3,5-diiodophenyl)acetic acid, general method (A) was used to give the desired product (3.4 mg, 31% yield). MS (ESI) m/z, [M+H] calculated, 689.85; observed, 689.64.

### 14. Preparation of 2-(4-(4-Hydroxy-3-iodophenoxy)-3,5-diiodophenyl)-N-pentylacetamide (MLAE-029-9)

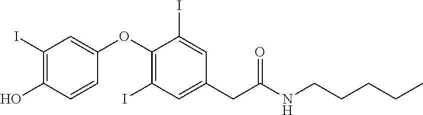 [SMILES: CCCCCNC(=O)Cc1cc(I)c(Oc2ccc(O)c(I)c2)c(I)c1]

Starting with 2-(4-(4-hydroxy-3-iodophenoxy)-3,5-diiodophenyl)acetic acid, general method (A) was used to give the desired product (4.0 mg, 36% yield). MS (ESI) m/z, [M+H] calculated, 691.87; observed, 691.65.

### 15. Preparation of 2-(4-(4-Hydroxy-3-iodophenoxy)-3,5-diiodophenyl)-N-methylacetamide (MLAE-030)

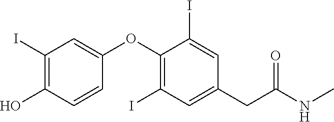 [SMILES: CNC(=O)Cc1cc(I)c(Oc2ccc(O)c(I)c2)c(I)c1]

Starting with 2-(4-(4-hydroxy-3-iodophenoxy)-3,5-diiodophenyl)acetic acid, general method (A) was used to give the desired product (8.4 mg, 40% yield). MS (ESI) m/z, [M+H] calculated, 635.80; observed, 635.61.

### 16. Preparation of 2-(4-(4-Hydroxy-3-iodophenoxy)-3,5-diiodophenyl)-N,N-dimethylacetamide (MLAE-031)

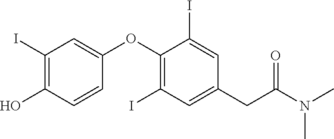 [SMILES: CN(C)C(=O)Cc1cc(I)c(Oc2ccc(O)c(I)c2)c(I)c1]

Starting with 2-(4-(4-hydroxy-3-iodophenoxy)-3,5-diiodophenyl)acetic acid, general method (A) was used to give the desired product (12.5 mg, 60% yield). MS (ESI) m/z, [M+H] calculated, 649.82; observed, 649.65.

### 17. Preparation of 2-(4-(4-Hydroxy-3-iodophenoxy)-3,5-diiodophenyl)-N-(methylsulfonyl)acetamide (MLAE-032)

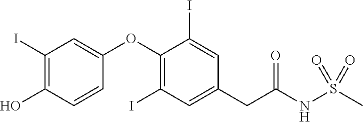 [SMILES: CS(=O)(=O)NC(=O)Cc1cc(I)c(Oc2ccc(O)c(I)c2)c(I)c1]

Starting with 2-(4-(4-hydroxy-3-iodophenoxy)-3,5-diiodophenyl)acetic acid, general method (B) was used to give the desired product (4.7 mg, 21% yield). ¹H NMR (400 MHz, MeOD) δ 7.88 (s, 2H), 7.04 (d, J=2.9 Hz, 1H), 6.77 (d, J=8.9 Hz, 1H), 6.63 (dd, J=8.9, 3.0 Hz, 1H), 3.61 (s, 2H), 3.21 (s, 3H); MS (ESI) m/z, [M+H] calculated, 699.76; observed, 699.53.

### 18. Preparation of 2-(4-(4-Hydroxy-3-iodophenoxy)-3,5-diiodophenyl)-N-(phenylsulfonyl)acetamide (MLAE-033)

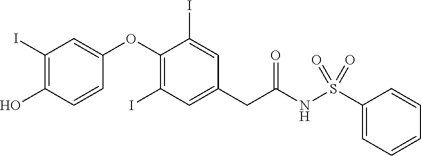 [SMILES: O=C(Cc1cc(I)c(Oc2ccc(O)c(I)c2)c(I)c1)NS(=O)(=O)c1ccccc1]

Starting with 2-(4-(4-hydroxy-3-iodophenoxy)-3,5-diiodophenyl)acetic acid, general method (B) was used to give the desired product (3.8 mg, 16% yield). MS (ESI) m/z, [M+H] calculated, 761.78; observed, 761.56.

### 19. Preparation of 4-(3-(4-(4-hydroxyphenoxy)-3,5-diiodophenyl)propanoyl)piperazin-2-one (MLAE-034)

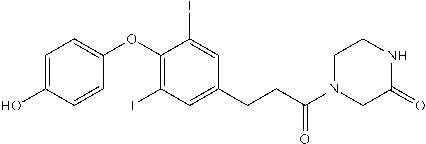 [SMILES: O=C1CN(C(=O)CCc2cc(I)c(Oc3ccc(O)cc3)c(I)c2)CCN1]

Starting with 3-(4-(4-hydroxyphenoxy)-3,5-diiodophenyl)propanoic acid, general method (A) was used to give the desired product (13.3 mg, 53% yield). MS (ESI) m/z, [M+H] calculated, 592.94; observed, 592.81.

### 20. Preparation of 3-(4-(4-hydroxyphenoxy)-3,5-diiodophenyl)-1-morpholinopropan-1-one (MLAE-035)

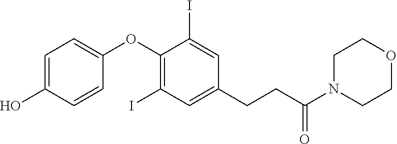 [SMILES: O=C(CCc1cc(I)c(Oc2ccc(O)cc2)c(I)c1)N1CCOCC1]

Starting with 3-(4-(4-hydroxyphenoxy)-3,5-diiodophenyl)propanoic acid, general method (A) was used to give the desired product (16.3 mg, 67% yield). MS (ESI) m/z, [M+H] calculated, 579.95; observed, 579.84.

### 21. Preparation of 3-(3,5-diiodo-4-(4-methoxyphenoxy)phenyl)-N-(methylsulfonyl)propanamide (MLAE-036)

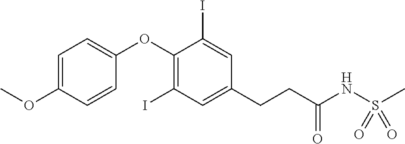

Starting with 3-(3,5-diiodo-4-(4-methoxyphenoxy)phenyl)propanoic acid, general method (B) was used to give the desired product (10 mg, 58% yield). MS (ESI) m/z, [M+H] calculated, 601.90; observed, 601.75.

### 22. Preparation of 2-(4-(4-Hydroxy-3-iodophenoxy)-3,5-diiodophenyl)-1-morpholinoethanone (MLAE-037)

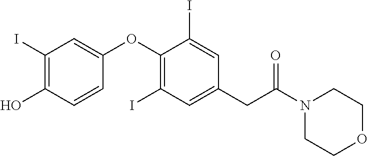 [SMILES: O=C(Cc1cc(I)c(Oc2ccc(O)c(I)c2)c(I)c1)N1CCOCC1]

Starting with 2-(4-(4-hydroxy-3-iodophenoxy)-3,5-diiodophenyl)acetic acid, general method (A) was used to give the desired product (9.6 mg, 43% yield). MS (ESI) m/z, [M+H] calculated, 691.83; observed, 691.53.

### 23. Preparation of 3-(4-(4-hydroxyphenoxy)-3,5-diiodophenyl)-N-(methylsulfonyl)propanamide (MLAE-038)

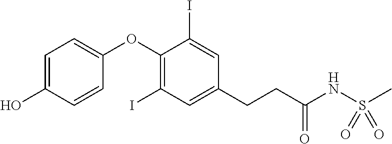 [SMILES: CS(=O)(=O)NC(=O)CCc1cc(I)c(Oc2ccc(O)cc2)c(I)c1]

Starting with MLAE-036, general method (C) was used to give the desired product (7.5 mg, 96% yield). ¹H NMR (400 MHz, MeOD) δ 7.79 (s, 2H), 6.76-6.62 (m, 2H), 6.59-6.48 (m, 2H), 3.20 (s, 3H), 2.90 (t, J=7.4 Hz, 3H), 2.62 (t, J=7.4 Hz, 3H); MS (ESI) m/z, [M+H] calculated, 691.83; observed, 691.53.

### 24. Preparation of 3-(3,5-Diiodo-4-(4-methoxyphenoxy)phenyl)-1-(piperidin-1-yl)propan-1-one (MLAE-039)

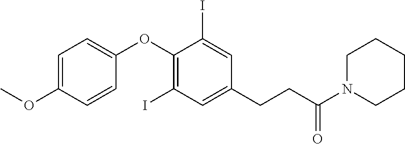 [SMILES: COc1ccc(Oc2c(I)cc(CCC(=O)N3CCCCC3)cc2I)cc1]

Starting with 3-(3,5-diiodo-4-(4-methoxyphenoxy)phenyl)propanoic acid, general method (A) was used to give the desired product (14.4 mg, 70% yield). MS (ESI) m/z, [M+H] calculated, 591.98; observed, 591.86.

### 25. Preparation of 3-(3,5-Diiodo-4-(4-methoxyphenoxy)phenyl)-N-methylpropanamide (MLAE-040)

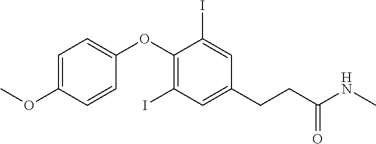

Starting with 3-(3,5-diiodo-4-(4-methoxyphenoxy)phenyl)propanoic acid, general method (A) was used to give the desired product (15.2 mg, 81% yield). MS (ESI) m/z, [M+H] calculated, 537.94; observed, 537.79.

### 26. Preparation of 3-(4-(4-hydroxyphenoxy)-3,5-diiodophenyl)-1-(piperidin-1-yl)propan-1-one (MLAE-041)

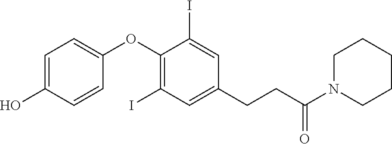 [SMILES: O=C(CCc1cc(I)c(Oc2ccc(O)cc2)c(I)c1)N1CCCCC1]

Starting with MLAE-039, general method (C) was used to give the desired product (11.2 mg, 93% yield). MS (ESI) m/z, [M+H] calculated, 577.97; observed, 577.83.

### 27. Preparation of 3-(4-(4-hydroxyphenoxy)-3,5-diiodophenyl)-N-methylpropanamide (MLAE-042)

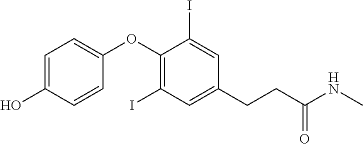 [SMILES: CNC(=O)CCc1cc(I)c(Oc2ccc(O)cc2)c(I)c1]

Starting with MLAE-040, general method (C) was used to give the desired product (6 mg, 47% yield). MS (ESI) m/z, [M+H] calculated, 523.92; observed, 523.69.

### 28. Preparation of 2-(3,5-Diiodo-4-(4-methoxyphenoxy)phenyl)acetic acid (MLAE-043)

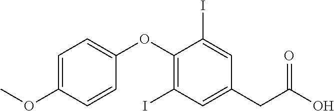 [SMILES: COc1ccc(Oc2c(I)cc(CC(=O)O)cc2I)cc1]

A solution of methyl 2-(3,5-diiodo-4-(4-methoxyphenoxy)phenyl)acetate (110 mg, 0.210 mmol), and 1M LiOH (1.049 mL), in THF (1 mL), was stirred for 1 h. The solution was acidified with 1M oxalic acid and extracted with ethyl acetate (3×5 mL). The combined organic layers were washed with saturated brine, dried over Na₂SO₄, filtered, and concentrated, to give a pale brown solid (104 mg, 97% yield).

### 29. Preparation of 2-(3,5-Diiodo-4-(4-methoxyphenoxy)phenyl)-1-morpholinoethanone (MLAE-044)

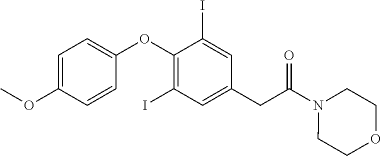 [SMILES: COc1ccc(Oc2c(I)cc(CC(=O)N3CCOCC3)cc2I)cc1]

Starting with MLAE-043, general method (A) was used to give the desired product (4 mg, 10% yield).

### 30. Preparation of 4-(2-(3,5-Diiodo-4-(4-methoxyphenoxy)phenyl)acetyl)piperazin-2-one (MLAE-045)

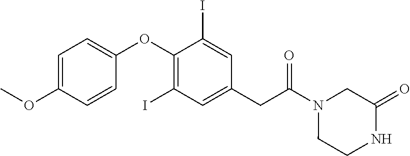 [SMILES: COc1ccc(Oc2c(I)cc(CC(=O)N3CCNC(=O)C3)cc2I)cc1]

Starting with MLAE-043, general method (A) was used to give the desired product (6.4 mg, 16% yield). MS (ESI) m/z, [M+H] calculated, 592.94; observed, 592.81.

### 31. Preparation of 2-(3,5-Diiodo-4-(4-methoxyphenoxy)phenyl)-N-(methylsulfonyl)acetamide (MLAE-046)

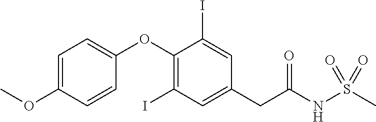

Starting with MLAE-043, general method (B) was used to give the desired product (16 mg, 41% yield). MS (ESI) m/z, [M+H] calculated, 587.88; observed, 587.65.

### 32. Preparation of 2-(4-(4-hydroxyphenoxy)-3,5-diiodophenyl)-1-morpholinoethanone (MLAE-047)

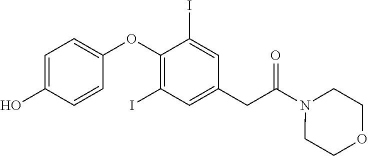 [SMILES: O=C(Cc1cc(I)c(Oc2ccc(O)cc2)c(I)c1)N1CCOCC1]

Starting with MLAE-044, general method (C) was used to give the desired product (1.2 mg, 32% yield). MS (ESI) m/z, [M+H] calculated, 565.93; observed, 565.80.

### 33. Preparation of 4-(2-(4-(4-Hydroxyphenoxy)-3,5-diiodophenyl)acetyl)piperazin-2-one (MLAE-048)

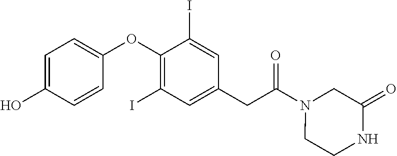 [SMILES: O=C1CN(C(=O)Cc2cc(I)c(Oc3ccc(O)cc3)c(I)c2)CCN1]

Starting with MLAE-045, general method (C) was used to give the desired product (2.2 mg, 45% yield). MS (ESI) m/z, [M+H] calculated, 578.93; observed, 578.83.

### 34. Preparation of 2-(4-(4-Hydroxyphenoxy)-3,5-diiodophenyl)-N-(methylsulfonyl)acetamide (MLAE-049)

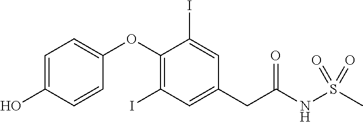 [SMILES: CS(=O)(=O)NC(=O)Cc1cc(I)c(Oc2ccc(O)cc2)c(I)c1]

Starting with MLAE-046, general method (C) was used to give the desired product (14 mg, 102% yield). ¹H NMR (400 MHz, DMSO) δ 11.96 (s, 1H), 9.11 (s, 1H), 7.84 (s, 2H), 6.70 (d, J=8.9 Hz, 2H), 6.53 (d, J=8.9 Hz, 2H), 3.65 (s, 2H), 3.26 (s, 3H); MS (ESI) m/z, [M+H] calculated, 573.87; observed, 573.67.

### 35. Preparation of 3-(4-(4-Hydroxyphenoxy)-3,5-diiodophenyl)-N-(2-methoxyethyl)propanamide (MLAE-051-1)

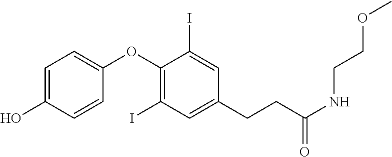 [SMILES: COCCNC(=O)CCc1cc(I)c(Oc2ccc(O)cc2)c(I)c1]

Starting with 3-(4-(4-hydroxyphenoxy)-3,5-diiodophenyl)propanoic acid, general method (A) was used to give the desired product (7.4 mg, 53% yield). MS (ESI) m/z, [M+H] calculated, 567.95; observed, 567.76.

### 36. Preparation of 3-(4-(4-Hydroxyphenoxy)-3,5-diiodophenyl)-N-((tetrahydrofuran-2-yl)methyl)propanamide (MLAE-051-2)

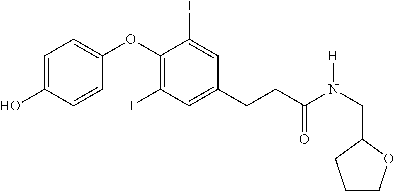 [SMILES: O=C(CCc1cc(I)c(Oc2ccc(O)cc2)c(I)c1)NCC1CCCO1]

Starting with 3-(4-(4-hydroxyphenoxy)-3,5-diiodophenyl)propanoic acid, general method (A) was used to give the desired product (7.1 mg, 49% yield). MS (ESI) m/z, [M+H] calculated, 593.96; observed, 593.81.

### 37. Preparation of 1-(4-(Ethylsulfonyl)piperazin-1-Yl)-3-(4-(4-hydroxyphenoxy)-3,5-diiodophenyl)propan-1-one (MLAE-051-3)

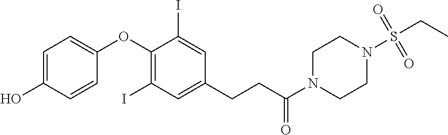 [SMILES: CCS(=O)(=O)N1CCN(C(=O)CCc2cc(I)c(Oc3ccc(O)cc3)c(I)c2)CC1]

Starting with 3-(4-(4-hydroxyphenoxy)-3,5-diiodophenyl)propanoic acid, general method (A) was used to give the desired product (5.1 mg, 31% yield). MS (ESI) m/z, [M+H] calculated, 670.96; observed, 670.74.

### 38. Preparation of N-(2-Hydroxyethyl)-3-(4-(4-hydroxyphenoxy)-3,5-diiodophenyl)propanamide (MLAE-054-1)

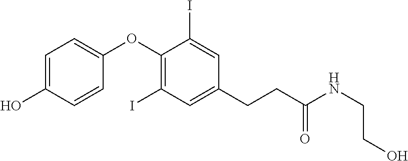 [SMILES: O=C(CCc1cc(I)c(Oc2ccc(O)cc2)c(I)c1)NCCO]

Starting with 3-(4-(4-hydroxyphenoxy)-3,5-diiodophenyl)propanoic acid, general method (A) was used to give the desired product (8 mg, 59% yield). MS (ESI) m/z, [M+H] calculated, 553.93; observed, 553.78.

### 39. Preparation of N-(2-Ethoxyethyl)-3-(4-(4-hydroxyphenoxy)-3,5-diiodophenyl)propanamide (MLAE-054-2)

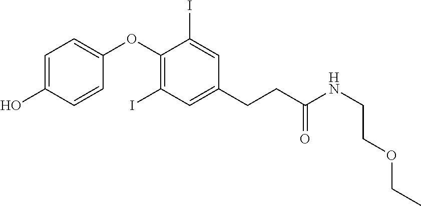 [SMILES: CCOCCNC(=O)CCc1cc(I)c(Oc2ccc(O)cc2)c(I)c1]

Starting with 3-(4-(4hydroxyphenoxy)-3,5-diiodophenyl)propanoic acid, general method (A) was used to give the desired product (7.5 mg, 53% yield). MS (ESI) m/z, [M+H] calculated, 581.96; observed, 581.78.

### 40. Preparation of 3-(4-(4-Hydroxyphenoxy)-3,5-diiodophenyl)-N-(2-propoxyethyl)propanamide (MLAE-054-3)

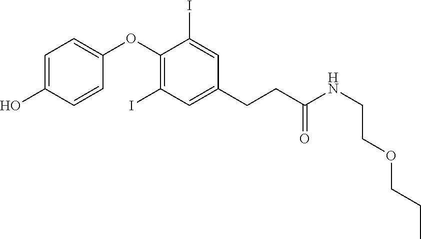 [SMILES: CCCOCCNC(=O)CCc1cc(I)c(Oc2ccc(O)cc2)c(I)c1]

Starting with 3-(4-(4-hydroxyphenoxy)-3,5-diiodophenyl)propanoic acid, general method (A) was used to give the desired product (16.3 mg, 112% yield). MS (ESI) m/z, [M+H] calculated, 595.98; observed, 595.77.

### 41. Preparation of 3-(4-(4-Hydroxyphenoxy)-3,5-diiodophenyl)-N-(3-methoxypropyl)propanamide (MLAE-054-4)

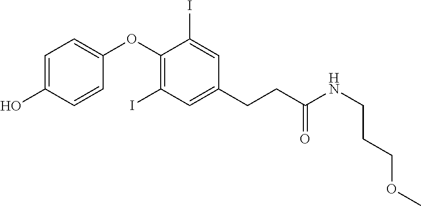 [SMILES: COCCCNC(=O)CCc1cc(I)c(Oc2ccc(O)cc2)c(I)c1]

Starting with 3-(4-(4-hydroxyphenoxy)-3,5-diiodophenyl)propanoic acid, general method (A) was used to give the desired product (5.4 mg, 38% yield). MS (ESI) m/z, [M+H] calculated, 581.96; observed, 581.95.

### 42. Preparation of (S)—N-(1-HYDROXY-3-(4-(4-hydroxyphenoxy)-3,5-diiodophenyl)propan-2-yl)methanesulfonamide (MLAE-058-1)

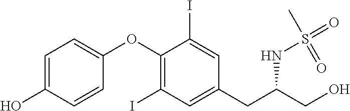 [SMILES: CS(=O)(=O)N[C@H](CO)Cc1cc(I)c(Oc2ccc(O)cc2)c(I)c1]

Starting with (S)-4-(4-(2-amino-3-hydroxypropyl)-2,6-diiodophenoxy)phenol, general method (D) was used to give the desired product (4.7 mg, 34% yield). MS (ESI) m/z, [M+H] calculated, 589.9; observed, 589.72.

### 43. Preparation of (S)—N-(1-Hydroxy-3-(4-(4-hydroxyphenoxy)-3,5-diiodophenyl)propan-2-yl)benzenesulfonamide (MLAE-058-2)

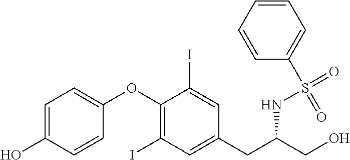 [SMILES: O=S(=O)(N[C@H](CO)Cc1cc(I)c(Oc2ccc(O)cc2)c(I)c1)c1ccccc1]

Starting with (S)-4-(4-(2-amino-3-hydroxypropyl)-2,6-diiodophenoxy)phenol, general method (D) was used to give the desired product (5 mg, 33% yield). MS (ESI) m/z, [M+H] calculated, 651.92; observed, 651.79.

### 44. Preparation of (S)—N-(1-Hydroxy-3-(4-(4-hydroxyphenoxy)-3,5-diiodophenyl)propan-2-yl)-4-methoxybenzenesulfonamide (MLAE-058-3)

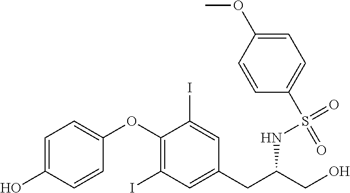 [SMILES: COc1ccc(S(=O)(=O)N[C@H](CO)Cc2cc(I)c(Oc3ccc(O)cc3)c(I)c2)cc1]

Starting with (S)-4-(4-(2-amino-3-hydroxypropyl)-2,6-diiodophenoxy)phenol, general method (D) was used to give the desired product (9.6 mg, 60% yield). MS (ESI) m/z, [M+H] calculated, 681.93; observed, 681.76.

### 45. Preparation of (S)—N-(1-Hydroxy-3-(4-(4-hydroxyphenoxy)-3,5-diiodophenyl)propan-2-yl)acetamide (MLAE-059)

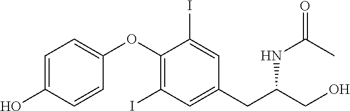 [SMILES: CC(=O)N[C@H](CO)Cc1cc(I)c(Oc2ccc(O)cc2)c(I)c1]

Pyridine (2.84 μL, 0.035 mmol) was added to a solution of (S)-4-(4-(2-amino-3-hydroxypropyl)-2,6-diiodophenoxy)phenol (12 mg, 0.023 mmol), and acetic anhydride (3.33 μL, 0.035 mmol), in DCM (200 μL). The solid did not go into solution, so DMF (100 μL) was added. 1M HCl (3 mL) was added to the reaction mixture and extracted with ethyl acetate (3×3 mL). The combined organic layers were washed with saturated brine, dried over Na₂SO₄, filtered, and concentrated. The crude product was purified by preparative TLC (eluting with DCM/MeOH, 9:1) to give the product (7.4 mg, 57% yield). MS (ESI) m/z, [M+H] calculated, 553.93; observed, 553.78.

### 46. Preparation OF (S)-4-Hydroxy-N-(1-hydroxy-3-(4-(4-hydroxyphenoxy)-3,5-diiodophenyl)propan-2-yl)benzenesulfonamide (MLAE-060)

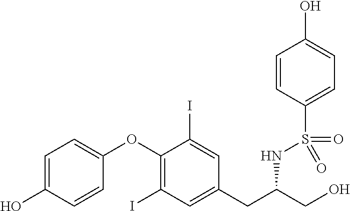 [SMILES: O=S(=O)(N[C@H](CO)Cc1cc(I)c(Oc2ccc(O)cc2)c(I)c1)c1ccc(O)cc1]

Starting with MLAE-058-3, general method (C) was used to give the desired product (0.7 mg, 11% yield). MS (ESI) m/z, [M+H] calculated, 667.91; observed, 667.78.

### 47. Preparation of (S)—N-(1-Hydroxy-3-(4-(4-hydroxyphenoxy)-3,5-diiodophenyl)propan-2-yl)-3,5-dimethylisoxazole-4-sulfonamide (MLAE-061)

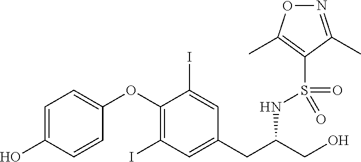 [SMILES: Cc1noc(C)c1S(=O)(=O)N[C@H](CO)Cc1cc(I)c(Oc2ccc(O)cc2)c(I)c1]

Starting with (S)-4-(4-(2-amino-3-hydroxypropyl)-2,6-diiodophenoxy)phenol, general method (D) was used to give the desired product (6.8 mg, 43% yield). MS (ESI) m/z, [M+H] calculated, 670.92; observed, 670.74.

### 48. Preparation of (S)-1-(1-Hydroxy-3-(4-(4-hydroxyphenoxy)-3,5-diiodophenyl)propan-2-yl)-3-isopropylurea (MLAE-062)

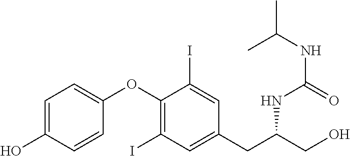 [SMILES: CC(C)NC(=O)N[C@H](CO)Cc1cc(I)c(Oc2ccc(O)cc2)c(I)c1]

2-Isocyanatopropane (2.54 μL, 0.026 mmol) was added to a solution of (S)-4-(4-(2-amino-3-hydroxypropyl)-2,6-diiodophenoxy)phenol (12 mg, 0.023 mmol), in THF (100 μL), and DMF (100 μL), at 0° C. After 30 min the reaction mixture was stirred at rt for 90 min. Water (3 mL) was added to the reaction mixture and extracted with ethyl acetate (3×3 mL). The combined organic layers were washed with saturated brine, dried over Na₂SO₄, filtered, and concentrated, to give the product as a colorless residue (12.6 mg, 90% yield). MS (ESI) m/z, [M+H] calculated, 596.97; observed, 596.90.

### 49. Preparation of (S)-Methyl (1-Hydroxy-3-(4-(4-hydroxyphenoxy)-3,5-diiodophenyl)propan-2-yl)carbamate (MLAE-063)

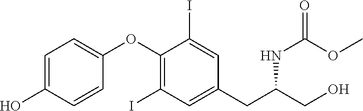 [SMILES: COC(=O)N[C@H](CO)Cc1cc(I)c(Oc2ccc(O)cc2)c(I)c1]

Methyl carbonochloridate (1.9 μL, 0.025 mmol) was added to a solution of (S)-4-(4-(2-amino-3-hydroxypropyl)-2,6-diiodophenoxy)phenol (12 mg, 0.023 mmol), sodium bicarbonate (5.92 mg, 0.070 mmol), in THF (100 μL), and Water (100 μL), at 0° C. After 30 min the ice bath was removed and the solution stirred for 90 min at rt. Water (3 mL) was added to the solution and extracted with ethyl acetate (3×3 mL). The combined organic layers were washed with saturated brine, dried over Na₂SO₄, filtered, and concentrated, to give the product as a colorless residue (12.1 mg, 91% yield). MS (ESI) m/z, [M+H] calculated, 569.93; observed, 569.83.

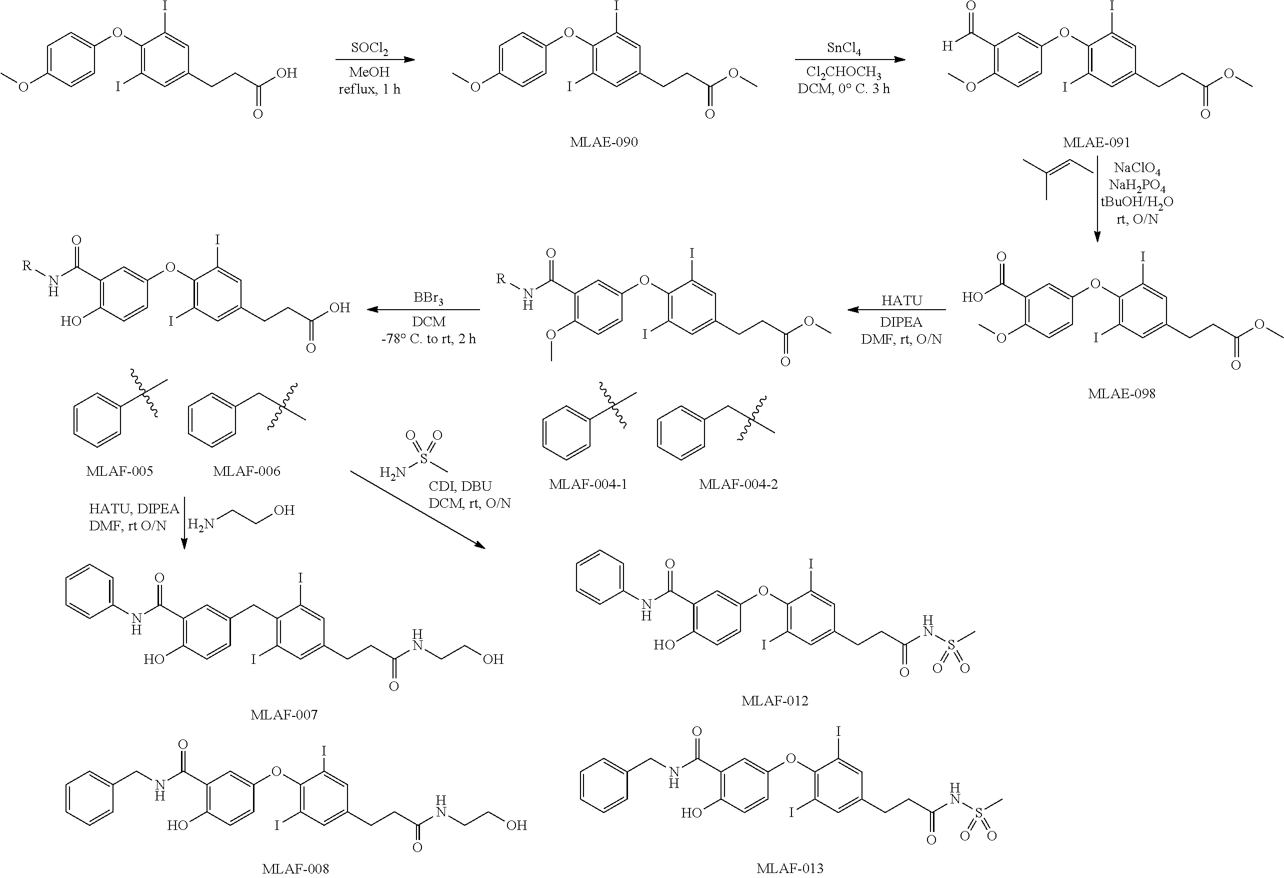

### 50. Preparation of Methyl 3-(3,5-diiodo-4-(4-methoxyphenoxy)phenyl)propanoate (MLAE-090)

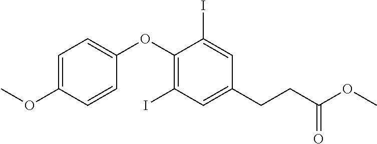

Thionyl chloride (0.027 mL, 0.372 mmol) was added dropwise to a solution of 3-(3,5-diiodo-4-(4-methoxyphenoxy)phenyl)propanoic acid (65 mg, 0.124 mmol) in MeOH (1 mL) at 0° C. The solution is heated to reflux for 1 h, and then concentrated to give a colorless residue (66 mg, 99%). MS (ESI) m/z, [M+H] calculated, 538.92; observed, 538.80.

### 51. Preparation of Methyl 3-(4-(3-formyl-4-methoxyphenoxy)-3,5-diiodophenyl)propanoate (MLAE-091)

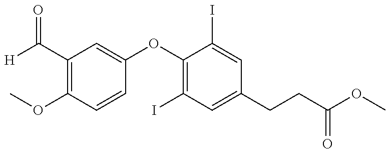 [SMILES: COC(=O)CCc1cc(I)c(Oc2ccc(OC)c(C=O)c2)c(I)c1]

A solution of 1M in DCM Tin(IV) chloride (0.159 mL, 0.159 mmol) was added dropwise to a solution of MLAE-090 (66 mg, 0.123 mmol), in DCM (1 mL), at 0° C. The solution was stirred for 1 h under N₂. Dichloromethyl methyl ether (0.013 mL, 0.147 mmol) was added to the pale brown reaction mixture and the solution was stirred for 3 h at 0° C. Water (5 mL) was added to the brown reaction mixture and extracted with ethyl acetate (3×5 mL). The combined organic layers were washed with saturated brine, dried over Na₂SO₄, filtered, and concentrated. The crude product was purified by flash column chromatography (Biotage SP4, 12+S column, eluting with hexanes/ethyl acetate, 0-20% gradient) to give the product as an opaque residue (48.3 mg, 70& yield). ¹H NMR (400 MHz, CDCl₃) δ 10.41 (s, 1H), 7.69 (s, 2H), 7.19-7.06 (m, 2H), 6.98 (d, J=9.0 Hz, 1H), 3.92 (s, 3H), 3.71 (s, 3H), 2.90 (t, J=7.7 Hz, 2H), 2.65 (t, J=7.7 Hz, 2H); MS (ESI) m/z, [M+H] calculated, 566.92; observed, 566.75.

### 52. Preparation of 5-(2,6-Diiodo-4-(3-Methoxy-3-oxopropyl)phenoxy)-2-methoxybenzoic acid (MLAF-003)

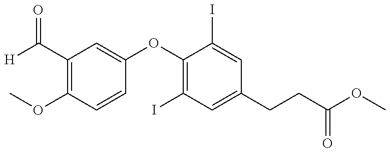 [SMILES: COC(=O)CCc1cc(I)c(Oc2ccc(OC)c(C=O)c2)c(I)c1]

Sodium chlorite (71.6 mg, 0.634 mmol) was added portionwise to a solution of methyl 3-(4-(3-formyl-4-methoxyphenoxy)-3,5-diiodophenyl)propanoate (102.5 mg, 0.181 mmol), sodium dihydrogen phosphate (23.89 mg, 0.199 mmol), and 2-methyl-2-butene (96 μL, 0.905 mmol), in water (360 μL), and tBuOH (612 μL). The cloudy white solution was stirred overnight. 1M HCl (3 mL) was added to the reaction mixture and extracted with ethyl acetate (3×3 mL). The combined organic layers were washed with saturated brine, dried over Na₂SO₄, filtered, and concentrated. The crude product was purified by flash column chromatography (Biotage SP4, 12+M column, eluting with DCM/MeOH, 0-5% gradient) to give a white solid (82.4 mg, 78% yield). ¹H NMR (400 MHz, CDCl₃) δ 7.70 (s, 2H), 7.49 (d, J=3.2 Hz, 1H), 7.11 (dd, J=9.1, 3.2 Hz, 1H), 7.04 (d, J=9.1 Hz, 1H), 4.07 (s, 3H), 3.71 (s, 3H), 2.91 (t, J=7.6 Hz, 2H), 2.66 (t, J=7.7 Hz, 2H); MS (ESI) m/z, [M+H] calculated, 582.91; observed, 582.80.

### 53. Preparation of Methyl 3-(3,5-diiodo-4-(4-Methoxy-3-(phenylcarbamoyl)phenoxy)phenyl)propanoate (MLAF-004-1)

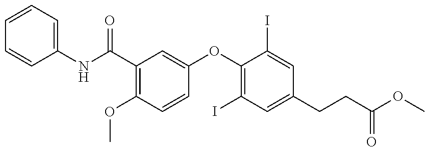 [SMILES: COC(=O)CCc1cc(I)c(Oc2ccc(OC)c(C(=O)Nc3ccccc3)c2)c(I)c1]

Starting with MLAF-003, general method (A) was used to give the desired product (32 mg, 74%). ¹H NMR (400 MHz, CDCl₃) δ 9.88 (s, 1H), 7.69 (s, 2H), 7.67-7.60 (m, 3H), 7.40-7.30 (m, 2H), 7.12 (dd, J=10.6, 4.2 Hz, 1H), 7.02 (s, 2H), 4.04 (s, 3H), 3.71 (s, 3H), 2.90 (t, J=7.7 Hz, 2H), 2.65 (t, J=7.7 Hz, 2H); MS (ESI) m/z, [M+H] calculated, 657.96; observed, 657.77.

### 54. Preparation of Methyl 3-(4-(3-(benzylcarbamoyl)-4-methoxyphenoxy)-3,5-diiodophenyl)propanoate (MLAF-004-2)

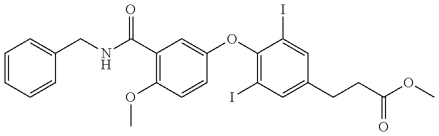 [SMILES: COC(=O)CCc1cc(I)c(Oc2ccc(OC)c(C(=O)NCc3ccccc3)c2)c(I)c1]

Starting with MLAF-003, general method (A) was used to give the desired product (39.2 mg, 83%). ¹H NMR (400 MHz, CDCl₃) δ 8.30 (t, J=5.4 Hz, 1H), 7.68 (s, 2H), 7.63 (d, J=2.8 Hz, 1H), 7.35 (d, J=4.3 Hz, 4H), 7.31-7.23 (m, 1H), 7.00-6.89 (m, 2H), 4.65 (d, J=5.7 Hz, 2H), 3.89 (s, 3H), 3.70 (s, 3H), 2.89 (t, J=7.7 Hz, 2H), 2.64 (t, J=7.7 Hz, 2H); MS (ESI) m/z, [M+H] calculated, 671.97; observed, 671.81.

### 55. Preparation of 3-(4-(4-Hydroxy-3-(phenylcarbamoyl)phenoxy)-3,5-diiodophenyl)propanoic acid (MLAF-005)

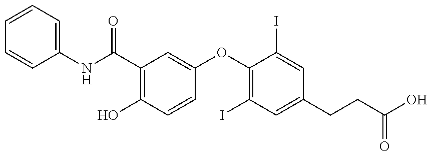 [SMILES: O=C(O)CCc1cc(I)c(Oc2ccc(O)c(C(=O)Nc3ccccc3)c2)c(I)c1]

Starting with MLAF-004-1, general method (C) was used to give the desired product (28.5 mg, 88%). ¹H NMR (400 MHz, CDCl₃) δ 11.56 (s, 1H), 7.92 (s, 1H), 7.56 (d, J=8.0 Hz, 2H), 7.40 (t, J=7.8 Hz, 2H), 7.21 (t, J=7.4 Hz, 1H), 7.09 (d, J=2.7 Hz, 1H), 6.95 (d, J=9.1 Hz, 1H), 6.78 (dd, J=9.1, 2.8 Hz, 1H), 2.91 (t, J=7.5 Hz, 2H), 2.71 (t, J=7.5 Hz, 2H); MS (ESI) m/z, [M+H] calculated, 629.93; observed, 629.69.

### 56. Preparation of 3-(4-(3-(benzylcarbamoyl)-4-hydroxyphenoxy)-3,5-diiodophenyl)propanoic acid (MLAF-006)

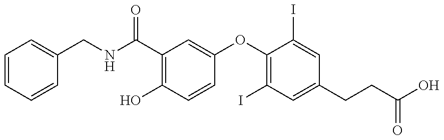 [SMILES: O=C(O)CCc1cc(I)c(Oc2ccc(O)c(C(=O)NCc3ccccc3)c2)c(I)c1]

Starting with MLAF-004-2, general method (C) was used to give the desired product (32 mg, 86%). ¹H NMR (400 MHz, CDCl₃) δ 11.90 (s, 1H), 7.68 (s, 2H), 7.42-7.30 (m, 5H), 6.96-6.85 (m, 2H), 6.71 (dd, J=9.1, 2.6 Hz, 1H), 6.59 (s, 1H), 4.62 (d, J=5.5 Hz, 2H), 2.88 (t, J=7.3 Hz, 2H), 2.68 (t, J=7.0 Hz, 2H); MS (ESI) m/z, [M+H] calculated, 643.94; observed, 643.80.

### 57. Preparation of 2-Hydroxy-5-(4-(3-((2-hydroxyethyl)amino)-3-oxopropyl)-2,6-diiodophenoxy)-N-phenylbenzamide (MLAF-007)

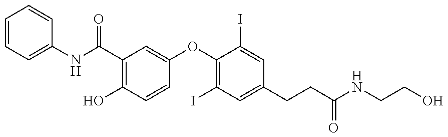 [SMILES: O=C(CCc1cc(I)c(Oc2ccc(O)c(C(=O)Nc3ccccc3)c2)c(I)c1)NCCO]

Starting with MLAF-005, general method (A) was used to give the desired product (3.6 mg, 24%). MS (ESI) m/z, [M+H] calculated, 672.97; observed, 672.88.

### 58. Preparation of N-Benzyl-2-hydroxy-5-(4-(3-((2-hydroxyethyl)amino)-3-oxopropyl)-2,6-diiodophenoxy)benzamide (MLAF-008)

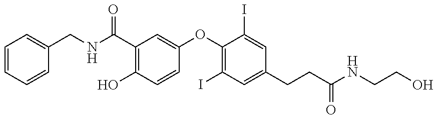 [SMILES: O=C(CCc1cc(I)c(Oc2ccc(O)c(C(=O)NCc3ccccc3)c2)c(I)c1)NCCO]

Starting with MLAF-006, general method (A) was used to give the desired product (0.9 mg, 5%). MS (ESI) m/z, [M+H] calculated, 686.99; observed, 686.87.

### 59. Preparation of 5-(2,6-diiodo-4-(3-(methylsulfonamido)-3-oxopropyl)phenoxy)-2-hydroxy-N-phenylbenzamide (MLAF-012)

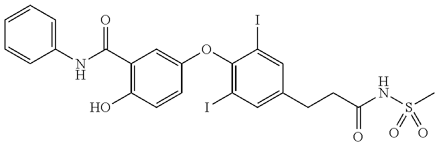 [SMILES: CS(=O)(=O)NC(=O)CCc1cc(I)c(Oc2ccc(O)c(C(=O)Nc3ccccc3)c2)c(I)c1]

Starting with MLAF-005, general method (B) was used to give the desired product (2.9 mg, 32%). MS (ESI) m/z, [M+H] calculated, 706.92; observed, 706.71.

### 60. Preparation of N-Benzyl-5-(2,6-diiodo-4-(3-(methylsulfonamido)-3-oxopropyl)phenoxy)-2-hydroxybenzamide (MLAF-013)

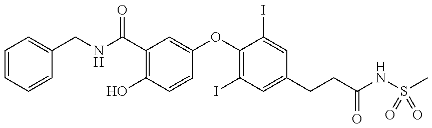 [SMILES: CS(=O)(=O)NC(=O)CCc1cc(I)c(Oc2ccc(O)c(C(=O)NCc3ccccc3)c2)c(I)c1]

Starting with MLAF-006, general method (B) was used to give the desired product (1.7 mg, 18%). MS (ESI) m/z, [M+H] calculated, 720.94; observed, 720.82.

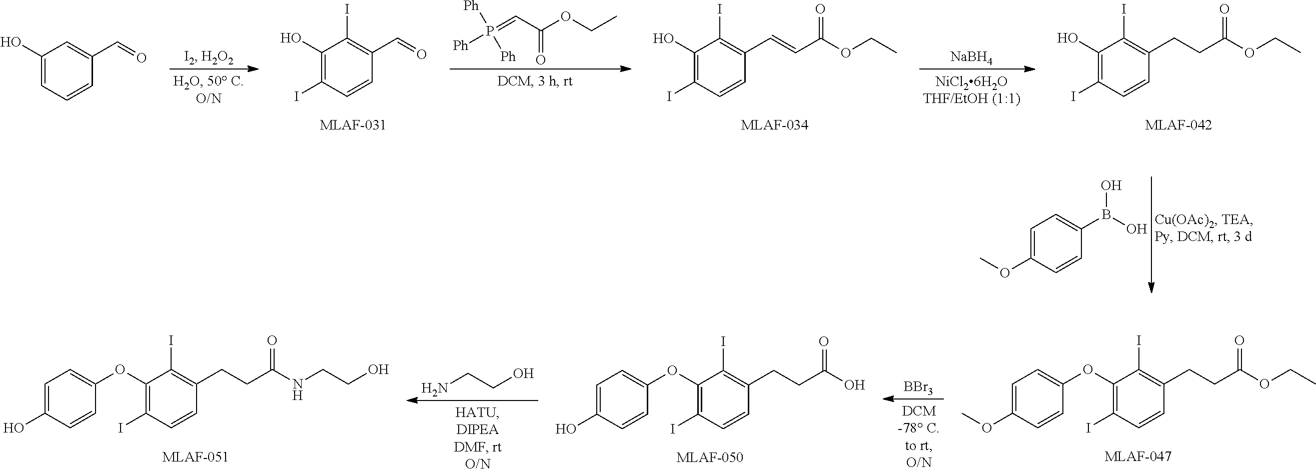

### 61. Preparation of 3-Hydroxy-2,4-Diiodobenzaldehyde (MLAF-031)

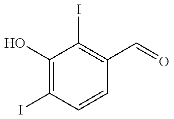 [SMILES: O=Cc1ccc(I)c(O)c1I]

Aqueous 30% hydrogen peroxide (1.255 mL, 12.28 mmol) was added dropwise to a stirring solution of 3-hydroxybenzaldehyde (0.5 g, 4.09 mmol), and crushed I₂ (1.559 g, 6.14 mmol) in water (20 mL). The solution was stirred at 50° C. for 24 h. Aqueous Na₂S₂O₃ (20 mL) was added to the rusty red/brown reaction mixture and extracted with ethyl acetate (3×40 mL). The combined organic layers were washed with saturated brine, dried over Na₂SO₄, filtered, and concentrated. The crude product was purified by flash column chromatography (Biotage SP4, 40+S column, eluting with hexanes/EtOAc, 0-15% gradient) to give a pale yellow solid (881 mg, 58% yield). ¹H NMR (400 MHz, CDCl₃) δ 9.98 (s, 1H), 7.83 (d, J=8.1 Hz, 1H), 7.16 (d, J=8.1 Hz, 1H), 6.18 (s, 1H).

### 62. Preparation of (E)-Ethyl 3-(3-hydroxy-2,4-diiodophenyl)acrylate (MLAF-036)

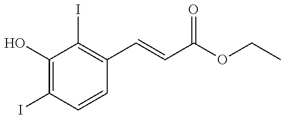 [SMILES: CCOC(=O)/C=C/c1ccc(I)c(O)c1I]

A solution of MLAF-031 (810 mg, 2.166 mmol), and ethyl 2-(triphenylphosphoranylidene)acetate (906 mg, 2.60 mmol), in DCM (15 mL), was allowed to stand overnight. The pale green/yellow solution was concentrated and purified by flash column chromatography (Biotage SP4, 40+M column, eluting with hexanes/EtOAc, 0-20% gradient) to give a pale brown solid (927 mg, 96% yield). ¹H NMR (400 MHz, CDCl₃) δ 7.83 (d, J=15.7 Hz, 1H), 7.68 (d, J=8.2 Hz, 1H), 6.86 (d, J=8.2 Hz, 1H), 6.31 (d, J=15.7 Hz, 1H), 5.98 (s, 1H), 4.29 (q, J=7.1 Hz, 2H), 1.35 (t, J=7.1 Hz, 4H); MS (ESI) m/z, [M+H] calculated, 444.88; observed, 444.72.

### 63. Preparation of Ethyl 3-(3-hydroxy-2,4-diiodophenyl)propanoate (MLAF-042)

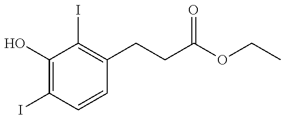 [SMILES: CCOC(=O)CCc1ccc(I)c(O)c1I]

Sodium borohydride (55.0 mg, 1.454 mmol) was added portionwise to a solution of MLAF-036 (615 mg, 1.385 mmol), and nickel(II) chloride hexahydrate (16.46 mg, 0.069 mmol), in EtOH (4 mL), and THF (4 mL), at 0° C. After 10 min the ice bath was removed and the solution stirred for 1 h at rt. 1M HCl (20 mL) was added to the golden yellow reaction mixture and extracted with ethyl acetate (3×20 mL). The combined organic layers were washed with saturated brine, dried over Na₂SO₄, filtered, and concentrated. The crude product was purified by flash column chromatography (Biotage SP4, 40+S column, eluting with hexanes/EtOAc, 0-20% gradient) to give a pale brown solid (101 mg, 16% yield). ¹H NMR (400 MHz, CDCl₃) δ 7.58 (d, J=8.1 Hz, 1H), 6.59 (d, J=8.1 Hz, 1H), 5.89 (s, 1H), 4.14 (q, J=7.1 Hz, 2H), 3.05 (t, J=7.8 Hz, 2H), 2.60 (t, J=7.8 Hz, 2H), 1.25 (t, J=7.1 Hz, 3H).

### 64. Preparation of Ethyl 3-(2,4-diiodo-3-(4-methoxyphenoxy)phenyl)propanoate (MLAF-047)

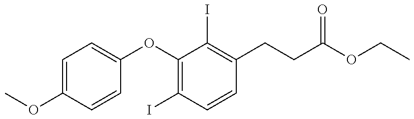 [SMILES: CCOC(=O)CCc1ccc(I)c(Oc2ccc(OC)cc2)c1I]

A flask was charged with ethyl 3-(3-hydroxy-2,4-diiodophenyl)propanoate (67 mg, 0.150 mmol), (4-methoxyphenyl)boronic acid (45.7 mg, 0.300 mmol), copper (II) acetate (82 mg, 0.451 mmol), 4 Å molecular sieve powder (180 mg), and DCM (2 mL). A mixture of TEA (0.126 mL, 0.901 mmol) and pyridine (0.073 mL, 0.901 mmol) was added dropwise to the stirring reaction mixture. A drying tube was attached the solution was stirred for 3 d. The dark brown solution was diluted with ethyl acetate and filtered through celite. The filtrate was concentrated and the crude product was purified by flash column chromatography (Biotage SP4, 12+S column, eluting with hexanes/EtOAc, 0-20% gradient) to give an impure solid (61 mg), which was used in the next step.

### 65. Preparation of 3-(3-(4-Hydroxyphenoxy)-2,4-diiodophenyl)propanoic acid (MLAF-050)

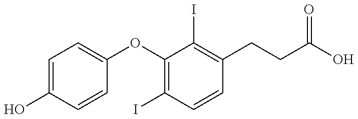 [SMILES: O=C(O)CCc1ccc(I)c(Oc2ccc(O)cc2)c1I]

A solution of 1M in DCM Boron tribromide (0.122 mL, 0.122 mmol) was added dropwise to a solution of MLAF-047 (61 mg, 0.110 mmol), in DCM (1 mL), at −78° C., stirring under N₂. After 30 min, the dry ice/acetone bath was removed and the solution stirred at rt. After 4 h, the reaction mixture was chilled to −78° C. and more boron tribromide (0.122 mL, 0.122 mmol) was added to the dark brown solution. The reaction mixture was stirred overnight allowing the solution to reach rt. Water (3 mL) was added to the reaction mixture and extracted with ethyl acetate (3×3 mL). The combined organic layers were washed with saturated brine, dried over Na₂SO₄, filtered, and concentrated. The crude product was purified by preparative TLC (eluting with DCM/MeOH, 95:5) to give a brown solid (33.2 mg, 59% yield). ¹H NMR (400 MHz, MeOD) δ 7.81 (d, J=8.0 Hz, 1H), 6.95 (d, J=8.1 Hz, 1H), 6.73-6.65 (m, 2H), 6.58-6.48 (m, 2H), 3.09 (t, J=7.6 Hz, 2H), 2.63 (t, J=7.5 Hz, 2H); MS (ESI) m/z, [M+H] calculated, 510.89; observed, 510.78.

### 66. Preparation of N-Hydroxyethyl)-3-(3-(4-hydroxyphenoxy)-2,4-diiodophenyl)propanamide (MLAF-051)

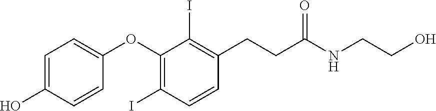 [SMILES: O=C(CCc1ccc(I)c(Oc2ccc(O)cc2)c1I)NCCO]

Starting with MLAF-050, general method (A) was used to give the desired product (4.7 mg, 14%). ¹H NMR (400 MHz, MeOD) δ 7.71 (d, J=8.1 Hz, 1H), 6.84 (d, J=8.1 Hz, 1H), 6.59 (d, J=9.0 Hz, 2H), 6.43 (d, J=9.0 Hz, 2H), 3.47 (t, J=5.8 Hz, 2H), 3.22-3.16 (m, 2H), 3.01 (t, J=7.7 Hz, 2H), 2.43 (t, J=7.7 Hz, 2H); MS (ESI) m/z, [M+H] calculated, 553.93; observed, 553.91.

### 67. Preparation of (S)-4-(4-(2-amino-3-hydroxypropyl)-2,6-diiodophenoxy)phenol ((S)-T2AA)

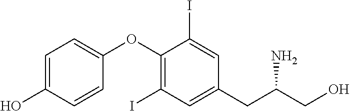 [SMILES: N[C@H](CO)Cc1cc(I)c(Oc2ccc(O)cc2)c(I)c1]

The synthetic scheme for the preparation of the title compound is given below, based on the procedure described by Skaanderup, P. R. and Jensen, T., Org. Lett. (2008), 10, 2821-2824. Based on R/S configuration rules, the compound is the (S) enantiomer, and referred to herein as (S)-T2AA. Alternatively, based on D/L configuration system, the compound is (L) enantiomer, and is alternatively referred to as (L)-T2AA.

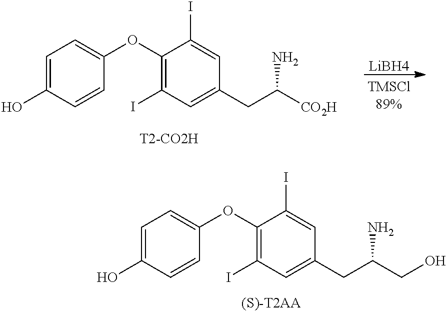

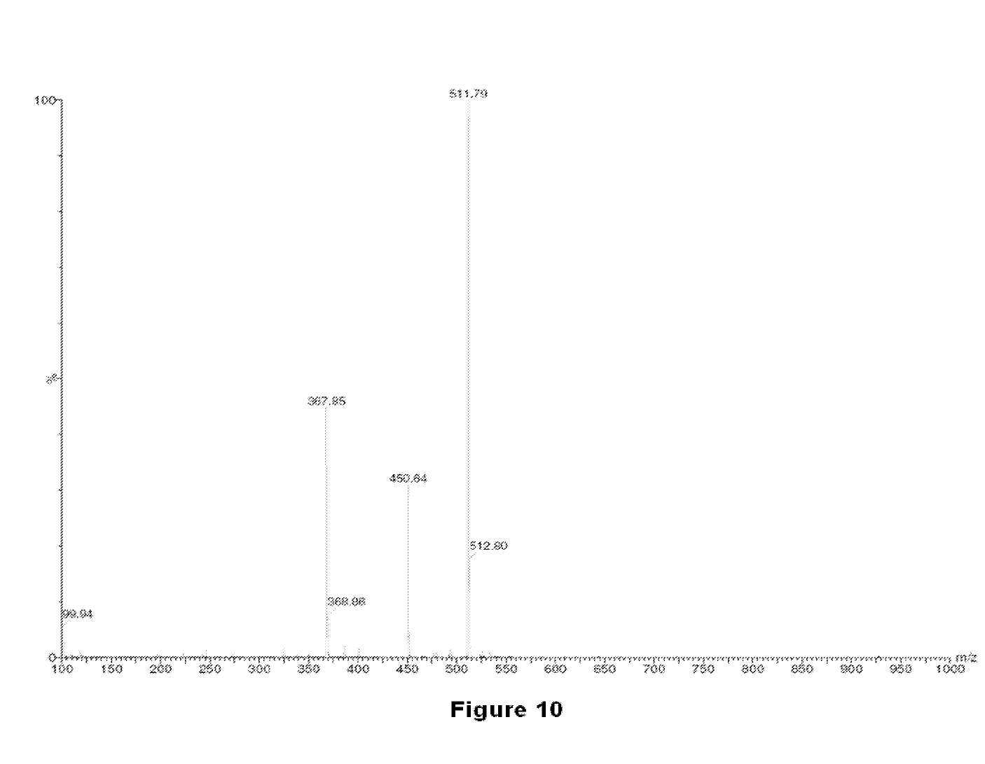

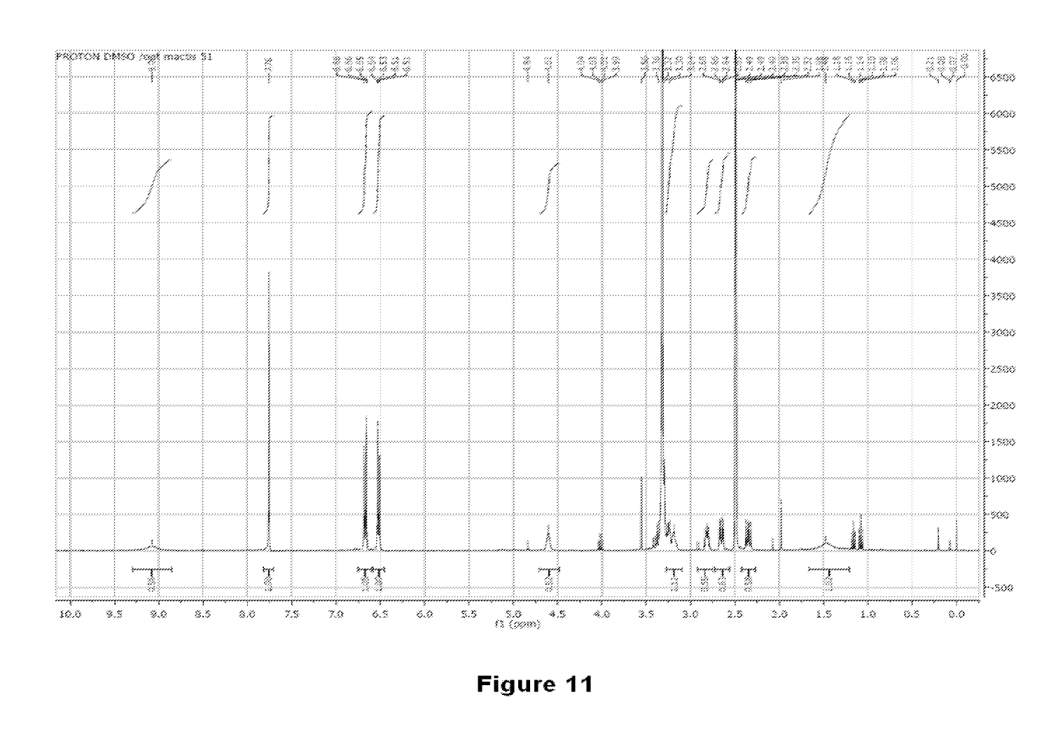

Briefly, in a 100 mL two-necked flask, a mixture of dry THF 5.5 mL, dry 1,4-dioxane 5.0 mL, and 2M LiBH₄ in THF (3.0 mL) was cooled by an ice-bath under nitrogen flow, and chlorotrimethylsilane (1.53 mL) was added dropwise The mixture was stirred at room temperature for 15 minutes, then cooled to ice-bath temperature, and T2-CO₂H (1.05 gram) was added at once. The mixture was stirred overnight, allowing the solution to reach ambient temperature. The mixture was heterogeneous throughout; however, by analyzing TLC and LC-MS, complete conversion of T2-CO₂H to T2AA was confirmed. The reaction mixture was carefully added to ice-water, adjusted the pH>9 by carefully adding 1M NaOH aqueous solution, and extracted by ethyl acetate. After drying the organic phase and evaporation under reduced pressure, the crude material was easily solidified and T2AA was isolated as white crystals by washing the crude material with a small amount of n-hexane containing a small amount of diethyl ether (0.91 gram). The sample for biological assays was prepared by dissolving the (S)-T2AA batch above in a small amount of 1,4-dioxane and gradually adding ethyl ether to allow recrystallization. Representative NMR and MS data are provided, respectively, in FIGS. 10 and 11.

### 68. Preparation of (R)-4-(4-(2-amino-3-hydroxypropyl)-2,6-diiodophenoxy)phenol ((R)-T2AA)

The synthetic scheme for the preparation of the title compound is given below. Based on R/S configuration rules, the compound is the (R) enantiomer, and referred to herein as (R)-T2AA. Alternatively, based on D/L configuration system, the compound is (L) enantiomer, and is alternatively referred to as (D)-T2AA.

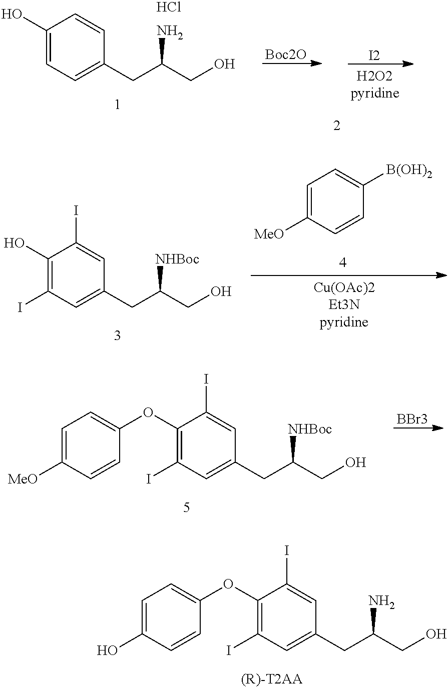

Briefly, compound 1 (406 mg) was Boc-protected with Boc-anhydride (720 mg) by a conventional Shotten reaction condition (water-THF, pH 8-10, 5-25° C.) to afford compound 2. This product, compound 2, was dissolved in pyridine (0.62 mL) and iodine (0.56 g) was carefully added in 40° C. oil-bath, followed by 30% aqueous hydrogen peroxide, and stirred for 1 hr. The reaction mixture was worked up (ethyl acetate/water, adjusted to pH˜3 by adding hydrochloric acid) and filtered by a short pad of Florisil® to isolate compound 3 (939 mg).

A mixture of compound 3 (156 mg), compound 4 (183 mg), anhydrous copper (II) acetate (60 mg), molecular sieve 4A powder (freshly dried by microwave; 0.9 g), dry dichloromethane (6 mL), triethylamine (0.42 mL), and dry pyridine (0.16 mL) was stirred under room temperature in a flask equipped with a calcium chloride tube (i.e., dry condition but open air condition) overnight. The reaction mixture was filtered through Celite®, concentrated, and silica-gel column chromatographed to afford compound 5 (68 mg). This product, compound 5, was treated with tribromoborane in dichloromethane (1M, 2.2.mL) overnight at 4° C. The reaction mixture was worked up in a conventional manner (ethyl acetate/saturated aqueous sodium bicarbonate solution, followed by evaporation). The crude material was washed with n-hexane and recrystallized by the same manner as described in above for (S)-T2AA to isolate the title compound (18 mg).

### 69. Preparation of 3-(4-(4-hydroxyphenoxy)-3,5-dimethylphenyl)propanoic acid (NF021111)

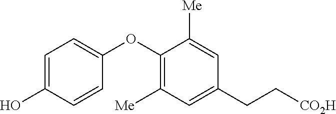 [SMILES: Cc1cc(CCC(=O)O)cc(C)c1Oc1ccc(O)cc1]

The synthetic scheme for the preparation of the title compound is given below.

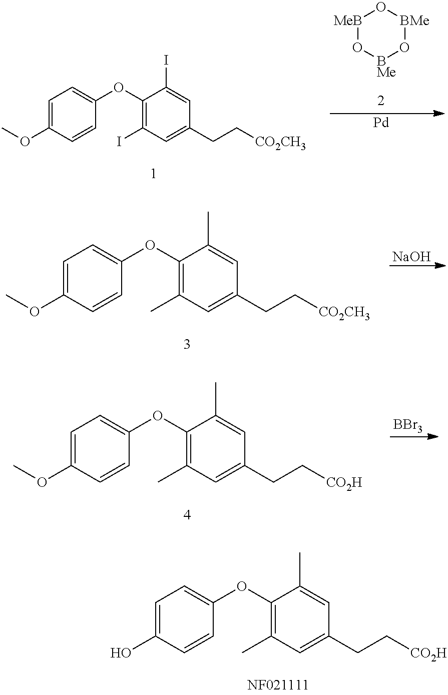

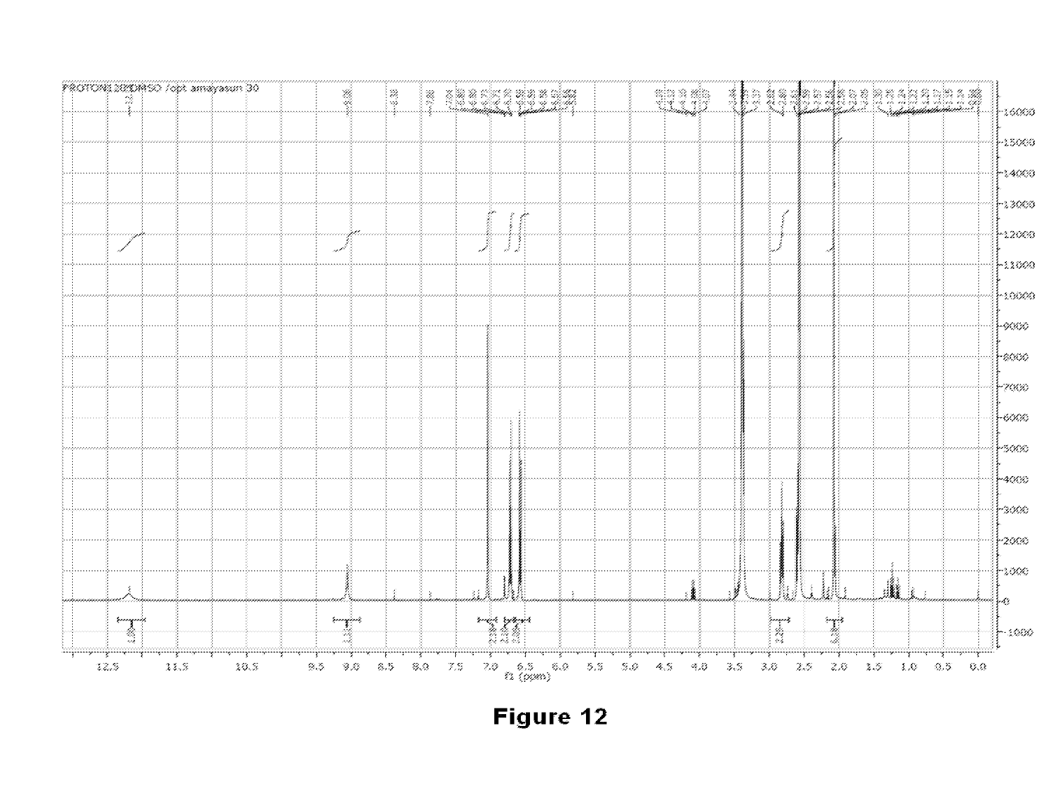

Briefly, a mixture of 1 (108 mg), 2 (84 μL), PdCl₂(dppf)-CH₂Cl₂ complex (32 mg), cesium carbonate (195 mg), and dry 1,4-dioxane (168 μL) was stirred at 85° C. in a sealed tube under nitrogen overnight. The reaction mixture was filtered through Celite®, concentrated, and silica-gel column chromatographed to afford 3 (46 mg). 3 was dissolved in methanol (0.3 mL), 1,4-dioxane (0.3 mL), and treated with 5M aqueous sodium hydroxide solution (0.3 mL) at ambient temperature for 1 hour. The reaction mixture was worked up (ethyl acetate/water, adjusted to pH˜3 by adding hydrochloric acid) to isolate 4. 4 was treated with tribromoborane in dichloromethane (1M, 2.0.mL) overnight at 4° C. The reaction mixture was worked up in a conventional manner (ethyl acetate/saturated aqueous sodium bicarbonate solution, followed by evaporation). The crude material was washed with n-hexane and purified by preparative TLC to isolate title compound (14 mg). NMR data are provided in given in FIG. 12.

### 70. Preparation of Dichloro((S)-4-(4-(2,3-diaminopropyl)-2,6-diiodophenoxy)phenol)platinum(II) (SWUA_{—}018)

The overall synthetic scheme for the preparation of the title compound is given below.

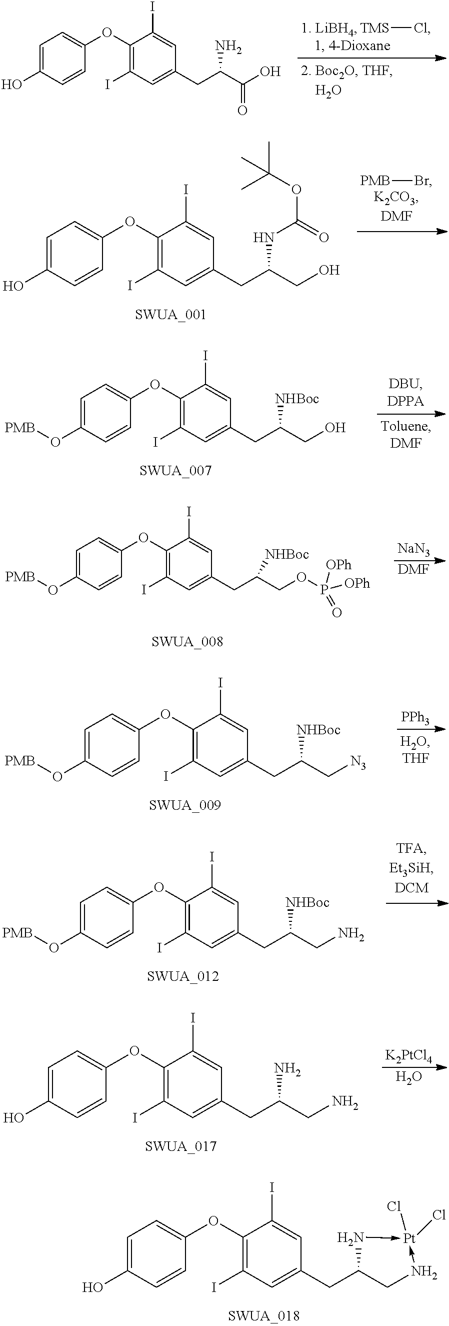

### a. Step 1: Preparation of (S)-tert-butyl (1-hydroxy-3-(4-(4-hydroxyphenoxy)-3,5-diiodophenyl)propan-2-yl)carbamate (SWUA_{—}001)

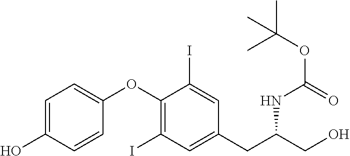 [SMILES: CC(C)(C)OC(=O)N[C@H](CO)Cc1cc(I)c(Oc2ccc(O)cc2)c(I)c1]

Briefly, 1,4-Dioxane (5 mL) in a round-bottom flask filled with nitrogen gas was chilled in an ice bath. LiBH₄ (3 mL, 2M in THF) and TMS-Cl (1.53 mL) were added dropwise by syringe. The solution was stirred and warmed to rt for 20 minutes before being chilled in an ice bath. (S)-2-Amino-3-(4-(4-hydroxyphenoxy)-3,5-diiodophenyl)propanoic acid (1.05 g, 2.000 mmol) was added through a powder funnel. The reaction continued in an ice bath overnight while stirring, gradually reaching room temperature LiBH₄ (3 mL, 2M in THF) with syringe and TMS-Cl (1.53 mL) were again added to the reaction mixture to push the reaction forward. The reaction mixture was put in an ice bath and stirred overnight, gradually reaching room temperature. The reaction mixture was transferred to a 250 mL beaker, using THF to rinse the flask. An aqueous 5M NaOH solution was added dropwise until the pH of the reaction mixture was 9. Boc₂O (0.564 mL, 2.428 mmol) was dissolved in THF (6 mL) in a separate scintillation vial. The Boc₂O solution was added to the reaction mixture dropwise, so that the pH of the reaction remained between 9 and 9.5. After 3 h, the reaction mixture was quenched with acid, and extracted with ethyl acetate. The ethyl acetate layer was washed with water three times and concentrate down. The crude product was purified by flash column chromatography (Biotage SP4, 40+M column, eluting with hexanes/ethyl acetate, 20-100% gradient) to yield a off-white solid. Yield: 834.1 mg (68.2%). ¹H NMR (400 MHz, DMSO) δ 9.05 (s, 1H), 7.71 (s, 2H), 6.70-6.58 (m, 3H), 6.47 (d, J=8.9 Hz, 2H), 4.73 (t, J=5.5 Hz, 1H), 3.61-3.47 (m, 1H), 3.37-3.31 (m, 1H), 3.29-3.21 (m, 1H), 2.80 (dd, J=13.5, 3.9 Hz, 1H), 2.41 (dd, J=13.3, 10.2 Hz, 1H), 1.29 (s, 9H).

### b. Step 2: Preparation of (S)-tert-butyl (1-(3,5-diiodo-4-(44(4-methoxybenzyl)oxy)phenoxy)phenyl)-3-hydroxypropan-2-yl)carbamate (SWUA_{—}007)

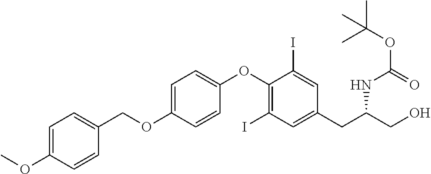 [SMILES: COc1ccc(COc2ccc(Oc3c(I)cc(C[C@@H](CO)NC(=O)OC(C)(C)C)cc3I)cc2)cc1]

Briefly, PMB-Br (144 μL , 0.988 mmol) and potassium carbonate (372 mg, 2.7 mmol) were added to a solution of (S)-tert-butyl (1-hydroxy-3-(4-(4-hydroxyphenoxy)-3,5-diiodophenyl)propan-2-yl)carbamate (549.1 mg, 0.898 mmol), prepared as described above, in DMF (3 mL) stirring at rt. Reaction continued at rt overnight. Additional PMB-Br (55 μL, 0.450 mmol) and potassium carbonate (185 mg, 1.35 mmol) were added to the reaction mixture, which stirred at rt for an additional 3.5 h. Aqueous ammonium chloride (10 mL) was added to the reaction mixture, and extracted with ethyl acetate (3×10 mL). The ethyl acetate layers were combined, washed with a saturated brine solution, dried with anhydrous sodium sulfate, and concentrated by rotovap. The crude product was purified by flash column chromatography (Biotage SP4, 40+M column, eluting with hexanes/ethyl acetate, 0-50% gradient) to yield of a white solid (240 mg). The flash chromatography was repeated on fractions from the first run which remain a mixture (Biotage SP4, 40+S column, eluting with hexanes/ethyl acetate, 0-50% gradient) to yield a white solid (110 mg). The two batches of the products were combined and dried on high vacuum overnight. Yield: 291.5 mg (44.4%). ¹H NMR (400 MHz, CDCl₃) δ 7.72 (s, 2H), 7.34 (d, J=8.5 Hz, 2H), 7.26 (s, 1H), 6.90 (t, J=8.5 Hz, 4H), 6.70 (d, J=9.1 Hz, 2H), 4.93 (s, 2H), 4.81 (d, J=6.5 Hz, 1H), 3.81 (s, 3H), 3.71 (d, J=11.0 Hz, 1H), 3.66-3.57 (m, 1H), 2.80 (d, J=7.1 Hz, 2H), 2.12 (s, 1H), 1.58 (s, 1H), 1.44 (s, 9H).

### c. Step 3: Preparation of (S)-tert-butyl (1-(3,5-diiodo-4-(4-((4-methoxybenzyl)oxy)phenoxy)phenyl)-3-(diphenoxyphosphoryl)oxy)propan-2-yl)carbamate (SWUA_{—}008)

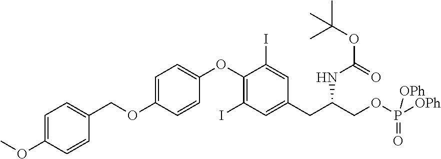 [SMILES: COc1ccc(COc2ccc(Oc3c(I)cc(C[C@@H](COP(=O)(Oc4ccccc4)Oc4ccccc4)NC(=O)OC(C)(C)C)cc3I)cc2)cc1]

Briefly, (S)-tert-butyl (1-(3,5-diiodo-4-(4-((4-methoxybenzyl)oxy)phenoxy)phenyl)-3-hydroxypropan-2-yl)carbamate (290 mg, 0.397 mmol), prepared as described above, was dissolved in dry toluene (2.65 mL) and DMF (350 μL). The solution was stirred in an ice bath for 10 min. DBU (89 μL, 0.595 mmol) and DPPA (128 μL, 0.595 mmol) were added to the cooled reaction mixture, which was allowed react while slowly warming to rt over 4.5 h. The reaction was quenched with water (10 mL) and extracted with ethyl acetate (3×10 mL). The ethyl acetate layers were combined and washed with a saturated brine, dried with anhydrous sodium sulfate, and concentrated by rotovap. The crude product was purified by flash column chromatography (Biotage SP4, 40+S column, eluting with hexanes/ethyl acetate, 0-40% gradient) to yield a white solid. Yield: 195 mg (51.0%). ¹H NMR (400 MHz, CDCl₃) δ 7.65 (s, 2H), 7.41-7.30 (m, 6H), 7.28-7.18 (m, 7H), 6.90 (dd, J=12.0, 8.9 Hz, 4H), 6.68 (d, J=9.1 Hz, 2H), 4.98-4.86 (m, 3H), 4.31 (s, 1H), 4.25-4.15 (m, 1H), 4.01 (s, 1H), 3.81 (s, 3H), 2.84-2.68 (m, 2H), 1.42 (s, 9H).

### d. Step 4: Preparation of (S)-tert-butyl (1-azido-3-(3,5-diiodo-4-(4-((4-methoxybenzyl)oxy)phenoxy)phenyl)propan-2-yl)carbamate (SWUA_{—}009)

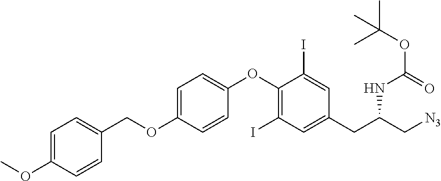 [SMILES: COc1ccc(COc2ccc(Oc3c(I)cc(C[C@@H](CN=[N+]=[N-])NC(=O)OC(C)(C)C)cc3I)cc2)cc1]

Briefly, sodium azide (152.8 mg, 2.350 mmol) was added to a solution of (S)-tert-butyl (1-(3,5-diiodo-4-(4-((4-methoxybenzyl)oxy)phenoxy)phenyl)-3-((diphenoxyphosphoryl)oxy)propan-2-yl)carbamate (224 mg, 0.232 mmol) in dry DMF (2.5 mL). The reaction mixture was stirred at 80° C. for 24 h. The reaction mixture was quenched with water (10 mL) and extracted with ethyl acetate (3×10 mL). The ethyl acetate layers were combined, washed with saturated brine, dried with anhydrous sodium sulfate, and concentrated into a scintillation vial. The crude product was filtered through a thin pad of silica gel (elute with hexane/ethyl acetate, 2:1). The filtrate was concentrated by rotovap and dried under high vacuum to yield a white solid. Yield: 138.8 mg (79%). ¹H NMR (400 MHz, CDCl₃) δ 7.70 (s, 2H), 7.34 (d, J=8.5 Hz, 2H), 7.26 (s, 1H), 6.93-6.86 (m, 4H), 6.70 (d, J=9.0 Hz, 2H), 4.93 (s, 2H), 4.69 (d, J=8.3 Hz, 1H), 3.92 (s, 1H), 3.82 (s, 3H), 3.45 (qd, J=12.4, 4.2 Hz, 2H), 2.85-2.64 (m, 2H), 1.44 (s, 9H). FT-IR: 3295, 2871, 2060, 1660, 1482, 1412, 1220, 1150, 1009 cm⁻¹.

### e. Step 5: Preparation of (S)-tert-butyl (1-amino-3-(3,5-diiodo-4-(4-((4-methoxybenzyl)oxy)phenoxy)phenyl)propan-2-yl)carbamate (SWUA_{—}012)

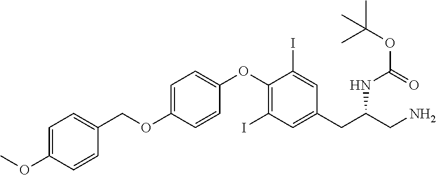 [SMILES: COc1ccc(COc2ccc(Oc3c(I)cc(C[C@@H](CN)NC(=O)OC(C)(C)C)cc3I)cc2)cc1]

Briefly, triphenylphosphine (40.4 mg, 0.154 mmol) was added to a solution of (S)-tert-butyl (1-azido-3-(3,5-diiodo-4-(4-((4-methoxybenzyl)oxy)phenoxy)phenyl)propan-2-yl)carbamate (106.9 mg, 0.141 mmol) in THF (1.6 mL) and water (161 μL). The reaction mixture was stirred at rt overnight. After 18 h, the reaction mixture was concentrated by rotovap. The crude product was purified by flash column chromatography (Biotage SP4, 25+S column, eluting with DCM/MeOH/NH₄OH, 100:0:0 to 85:14.8:0.2 gradient) to yield a pale brown solid. Yield: 89.7 mg (87.0%). ¹H NMR (400 MHz, CDCl₃) δ 7.70 (s, 1H), 7.35 (d, J=8.6 Hz, 1H), 6.90 (t, J=8.9 Hz, 2H), 6.70 (d, J=9.0 Hz, 1H), 4.93 (s, 1H), 4.06-3.55 (m, 2H), 2.72 (br s, 1H), 1.59-1.10 (m, 7H). FT-IR: 3921, 1653, 1487, 1411, 1225, 1210, 1169, 1150 cm⁻¹.

### f. Step 6: Preparation of (S)-4-(4-(2,3-diaminopropyl)-2,6-diiodophenoxy)phenol (SWUA_{—}017)

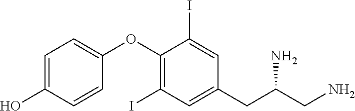 [SMILES: NC[C@@H](N)Cc1cc(I)c(Oc2ccc(O)cc2)c(I)c1]

Briefly, triethylsilane (65 μL, 0.407 mmol) was added to (S)-tert-butyl (1-amino-3-(3,5-diiodo-4-(4-((4-methoxybenzyl)oxy)phenoxy)phenyl)propan-2-yl)carbamate (75.5 mg, 0.103 mmol) in DCM (585 μL). The reaction mixture was cooled to 0° C. in an ice bath. TFA (650 μL) was added to the reaction mixture, which was swirled to make sure the solution was homogeneous. The reaction mixture reacted in ice for 1 h before being moved to rt for 1.5 h. The solvent was removed by blowing with nitrogen gas. Aqueous NaOH (5M, 700 μL) and water were used to quench the reaction, and ethyl acetate (3×3 mL) was used to extract from the aqueous solution. The ethyl acetate layers were combined, wash with a saturated brine, dried with anhydrous sodium sulfate, and concentrated into a scintillation vial. The crude product was dissolved in aqueous HCl and washed with ethyl acetate. The aqueous layer was neutralized with aqueous NaOH and then extracted with ethyl acetate. The ethyl acetate layer was concentrated by rotovap and dried on a high vacuum pump to yield a pale yellow solid. Yield: 38.3 mg (72.6%) ¹H NMR (400 MHz, DMSO) δ 7.81 (s, 2H), 6.69 (d, J=8.9 Hz, 2H), 6.55 (d, J=8.9 Hz, 2H), 3.00 (br s, 1H), 2.84-2.66 (m, 2H), 2.61-2.50 (m, 2H), 1.91 (s, 1H).

### g. Step 7: Preparation of Dichloro((S)-4-(4-(2,3-diaminopropyl)-2,6-diiodophenoxy)phenol)platinum(II) (SWUA_{—}018)

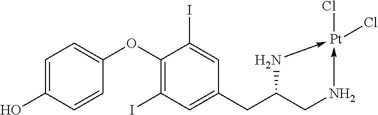

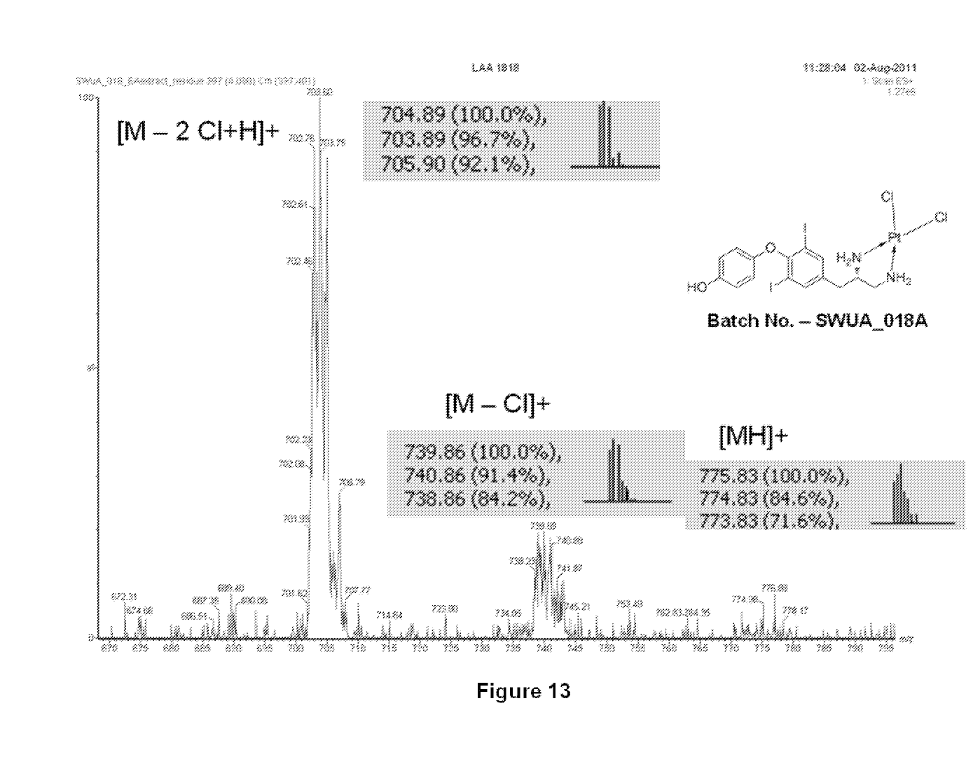

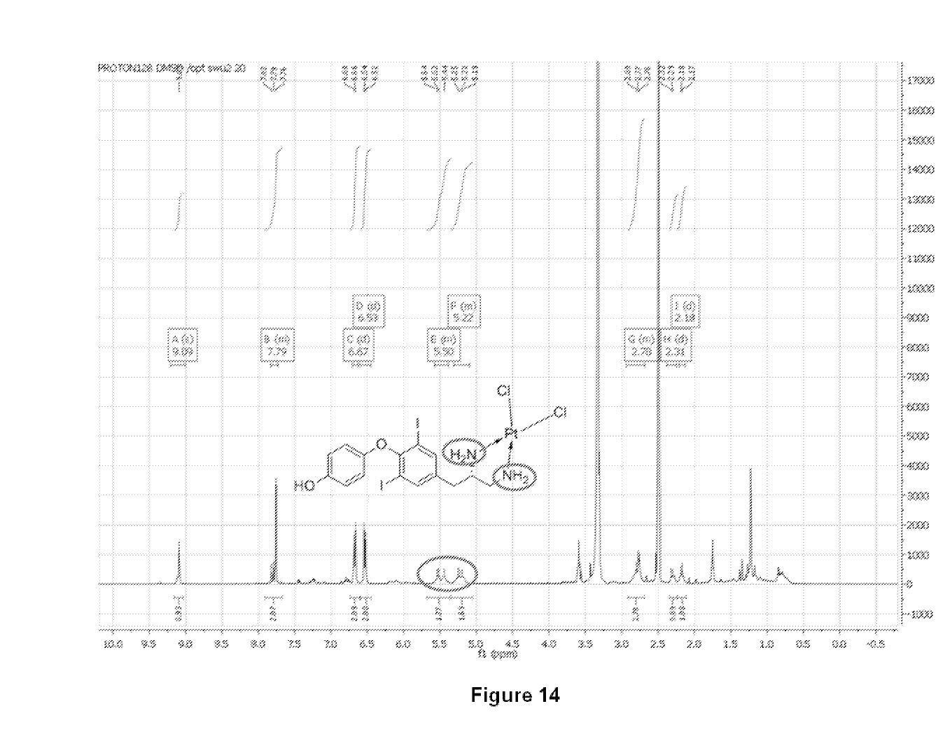

Briefly, Potassium tetrachloroplatinate(II) (31.4 mg, 0.076 mmol) was added to a solution of (S)-4-(4-(2,3-diaminopropyl)-2,6-diiodophenoxy)phenol (38.3 mg, 0.075 mmol) in water (417 μL) and HCl (1M, 12.6 μL, 0.151 mmol). An ultrasound was used to help SWUA_{—}017 dissolve completely. The reaction mixture was stirred at rt, and aqueous NaOH (5M, 53 μL) was added to neutralize the reaction mixture to pH 7. Additional NaOH (5M, 21 μL) was used to neutralize solution every few hours over the course of two days. After the starting material was used up, according to LC-MS, ethyl acetate (2×6 mL) and THF/ethyl acetate (1:1, 3×6 mL) was used to extract the aqueous layer and precipitate. All extractions were combined and dried to form a crude mixture. LC-MS of the crude mixture suggest that it is a 2:1 mixture of a product with a 703 molecular mass (“Product A”) and a product with 816 molecular mass (“Product B”). Product A was identified as dichloro((S)-4-(4-(2,3-diaminopropyl)-2,6-diiodophenoxy)phenol)platinum(II) due to an observation that in addition to the M=703 peak, a weaker M=739 and a very weak M=776 peak were observed on the mass spectroscopy. The M=703 peak is most likely the [M−2 Cl]⁺ peak of the title compound above. The M=739 peak is most likely the [M−Cl]⁺ peak. The very weak M=776 peak is most likely the [M+H]⁺ peak, each based on their isotopic peak patterns which are unique to their platinum and chloride composition. The precipitate of the reaction mixture was extracted again with ethyl acetate/THF (1:1, 6 mL) and THF (6 mL). LCMS suggested that both layers were >95% pure in Product A. The two layers were combined, concentrated on a rotovap, and dried on a high vacuum pump to yield a white solid. Representative mass spectrometry and ¹H NMR data for the title compound is shown in FIGS. 13 and 14, respectively. Mass of dried product A: 3.8 mg (6.5% yield). ¹H NMR (400 MHz, DMSO) δ 9.09 (s, 1H), 7.83-7.73 (m, 2H), 6.67 (d, J=8.1 Hz, 2H), 6.53 (d, J=8.1 Hz, 2H), 5.58-5.38 (m, 2H), 5.31-5.09 (m, 2H), 2.94-2.69 (m, 3H), 2.31 (d, J=9.3 Hz, 1H), 2.18 (d, J=6.4 Hz, 1H).

### 71. Preparation of (S)—N-(2-amino-3-(4-(4-hydroxyphenoxy)-3,5-diiodophenyl)propyl)acetamide (MLAF-065)

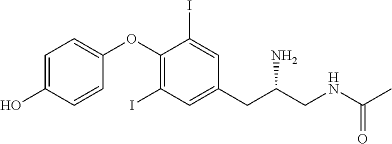 [SMILES: CC(=O)NC[C@@H](N)Cc1cc(I)c(Oc2ccc(O)cc2)c(I)c1]

The synthetic scheme for the preparation of the title compound, as well as MLAF-066, MLAF-074, MLAF-075, MLAF-081, MLAF-083, and MLAF-085, beginning with intermediate SWUA_{—}012 is as given below.

Briefly, the preparation of the title compound was carried out as follows: Pyridine (2 μL, 0.025 mmol) was added to a solution of SWUA_{—}012 (10 mg, 0.014 mmol), and Ac₂O (2 μL, 0.021 mmol), in DMF (200 μL). After 15 min, the brown reaction mixture was added to water (3 mL) and extracted with ethyl acetate (3×3 mL). The combined organic layers were washed with saturated brine, dried over Na₂SO₄, filtered, and concentrated, to give a white solid. The solid was dissolved in DCM (200 μL) and triethylsilane (10 μL). TFA (200 μL) was added to the reaction mixture and the solution was allowed to stand for 1 h before being concentrated. The crude product was purified by preparative TLC (10×20 cm plate, 1 mm thick, eluting with DCM/MeOH/NH₄OH, 9:1:0.1) to give a white solid (4.5 mg, 60% yield). ¹H NMR (400 MHz, MeOD) δ 7.83 (s, 2H), 6.76-6.68 (m, 2H), 6.63-6.55 (m, 2H), 3.32-3.25 (m, 1H), 3.19-3.07 (m, 2H), 2.77 (dd, J=13.7, 5.1 Hz, 1H), 2.53 (dd, J=13.7, 7.6 Hz, 1H), 1.99 (s, 3H); HRMS (ESI) m/z, [M+H] calculated for C₁₇H₁₉I₂N₂O₃, 552.9485; observed, 552.9481.

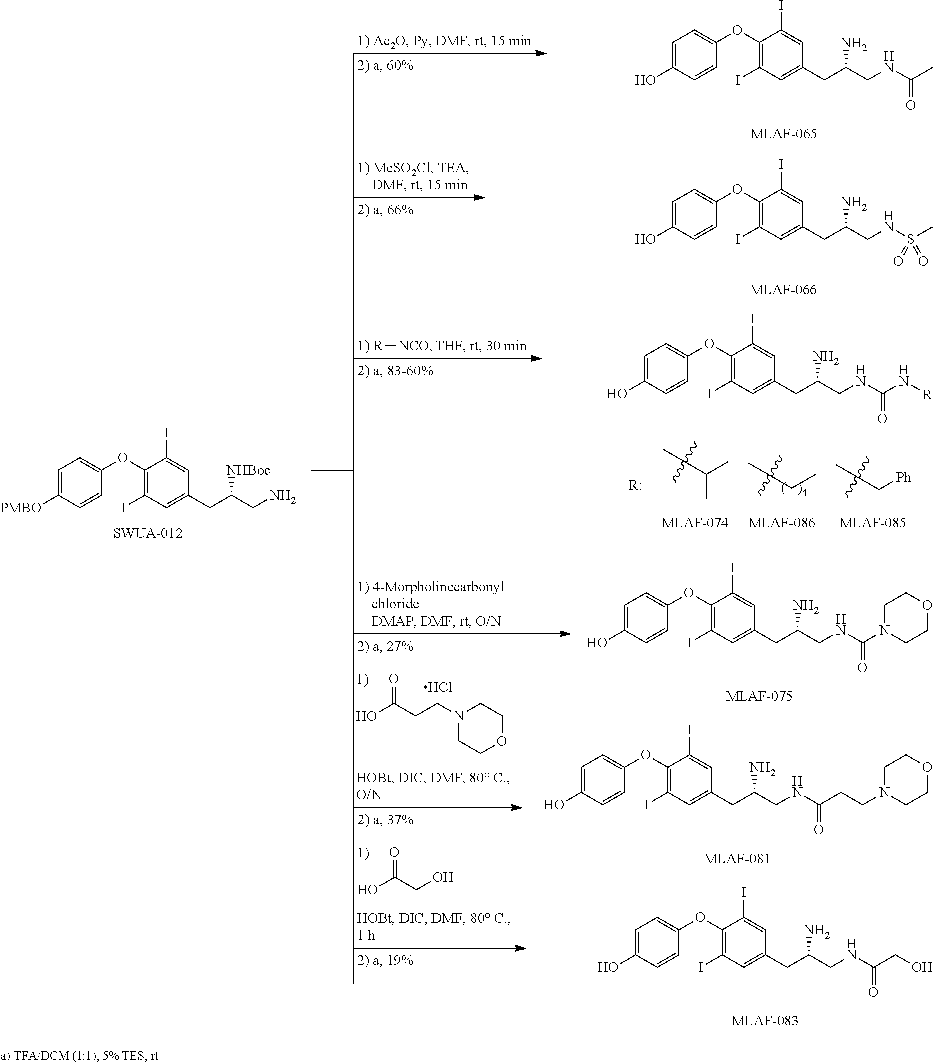

### 72. Preparation of (S)—N-(2-amino-3-(4-(4-hydroxyphenoxy)-3,5-diiodophenyl)propyl)methanesulfonamide (MLAF-066)

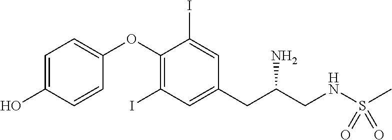 [SMILES: CS(=O)(=O)NC[C@@H](N)Cc1cc(I)c(Oc2ccc(O)cc2)c(I)c1]

Triethylamine (2 μL, 0.014 mmol) was added to a solution of SWUA_{—}012 (8 mg, 10.95 μmol), and methanesulfonyl chloride (1.5 μL, 0.019 mmol), in DMF (200 μL). After 15 min, the reaction mixture was added to water (3 mL) and extracted with ethyl acetate (3×3 mL). The combined organic layers were washed with saturated brine, dried over Na₂SO₄, filtered, and concentrated, to give a white solid. The solid was dissolved in DCM (200 μL) and triethylsilane (10 μL). TFA (200 μL) was added to the reaction mixture and the solution was allowed to stand for 1 h before being concentrated. The crude product was purified by preparative TLC (10×20 cm plate, 1 mm thick, eluting with DCM/MeOH/NH₄OH, 9:1:0.1) to give a white solid (4.3 mg, 70% yield). ¹H NMR (400 MHz, MeOD) δ 7.84 (s, 2H), 6.71 (d, J=8.9 Hz, 2H), 6.60 (d, J=9.0 Hz, 2H), 3.20-3.07 (m, 2H), 3.06-2.95 (m, 4H), 2.83 (dd, J=13.6, 5.1 Hz, 1H), 2.60-2.49 (m, 1H); HRMS (ESI) m/z, [M+H] calculated for C₁₆H₁₉I₂N₂O₄S, 588.9155; observed, 588.9152.

### 73. Preparation of (S)-1-(2-amino-3-(4-(4-hydroxyphenoxy)-3,5-diiodophenyl)propyl)-3-isopropylurea (MLAF-074)

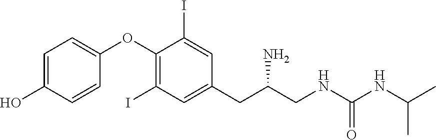 [SMILES: CC(C)NC(=O)NC[C@@H](N)Cc1cc(I)c(Oc2ccc(O)cc2)c(I)c1]

A solution of SWUA_{—}012 (15 mg, 0.021 mmol), and 2-isocyanatopropane (2.22 μL, 0.023 mmol), in DMF (200 μL), was allowed to stand for 15 min. The colorless reaction mixture was added to water (3 mL) and extracted with ethyl acetate (3×3 mL). The combined organic layers were washed with saturated brine, dried over Na₂SO₄, filtered, and concentrated, to give a white solid. The solid was dissolved in DCM (100 μL) and triethylsilane (10 μL). TFA (100 μL) was added to the reaction mixture and the solution was allowed to stand for 30 min before being concentrated. The crude product was purified by flash column chromatography (Biotage SP4, 12+S KP-NH amine column, eluting with DCM/MeOH, 0-10% gradient) to give a white solid (9.9 mg, 83% yield). ¹H NMR (400 MHz, MeOD) δ 7.81 (s, 2H), 6.76-6.67 (m, 2H), 6.65-6.52 (m, 2H), 3.87-3.75 (m, 1H), 3.23 (d, J=8.5 Hz, 1H), 3.13-2.99 (m, 2H), 2.77 (dd, J=13.6, 4.9 Hz, 1H), 2.57-2.45 (m, 1H), 1.15 (d, J=6.5 Hz, 6H); HRMS (ESI) m/z, [M+H] calculated for C₁₉H₂₄I₂N₃O₃, 595.9907; observed, 595.9894.

### 74. Preparation of (S)—N-(2-amino-3-(4-(4-hydroxyphenoxy)-3,5-diiodophenyl)propyl)morpholine-4-carboxamide (MLAF-075)

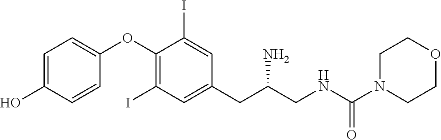 [SMILES: N[C@H](CNC(=O)N1CCOCC1)Cc1cc(I)c(Oc2ccc(O)cc2)c(I)c1]

DMAP (0.2 mg, 1.637 μmol) was added to a solution of SWUA_{—}012 (15 mg, 0.021 mmol), and morpholine-4-carbonyl chloride (3.59 μL, 0.031 mmol), in DMF (200 μL). After 3 h, more DMAP (0.2 mg, 1.637 mmol) and morpholine-4-carbonyl chloride (3.59 μL, 0.031 mmol) were added, and the solution was allowed to stand overnight. The solution was added to water (3 mL) and extracted with ethyl acetate (3×3 mL). The combined organic layers were washed with saturated brine, dried over Na₂SO₄, filtered, and concentrated. The crude product was purified by flash column chromatography (Biotage SP4, 12+S column, eluting with DCM/MeOH, 0-8% gradient) to give a white solid. The solid was dissolved in DCM (100 μL) and triethylsilane (10 μL). TFA (100 μL) was added to the reaction mixture and the solution was allowed to stand for 30 min before being concentrated. The crude product was purified by flash column chromatography (Biotage SP4, 12+S KP-NH amine column, eluting with DCM/MeOH, 0-10% gradient) to give a white solid (3.5 mg, 27% yield). ¹H NMR (400 MHz, MeOD) δ 7.80 (s, 2H), 6.72-6.66 (m, 2H), 6.60-6.54 (m, 2H), 3.69-3.62 (m, 4H), 3.39-3.33 (m, 5H), 3.16-3.05 (m, 2H), 2.74 (dd, J=13.7, 5.2 Hz, 1H), 2.53 (dd, J=13.8, 7.4 Hz, 1H); HRMS (ESI) m/z, [M+H] calculated for C₂₀H₂₄I₂N₃O₄, 623.9856; observed, 623.9855.

### 75. Preparation of (S)—N-(2-amino-3-(4-(4-hydroxyphenoxy)-3,5-diiodophenyl)propyl)-3-morpholinopropanamide (MLAF-081)

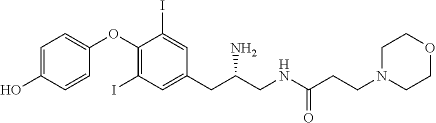 [SMILES: N[C@H](CNC(=O)CCN1CCOCC1)Cc1cc(I)c(Oc2ccc(O)cc2)c(I)c1]

A solution of SWUA_{—}012 (15 mg, 0.021 mmol), 3-morpholinepropanoic acid hydrochloride (4.4 mg, 0.023 mmol), HOBt (3.1 mg, 0.023 mmol), and DIC (3.84 μL, 0.025 mmol), in DMF (200 μL), was heated to 80° C. overnight. 1M NaOH (3 mL) was added to the yellow reaction mixture and extracted with ethyl acetate (3×3 mL). The combined organic layers were washed with saturated brine, dried over Na₂SO₄, filtered, and concentrated. The crude product was purified by preparative TLC (20×20 cm plate, 1 mm thick, eluting with 9:1 DCM/MeOH) to give a white solid (10.4 mg). The white solid was dissolved in DCM (100 μL) and triethylsilane (10 μL). TFA (100 μL) was added to the reaction mixture and the solution was allowed to stand for 1 h before being concentrated. The crude product was purified by flash column chromatography (Biotage SP4, 12+S KP-NH amine column, eluting with DCM/MeOH, 0-10% gradient) to give a colorless residue (5 mg, 37% yield). ¹H NMR (400 MHz, MeOD) δ 7.72 (s, 2H), 6.65-6.55 (m, 2H), 6.53-6.43 (m, 2H), 3.63-3.55 (m, 4H), 3.17 (d, J=7.8 Hz, 1H), 3.13-3.03 (m, 2H), 2.68 (dd, J=13.8, 5.3 Hz, 1H), 2.61-2.53 (m, 2H), 2.47 (dd, J=13.7, 7.0 Hz, 1H), 2.40 (bs, 4H), 2.32 (t, J=7.0 Hz, 2H); HRMS (ESI) m/z, [M+H] calculated for C₂₂H₂₈I₂N₃O₄, 652.0169; observed, 652.0154.

### 76. Preparation of (S)—N-(2-amino-3-(4-(4-hydroxyphenoxy)-3,5-diiodophenyl)propyl)-2-hydroxyacetamide (MLAF-083)

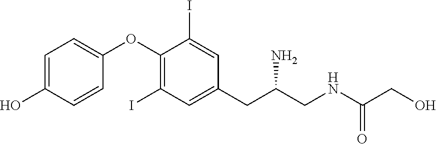 [SMILES: N[C@H](CNC(=O)CO)Cc1cc(I)c(Oc2ccc(O)cc2)c(I)c1]

A solution of SWUA_{—}012 (15 mg, 0.021 mmol), 2-hydroxyacetic acid (1.7 mg, 0.023 mmol), HOBt (3 mg, 0.023 mmol), and DIC (3.84 μL, 0.025 mmol), in DMF (200 μL), was heated to 80° C. and stirred for 1 h. The yellow reaction mixture was added to water (3 mL) and extracted with ethyl acetate (3×3 mL). The combined organic layers were washed with saturated brine, dried over Na₂SO₄, filtered, and concentrated, to give a pale brown solid. The solid was dissolved in DCM (100 μL) and triethylsilane (10 μL). TFA (100 μL) was added to the reaction mixture and the solution was allowed to stand for 1 h before being concentrated. The crude product was purified by flash column chromatography (Biotage SP4, 12+S KP-NH amine column, eluting with DCM/MeOH, 0-10% gradient) to give a colorless residue (9 mg, 77% yield). ¹H NMR (400 MHz, MeOD) δ 7.81 (s, 2H), 6.69 (d, J=8.8 Hz, 2H), 6.58 (d, J=9.0 Hz, 2H), 4.01 (s, 2H), 3.40-3.32 (m, 1H), 3.27-3.11 (m, 2H), 2.77 (dd, J=13.7, 5.3 Hz, 1H), 2.53 (dd, J=13.7, 7.9 Hz, 1H); HRMS (ESI) m/z, [M+H] calculated for C₁₇H₁₉I₂N₂O₄, 568.9434; observed, 568.9435.

### 77. Preparation of (S)-1-(2-amino-3-(4-(4-hydroxyphenoxy)-3,5-diiodophenyl)propyl)-3-benzylurea (MLAF-085)

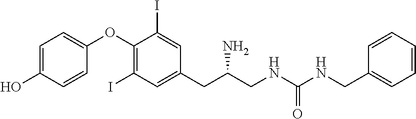 [SMILES: N[C@H](CNC(=O)NCc1ccccc1)Cc1cc(I)c(Oc2ccc(O)cc2)c(I)c1]

A heterogeneous solution of SWUA_{—}012 (15 mg, 0.021 mmol), and (isocyanatomethyl)benzene (2.77 μL, 0.023 mmol), in THF (200 μL), was allowed to stand for 30 min at rt. The now homogeneous reaction mixture was concentrated, then DCM (100 μL), TES (10 μL), and TFA (100 μL) were added, and the solution was allowed to stand for 15 min. The yellow reaction mixture was concentrated and purified by flash column chromatography (Biotage SP4, 12+S KP-NH amine column, eluting with DCM/MeOH, 0-9% gradient) to give a colorless residue (9 mg, 68% yield). ¹H NMR (400 MHz, MeOD) δ 7.81 (s, 2H), 7.37-7.29 (m, 4H), 7.29-7.22 (m, 1H), 6.75-6.68 (m, 2H), 6.63-6.57 (m, 2H), 4.35 (s, 2H), 3.26 (dd, J=16.1, 7.7 Hz, 1H), 3.14-3.05 (m, 2H), 2.77 (dd, J=13.7, 4.8 Hz, 1H), 2.49 (dd, J=13.7, 7.6 Hz, 1H); MS (ESI) m/z, [M+H] calculated for C₂₃H₂₄I₂N₃O₃,643.99; observed, 644.51.

### 78. Preparation of (S)-1-(2-amino-3-(4-(4-hydroxyphenoxy)-3,5-diiodophenyl)propyl)-3-hexylurea (MLAF-086)

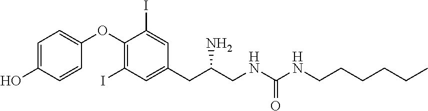 [SMILES: CCCCCCNC(=O)NC[C@@H](N)Cc1cc(I)c(Oc2ccc(O)cc2)c(I)c1]

A heterogeneous solution of SWUA_{—}012 (15 mg, 0.021 mmol), and 1-isocyanatohexane (3.29 μL, 0.023 mmol), in THF (200 μL), was allowed to stand for 30 min. The now homogeneous solution was concentrated, and the residue was dissolved in DCM (100 μL), TES (10 μL), and TFA (100 μL). The yellow reaction mixture was concentrated after 15 min and purified by flash column chromatography (Biotage SP4, 12+S KP-NH amine column, eluting with DCM/MeOH, 0-9% gradient) to give a colorless residue (7.9 mg, 60% yield). ¹H NMR (400 MHz, MeOD) δ 7.82 (s, 2H), 6.71 (dd, J=8.9, 1.7 Hz, 2H), 6.60 (dd, J=8.9, 1.7 Hz, 2H), 3.30-3.18 (m, 1H), 3.17-3.03 (m, 4H), 2.78 (dd, J=13.6, 4.1 Hz, 1H), 2.51 (dd, J=13.1, 7.1 Hz, 1H), 1.57-1.44 (m, 2H), 1.42-1.30 (m, 6H), 0.93 (t, J=6.0 Hz, 3H); MS (ESI) m/z, [M+H] calculated for C₂₂H₃₀I₂N₃O₃, 638.04; observed, 638.53.

### 79. Preparation of (S)-4-(4-(4-hydroxyphenoxy)-3,5-diiodobenzyl)imidazolidin-2-one (MLAG-060)

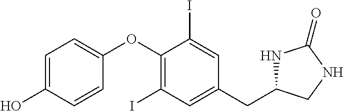 [SMILES: O=C1NC[C@H](Cc2cc(I)c(Oc3ccc(O)cc3)c(I)c2)N1]

The synthetic scheme for the preparation of the title compound from SWUA_{—}017 is given below.

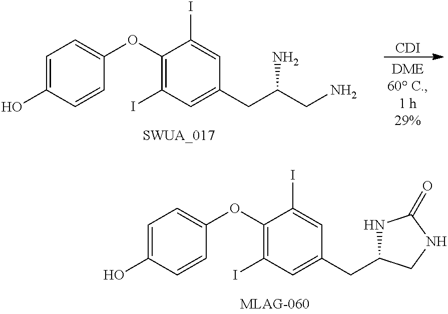 [SMILES: NC[C@@H](N)Cc1cc(I)c(Oc2ccc(O)cc2)c(I)c1 ; "CDI" ; O=C1NC[C@H](Cc2cc(I)c(Oc3ccc(O)cc3)c(I)c2)N1]

Briefly, a solution of SWUA_{—}017 (10.5 mg, 0.021 mmol), and CDI (4.34 mg, 0.027 mmol), in DME (200 μL), was stirred at 60° C. for 1 h. The crude mixture was purified by flash column chromatography (Biotage SP4, 12+S column, eluting with DCM/MeOH, O-10% gradient) to give the product (3.2 mg, 29% yield). ¹H NMR (400 MHz, DMSO) δ 9.09 (s, 1H), 7.79 (s, 2H), 6.68 (dd, J=9.0, 3.1 Hz, 2H), 6.56 (dd, J=9.0, 3.2 Hz, 2H), 6.43 (s, 1H), 6.11 (s, 1H), 3.98-3.84 (m, 1H), 2.99 (t, J=6.4 Hz, 1H), 2.81-2.63 (m, 2H); MS (ESI) m/z, [M+H] calculated for C₁₆H₁₅I₂N₂O₃, 536.92; observed, 537.32.

### 80. Preparation of Methyl 2-(3,5-diiodo-4-(4-methoxyphenoxy)phenyl)acetate (MLAG-063)

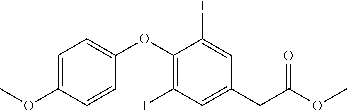

The synthetic scheme for the preparation of the title compound, as well as MLAG-064, MLAG-066-1, MLAG-066-2, and MLAG-066-3, beginning with methyl 2-(4-hydroxy-3,5-diiodophenyl)acetateis given below.

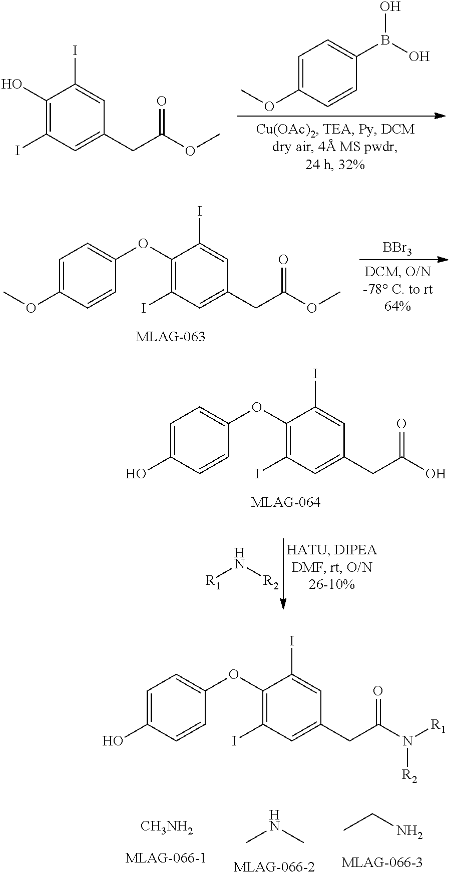

Briefly, preparation of MLAG-064 is carried out as follows: A mixture of TEA (800 μL, 5.74 mmol) and pyridine (464 μL, 5.74 mmol) was added to a solution containing methyl 2-(4-hydroxy-3,5-diiodophenyl)acetate (400 mg, 0.957 mmol), (4-methoxyphenyl)boronic acid (291 mg, 1.91 mmol), copper (II) acetate (521 mg, 2.87 mmol), and 4 Å molecular sieve powder (800 mg), in dry DCM (10 mL). A drying tube was attached and the reaction mixture was stirred overnight. Additional (4-methoxyphenyl)boronic acid (146 mg, 0.957 mmol) and copper (II) acetate (261 mg, 1.44 mmol) were added and the mixture stirred for an additional 24 h. The solution was diluted with ethyl acetate and filtered through celite. The filtrate was concentrated and purified by flash column chromatography (Biotage SP4, 40+S column, eluting with hexanes/ethyl acetate, 0-15% gradient) to give an oil (158 mg, 32% yield). ¹H NMR (400 MHz, CDCl₃) δ 7.77 (s, 2H), 6.87-6.78 (m, 2H), 6.74-6.67 (m, 2H), 3.77 (s, 3H), 3.74 (s, 3H), 3.56 (s, 2H).

### 81. Preparation of 2-(4-(4-hydroxyphenoxy)-3,5-diiodophenyl)acetic acid (MLAG-064)

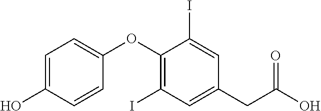 [SMILES: O=C(O)Cc1cc(I)c(Oc2ccc(O)cc2)c(I)c1]

A 1M in DCM solution of BBr₃ (1.2 mL, 1.2 mmol) was added dropwise to a solution of MLAG-063 (124 mg, 0.237 mmol), in DCM (2.4 mL), at −78° C. After 3 h, additional BBr₃ (1.2 mL, 1.2 mmol) was added dropwise and the solution was allowed to warm to rt overnight. The dark brown reaction mixture was added to 1M HCl (10 mL) and extracted with ethyl acetate (3×10 mL). The combined organic layers were washed with saturated brine, dried over Na₂SO₄, filtered, and concentrated, to give the product (75 mg, 64% yield), which was used in the next step without further purification.

### 82. Preparation of 2-(4-(4-hydroxyphenoxy)-3,5-diiodophenyl)-N-Methylacetamide (MLAG-066-1)

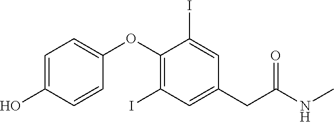 [SMILES: CNC(=O)Cc1cc(I)c(Oc2ccc(O)cc2)c(I)c1]

Starting with MLAG-064, general procedure (A) was used to give the desired product (3.3 mg, 21% yield). ¹H NMR (400 MHz, DMSO) δ 9.17 (s, 1H), 8.05 (s, 1H), 7.86 (s, 2H), 6.76 (d, J=8.8 Hz, 2H), 6.60 (d, J=8.6 Hz, 2H), 3.45 (d, J=6.7 Hz, 2H), 2.66 (t, J=5.7 Hz, 3H); MS (ESI) m/z, [M+H] calculated for C₁₅H₁₄I₂NO₃, 509.91; observed, 510.36.

### 83. Preparation of 2-(4-(4-hydroxyphenoxy)-3,5-diiodophenyl)-N,N-dimethylacetamide (MLAG-066-2)

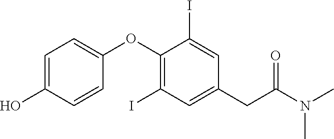 [SMILES: CN(C)C(=O)Cc1cc(I)c(Oc2ccc(O)cc2)c(I)c1]

Starting with MLAG-064, general procedure (A) was used to give the desired product (2.5 mg, 16% yield). ¹H NMR (400 MHz, DMSO) δ 9.11 (d, J=5.5 Hz, 1H), 7.77 (d, J=5.3 Hz, 2H), 6.75-6.65 (m, 2H), 6.58-6.49 (m, 2H), 3.70 (d, J=4.6 Hz, 2H), 3.04 (d, J=5.4 Hz, 3H), 2.85 (d, J=5.3 Hz, 3H); MS (ESI) m/z, [M+H] calculated for C₁₆H₁₆I₂NO₃, 523.92; observed, 524.41.

### 84. Preparation of N-ethyl-2-(4-(4-hydroxyphenoxy)-3,5-diiodophenyl)acetamide (MLAG-066-3)

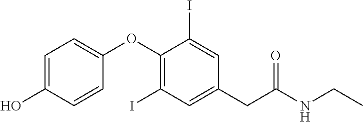 [SMILES: CCNC(=O)Cc1cc(I)c(Oc2ccc(O)cc2)c(I)c1]

Starting with MLAG-064, general procedure (A) was used to give the desired product (4.1 mg, 26% yield). ¹H NMR (400 MHz, DMSO) δ 9.11 (d, J=6.3 Hz, 1H), 8.08 (d, J=4.4 Hz, 1H), 7.80 (d, J=6.3 Hz, 2H), 6.75-6.65 (m, 2H), 6.58-6.48 (m, 2H), 3.38 (d, J=5.8 Hz, 2H), 3.14-3.02 (m, 2H), 2.55 (d, J=6.4 Hz, 3H); MS (ESI) m/z, [M+H] calculated for C₁₆H₁₆I₂NO₃, 523.92; observed, 524.28.

### 85. Preparation of tert-butyl 4-hydroxybenzyl carbamate (MLAG-067)

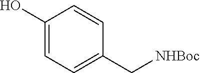 [SMILES: CC(C)(C)OC(=O)NCc1ccc(O)cc1]

The synthetic scheme for the preparation of the title compound, as well as MLAG-070, MLAG-070, and MLAG-082, beginning with 4-(aminomethyl)phenol is given below.

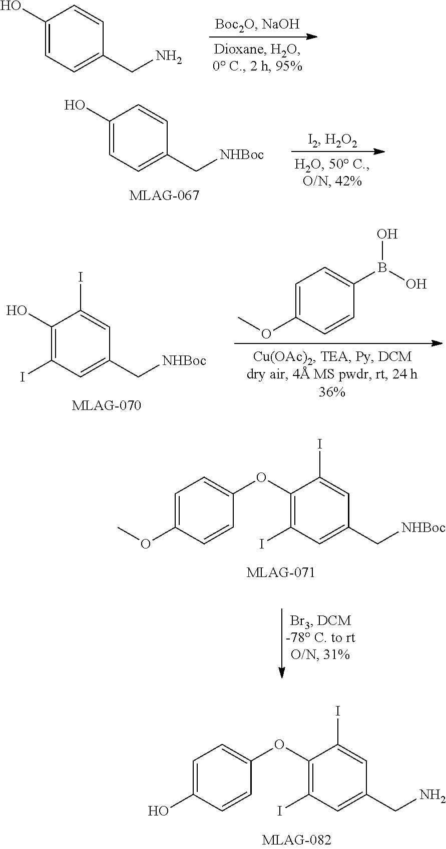

Briefly, the title compound was prepared as follows: A mixture of 4-(aminomethyl)phenol (500 mg, 4.06 mmol), ind (10 mL), and water (10 mL), at 0° C., was adjusted to pH˜10 using 1M NaOH. Boc₂O (1.131 mL, 4.87 mmol) was added and the solution stirred for 2 h. 1M HCl was added to the reaction mixture to a pH 2.5 and extracted with ethyl acetate (3×20 mL). The combined organic layers were washed with saturated brine, dried over Na₂SO₄, filtered, and concentrated to give the desired product (1.02 g), which was used in the next step without further purification.

### 86. Preparation of tert-butyl 4-hydroxy-3,5-diiodobenzylcarbamate (MLAG-070)

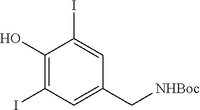 [SMILES: CC(C)(C)OC(=O)NCc1cc(I)c(O)c(I)c1]

A 30% aqueous solution of hydrogen peroxide (55 μL, 0.537 mmol) was added dropwise to a stirring solution of MLAG-067 (40 mg, 0.179 mmol), and I₂ (68.2 mg, 0.269 mmol), in water (2 mL). The reaction mixture was heated to 50° C. and stirred overnight. 1M Na₂S₂O₃ (5 mL) was added to the reaction mixture and extracted with ethyl acetate (3×5 mL). The combined organic layers were washed with saturated brine, dried over Na₂SO₄, filtered, and concentrated. The crude product was purified by flash column chromatography (Biotage SP4, 12+M column, eluting with hexanes/ethyl acetate, 0-30% gradient) to give a white solid (36 mg, 42% yield). ¹H NMR (400 MHz, CDCl₃) δ 7.59 (s, 2H), 5.73 (s, 1H), 4.83 (bs, 1H), 4.17 (d, J=5.4 Hz, 2H), 1.46 (s, 9H).

### 87. Preparation of tert-butyl 3,5-diiodo-4-(4-methoxyphenoxy)benzylcarbamate (MLAG-071)

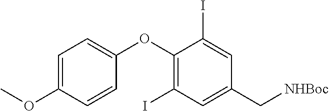 [SMILES: COc1ccc(Oc2c(I)cc(CNC(=O)OC(C)(C)C)cc2I)cc1]

A mixture of TEA (63 μL, 0.455 mmol) and pyridine (37 μL, 0.455 mmol) was added to a stirring solution of MLAG-070 (36 mg, 0.076 mmol), (4-methoxyphenyl)boronic acid (23 mg, 0.152 mmol), copper (II) acetate (41.3 mg, 0.227 mmol), and 4 Å molecular sieve powder (80 mg), in dry DCM (1 mL). A drying tube was attached and the mixture stirred overnight. The reaction mixture was diluted with ethyl acetate and filtered through celite. The filtrate was concentrated and purified by flash column chromatography (Biotage SP4, 12+S column, eluting with hexanes/ethyl acetate, 0-30% gradient) to give the desired product (15.9 mg, 36% yield). ¹H NMR (400 MHz, CDCl₃) δ 7.75 (s, 2H), 6.83 (d, J=9.0 Hz, 2H), 6.71 (d, J=9.1 Hz, 2H), 4.91 (bs, 1H), 4.27 (d, J=4.9 Hz, 2H), 3.77 (s, 3H), 1.48 (s, 9H).

### 88. Preparation of 4-(4-(aminomethyl)-2,6-diiodophenoxy)phenol (MLAG-082)

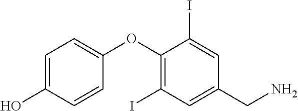 [SMILES: NCc1cc(I)c(Oc2ccc(O)cc2)c(I)c1]

A 1M in DCM solution of BBr₃ (82 μL, 0.082 mmol) was added dropwise to a solution of MLAG-071 (15.9 mg, 0.027 mmol) in DCM (200 μL) at −78° C. The reaction mixture was stirred overnight under N2 and allowed to reach rt. 1M HCl (3 mL) was added to the reaction mixture, basified with NaOH and extracted with ethyl acetate (3×5 mL). The combined organic layers were washed with saturated brine, dried over Na₂SO₄, filtered, and concentrated. The crude product was purified by flash column chromatography (Biotage SP4, 12+S column, eluting with DCM/MeOH, 0-14% gradient) to give the desired product (4 mg, 31% yield). ¹H NMR (400 MHz, DMSO) δ 9.09 (s, 1H), 7.88 (s, 2H), 6.68 (d, J=8.9 Hz, 2H), 6.52 (d, J=8.9 Hz, 2H), 3.68 (s, 2H); MS (ESI) m/z, [M+H] calculated for C₁₃H₁₂I₂NO₂, 467.90; observed, 468.36.

### 89. Preparation of (S)-4-(4-(2-amino-3-hydroxypropyl)-2,6-diiodophenoxy)-2-iodophenol (MLAG-083)

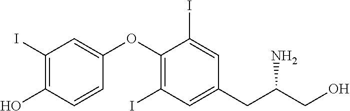 [SMILES: N[C@H](CO)Cc1cc(I)c(Oc2ccc(O)c(I)c2)c(I)c1]

The synthetic scheme for the preparation of the title compound from sodium (S)-2-amino-3-(4-(4-hydroxy-3-iodophenoxy)-3,5-diiodophenyl)propanoate is given below.

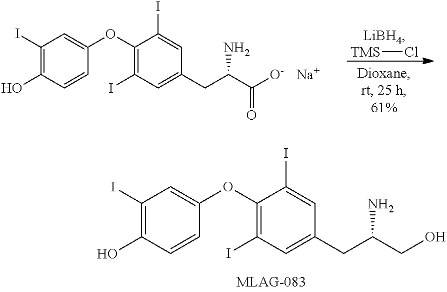 [SMILES: N[C@@H](Cc1cc(I)c(Oc2ccc(O)c(I)c2)c(I)c1)C(=O)[O-] ; [Na+] ; C[Si](C)(C)Cl ; N[C@H](CO)Cc1cc(I)c(Oc2ccc(O)c(I)c2)c(I)c1]

TMS-Cl (57 μL, 0.446 mmol) was added dropwise to a stirring solution of 2M in THF LiBH₄ (149 μL, 0.297 mmol) in dry 1,4-dioxane (1 mL), under a N₂. After 30 min, the solution was cooled to 0° C. and sodium (S)-2-amino-3-(4-(4-hydroxy-3-iodophenoxy)-3,5-diiodophenyl)propanoate (100 mg, 0.149 mmol) was added. The reaction mixture was vigorously stirred overnight. Additional LiBH₄ (149 μL, 0.297 mmol) and TMS-Cl (57 μL, 0.446 mmol) were added to the reaction mixture, and stirred for an additional 5 h. The thick white mixture was added to ice and basified with 1M NaOH, then extracted with ethyl acetate (3×10 mL). The combined organic layers were washed with saturated brine, dried over Na₂SO₄, filtered, and concentrated. The crude product was purified by flash column chromatography (Biotage SP4, 25+S KP-NH amine column, eluting with DCM/MeOH, O-12% gradient) to give a white solid (58 mg, 61% yield). ¹H NMR (400 MHz, DMSO) δ 7.78 (s, 2H), 7.01 (d, J=2.9 Hz, 1H), 6.81 (d, J=8.9 Hz, 1H), 6.58 (dd, J=8.8, 2.9 Hz, 1H), 3.26 (dd, J=10.5, 5.2 Hz, 1H), 3.23-3.18 (m, 1H), 2.87-2.79 (m, 1H), 2.67 (dd, J=13.3, 4.7 Hz, 1H), 2.37 (dd, J=13.2, 8.2 Hz, 1H); MS (ESI) m/z, [M+H] calculated for C₁₅H₁₅I₃NO₃, 637.82; observed, 638.22.

### 90. Preparation of tert-butyl 4-hydroxyphenethylcarbamate (MLAG-084)

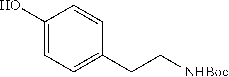 [SMILES: CC(C)(C)OC(=O)NCCc1ccc(O)cc1]

The synthetic scheme for the preparation of the title compound, as well as MLAG-085, MLAG-086, and MLAG-087, beginning with 4-(aminoethyl)phenol is given below.

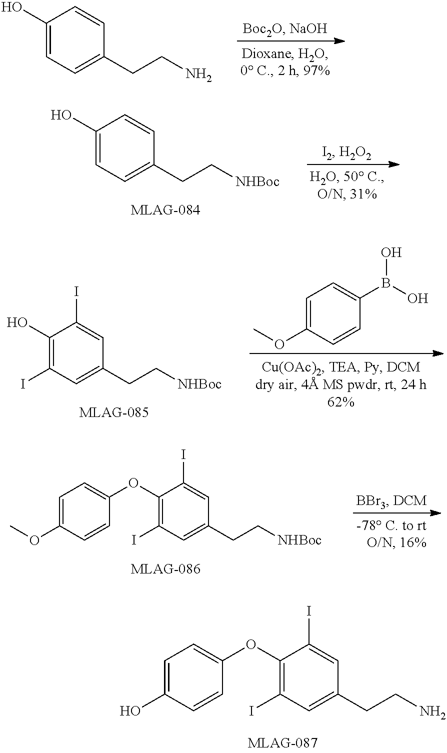

Briefly, the title compound is prepared as follows: A mixture of 4-(2-aminoethyl)phenol (200 mg, 1.458 mmol), in dioxane (3 mL), and water (3 mL), at 0° C., was adjusted to pH˜10 using 1M NaOH. Boc₂O (406 μL, 1.750 mmol) was added and the solution stirred for 2 h. 1M HCl was added to the reaction mixture to pH 3 and extracted with ethyl acetate (3×20 mL). The combined organic layers were washed with saturated brine, dried over Na₂SO₄, filtered, and concentrated. The crude product was purified by flash column chromatography (Biotage SP4, 25+M column, eluting with hexanes/ethyl acetate, 0-50% gradient) to give a colorless oil (337 mg, 97% yield). ¹H NMR (400 MHz, CDCl₃) δ 7.03 (d, J=8.2 Hz, 2H), 6.77 (d, J=8.4 Hz, 2H), 5.57 (s, 1H), 4.57 (bs, 1H), 3.33 (dd, J=12.4, 5.9 Hz, 2H), 2.71 (t, J=7.0 Hz, 2H), 1.44 (s, 9H).

### 91. Preparation of tert-butyl 4-hydroxy-3,5-diiodophenethylcarbamate (MLAG-085)

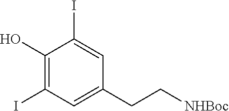 [SMILES: CC(C)(C)OC(=O)NCCc1cc(I)c(O)c(I)c1]

A 30% aqueous solution of hydrogen peroxide (430 μL, 4.17 mmol) was added dropwise to a stirring solution of MLAG-084 (330 mg, 1.391 mmol), and I₂ (529 mg, 2.086 mmol), in water (10 mL). The reaction mixture was heated to 50° C. and stirred overnight. 1M Na₂S₂O₃ (10 mL) was added to the reaction mixture and extracted with ethyl acetate (3×10 mL). The combined organic layers were washed with saturated brine, dried over Na₂SO₄, filtered, and concentrated. The crude product was purified by flash column chromatography (Biotage SP4, 25+M column, eluting with hexanes/ethyl acetate, 0-30% gradient) to give a white solid (212 mg, 31% yield). ¹H NMR (400 MHz, CDCl₃) δ 7.50 (s, 2H), 5.65 (s, 1H), 4.53 (bs, 1H), 3.29 (dd, J=13.4, 6.7 Hz, 2H), 2.67 (t, J=6.8 Hz, 2H), 1.45 (s, 9H).

### 92. Preparation of tert-butyl 3,5-diiodo-4-(4-methoxyphenoxy)phenethylcarbamate (MLAG-086)

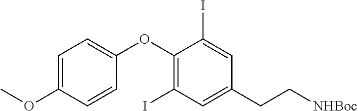 [SMILES: COc1ccc(Oc2c(I)cc(CCNC(=O)OC(C)(C)C)cc2I)cc1]

A mixture of TEA (234 μL, 1.677 mmol) and pyridine (136 μL, 1.677 mmol) was added to a stirring solution of MLAG-085 (205 mg, 0.419 mmol), (4-methoxyphenyl)boronic acid (96 mg, 0.629 mmol), copper (II) acetate (228 mg, 1.257 mmol), and 4 Å molecular sieve powder, in dry DCM (5 mL). A drying tube was attached and the solution stirred overnight. Additional (4-methoxyphenyl)boronic acid (63 mg, 0.415 mmol) and copper (II) acetate (150 mg, 0.830 mmol) were added and the reaction mixture stirred for an additional 4 h. The solution was diluted with ethyl acetate and filtered through celite. The filtrate was concentrated and purified by flash column chromatography (Biotage SP4, 25+S column, eluting with hexanes/ethyl acetate, 0-30% gradient) to give a white solid (155 mg, 62% yield). ¹H NMR (400 MHz, CDCl₃) δ 7.68 (s, 2H), 6.86-6.80 (m, 2H), 6.75-6.68 (m, 2H), 4.60 (bs, 1H), 3.77 (s, 3H), 3.36 (q, J=6.7 Hz, 2H), 2.75 (t, J=7.0 Hz, 2H), 1.46 (s, 9H).

### 93. Preparation of 4-(4-(2-aminoethyl)-2,6-diiodophenoxy)phenol (MLAG-087)

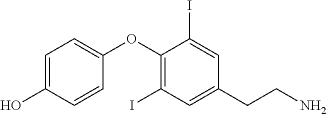 [SMILES: NCCc1cc(I)c(Oc2ccc(O)cc2)c(I)c1]

A 1M in DCM solution of BBr₃ (1.25 mL, 1.25 mmol) was added dropwise to a solution of MLAG-086 (149 mg, 0.250 mmol), in DCM (3 mL), at −78° C. The reaction mixture was stirred overnight under N₂ and allowed to reach rt. 1M NaOH (10 mL) was added to the reaction mixture and extracted with ethyl acetate (3×10 mL). The combined organic layers were washed with saturated brine, dried over Na₂SO₄, filtered, and concentrated. The crude product was purified by flash column chromatography (Biotage SP4, 25+S KP-NH amine column, eluting with DCM/MeOH, 0-10% gradient) to give the desired product (18.8 mg, 16% yield). ¹H NMR (400 MHz, DMSO) δ 9.09 (bs, 1H), 7.76 (s, 2H), 6.68 (d, J=8.9 Hz, 2H), 6.53 (d, J=8.9 Hz, 2H), 2.77 (t, J=6.8 Hz, 2H), 2.59 (t, J=6.9 Hz, 2H), 1.44 (bs, 2H); MS (ESI) m/z, [M+H] calculated for C₁₄H₁₄I₂NO₂, 481.91; observed, 482.40.

### 94. Preparation of 2-(4-(4-Hydroxy-3-Iodophenoxy)-3,5-diiodophenyl)acetamide (MLAG-088)

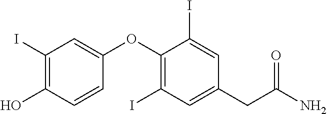 [SMILES: NC(=O)Cc1cc(I)c(Oc2ccc(O)c(I)c2)c(I)c1]

The synthetic scheme for the preparation of the title compound from 2-(4-(4-hydroxy-3-iodophenoxy)-3,5-diiodophenyl)acetic acid is given below.

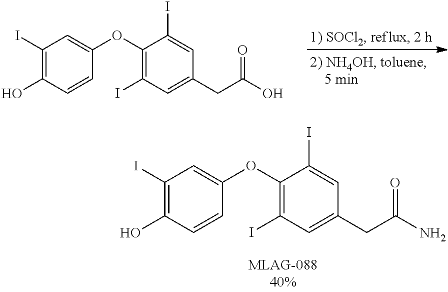 [SMILES: O=C(O)Cc1cc(I)c(Oc2ccc(O)c(I)c2)c(I)c1 ; NC(=O)Cc1cc(I)c(Oc2ccc(O)c(I)c2)c(I)c1]

Briefly, the title compound is prepared as follows: A solution of 2-(4-(4-hydroxy-3-iodophenoxy)-3,5-diiodophenyl)acetic acid (20 mg, 0.032 mmol) in SOCl₂ (500 μL, 6.85 mmol) was heated to reflux and stirred for 2 h. The reaction mixture was concentrated and dried under high vacuum. Toluene (100 μL) and aqueous NH₄OH (300 μL) were added and the solution stirred for 5 min. Water (5 mL) was added and extracted with ethyl acetate (3×5 mL). The combined organic layers were washed with saturated brine, dried over Na₂SO₄, filtered, and concentrated. The crude product was purified by flash column chromatography (Biotage SP4, 12+S column, eluting with DCM/MeOH, 0-10% gradient) to give a while solid (8 mg, 40% yield). ¹H NMR (400 MHz, DMSO) δ 10.00 (bs, 1H), 7.81 (s, 2H), 7.52 (s, 1H), 7.00 (d, J=2.9 Hz, 2H), 6.82 (d, J=8.8 Hz, 1H), 6.59 (dd, J=8.8, 2.9 Hz, 1H), 3.38 (s, 2H); MS (ESI) m/z, [M+H] calculated for C₁₄H₁₁I₃NO₃, 621.79; observed, 622.16.

### 95. Preparation of (S)-methyl 2-((tert-butoxycarbonyl)amino)-3-(4-hydroxy-3,5-diiodophenyl)propanoate (MLAG-089)

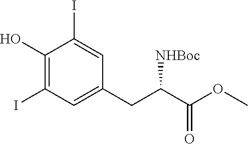 [SMILES: COC(=O)[C@H](Cc1cc(I)c(O)c(I)c1)NC(=O)OC(C)(C)C]

The synthetic scheme for the preparation of the title compound, as well as MLAG-090, MLAG-091, MLAG-092, and MLAG-093, beginning with (S)-2-amino-3-(4-hydroxy-3,5-diiodophenyl)propanoic acid is given below.

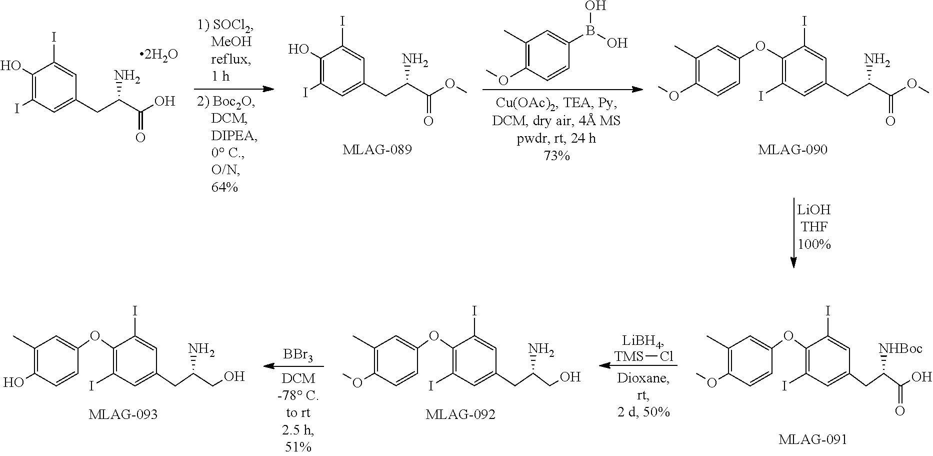

Briefly, the title compound is prepared as follows: SOCl₂ (233 μL, 3.20 mmol) was added dropwise to a solution of (S)-2-amino-3-(4-hydroxy-3,5-diiodophenyl)propanoic acid dihydrate (500 mg, 1.066 mmol), in MeOH (8 mL) at 0° C. The solution was heated to reflux and stirred for 2 h. The reaction mixture was concentrated and dried under high vacuum. DCM (8 mL) and DIPEA (242 μL, 1.386 mmol) were added to the dried solid and stirred at 0° C. Boc₂O (297 μL, 1.279 mmol) was added to the reaction mixture and stirred overnight, allowing the mixture to reach rt. The heterogeneous solution was concentrated and purified by flash column chromatography (Biotage SP4, 40+S column, eluting with hexanes/ethyl acetate, 0-40% yield) to give a white solid (372 mg, 64% yield). ¹H NMR (400 MHz, CDCl₃) δ 7.44 (s, 2H), 5.68 (s, 1H), 5.01 (d, J=6.9 Hz, 1H), 4.49 (dd, J=12.7, 6.2 Hz, 1H), 3.74 (d, J=1.1 Hz, 3H), 3.02 (dd, J=13.9, 5.7 Hz, 1H), 2.90 (dd, J=13.5, 5.3 Hz, 1H), 1.45 (s, 8H).

### 96. Preparation of (S)-methyl 2-((tert-butoxycarbonyl)amino)-3-(3,5-diiodo-4-(4-methoxy-3-methylphenoxy)phenyl)propanoate (MLAG-090)

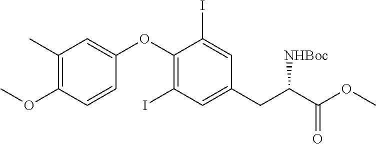

A mixture of TEA (204 μL, 1.462 mmol) and Py (118 μL, 1.462 mmol) were added to a stirring solution of MLAG-089 (200 mg, 0.366 mmol), (4-methoxy-3-methylphenyl)boronic acid (91 mg, 0.548 mmol), copper (II) acetate (199 mg, 1.097 mmol), and 4 Å molecular sieve powder (400 mg), in dry DCM (4 mL). A drying tube was attached and the solution stirred overnight. Additional (4-methoxy-3-methylphenyl)boronic acid (91 mg, 0.548 mmol), copper (II) acetate (199 mg, 1.097 mmol) were added to the reaction mixture and the solution stirred for an additional 5 h. The solution was diluted with ethyl acetate and filtered through celite. The filtrate was concentrated and purified by flash column chromatography (Biotage SP4, 25+M column, eluting with hexanes/ethyl acetate, 0-40% gradient) to give the desired product (179 mg, 73% yield). ¹H NMR (400 MHz, CDCl₃) δ 7.63 (s, 2H), 6.73-6.62 (m, 2H), 6.44 (dd, J=8.8, 3.0 Hz, 1H), 5.09 (d, J=7.8 Hz, 1H), 4.55 (dd, J=13.0, 6.5 Hz, 1H), 3.78 (s, 3H), 3.76 (s, 3H), 3.10 (dd, J=13.7, 5.6 Hz, 1H), 2.93 (dd, J=13.9, 6.3 Hz, 1H), 2.19 (s, 3H), 1.45 (s, 9H).

### 97. Preparation of (S)-2-((tert-butoxycarbonyl)amino)-3-(3,5-diiodo-4-(4-methoxy-3-methylphenoxy)phenyl)propanoic acid (MLAG-091)

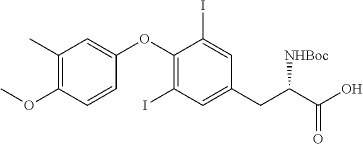 [SMILES: COc1ccc(Oc2c(I)cc(C[C@H](NC(=O)OC(C)(C)C)C(=O)O)cc2I)cc1C]

1M LiOH (1 mL, 1 mmol) was added to a solution of MLAG-090 (174 mg, 0.261 mmol) in THF (1 mL), and stirred for 2 h. The reaction mixture was acidified with 1M oxalic acid and extracted with ethyl acetate (3×5 mL). The combined organic layers were washed with saturated brine, dried over Na₂SO₄, filtered, and concentrated to give a pale brown solid (170 mg), which was used in the next step without further purification.

### 98. Preparation of (S)-2-amino-3-(3,5-diiodo-4-(4-methoxy-3-methylphenoxy)phenyl)propan-1-ol (MLAG-092)

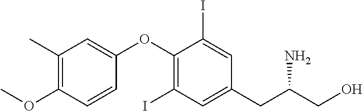 [SMILES: COc1ccc(Oc2c(I)cc(C[C@H](N)CO)cc2I)cc1C]

TMS-Cl (100 μL, 0.781 mmol) was added dropwise to a stirring solution of 2M in THF LiBH₄ (260 μL, 0.520 mmol) in dioxane (2 mL), under N₂. After 30 min the reaction mixture was cooled to 0° C. and MLAG-091 (170 mg, 0.260 mmol) was added. The reaction mixture was vigorously stirred overnight and allowed to reach rt. Additional TMS-Cl (100 μL, 0.781 mmol) and LiBH₄ (260 μL, 0.520 mmol) were added and the solution and stirred for an additional 24 h. The reaction mixture was added to 1M NaOH (10 mL) and extracted with ethyl acetate (3×10 mL). The combined organic layers were washed with saturated brine, dried over Na₂SO₄, filtered, and concentrated. The crude product was purified by flash column chromatography (Biotage SP4, 25+S column, eluting with DCM/MeOH, 0-14% gradient) to give a colorless oil (70 mg, 50% yield). ¹H NMR (400 MHz, DMSO) δ 7.79 (s, 2H), 6.82 (d, J=9.0 Hz, 1H), 6.66 (d, J=3.0 Hz, 1H), 6.37 (dd, J=8.9, 3.0 Hz, 1H), 4.75 (bs, 1H), 3.73 (s, 3H), 3.27-3.20 (m, 2H), 2.97-2.89 (m, 1H), 2.70 (dd, J=13.5, 5.3 Hz, 1H), 2.44 (dd, J=13.4, 8.0 Hz, 1H), 2.11 (s, 3H).

### 99. Preparation of (S)-4-(4-(2-amino-3-hydroxypropyl)-2,6-diiodophenoxy)-2-methylphenol (MLAG-093)

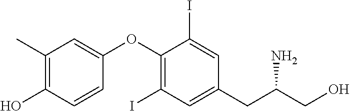 [SMILES: Cc1cc(Oc2c(I)cc(C[C@H](N)CO)cc2I)ccc1O]

A 1M in DCM solution of BBr₃ (230 μL, 0.230 mmol) was added dropwise to a solution of MLAG-092 (62 mg, 0.115 mmol), in DCM (1 mL), at −78° C. After 15 min, the cooling bath was removed and the solution stirred for 2 h at rt. 1M HCl (5 mL) was added and stirred for 5 min. The solution was basified with 2M NaOH and extracted with ethyl acetate (3×5 mL). The combined organic layers were washed with saturated brine, dried over Na₂SO₄, filtered, and concentrated. The crude product was purified by flash column chromatography (Biotage SP4, 12+S KP-NH amine column, eluting with DCM/MeOH, 0-8% gradient) to give the desired product (31 mg, 51% yield). ¹H NMR (400 MHz, DMSO) δ 8.97 (bs, 1H), 7.76 (s, 2H), 6.66 (d, J=8.7 Hz, 1H), 6.52 (s, 1H), 6.26 (dd, J=8.7, 2.9 Hz, 1H), 4.62 (bs, 1H), 3.29-3.24 (m, 1H), 3.21 (d, J=6.2 Hz, 1H), 2.83 (dt, J=11.5, 5.7 Hz, 1H), 2.66 (dd, J=13.4, 4.8 Hz, 1H), 2.36 (dd, J=13.3, 8.2 Hz, 1H), 2.07 (s, 3H), 1.59 (bs, 2H); MS (ESI) m/z, [M+H] calculated for C₁₆H₁₈I₂NO₃, 525.94; observed, 526.49.

### 100. Compound Summary

The compounds described above are summarized below in Table 1a, which provides the compound ID, structure, and [M+H] information, and in Table 1b, which provides a summary of the ¹H NMR data.

TABLE 1a

| | | | [M + H]* | [M + H]* |
|---|---|---|---|---|
| No. | Compound ID | Structure | Calc | Exptl |
| 1 | MLAF-007 | 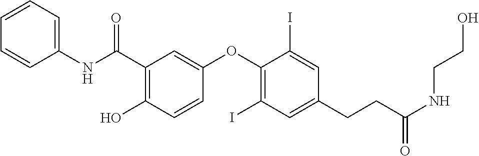 [SMILES: O=C(CCc1cc(I)c(Oc2ccc(O)c(C(=O)Nc3ccccc3)c2)c(I)c1)NCCO] | 672.9696 | 672.9711 |
| 2 | MLAF-008 | 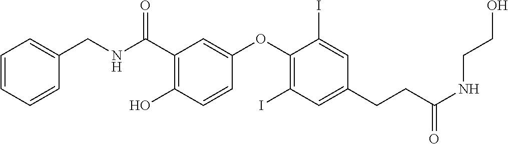 [SMILES: O=C(CCc1cc(I)c(Oc2ccc(O)c(C(=O)NCc3ccccc3)c2)c(I)c1)NCCO] | 686.9853 | 686.9857 |
| 3 | MLAE-062 | 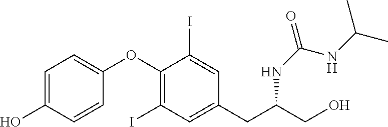 [SMILES: CC(C)NC(=O)N[C@H](CO)Cc1cc(I)c(Oc2ccc(O)cc2)c(I)c1] | 596.9747 | 596.975 |
| 4 | MLAF-013 | 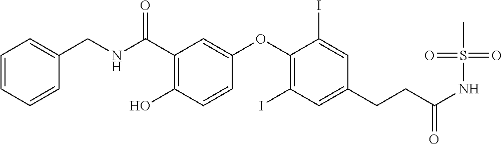 [SMILES: CS(=O)(=O)NC(=O)CCc1cc(I)c(Oc2ccc(O)c(C(=O)NCc3ccccc3)c2)c(I)c1] | 720.9366 | 720.9367 |
| 5 | MLAE-030 | 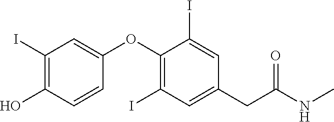 [SMILES: CNC(=O)Cc1cc(I)c(Oc2ccc(O)c(I)c2)c(I)c1] | 635.8029 | 635.8026 |
| 6 | MLAE-045 | 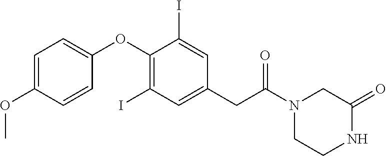 [SMILES: COc1ccc(Oc2c(I)cc(CC(=O)N3CCNC(=O)C3)cc2I)cc1] | 592.9434 | 592.9420 |
| 7 | MLAE-041 | 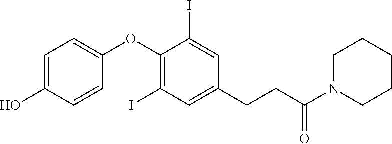 [SMILES: O=C(CCc1cc(I)c(Oc2ccc(O)cc2)c(I)c1)N1CCCCC1] | 577.9689 | 577.9675 |
| 8 | MLAE-037 | 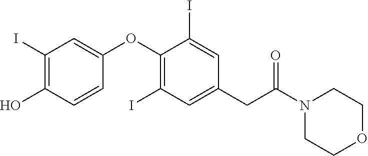 [SMILES: O=C(Cc1cc(I)c(Oc2ccc(O)c(I)c2)c(I)c1)N1CCOCC1] | 691.8292 | 691.8292 |
| 9 | MLAE-051-3 | 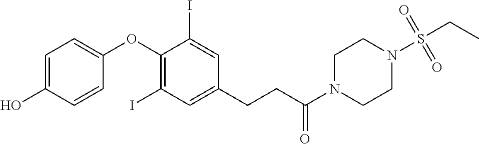 [SMILES: CCS(=O)(=O)N1CCN(C(=O)CCc2cc(I)c(Oc3ccc(O)cc3)c(I)c2)CC1] | 670.9574 | 670.9571 |
| 10 | MLAE-051-2 | 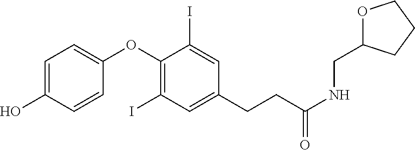 [SMILES: O=C(CCc1cc(I)c(Oc2ccc(O)cc2)c(I)c1)NCC1CCCO1] | 593.9638 | 593.9641 |
| 11 | MLAE-063 | 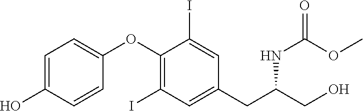 [SMILES: COC(=O)N[C@H](CO)Cc1cc(I)c(Oc2ccc(O)cc2)c(I)c1] | 569.9274 | 569.9281 |
| 12 | MLAE-031 | 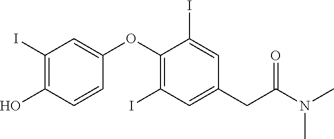 [SMILES: CN(C)C(=O)Cc1cc(I)c(Oc2ccc(O)c(I)c2)c(I)c1] | 649.8186 | 649.8173 |
| 13 | MLAE-059 | 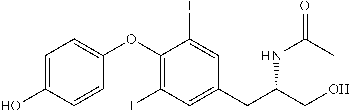 [SMILES: CC(=O)N[C@H](CO)Cc1cc(I)c(Oc2ccc(O)cc2)c(I)c1] | 553.9325 | 553.9333 |
| 14 | MLAE-051-1 | 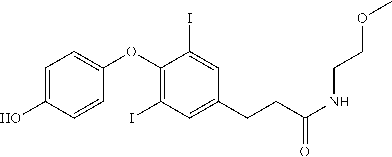 [SMILES: COCCNC(=O)CCc1cc(I)c(Oc2ccc(O)cc2)c(I)c1] | 567.9482 | 567.9489 |
| 15 | MLAE-054-4 | 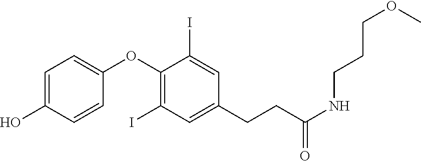 [SMILES: COCCCNC(=O)CCc1cc(I)c(Oc2ccc(O)cc2)c(I)c1] | 581.9638 | 581.9623 |
| 16 | MLAE-054-1 | 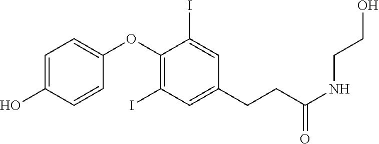 [SMILES: O=C(CCc1cc(I)c(Oc2ccc(O)cc2)c(I)c1)NCCO] | 553.9325 | 553.9322 |
| 17 | MLAE-058-1 | 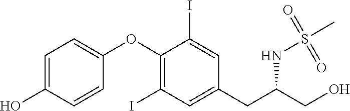 [SMILES: CS(=O)(=O)N[C@H](CO)Cc1cc(I)c(Oc2ccc(O)cc2)c(I)c1] | 589.8995 | 589.8994 |
| 18 | MLAE-058-2 | 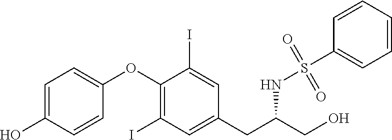 [SMILES: O=S(=O)(N[C@H](CO)Cc1cc(I)c(Oc2ccc(O)cc2)c(I)c1)c1ccccc1] | 651.9152 | 651.9167 |
| 20 | MLAE-047 | 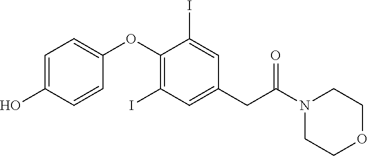 [SMILES: O=C(Cc1cc(I)c(Oc2ccc(O)cc2)c(I)c1)N1CCOCC1] | 565.9325 | 565.9327 |
| 21 | MLAE-054-2 | 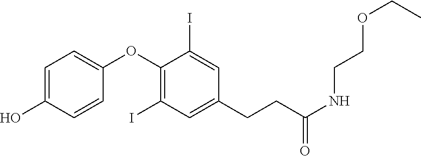 [SMILES: CCOCCNC(=O)CCc1cc(I)c(Oc2ccc(O)cc2)c(I)c1] | 581.9638 | 581.9640 |
| 22 | MLAE-054-3 | 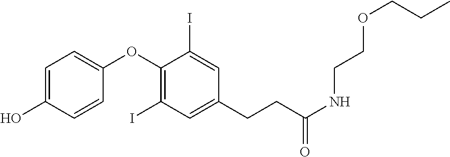 [SMILES: CCCOCCNC(=O)CCc1cc(I)c(Oc2ccc(O)cc2)c(I)c1] | 595.9795 | 595.9788 |
| 23 | MLAE-033 | 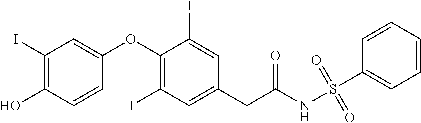 [SMILES: O=C(Cc1cc(I)c(Oc2ccc(O)c(I)c2)c(I)c1)NS(=O)(=O)c1ccccc1] | 761.7805 | 761.7809 |
| 24 | MLAE-048 | 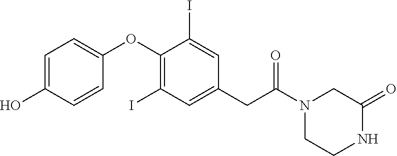 [SMILES: O=C1CN(C(=O)Cc2cc(I)c(Oc3ccc(O)cc3)c(I)c2)CCN1] | 578.9278 | 578.9272 |
| 25 | MLAE-049 | 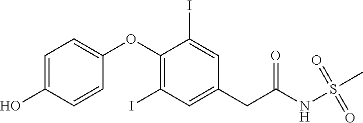 [SMILES: CS(=O)(=O)NC(=O)Cc1cc(I)c(Oc2ccc(O)cc2)c(I)c1] | 573.8682 | 573.8688 |
| 26 | MLAE-039 | 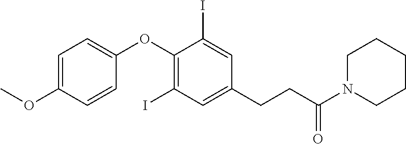 [SMILES: COc1ccc(Oc2c(I)cc(CCC(=O)N3CCCCC3)cc2I)cc1] | 591.9846 | 591.9837 |
| 27 | MLAF-051 | 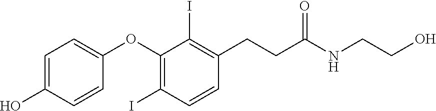 [SMILES: O=C(CCc1ccc(I)c(Oc2ccc(O)cc2)c1I)NCCO] | 553.9325 | 553.9325 |
| 28 | MLAE-035 | 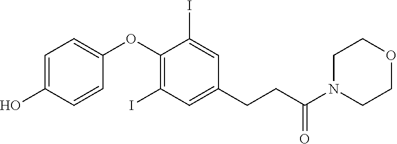 [SMILES: O=C(CCc1cc(I)c(Oc2ccc(O)cc2)c(I)c1)N1CCOCC1] | 579.9482 | 579.9477 |
| 29 | MLAE-058-3 | 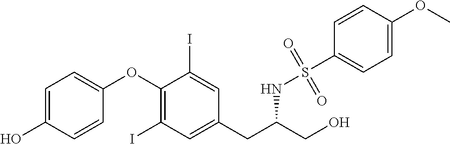 [SMILES: COc1ccc(S(=O)(=O)N[C@H](CO)Cc2cc(I)c(Oc3ccc(O)cc3)c(I)c2)cc1] | 681.9257 | 681.925 |
| 30 | MLAE-042 | 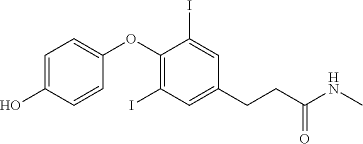 [SMILES: CNC(=O)CCc1cc(I)c(Oc2ccc(O)cc2)c(I)c1] | 523.922 | 523.9211 |
| 31 | MLAE-032 | 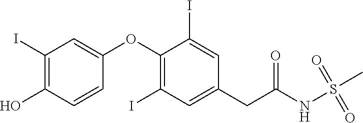 [SMILES: CS(=O)(=O)NC(=O)Cc1cc(I)c(Oc2ccc(O)c(I)c2)c(I)c1] | 699.7648 | 699.7664 |
| 32 | MLAE-061 | 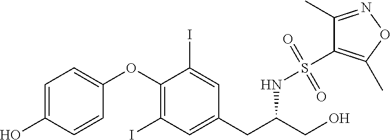 [SMILES: Cc1noc(C)c1S(=O)(=O)N[C@H](CO)Cc1cc(I)c(Oc2ccc(O)cc2)c(I)c1] | 670.921 | 670.9192 |
| 33 | MLAE-040 | 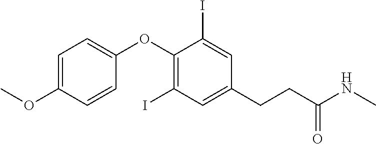 | 537.9376 | 537.9360 |
| 34 | MLAF-050 | 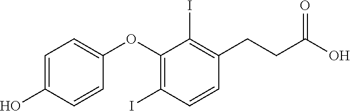 [SMILES: O=C(O)CCc1ccc(I)c(Oc2ccc(O)cc2)c1I] | 510.89* | 510.78* |
| 35 | MLAE-038 | 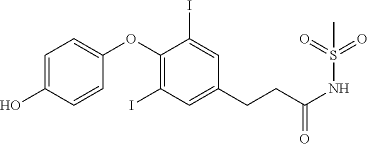 [SMILES: CS(=O)(=O)NC(=O)CCc1cc(I)c(Oc2ccc(O)cc2)c(I)c1] | 587.8839 | 587.8830 |
| 36 | MLAE-060 | 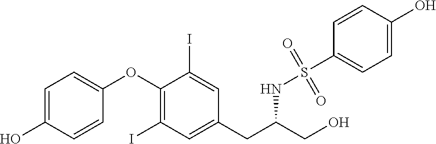 [SMILES: O=S(=O)(N[C@H](CO)Cc1cc(I)c(Oc2ccc(O)cc2)c(I)c1)c1ccc(O)cc1] | 667.91* | 667.78* |
| 37 | MLAE-029-1 | 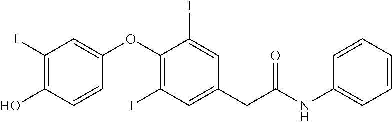 [SMILES: O=C(Cc1cc(I)c(Oc2ccc(O)c(I)c2)c(I)c1)Nc1ccccc1] | 697.8186 | 697.8199 |
| 38 | MLAE-029-2 | 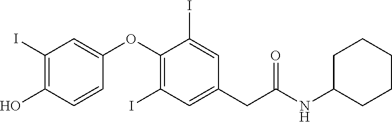 [SMILES: O=C(Cc1cc(I)c(Oc2ccc(O)c(I)c2)c(I)c1)NC1CCCCC1] | 703.8655 | 703.8671 |
| 39 | MLAE-029-3 | 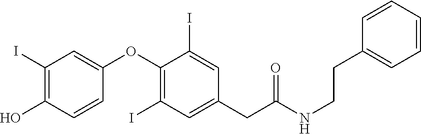 [SMILES: O=C(Cc1cc(I)c(Oc2ccc(O)c(I)c2)c(I)c1)NCCc1ccccc1] | 725.8499 | 725.8491 |
| 40 | MLAE-029-4 | 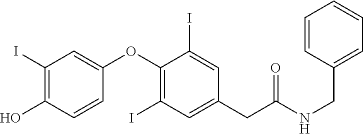 [SMILES: O=C(Cc1cc(I)c(Oc2ccc(O)c(I)c2)c(I)c1)NCc1ccccc1] | 711.8342 | 711.8357 |
| 41 | MLAE-029-6 | 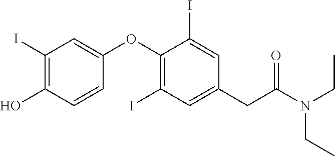 [SMILES: CCN(CC)C(=O)Cc1cc(I)c(Oc2ccc(O)c(I)c2)c(I)c1] | 677.8499 | 677.8494 |
| 42 | MLAE-029-7 | 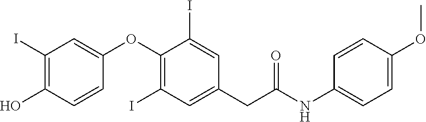 [SMILES: COc1ccc(NC(=O)Cc2cc(I)c(Oc3ccc(O)c(I)c3)c(I)c2)cc1] | 727.8292 | 727.8312 |
| 43 | MLAE-029-8 | 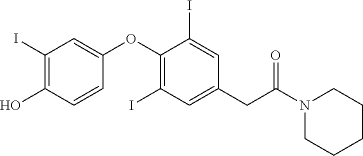 [SMILES: O=C(Cc1cc(I)c(Oc2ccc(O)c(I)c2)c(I)c1)N1CCCCC1] | 689.8499 | 689.8495 |
| 44 | MLAE-029-9 | 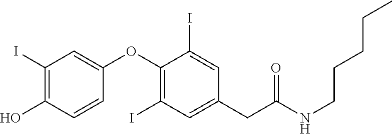 [SMILES: CCCCCNC(=O)Cc1cc(I)c(Oc2ccc(O)c(I)c2)c(I)c1] | 691.8655 | 691.8654 |
| 45 | MLAE-034 | 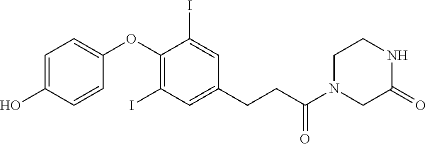 [SMILES: O=C1CN(C(=O)CCc2cc(I)c(Oc3ccc(O)cc3)c(I)c2)CCN1] | 592.9434 | 592.9433 |
| 46 | MLAE-036 | 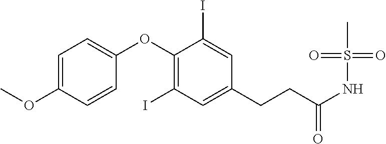 [SMILES: COc1ccc(Oc2c(I)cc(CCC(=O)NS(C)(=O)=O)cc2I)cc1] | 601.8995 | 601.9005 |
| 47 | MLAE-046 | 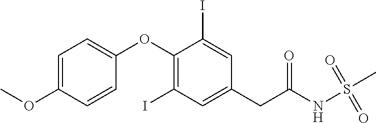 | 587.8839 | 587.8825 |
| 48 | MLAF-012 | 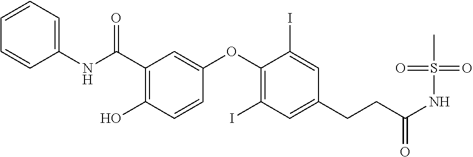 [SMILES: CS(=O)(=O)NC(=O)CCc1cc(I)c(Oc2ccc(O)c(C(=O)Nc3ccccc3)c2)c(I)c1] | 706.921 | 706.9208 |
| 49 | (S)-T2AA | 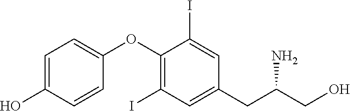 [SMILES: N[C@H](CO)Cc1cc(I)c(Oc2ccc(O)cc2)c(I)c1] | 511.92 | — |
| 50 | (R)-T2AA | 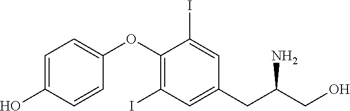 [SMILES: N[C@@H](CO)Cc1cc(I)c(Oc2ccc(O)cc2)c(I)c1] | 511.92 | — |
| 51 | T3 | 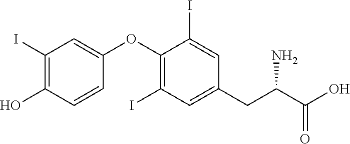 [SMILES: N[C@@H](Cc1cc(I)c(Oc2ccc(O)c(I)c2)c(I)c1)C(=O)O] | 651.8 | — |
| 52 | NF021111 | 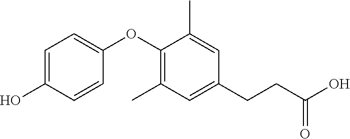 [SMILES: Cc1cc(CCC(=O)O)cc(C)c1Oc1ccc(O)cc1] | 287.13 | — |
| 53 | SWUA_017 | 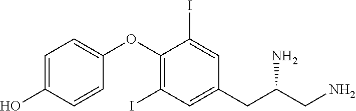 [SMILES: NC[C@@H](N)Cc1cc(I)c(Oc2ccc(O)cc2)c(I)c1] | 552.9485 | 552.9481 |
| 54 | SWUA_018 | 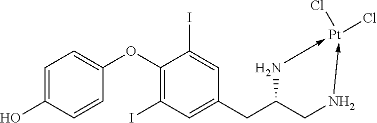 | 588.9155 | 588.9152 |
| 55 | MLAF-065 | 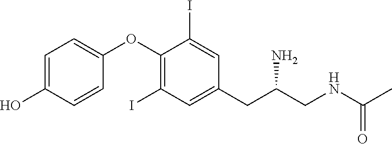 [SMILES: CC(=O)NC[C@@H](N)Cc1cc(I)c(Oc2ccc(O)cc2)c(I)c1] | 595.9907 | 595.9894 |
| 56 | MLAF-066 | 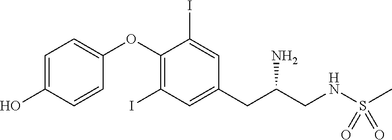 [SMILES: CS(=O)(=O)NC[C@@H](N)Cc1cc(I)c(Oc2ccc(O)cc2)c(I)c1] | 623.9856 | 623.9855 |
| 57 | MLAF-074 | 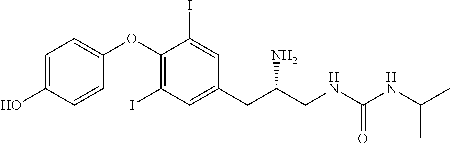 [SMILES: CC(C)NC(=O)NC[C@@H](N)Cc1cc(I)c(Oc2ccc(O)cc2)c(I)c1] | 652.0169 | 652.0154 |
| 58 | MLAF-075 | 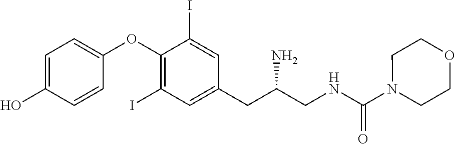 [SMILES: N[C@H](CNC(=O)N1CCOCC1)Cc1cc(I)c(Oc2ccc(O)cc2)c(I)c1] | 568.9434 | 568.9435 |
| 59 | MLAF-081 | 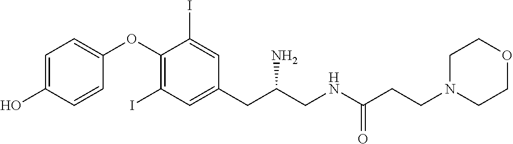 [SMILES: N[C@H](CNC(=O)CCN1CCOCC1)Cc1cc(I)c(Oc2ccc(O)cc2)c(I)c1] | 552.9485 | 552.9481 |
| 60 | MLAF-083 | 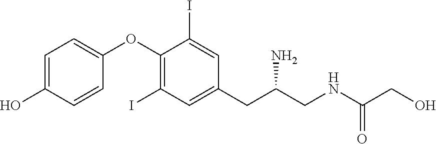 [SMILES: N[C@H](CNC(=O)CO)Cc1cc(I)c(Oc2ccc(O)cc2)c(I)c1] | 588.9155 | 588.9152 |
| 61 | MLAF-085 | 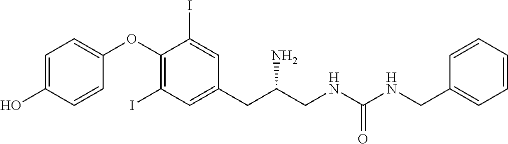 [SMILES: N[C@H](CNC(=O)NCc1ccccc1)Cc1cc(I)c(Oc2ccc(O)cc2)c(I)c1] | 643.99* | 644.51* |
| 62 | MLAF-086 | 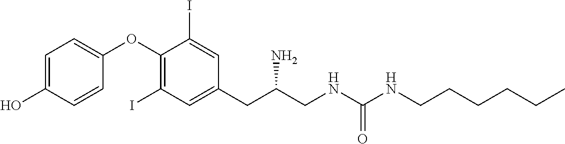 [SMILES: CCCCCCNC(=O)NC[C@@H](N)Cc1cc(I)c(Oc2ccc(O)cc2)c(I)c1] | 638.04* | 638.53* |
| 63 | MLAG-060 | 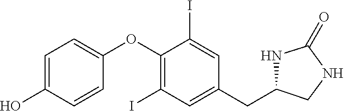 [SMILES: O=C1NC[C@H](Cc2cc(I)c(Oc3ccc(O)cc3)c(I)c2)N1] | 536.92* | 537.32* |
| 64 | MLAG-066-1 | 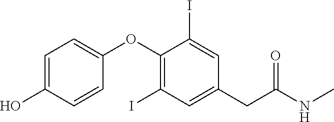 [SMILES: CNC(=O)Cc1cc(I)c(Oc2ccc(O)cc2)c(I)c1] | 509.91* | 510.36* |
| 65 | MLAG-066-2 | 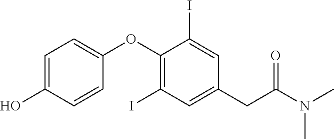 [SMILES: CN(C)C(=O)Cc1cc(I)c(Oc2ccc(O)cc2)c(I)c1] | 523.92* | 524.41* |
| 66 | MLAG-066-3 | 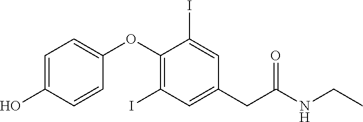 [SMILES: CCNC(=O)Cc1cc(I)c(Oc2ccc(O)cc2)c(I)c1] | 523.92* | 524.28* |
| 67 | MLAG-082 | 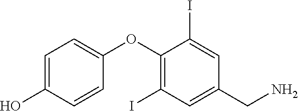 [SMILES: NCc1cc(I)c(Oc2ccc(O)cc2)c(I)c1] | 467.90* | 468.36* |
| 68 | MLAG-083 | 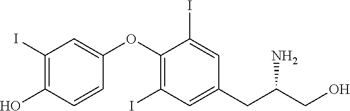 [SMILES: N[C@H](CO)Cc1cc(I)c(Oc2ccc(O)c(I)c2)c(I)c1] | 637.82* | 638.22* |
| 69 | MLAG-087 | 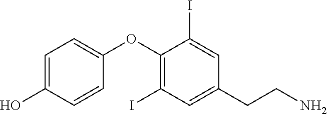 [SMILES: NCCc1cc(I)c(Oc2ccc(O)cc2)c(I)c1] | 481.91* | 482.40* |
| 70 | MLAG-088 | 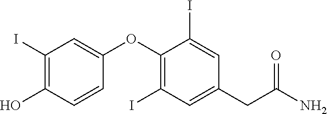 [SMILES: NC(=O)Cc1cc(I)c(Oc2ccc(O)c(I)c2)c(I)c1] | 621.79* | 622.16* |
| 71 | MLAG-093 | 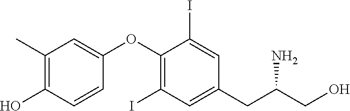 [SMILES: Cc1cc(Oc2c(I)cc(C[C@H](N)CO)cc2I)ccc1O] | 525.94* | 526.49* |

*Data were obtained from high resolution mass spectrometry unless indicated by “*,” which indicates that data were obtained from low resolution mass spectrometry.

TABLE 1b

| No. | Compound ID | ¹H NMR* |
|---|---|---|
| 1 | MLAF-007 | ¹H NMR (400 MHz, DMSO) δ 11.25 (s, 1H), 10.27 (s, 1H), |
| | | 7.66 (t, J = 5.3 Hz, 1H), 7.59 (s, 2H), 7.48 (d, J = 8.2 Hz, 2H), |
| | | 7.24-7.11 (m, 3H), 6.93 (t, J = 7.4 Hz, 1H), 6.76 (d, J = 8.9 Hz, |
| | | 1H), 6.58 (dd, J = 8.2, 2.3 Hz, 1H), 4.45 (t, J = 5.2 Hz, 1H), |
| | | 3.21-3.14 (m, 2H), 2.91 (q, J = 5.9 Hz, 2H), 2.59 (t, J = 7.4 Hz, |
| | | 2H), 2.20 (t, J = 7.5 Hz, 2H). |
| 2 | MLAF-008 | ¹H NMR (400 MHz, DMSO) δ 11.77 (s, 1H), 9.15 (t, J = 5.8 Hz, |
| | | 1H), 7.71 (dd, J = 10.1, 4.4 Hz, 1H), 7.64 (s, 2H), 7.30 (d, J = 2.8 Hz, |
| | | 1H), 7.24-7.15 (m, 4H), 7.16-7.09 (m, 1H), |
| | | 6.74 (d, J = 9.0 Hz, 1H), 6.54 (dd, J = 8.9, 2.9 Hz, 1H), 4.51 (t, J = 5.4 Hz, |
| | | 1H), 4.36 (d, J = 5.4 Hz, 2H), 3.25-3.20 (m, 2H), |
| | | 2.97 (q, J = 6.0 Hz, 2H), 2.64 (t, J = 7.5 Hz, 2H), 2.25 (t, J = 7.5 Hz, |
| | | 2H). |
| 3 | MLAE-062 | ¹H NMR (400 MHz, DMSO) δ 9.09 (s, 1H), 7.96 (s, 1H), |
| | | 7.74 (s, 2H), 6.71-6.63 (m, 2H), 6.56-6.47 (m, 2H), 5.69 (dd, J = 16.9, |
| | | 8.1 Hz, 2H), 4.84 (t, J = 5.2 Hz, 1H), 3.80-3.66 (m, 1H), |
| | | 3.65-3.54 (m, 1H), 3.42-3.35 (m, 1H), 3.32-3.23 (m, 1H), |
| | | 2.90 (s, 3H), 2.84-2.72 (m, 4H), 2.60-2.53 (m, 1H). |
| 4 | MLAF-013 | ¹H NMR (400 MHz, DMSO) δ 11.66 (s, 1H), 11.48 (bs, 1H), |
| | | 9.04 (t, J = 5.8 Hz, 1H), 7.58 (s, 2H), 7.18 (d, J = 2.5 Hz, 1H), |
| | | 7.14-7.06 (m, 4H), 7.05-6.98 (m, 1H), 6.64 (d, J = 9.0 Hz, |
| | | 1H), 6.45 (dd, J = 8.5, 2.3 Hz, 1H), 4.26 (d, J = 5.6 Hz, 2H), |
| | | 2.97 (s, 3H), 2.57 (t, J = 7.4 Hz, 2H), 2.36 (t, J = 7.6 Hz, 2H). |
| 5 | MLAE-030 | ¹H NMR (400 MHz, DMSO) δ 8.00 (d, J = 4.4 Hz, 1H), 7.81 (s, |
| | | 2H), 7.00 (d, J = 3.0 Hz, 1H), 6.82 (d, J = 8.8 Hz, 1H), 6.59 (dd, |
| | | J = 8.8, 3.0 Hz, 1H), 3.40 (s, 2H), 2.60 (d, J = 4.5 Hz, 3H). |
| 6 | MLAE-045 | ¹H NMR (400 MHz, DMSO) δ 7.80 (d, J = 7.7 Hz, 2H), |
| | | 6.97-6.85 (m, 3H), 6.65 (d, J = 7.5 Hz, 2H), 3.79-3.67 (m, 7H), |
| | | 3.66-3.56 (m, 2H), 3.22-3.10 (m, 2H). |
| 7 | MLAE-041 | ¹H NMR (400 MHz, DMSO) δ 8.91 (bs, 1H), 7.63 (s, 2H), |
| | | 6.50 (d, J = 7.5 Hz, 2H), 6.34 (d, J = 7.5 Hz, 2H), 3.29-3.14 (m, |
| | | 4H), 2.60 (t, J = 6.8 Hz, 2H), 2.45 (t, J = 7.3 Hz, 2H), |
| | | 1.43-1.33 (m, 2H), 1.29-1.17 (m, 4H). |
| 8 | MLAE-037 | ¹H NMR (400 MHz, DMSO) δ 10.00 (s, 1H), 7.79 (s, 2H), |
| | | 6.99 (d, J = 3.0 Hz, 1H), 6.83 (d, J = 8.9 Hz, 1H), 6.60 (dd, J = 8.8, |
| | | 2.9 Hz, 1H), 3.74 (s, 2H), 3.63-3.39 (m, 8H). |
| 9 | MLAE-051-3 | ¹H NMR (400 MHz, DMSO) δ 9.10 (s, 1H), 7.83 (s, 2H), |
| | | 6.75-6.64 (m, 2H), 6.55-6.47 (m, 2H), 3.54 (bs, 4H), 3.13 (bs, 4H), |
| | | 3.06 (q, J = 7.4 Hz, 2H), 2.79 (t, J = 6.9 Hz, 2H), |
| | | 2.72-2.65 (m, 2H), 1.22 (t, J = 7.4 Hz, 3H). |
| 10 | MLAE-051-2 | ¹H NMR (400 MHz, DMSO) δ 9.09 (s, 1H), 7.92 (s, 1H), |
| | | 7.76 (s, 2H), 6.78-6.60 (m, 2H), 6.56-6.47 (m, 2H), |
| | | 3.80-3.67 (m, 2H), 3.60 (dd, J = 13.3, 6.0 Hz, 1H), 3.31 (s, 1H), 3.11 (t, J = 4.7 Hz, |
| | | 2H), 2.79 (t, J = 6.8 Hz, 2H), 2.41 (t, J = 7.0 Hz, 2H), |
| | | 1.84-1.70 (m, 3H). |
| 11 | MLAE-063 | ¹H NMR (400 MHz, DMSO) δ 9.09 (s, 1H), 7.76 (s, 2H), |
| | | 7.02 (d, J = 8.7 Hz, 1H), 6.68 (dd, J = 8.9, 2.2 Hz, 2H), 6.52 (dd, J = 8.9, |
| | | 2.3 Hz, 2H), 4.80 (t, J = 4.4 Hz, 1H), 3.66-3.54 (m, 1H), |
| | | 3.47 (s, 3H), 3.43-3.36 (m, 1H), 2.83 (dd, J = 13.6, 3.6 Hz, |
| | | 1H). |
| 12 | MLAE-031 | ¹H NMR (400 MHz, DMSO) δ 7.78 (s, 2H), 7.00 (d, J = 3.0 Hz, |
| | | 1H), 6.83 (d, J = 8.8 Hz, 1H), 6.60 (dd, J = 8.8, 3.0 Hz, 1H), |
| | | 3.71 (s, 2H), 3.04 (s, 3H), 2.85 (s, 3H). |
| 13 | MLAE-059 | ¹H NMR (400 MHz, DMSO) δ 9.09 (s, 1H), 7.80-7.70 (m, |
| | | 3H), 6.73-6.65 (m, 2H), 6.57-6.45 (m, 2H), 4.81 (t, J = 4.8 Hz, |
| | | 1H), 3.92-3.80 (m, 1H), 3.42-3.33 (m, 3H), 2.81 (dd, J = 13.4, |
| | | 4.9 Hz, 1H), 1.77 (s, 3H). |
| 14 | MLAE-051-1 | ¹H NMR (400 MHz, DMSO) δ 9.09 (s, 1H), 7.93 (t, J = 5.4 Hz, |
| | | 1H), 7.76 (s, 2H), 6.72-6.65 (m, 2H), 6.55-6.44 (m, 2H), |
| | | 3.32-3.26 (m, 2H), 3.26-3.15 (m, 5H), 2.78 (t, J = 7.2 Hz, 2H), |
| | | 2.39 (t, J = 7.2 Hz, 2H). |
| 15 | MLAE-054-4 | ¹H NMR (400 MHz, DMSO) δ 9.09 (s, 1H), 7.83 (t, J = 5.4 Hz, |
| | | 1H), 7.75 (s, 2H), 6.73-6.63 (m, 2H), 6.58-6.46 (m, 2H), |
| | | 3.29-3.22 (m, 2H), 3.21 (s, 3H), 3.12-3.02 (m, 2H), 2.78 (t, J = 7.2 Hz, |
| | | 2H), 2.37 (t, J = 7.3 Hz, 2H), 1.64-1.50 (m, 2H). |
| 16 | MLAE-054-1 | ¹H NMR (400 MHz, DMSO) δ 9.09 (s, 1H), 7.86 (s, 1H), |
| | | 7.76 (s, 2H), 6.71-6.64 (m, 2H), 6.58-6.47 (m, 2H), |
| | | 4.69-4.60 (m, 1H), 3.42-3.26 (m, 2H), 3.11 (dd, J = 11.9, 6.0 Hz, 2H), |
| | | 2.78 (t, J = 7.3 Hz, 2H), 2.39 (t, J = 7.4 Hz, 2H). |
| 17 | MLAE-058-1 | ¹H NMR (400 MHz, DMSO) δ 9.10 (s, 1H), 7.85 (s, 2H), |
| | | 7.16 (d, J = 7.5 Hz, 1H), 6.77-6.64 (m, 2H), 6.59-6.40 (m, 2H), |
| | | 4.91 (t, J = 4.9 Hz, 1H), 3.47-3.36 (m, 2H), 3.18 (dd, J = 5.2, |
| | | 1.7 Hz, 1H), 2.85 (dd, J = 13.4, 3.8 Hz, 1H), 2.61 (s, 3H). |
| 18 | MLAE-058-2 | ¹H NMR (400 MHz, DMSO) δ 9.09 (s, 1H), 7.75 (d, J = 7.3 Hz, |
| | | 1H), 7.65-7.51 (m, 5H), 7.45 (t, J = 7.8 Hz, 2H), 6.68 (d, J = 8.8 Hz, |
| | | 2H), 6.49 (d, J = 8.8 Hz, 2H), 4.87 (t, J = 5.1 Hz, 1H), |
| | | 3.43-3.35 (m, 1H), 3.27-3.14 (m, 2H), 2.81 (dd, J = 13.6, 3.4 Hz, |
| | | 1H), 2.41 (dd, J = 13.7, 8.4 Hz, 1H). |
| 20 | MLAE-047 | ¹H NMR (400 MHz, DMSO) δ 9.11 (s, 1H), 7.78 (s, 2H), |
| | | 6.70 (dd, J = 8.8, 1.7 Hz, 2H), 6.53 (dd, J = 8.7, 1.5 Hz, 2H), 3.73 (s, |
| | | 2H), 3.62-3.42 (m, 8H). |
| 21 | MLAE-054-2 | ¹H NMR (400 MHz, DMSO) δ 9.09 (s, 1H), 7.92 (s, 1H), |
| | | 7.76 (s, 2H), 6.73-6.63 (m, 2H), 6.55-6.49 (m, 2H), |
| | | 3.53-3.36 (m, 4H), 3.22-3.13 (m, 2H), 2.78 (t, J = 7.3 Hz, 2H), 2.39 (t, J = 7.1 Hz, |
| | | 2H), 1.10 (t, J = 7.0 Hz, 3H). |
| 22 | MLAE-054-3 | ¹H NMR (400 MHz, DMSO) δ 9.09 (s, 1H), 7.91 (t, J = 5.5 Hz, |
| | | 1H), 7.75 (s, 2H), 6.69 (d, J = 8.9 Hz, 2H), 6.52 (d, J = 8.9 Hz, |
| | | 2H), 3.48 (t, J = 4.7 Hz, 2H), 3.32-3.25 (m, 2H), 3.19 (q, J = 5.7 Hz, |
| | | 2H), 2.78 (t, J = 7.3 Hz, 2H), 2.39 (t, J = 7.3 Hz, 2H), |
| | | 1.62-1.41 (m, 2H), 0.91-0.79 (m, 3H). |
| 23 | MLAE-033 | ¹H NMR (400 MHz, DMSO) δ 12.41 (s, 1H), 10.01 (s, 1H), |
| | | 7.92-7.82 (m, 2H), 7.72 (s, 2H), 7.68 (t, J = 6.7 Hz, 1H), |
| | | 7.59 (t, J = 7.2 Hz, 2H), 6.98 (d, J = 2.7 Hz, 1H), 6.82 (d, J = 8.9 Hz, |
| | | 1H), 6.56 (dd, J = 8.9, 2.8 Hz, 1H), 3.54 (s, 2H). |
| 24 | MLAE-048 | ¹H NMR (400 MHz, DMSO) δ 9.17 (s, 1H), 7.84 (d, J = 8.5 Hz, |
| | | 2H), 6.86 (dd, J = 23.1, 8.9 Hz, 1H), 6.76 (d, J = 8.8 Hz, 2H), |
| | | 6.60 (d, J = 8.9 Hz, 2H), 4.02 (s, 1H), 3.87-3.72 (m, 4H), |
| | | 3.72-3.65 (m, 1H), 3.35-3.29 (m, 1H), 3.28-3.22 (m, 1H). |
| 25 | MLAE-049 | ¹H NMR (400 MHz, DMSO) δ 11.96 (s, 1H), 9.11 (s, 1H), |
| | | 7.84 (s, 2H), 6.70 (d, J = 8.9 Hz, 2H), 6.53 (d, J = 8.9 Hz, 2H), |
| | | 3.65 (s, 2H), 3.26 (s, 3H). |
| 26 | MLAE-039 | ¹H NMR (400 MHz, DMSO) δ 7.82 (s, 2H), 6.88 (d, J = 9.1 Hz, |
| | | 2H), 6.64 (d, J = 9.1 Hz, 2H), 3.71 (s, 4H), 3.46-3.35 (m, 2H), |
| | | 2.79 (t, J = 7.3 Hz, 2H), 2.64 (t, J = 7.5 Hz, 2H), 1.61-1.51 (m, |
| | | 2H), 1.47-1.35 (m, 4H). |
| 27 | MLAF-051 | ¹H NMR (400 MHz, MeOD) δ 7.71 (d, J = 8.1 Hz, 1H), 6.84 (d, |
| | | J = 8.1 Hz, 1H), 6.59 (d, J = 9.0 Hz, 2H), 6.43 (d, J = 9.0 Hz, |
| | | 2H), 3.47 (t, J = 5.8 Hz, 2H), 3.22-3.16 (m, 2H), 3.01 (t, J = 7.7 Hz, |
| | | 2H), 2.43 (t, J = 7.7 Hz, 2H). |
| 28 | MLAE-035 | ¹H NMR (400 MHz, DMSO) δ 9.09 (s, 1H), 7.82 (s, 2H), |
| | | 6.69 (d, J = 8.8 Hz, 2H), 6.52 (d, J = 8.8 Hz, 2H), 3.57-3.48 (m, |
| | | 4H), 3.48-3.41 (m, 4H), 2.83-2.72 (m, 2H), 2.69-2.61 (m, |
| | | 2H). |
| 29 | MLAE-058-3 | ¹H NMR (400 MHz, DMSO) δ 9.08 (s, 1H), 7.58 (s, 3H), |
| | | 7.52-7.41 (m, 2H), 6.97-6.87 (m, 2H), 6.68 (d, J = 8.9 Hz, 2H), |
| | | 6.49 (d, J = 8.9 Hz, 2H), 4.87 (t, J = 4.8 Hz, 1H), 3.88 (s, 3H), |
| | | 3.43 (t, J = 5.3 Hz, 1H), 3.27-3.13 (m, 2H), 2.81 (d, J = 11.5 Hz, |
| | | 1H), 2.36 (dd, J = 13.9, 8.5 Hz, 1H). |
| 30 | MLAE-042 | ¹H NMR (400 MHz, DMSO) δ 9.08 (bs, 1H), 7.76 (s, 3H), |
| | | 6.75-6.64 (m, 2H), 6.57-6.46 (m, 2H), 2.78 (t, J = 7.4 Hz, 2H), |
| | | 2.57 (d, J = 4.5 Hz, 3H), 2.37 (t, J = 7.5 Hz, 2H). |
| 31 | MLAE-032 | ¹H NMR (400 MHz, MeOD) δ 7.88 (s, 2H), 7.04 (d, J = 2.9 Hz, |
| | | 1H), 6.77 (d, J = 8.9 Hz, 1H), 6.63 (dd, J = 8.9, 3.0 Hz, 1H), |
| | | 3.61 (s, 2H), 3.21 (s, 3H). |
| 32 | MLAE-061 | ¹H NMR (400 MHz, DMSO) δ 9.07 (s, 1H), 8.06 (bs, 1H), |
| | | 7.68 (s, 2H), 6.69 (dd, J = 8.8, 1.8 Hz, 2H), 6.54 (dd, J = 8.8, 1.8 Hz, |
| | | 2H), 4.92 (t, J = 4.7 Hz, 1H), 3.50 (dd, J = 9.9, 4.1 Hz, 1H), |
| | | 3.32-3.23 (m, 3H), 2.84 (d, J = 13.7 Hz, 1H), 2.43 (dd, J = 13.0, |
| | | 10.0 Hz, 1H), 2.18 (s, 3H). |
| 33 | MLAE-040 | ¹H NMR (400 MHz, DMSO) δ 7.81-7.72 (m, 3H), 6.89 (d, J = 9.1 Hz, |
| | | 2H), 6.64 (d, J = 9.0 Hz, 2H), 3.71 (s, 3H), 2.78 (t, J = 7.6 Hz, |
| | | 2H), 2.57 (d, J = 4.5 Hz, 3H), 2.38 (t, J = 7.5 Hz, 2H). |
| 34 | MLAF-050 | ¹H NMR (400 MHz, MeOD) δ 7.81 (d, J = 8.0 Hz, 1H), 6.95 (d, |
| | | J = 8.1 Hz, 1H), 6.73-6.65 (m, 2H), 6.58-6.48 (m, 2H), |
| | | 3.09 (t, J = 7.6 Hz, 2H), 2.63 (t, J = 7.5 Hz, 2H). |
| 35 | MLAE-038 | ¹H NMR (400 MHz, MeOD) δ 7.79 (s, 2H), 6.76-6.62 (m, |
| | | 2H), 6.59-6.48 (m, 2H), 3.20 (s, 3H), 2.90 (t, J = 7.4 Hz, 3H), |
| | | 2.62 (t, J = 7.4 Hz, 3H). |
| 36 | MLAE-060 | n.d. |
| 37 | MLAE-029-1 | ¹H NMR (400 MHz, DMSO) δ 10.00 (s, 1H), 7.89 (s, 1H), |
| | | 7.74-7.69 (m, 1H), 7.68-7.64 (m, 1H), 7.59 (d, J = 8.1 Hz, 1H), |
| | | 7.31 (t, J = 7.9 Hz, 2H), 7.05 (t, J = 7.0 Hz, 1H), 6.99 (d, J = 10.7 Hz, |
| | | 1H), 6.82 (d, J = 8.9 Hz, 1H), 6.59 (d, J = 9.0 Hz, 1H), |
| | | 3.66 (s, 2H). |
| 38 | MLAE-029-2 | ¹H NMR (400 MHz, DMSO) δ 10.00 (s, 1H), 7.99 (d, J = 7.7 Hz, |
| | | 1H), 7.81 (s, 2H), 7.01-6.96 (m, 1H), 6.83 (d, J = 8.9 Hz, |
| | | 1H), 6.59 (dd, J = 8.8, 2.9 Hz, 1H), 3.58-3.45 (m, 1H), 3.39 (s, |
| | | 2H), 1.78-1.62 (m, 4H), 1.33-1.08 (m, 6H). |
| 39 | MLAE-029-3 | ¹H NMR (400 MHz, DMSO) δ 10.01 (d, J = 2.1 Hz, 1H), |
| | | 8.15 (t, J = 4.9 Hz, 1H), 7.79 (d, J = 2.2 Hz, 2H), 7.34-7.24 (m, |
| | | 2H), 7.25-7.12 (m, 3H), 7.01 (d, J = 2.7 Hz, 1H), 6.83 (dd, J = 8.9, |
| | | 2.3 Hz, 1H), 6.60 (dd, J = 8.9, 2.8 Hz, 1H), 3.39 (s, 2H), |
| | | 2.72 (t, J = 6.9 Hz, 2H). |
| 40 | MLAE-029-4 | ¹H NMR (400 MHz, DMSO) δ 10.01 (s, 1H), 8.62 (t, J = 5.6 Hz, |
| | | 1H), 7.85 (s, 2H), 7.36-7.28 (m, 2H), 7.28-7.20 (m, 3H), |
| | | 7.01 (d, J = 2.7 Hz, 1H), 6.83 (dd, J = 8.9, 2.1 Hz, 1H), |
| | | 6.63-6.54 (m, 1H), 4.30 (d, J = 5.9 Hz, 2H), 3.49 (s, 2H). |
| 41 | MLAE-029-6 | ¹H NMR (400 MHz, DMSO) δ 10.01 (s, 1H), 7.80 (s, 2H), |
| | | 6.98 (d, J = 3.0 Hz, 1H), 6.83 (d, J = 8.8 Hz, 1H), 6.61 (dd, J = 8.8, |
| | | 2.9 Hz, 1H), 3.70 (s, 2H), 3.62 (dd, J = 13.9, 7.3 Hz, 3H), |
| | | 1.12 (t, J = 7.0 Hz, 3H), 1.04 (t, J = 7.0 Hz, 3H). |
| 42 | MLAE-029-7 | ¹H NMR (400 MHz, DMSO) δ 10.05 (s, 1H), 10.01 (s, 1H), |
| | | 7.89 (s, 1H), 7.51 (d, J = 9.0 Hz, 2H), 7.04-6.98 (m, 1H), |
| | | 6.91-6.86 (m, 2H), 6.83 (dd, J = 9.4, 2.7 Hz, 2H), 6.59 (dd, J = 8.8, |
| | | 2.9 Hz, 1H), 3.73 (s, 3H), 3.63 (s, 2H). |
| 43 | MLAE-029-8 | ¹H NMR (400 MHz, DMSO) δ 10.01 (s, 1H), 7.79 (s, 2H), |
| | | 6.96 (t, J = 2.7 Hz, 1H), 6.83 (dd, J = 8.9, 2.5 Hz, 1H), 6.62 (dt, J = 9.1, |
| | | 2.7 Hz, 1H), 3.72 (s, 2H), 3.50-3.40 (m, 4H), |
| | | 1.63-1.54 (m, 2H), 1.49-1.38 (m, 4H). |
| 44 | MLAE-029-9 | ¹H NMR (400 MHz, DMSO) δ 10.00 (s, 1H), 8.06 (t, J = 4.7 Hz, |
| | | 1H), 7.82 (s, 2H), 6.99 (s, 1H), 6.82 (dd, J = 8.8, 2.0 Hz, |
| | | 1H), 6.60 (dd, J = 8.8, 2.8 Hz, 1H), 3.39 (s, 2H), 3.10-3.01 (m, |
| | | 2H), 1.47-1.36 (m, 2H), 1.32-1.15 (m, 4H), 0.86 (t, J = 6.8 Hz, |
| | | 3H). |
| 45 | MLAE-034 | ¹H NMR (400 MHz, DMSO) δ 9.09 (s, 1H), 7.83 (s, 2H), |
| | | 6.69 (d, J = 8.8 Hz, 2H), 6.52 (d, J = 8.8 Hz, 2H), 4.03 (s, 1H), |
| | | 3.94 (s, 1H), 3.69-3.55 (m, 2H), 3.28-3.07 (m, 3H), |
| | | 2.86-2.64 (m, 3H). |
| 46 | MLAE-036 | ¹H NMR (400 MHz, DMSO) δ 11.73 (bs, 1H), 7.82 (s, 2H), |
| | | 6.94-6.79 (m, 2H), 6.72-6.57 (m, 2H), 3.71 (s, 3H), 3.21 (s, |
| | | 3H), 2.82 (t, J = 7.4 Hz, 2H), 2.60 (t, J = 7.5 Hz, 2H). |
| 47 | MLAE-046 | ¹H NMR (400 MHz, DMSO) δ 11.96 (s, 1H), 7.85 (s, 2H), |
| | | 6.90 (dd, J = 9.0, 1.5 Hz, 2H), 6.65 (dd, J = 8.9, 1.5 Hz, 2H), 3.72 (s, |
| | | 3H), 3.63 (s, 2H), 3.24 (s, 3H). |
| 48 | MLAF-012 | ¹H NMR (400 MHz, DMSO) δ 12.19 (bs, 1H), 7.89 (s, 2H), |
| | | 7.58 (dd, J = 9.0, 1.4 Hz, 1H), 7.54-7.39 (m, 6H), 6.99 (s, 1H), |
| | | 2.82 (t, J = 7.1 Hz, 2H), 2.59 (t, J = 7.6 Hz, 2H). |
| 49 | (S)-T2AA | n.d. |
| 50 | (R)-T2AA | n.d. |
| 51 | T3 | n.d. |
| 52 | NF021111 | n.d. |
| 53 | SWUA_017 | n.d. |
| 54 | SWUA_018 | n.d. |
| 55 | MLAF-065 | ¹H NMR (400 MHz, MeOD) δ 7.83 (s, 2H), 6.76-6.68 (m, |
| | | 2H), 6.63-6.55 (m, 2H), 3.32-3.25 (m, 1H), 3.19-3.07 (m, |
| | | 2H), 2.77 (dd, J = 13.7, 5.1 Hz, 1H), 2.53 (dd, J = 13.7, 7.6 Hz, |
| | | 1H), 1.99 (s, 3H). |
| 56 | MLAF-066 | ¹H NMR (400 MHz, MeOD) δ 7.84 (s, 2H), 6.71 (d, J = 8.9 Hz, |
| | | 2H), 6.60 (d, J = 9.0 Hz, 2H), 3.20-3.07 (m, 2H), |
| | | 3.06-2.95 (m, 4H), 2.83 (dd, J = 13.6, 5.1 Hz, 1H), 2.60-2.49 (m, 1H). |
| 57 | MLAF-074 | ¹H NMR (400 MHz, MeOD) δ 7.81 (s, 2H), 6.76-6.67 (m, |
| | | 2H), 6.65-6.52 (m, 2H), 3.87-3.75 (m, 1H), 3.23 (d, J = 8.5 Hz, |
| | | 1H), 3.13-2.99 (m, 2H), 2.77 (dd, J = 13.6, 4.9 Hz, 1H), |
| | | 2.57-2.45 (m, 1H), 1.15 (d, J = 6.5 Hz, 6H). |
| 58 | MLAF-075 | ¹H NMR (400 MHz, MeOD) δ 7.80 (s, 2H), 6.72-6.66 (m, |
| | | 2H), 6.60-6.54 (m, 2H), 3.69-3.62 (m, 4H), 3.39-3.33 (m, |
| | | 5H), 3.16-3.05 (m, 2H), 2.74 (dd, J = 13.7, 5.2 Hz, 1H), |
| | | 2.53 (dd, J = 13.8, 7.4 Hz, 1H). |
| 59 | MLAF-081 | ¹H NMR (400 MHz, MeOD) δ 7.72 (s, 2H), 6.65-6.55 (m, |
| | | 2H), 6.53-6.43 (m, 2H), 3.63-3.55 (m, 4H), 3.17 (d, J = 7.8 Hz, |
| | | 1H), 3.13-3.03 (m, 2H), 2.68 (dd, J = 13.8, 5.3 Hz, 1H), |
| | | 2.61-2.53 (m, 2H), 2.47 (dd, J = 13.7, 7.0 Hz, 1H), 2.40 (bs, |
| | | 4H), 2.32 (t, J = 7.0 Hz, 2H). |
| 60 | MLAF-083 | ¹H NMR (400 MHz, MeOD) δ 7.81 (s, 2H), 6.69 (d, J = 8.8 Hz, |
| | | 2H), 6.58 (d, J = 9.0 Hz, 2H), 4.01 (s, 2H), 3.40-3.32 (m, 1H), |
| | | 3.27-3.11 (m, 2H), 2.77 (dd, J = 13.7, 5.3 Hz, 1H), 2.53 (dd, J = 13.7, |
| | | 7.9 Hz, 1H). |
| 61 | MLAF-085 | ¹H NMR (400 MHz, MeOD) δ 7.81 (s, 2H), 7.37-7.29 (m, |
| | | 4H), 7.29-7.22 (m, 1H), 6.75-6.68 (m, 2H), 6.63-6.57 (m, |
| | | 2H), 4.35 (s, 2H), 3.26 (dd, J = 16.1, 7.7 Hz, 1H), |
| | | 3.14-3.05 (m, 2H), 2.77 (dd, J = 13.7, 4.8 Hz, 1H), 2.49 (dd, J = 13.7, 7.6 Hz, |
| | | 1H). |
| 62 | MLAF-086 | ¹H NMR (400 MHz, MeOD) δ 7.82 (s, 2H), 6.71 (dd, J = 8.9, |
| | | 1.7 Hz, 2H), 6.60 (dd, J = 8.9, 1.7 Hz, 2H), 3.30-3.18 (m, 1H), |
| | | 3.17-3.03 (m, 4H), 2.78 (dd, J = 13.6, 4.1 Hz, 1H), 2.51 (dd, J = 13.1, |
| | | 7.1 Hz, 1H), 1.57-1.44 (m, 2H), 1.42-1.30 (m, 6H), |
| | | 0.93 (t, J = 6.0 Hz, 3H). |
| 63 | MLAG-060 | ¹H NMR (400 MHz, DMSO) δ 9.09 (s, 1H), 7.79 (s, 2H), |
| | | 6.68 (dd, J = 9.0, 3.1 Hz, 2H), 6.56 (dd, J = 9.0, 3.2 Hz, 2H), 6.43 (s, |
| | | 1H), 6.11 (s, 1H), 3.98-3.84 (m, 1H), 2.99 (t, J = 6.4 Hz, 1H), |
| | | 2.81-2.63 (m, 2H). |
| 64 | MLAG-066-1 | ¹H NMR (400 MHz, DMSO) δ 9.17 (s, 1H), 8.05 (s, 1H), |
| | | 7.86 (s, 2H), 6.76 (d, J = 8.8 Hz, 2H), 6.60 (d, J = 8.6 Hz, 2H), |
| | | 3.45 (d, J = 6.7 Hz, 2H), 2.66 (t, J = 5.7 Hz, 3H). |
| 65 | MLAG-066-2 | ¹H NMR (400 MHz, DMSO) δ 9.11 (d, J = 5.5 Hz, 1H), 7.77 (d, |
| | | J = 5.3 Hz, 2H), 6.75-6.65 (m, 2H), 6.58-6.49 (m, 2H), |
| | | 3.70 (d, J = 4.6 Hz, 2H), 3.04 (d, J = 5.4 Hz, 3H), 2.85 (d, J = 5.3 Hz, |
| | | 3H). |
| 66 | MLAG-066-3 | ¹H NMR (400 MHz, DMSO) δ 9.11 (d, J = 6.3 Hz, 1H), 8.08 (d, |
| | | J = 4.4 Hz, 1H), 7.80 (d, J = 6.3 Hz, 2H), 6.75-6.65 (m, 2H), |
| | | 6.58-6.48 (m, 2H), 3.38 (d, J = 5.8 Hz, 2H), 3.14-3.02 (m, |
| | | 2H), 2.55 (d, J = 6.4 Hz, 3H). |
| 67 | MLAG-082 | ¹H NMR (400 MHz, DMSO) δ 9.09 (s, 1H), 7.88 (s, 2H), |
| | | 6.68 (d, J = 8.9 Hz, 2H), 6.52 (d, J = 8.9 Hz, 2H), 3.68 (s, 2H). |
| 68 | MLAG-083 | ¹H NMR (400 MHz, DMSO) δ 7.78 (s, 2H), 7.01 (d, J = 2.9 Hz, |
| | | 1H), 6.81 (d, J = 8.9 Hz, 1H), 6.58 (dd, J = 8.8, 2.9 Hz, 1H), |
| | | 3.26 (dd, J = 10.5, 5.2 Hz, 1H), 3.23-3.18 (m, 1H), |
| | | 2.87-2.79 (m, 1H), 2.67 (dd, J = 13.3, 4.7 Hz, 1H), 2.37 (dd, J = 13.2, 8.2 Hz, |
| | | 1H). |
| 69 | MLAG-087 | ¹H NMR (400 MHz, DMSO) δ 9.09 (bs, 1H), 7.76 (s, 2H), |
| | | 6.68 (d, J = 8.9 Hz, 2H), 6.53 (d, J = 8.9 Hz, 2H), 2.77 (t, J = 6.8 Hz, |
| | | 2H), 2.59 (t, J = 6.9 Hz, 2H), 1.44 (bs, 2H). |
| 70 | MLAG-088 | ¹H NMR (400 MHz, DMSO) δ 10.00 (bs, 1H), 7.81 (s, 2H), |
| | | 7.52 (s, 1H), 7.00 (d, J = 2.9 Hz, 2H), 6.82 (d, J = 8.8 Hz, 1H), |
| | | 6.59 (dd, J = 8.8, 2.9 Hz, 1H), 3.38 (s, 2H). |
| 71 | MLAG-093 | ¹H NMR (400 MHz, DMSO) δ 8.97 (bs, 1H), 7.76 (s, 2H), |
| | | 6.66 (d, J = 8.7 Hz, 1H), 6.52 (s, 1H), 6.26 (dd, J = 8.7, 2.9 Hz, 1H), |
| | | 4.62 (bs, 1H), 3.29-3.24 (m, 1H), 3.21 (d, J = 6.2 Hz, 1H), |
| | | 2.83 (dt, J = 11.5, 5.7 Hz, 1H), 2.66 (dd, J = 13.4, 4.8 Hz, 1H), |
| | | 2.36 (dd, J = 13.3, 8.2 Hz, 1H), 2.07 (s, 3H), 1.59 (bs, 2H). |

*“n.d.” indicates that the data were not determined for the indicated compound.

### 101. Fluorescent Polarization Assay

To determine the binding of the disclosed compounds to the human PCNA recombinant protein, a fluorescence polarization assay was employed. The assay is based on competition between a fluorescent tracer and increasing amounts of competing compound, i.e. a disclosed compound, determined as fluorescence polarization intensity (mP). The fluorescent tracer was 5-carboxyfluorescein (5-FAM) labeled PL peptide (“FAM-PL”). The PL peptide sequence is SAVLQKKITDYFHPKK (Kontopidis, et al. PNAS (2005) 102:1871-1876). PCNA was a recombinant protein purified using standard methods known to one skilled in the art using an expression construct obtained from Dr. Toshiki Tsurimoto, Kyusyu University, Japan.

Briefly, 2 μL of 10 μM FAM-PL and 23.5 μL of 17 μM PCNA were added to 2 mL of assay buffer (35 mM HEPES, pH 7.4, 10% glycerol and 0.01% Titon X-100). The volumes of PCNA and FAM-PL were adjusted to ensure that the final concentration of PCNA was 200 nM and FAM-PL was 10 nM. Assays were carried out in 384 well plates in a total volume of about 20-30 μL. Compounds assayed at six concentrations (made by serial dilution; 330 μM, 100 μM, 33 μM, 10 μM, 3.3 μM, and 1 μM) as single-point measurements (i.e. not in duplicate). Fluorescence measurements were made immediately after dilution in the assay plate. Fluorescein FP readings were with 485-nm excitation (20-nm bandpass) and 535-nm emission (20-nm bandpass) filters, and the supplied 510-nm dichroic mirror. FP values (P) were calculated in millipolarization units (mP),

$$P = 1000 \times \frac{{I}_{\vert \vert} - G \times {I}_{⊥}}{{I}_{\backslash \backslash} + G \times {I}_{⊥}} ,$$

where I_{∥} and I_{⊥} are the emission intensities recorded, respectively, in the parallel and perpendicular directions relative to the plane of excitation polarization, after background intensity correction. G-factor values (G) for the fluorescein spectral window were measured using 1 nM fluorescein.

Alternatively, the fluorescent polarization assay can be carried out as described in the following. Briefly, the disclosed compounds were assayed in a solution consisting of 100 nM PCNA protein and 10 nM N-terminal 5-carboxyfluorescein-labeled PL-peptide (SAVLQKKITDYFHPKK) (Kontopidis, G., Wu, S. Y., Zheleva, D. I., Taylor, P., McInnes, C., Lane, D. P., Fischer, P. M., and Walkinshaw, M. D. (2005) Structural and biochemical studies of human proliferating cell nuclear antigen complexes provide a rationale for cyclin association and inhibitor design. Proc. Natl. Acad. Sci. U.S.A. 102, 1871-1876) in FP buffer (35 mM HEPES, pH 7.4, 10% glycerol, and 0.01% Triton X-100) for 38,035 compounds. Briefly, 20 μl of the assay solution was transferred into each well of a black 384-well plate using Wellmate (Matrix). Twenty nanoliters of the test compounds in DMSO solution were pin-transferred (V&P Scientific) by Biomek (Beckman Coulter) into the PCNA-PL solution in triplicate to give a final drug concentration of 10 μM in each well. The negative control in each plate was DMSO, whereas the positive control was unlabeled PL-peptide as a self-competitor. After 30 min, the fluorescence was read using an EnVision plate reader (PerkinElmer Life Sciences) with 485-nm excitation (20-nm bandpass) and 535-nm emission (20-nm bandpass) filters and the supplied 510-nm dichroic mirror. FP values were calculated in millipolarization units. All data processing was performed using in-house programs in the Pipeline Pilot (Accelrys, version 7.0.1). The quality of the primary screen was assessed by Z-prime and other screening quality metrics. 91 compounds (0.24% hit rate) showing >40% decrease in fluorescence fluorescence polarization were further validated by establishing dose response (10 μM to 0.5 μM final concentration) in triplicate using a freshly prepared batch of each compound in at least two different FP buffers. Among compounds giving regenerative saturating sigmoidal dose-response curves, T3 was selected for validation in this study.

### 102. Compound Activity in Fluorescent Polarization Assay

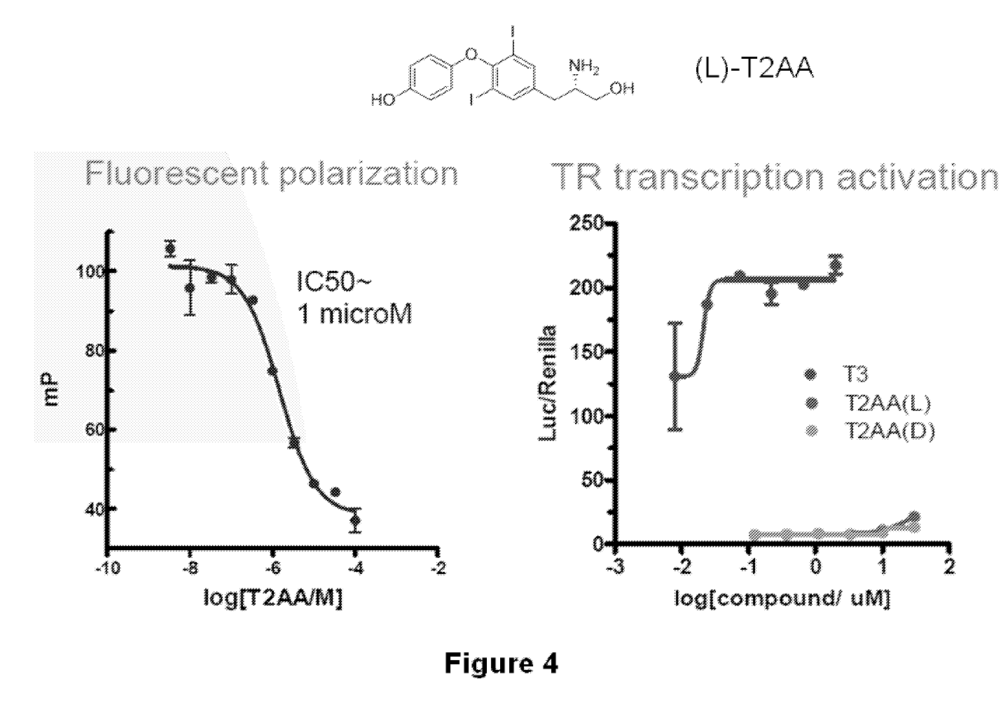

The fluorescent polarization assay as described above was used to determine the IC₅₀ values for representative disclosed compounds, and the data are given below in Table 2. The compound numbers correspond to those used in Table 1a, and thus the corresponding structures as indicated in Table 1a. An example of the dose response effect of the disclosed compounds in the fluorescent polarization assay is shown in FIG. 4 (left panel) using the assay method described above. The data show that the compound (L)-T2AA ((S)-4-(4-(2-amino-3-hydroxypropyl)-2,6-diiodophenoxy)phenol) exhibited an IC₅₀ of about 1 μM under these assay conditions.

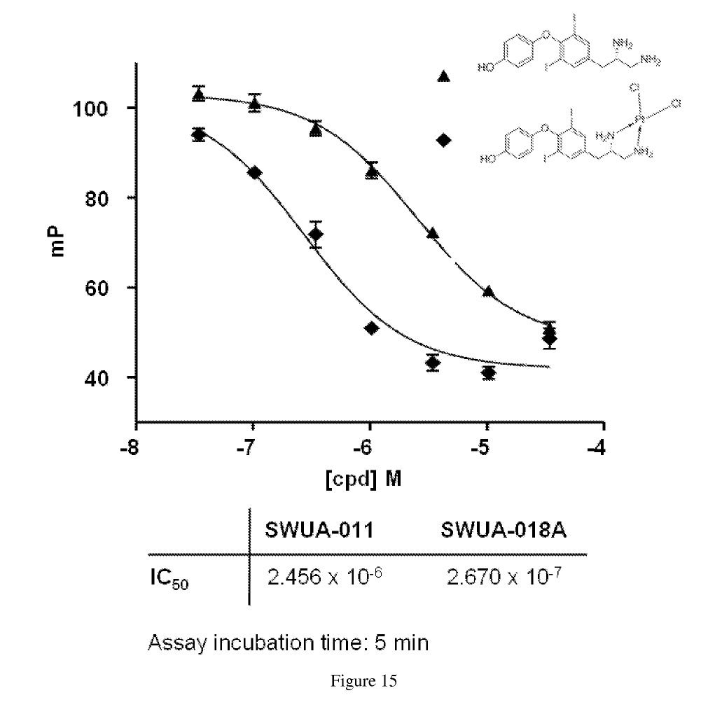

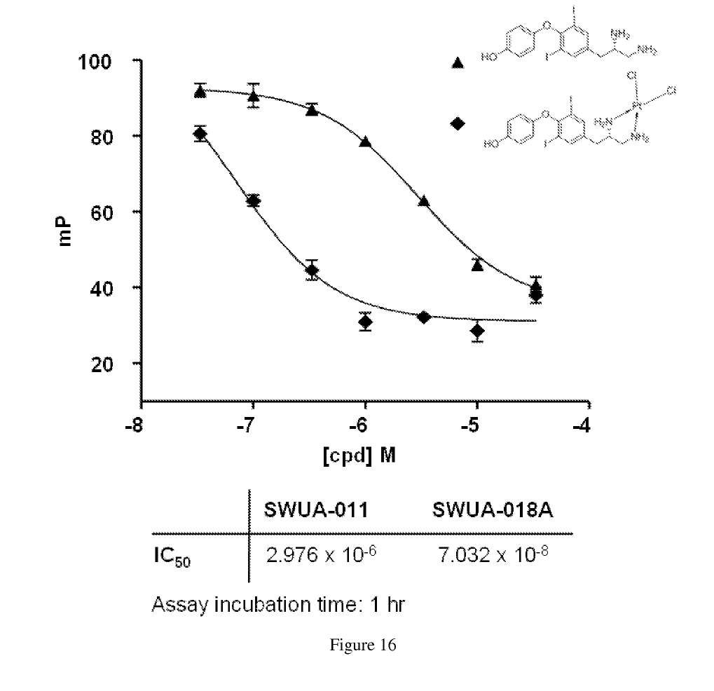

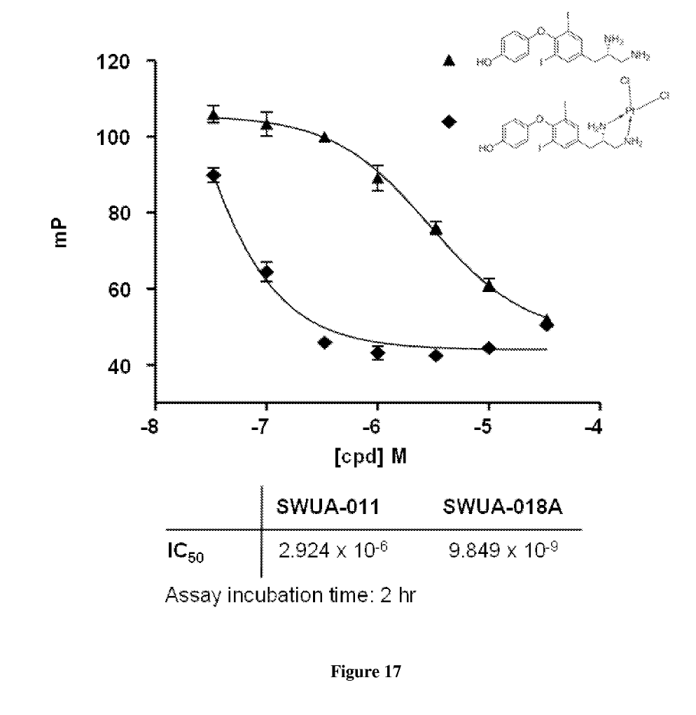

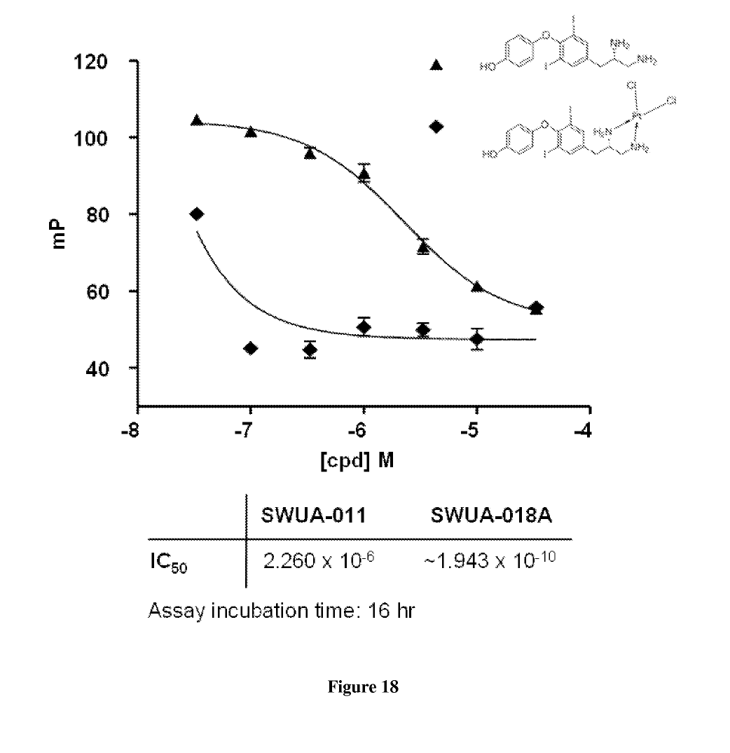

Further examples of the dose response effect of the disclosed compounds as a function of incubation time in the fluorescent polarization assay is shown in FIGS. 15-18 using the assay described above. The data show that as incubation time increased, the IC₅₀ decreased for dichloro((S)-4-(4-(2,3-diaminopropyl)-2,6-diiodophenoxy)phenol)platinum(II), but not for the analogous compound that was not complexed with platinum (II) chloride, (S)-4-(4-(2,3-diaminopropyl)-2,6-diiodophenoxy)phenol. The data in FIGS. 15-18 show that the IC₅₀ for dichloro((S)-4-(4-(2,3-diaminopropyl)-2,6-diiodophenoxy)phenol)platinum(II) as determined in this assay decreased from about 0.27 μM when the incubation time was 5 min to about 0.19 nM when the incubation time was 16 hr. Without wishing to be bound by a particular theory, when the presence of platinum (II) chloride allowed for the formation of crosslinks to PCNA.

TABLE 2

| | | IC₅₀ |
|---|---|---|
| No. | Compound ID | (μM) |
| 1 | MLAF-007 | 0.4 |
| 2 | MLAF-008 | 0.4 |
| 3 | MLAE-062 | 1.2 |
| 4 | MLAF-013 | 1.4 |
| 5 | MLAE-030 | 1.9 |
| 6 | MLAE-045 | 2.1 |
| 7 | MLAE-041 | 2.4 |
| 8 | MLAE-037 | 2.7 |
| 9 | MLAE-051-3 | 2.7 |
| 10 | MLAE-051-2 | 2.9 |
| 11 | MLAE-063 | 2.9 |
| 12 | MLAE-031 | 3.0 |
| 13 | MLAE-059 | 3.7 |
| 14 | MLAE-051-1 | 4.9 |
| 15 | MLAE-054-4 | 5.2 |
| 16 | MLAE-054-1 | 6.0 |
| 17 | MLAE-058-1 | 6.0 |
| 18 | MLAE-058-2 | 6.0 |
| 20 | MLAE-047 | 6.3 |
| 21 | MLAE-054-2 | 6.5 |
| 22 | MLAE-054-3 | 8.3 |
| 23 | MLAE-033 | 8.6 |
| 24 | MLAE-048 | 9.1 |
| 25 | MLAE-049 | 9.1 |
| 26 | MLAE-039 | 9.5 |
| 27 | MLAF-051 | 9.5 |
| 28 | MLAE-035 | 9.7 |
| 29 | MLAE-058-3 | 9.7 |
| 30 | MLAE-042 | 11.0 |
| 31 | MLAE-032 | 14.0 |
| 32 | MLAE-061 | 16.0 |
| 33 | MLAE-040 | 17.0 |
| 34 | MLAF-050 | 19.0 |
| 35 | MLAE-038 | 20.0 |
| 36 | MLAE-060 | 32.0 |
| 37 | MLAE-029-1 | >50 |
| 38 | MLAE-029-2 | >50 |
| 39 | MLAE-029-3 | >50 |
| 40 | MLAE-029-4 | >50 |
| 41 | MLAE-029-6 | >50 |
| 42 | MLAE-029-7 | >50 |
| 43 | MLAE-029-8 | >50 |
| 44 | MLAE-029-9 | >50 |
| 45 | MLAE-034 | >50 |
| 46 | MLAE-036 | >50 |
| 47 | MLAE-046 | >50 |
| 48 | MLAF-012 | >50 |
| 49 | (S)-T2AA | 0.8 |
| 50 | (R)-T2AA | 0.8 |
| 51 | T3 | 1.7 |
| 52 | NF021111 | >50 |
| 53 | SWUA_017 | 2.5 |
| 54 | SWUA_018 | 0.27 |
| 55 | MLAF-065 | 1.5 |
| 56 | MLAF-066 | 1.0 |
| 57 | MLAF-074 | 1.3 |
| 58 | MLAF-075 | 1.4 |
| 59 | MLAF-081 | 1.2 |
| 60 | MLAF-083 | 1.4 |
| 61 | MLAF-085 | 1.0 |
| 62 | MLAF-086 | 1.3 |
| 63 | MLAG-060 | 5.7 |
| 64 | MLAG-066-1 | 8.3 |
| 65 | MLAG-066-2 | 7.5 |
| 66 | MLAG-066-3 | 6.6 |
| 67 | MLAG-082 | 1.6 |
| 68 | MLAG-083 | 0.6 |
| 69 | MLAG-087 | 0.9 |
| 70 | MLAG-088 | 6.6 |
| 71 | MLAG-093 | 1.8 |

Further examples of the dose response effect of the disclosed compounds as a function of incubation time in the fluorescent polarization assay is shown in FIGS. 15-18 using the assay described above. The data show that as incubation time increased, the IC₅₀ decreased for dichloro((S)-4-(4-(2,3-diaminopropyl)-2,6-diiodophenoxy)phenol)platinum(II), but not for the analogous compound that was not complexed with platinum (II) chloride, (S)-4-(4-(2,3-diaminopropyl)-2,6-diiodophenoxy)phenol. The data in FIGS. 15-18 show that the IC₅₀ for dichloro((S)-4-(4-(2,3-diaminopropyl)-2,6-diiodophenoxy)phenol)platinum(II) as determined in this assay decreased from about 0.27 μM when the incubation time was 5 min to about 0.19 nM when the incubation time was 16 hr. Without wishing to be bound by a particular theory, when the presence of platinum (II) chloride allowed for the formation of crosslinks to PCNA.

### 103. Characterization of Biological Activity (I)

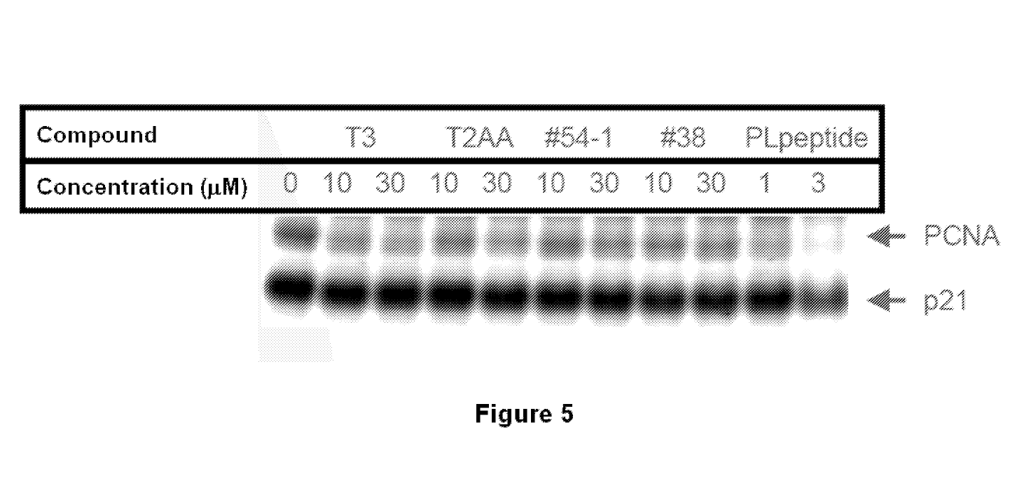

The biological activity of a representative disclosed compound, (S)-T2AA ((S)-4-(4-(2-amino-3-hydroxypropyl)-2,6-diiodophenoxy)phenol), was assessed in various biological assays. For example, the compound was test in a competition assay wherein the ability of a His 6 form of the full-length protein p21 to “pull-down” PCNA is assessed as a function of compound concentration. The data in FIG. 5 show the results for (S)-T2AA, T3 (3,5,3′-triiodothyronine or (S)-2-amino-3-(4-(4-hydroxy-3-iodophenoxy)-3,5-diiodophenyl)propanoic acid), MLAE-054-1 (labeled as “#54-1” in the figure; N-(2-hydroxyethyl)-3-(4-(4-hydroxyphenoxy)-3,5-diiodophenyl)propanamide), MLAE-038 (labeled as “#38” in the figure; 3-(4-(4-hydroxyphenoxy)-3,5-diiodophenyl)-N-(methylsulfonyl)propanamide), and PL peptide (the Pogo-Ligase peptide described above). The figure shows the results of the pull-down experiment as analyzed by gel electrophoresis (the positions of PCNA and p21 are indicated). The level of PCNA that is captured is shown in the absence of compound (leftmost lane), which is decreased significantly by the added compounds. It is noteworthy that the affinity of p21 for PCNA is about 50-100 nM, and that the disclosed compounds appear from the data shown in FIG. 5 to be able to disrupt this high-affinity interaction.

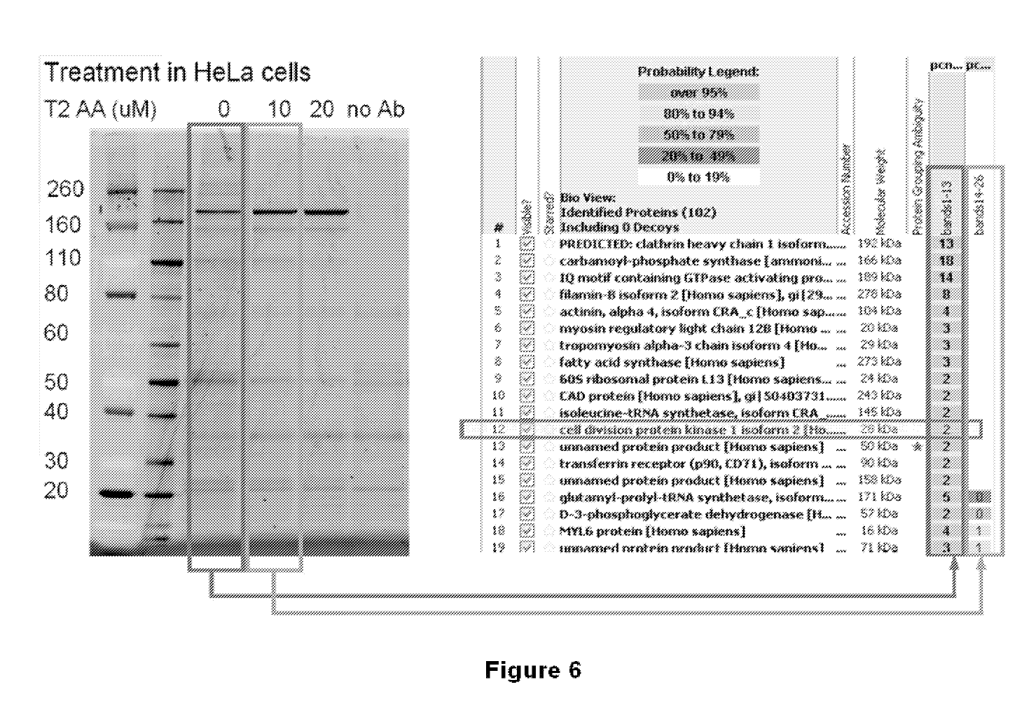

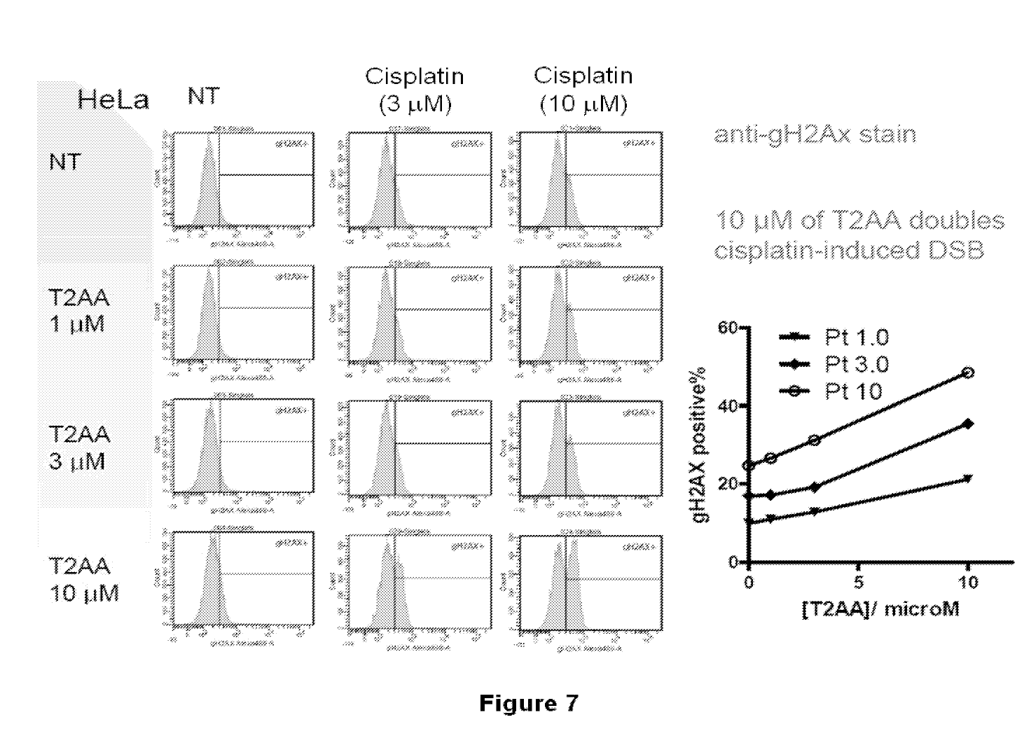

In a further experiment, the proteins which are co-immunoprecipitated from with PCNA, then fractionated by SDS polyacrylamide gel electrophoresis (SDS-PAGE). The identification of the proteins that co-immunoprecipitated with PCNA was accomplished by trypsin digestion of the co-immunoprecipitates, followed by mass spectrometry. A representative SDS-PAGE fractionation is shown in FIG. 6 (left panel), along with the results mass spectrometry analysis of the trypsin digest of the co-immunoprecipitates (right panel). The data show in the absence of added compound, among the proteins which are co-immunoprecipitated with PCNA, the protein cell division protein kinase 1, isoform 2 is in the co-immunoprecipitate. The interaction of this protein with PCNA appears to be disrupted by the presence of 10-20 μM (S)-T2AA.

In a further experiment, the effect of the disclosed compound (S)-T2AA on cisplatin-induced DNA double-strand breaks (“DSB”) was examined by flow cytometry. The data show that increasing concentration of cisplain (indicated as Pt in the graph to the right) result in increased number of DSB (as represented by percentage of cells which are gH2Ax positive). The level of DSB is further increased in a dose-dependent manner by (S)-T2AA, with the compound resulting in at least a doubling of cisplatin-induced DNA ds breaks.

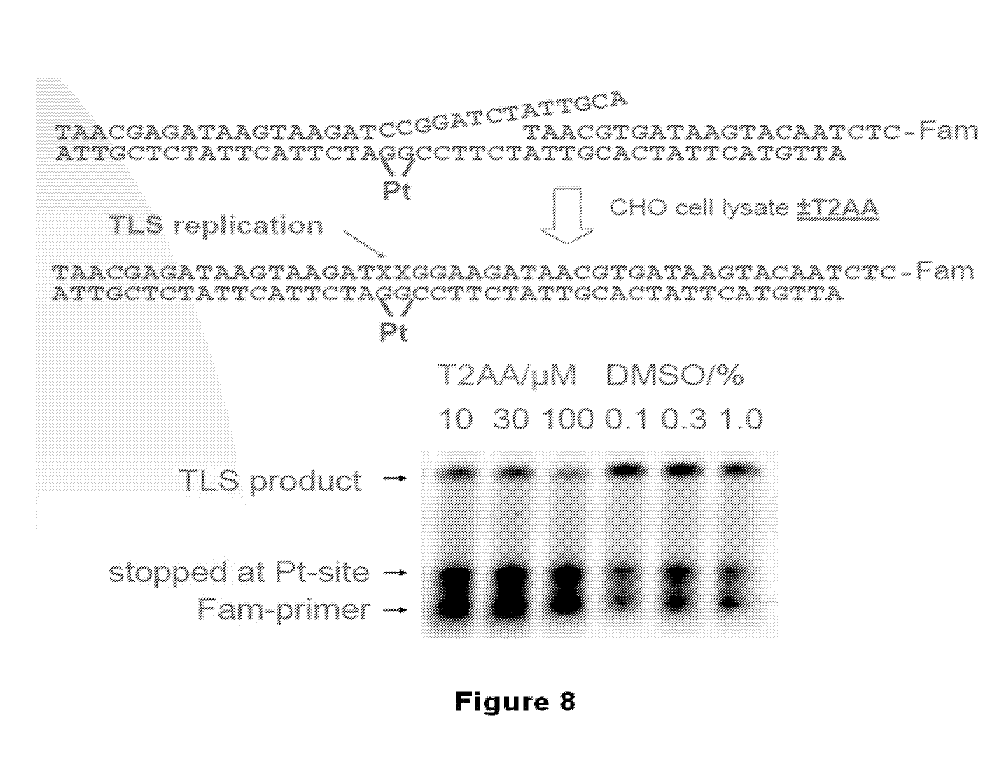

In a further experiment, an in vitro translation DNA polymerization assay was used (e.g. see Hoffman, et al., Proc Natl Acad Sci USA (1996) 93(24), 13766-9). The assay measures the ability of DNA polymerase present in a CHO lysate to catalyze synthesis of a DNA strand past a cisplatin adduct in an oligonucleotide template. The data in FIG. 8 outline the assay approach in the upper panel, and the gel electrophoresis data are shown in the lower panel. The data show that (S)-T2AA inhibits the ability of DNA synthesis to occur past the adduct (i.e. the “TLS” product, with TLS meaning “translesion DNA synthesis”). The negative control lanes (right side of gel image showing the levels of DMSO contributed by the compound) show not effect on DNA synthesis.

The specificity of the disclosed compounds was assessed in a TR (thyroid hormone receptor) transcription activation assay. In the data shown in FIG. 4 (right panel), the compounds (S)-T2AA, (R)-T2AA ((R)-4-(4-(2-amino-3-hydroxypropyl)-2,6-diiodophenoxy)phenol), and T3 were tested in this transcription assay. The selectivity in this assay was of interest since T3 is a thyroid hormone ligand of the T3 hormone receptor. In one aspect, the disclosed compounds are selective for PCNA and do not interact with the T3 hormone receptor. The data show that both (S)-T2AA and (R)-T2AA do not have any apparent affinity for the T3 hormone receptor in this assay, in contrast to the natural ligand, T3.

### 104. Cell Viability Assay

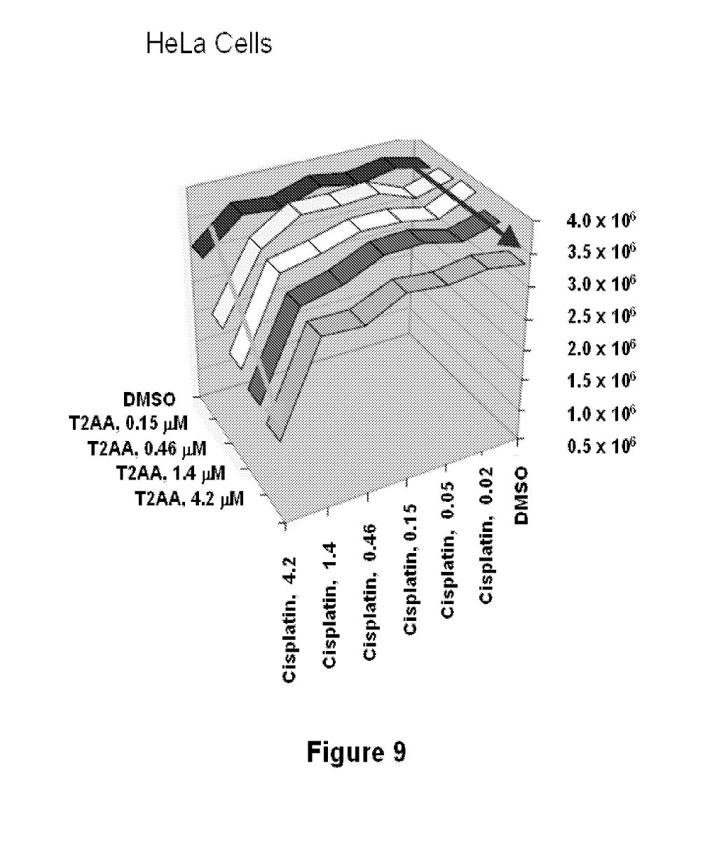

Cell viability was determined using CellTiter-Glo® Luminescent Cell Viability Assay (Promega Corporation, Madison, Wis.). Cell viability assays were carried out in 384-well plates after cells were treated for 24 or 48 hours with the disclosed compounds. Representative data showing the sensitizing effect of a representative disclosed compound, (S)-T2AA ((S)-4-(4-(2-amino-3-hydroxypropyl)-2,6-diiodophenoxy)phenol), are shown in FIG. 9. The data show that an increasing concentration of (S)-T2AA is associated with a greater loss of cell viability for a given concentration of cisplatin.

### 105. Effect of Compounds on DNA Replication and Activation of Gamma-H2AX

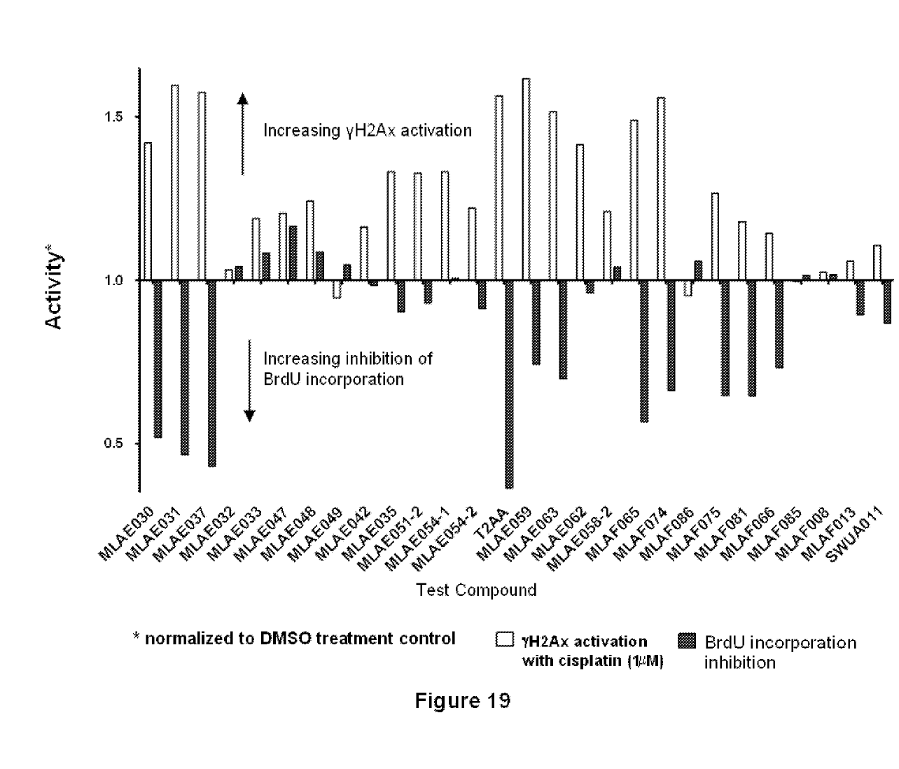

Data in FIG. 19 show the effect of representative compounds on inhibition of DNA replication, as determined from inhibition of BrdU incorporation, and activation of γH2Ax by cisplatin. The data indicate that the inhibitory activity compounds on DNA replication (see closed colored bars in FIG. 19) correlates with activity on chemosensitivity activity, i.e. increased activation of γH2Ax (see open bars in FIG. 19). Without wishing to be bound by a particular theory, a common mechanism may exist for the activity of the compounds on DNA replication and sensitization of cells to cisplatin.

The data in FIG. 19 were obtained using HeLa cells. BrdU incorporation was measured with the APC BrdU Flow Kit (BD Pharmingen) following the manufacturer's instructions. Cells were pulsed with 20 μM BrdU 15 min before harvesting. The populations of BrdU-positive cells and mean BrdU intensity in S-phase cells were recorded. For γH2AX staining, cells were cultured at 4×10⁵ cells/well of standard culture 12-well plates in 1 ml of medium, treated with drugs as indicated, trypsin-lifted and stained using the FlowCellect H2A.X DNA Damage kit (FCCH025142, Millipore) according to the manufacturer's recommendations. All samples were filtered through 40-1 μm nylon mesh prior to flow cytometry analysis. The flow data were acquired using an LSRII flow cytometer equipped with 405-, 488-, 561-, and 640-nm lasers (BD Biosciences).

### 106. Effect of Compounds in a Translession Synthesis Assay

One of the important roles of PCNA is polymerase switching from regular replication polymerases (such as DNA Polδ) to translesion DNA synthesis polymerases (such as DNA Polη) when the replication fork meets a damaged site on the DNA template (such as cisplatin intrastrand cross-links, the major reaction product of DNA and cisplatin; see Lehmann, A. R. (2006) Exp. Cell Res. 312, 2673-2676). The cellular TLS assay uses replications of plasmid DNA encoding firefly luciferase and containing cisplatin cross-links in the plasmid.

To prepare cisplatin-damaged plasmid DNA, cisplatin (1.52 mg) and silver nitrate (1.70 mg) were dissolved in deionized water (dH₂O, 500 μL), stirred in dark overnight, centrifuged, and 0.84 μL of the supernatant (corresponding 1 platinum per 22 bp) was incubated with 7.6 μL of 790 μg/μL solution (6.0 μg) of pGL4.5 (Promega, GenBank # EU921840) in 10 mM Tris-1 mM EDTA pH7 buffer for 2 hours at 37° C. The platinated pGL4.5 plasmid (refereed as Pt-pGL4.5 hereafter) was isolated by standard ethanol precipitation. Property of the Pt-pGL4.5 was verified by agarose gel confirming crosslinked: supercoil˜1:2. Preliminary experiment by the following procedure showed that the Pt-pGL4.5 affords˜20% luciferase activity compared to intact pGL4.5.

GM14931 fibroblast (Coriell Cell Repositories) is an SV40-transformed cell line lacking XPG gene, thus unable to repair cisplatin-damaged DNA by nucleotide excision repair (NER). The pGL4.5 possesses an SV40 origin sequence in its SV40 early enhancer/promoter region, thus can be replicated in GM14931 cells. GM14931 cultured in DMEM (400,000 cells) were transfected by a mixture of the Pt-pGL4.5 (400 ng) and pRL-TK (Promega, 1.8 μg) using Opti-MEM and FuGene6 (Roche) per manufacturer's recommendation. After 5 hours, the cells were re-plated into a 96-well plate (4000 cells/100 μL per well), and dilution of each test compound (50 μL of 30 μM in DMEM) was added triplicate. After 72 hours of the transfection, culture media was removed, and activities of firefly luciferase (FL) and Renilla luciferase (RL) were measured by DLR reagent (Promega) per manufacturer's recommendation. FL/RL values represent replication of plasmids that were produced by TLS from the Pt-pGL4.5, thus showing TLS activities in the cells treated by the compounds, which are indicated as 1 for that of cells treated by DMSO. A lower FL/RL ratio, after normalization, is indicative of decreased translesion DNA synthesis.

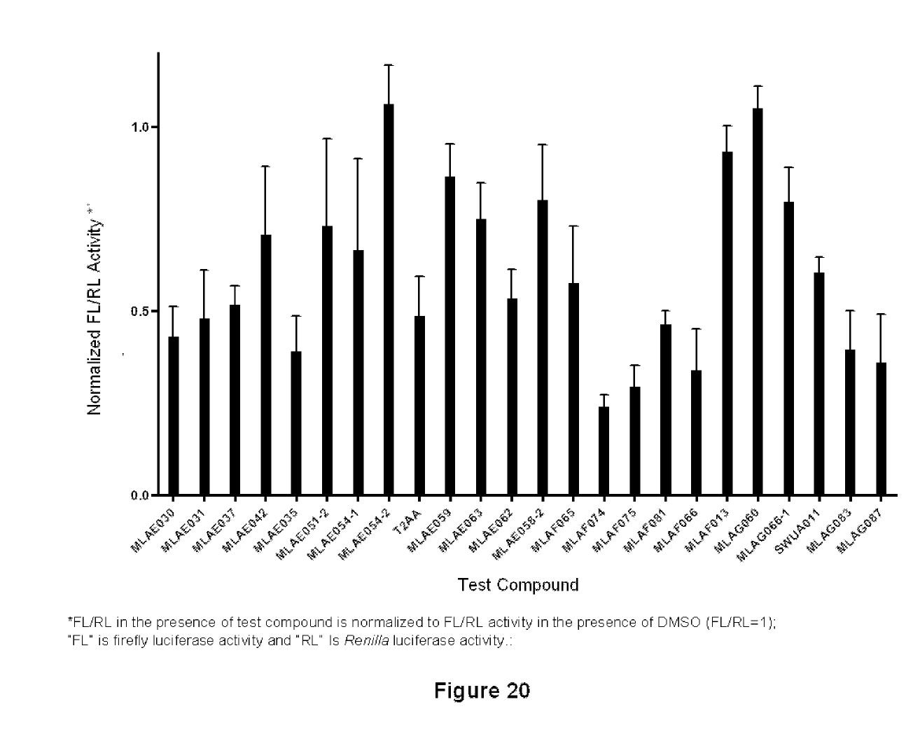

The data in FIG. 20 show the effect of the indicated representative disclosed compounds on translesion DNA synthesis.

### 107. Effect of Compounds on U2OS Cell Viability

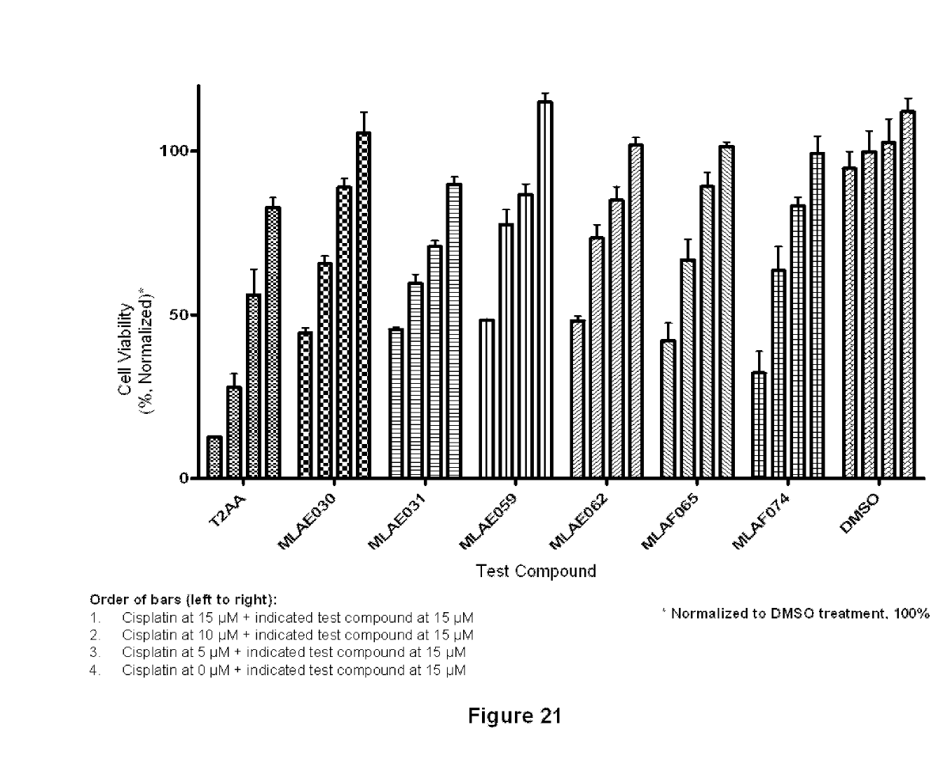

U2OS cells were cultured in DMEM in a 384-well plate (3000 cells/20 μL per well), treated with indicated dilution of cisplatin (see FIG. 21) and each indicated test compound (15 μM; 20 μL per well in DMEM), and cultured for 4-days. AlamarBlue reagent (Invitrogen, 4 μL per well) was added and signals were read per manufacturer's recommendation. Signals of wells contains no cells and cells with no drugs served as 0% and 100% viability, respectively.

U2OS cells are relatively resistant to cisplatin, and their growth is not significantly affected by the presence of up to 10 μM cisplatin (see FIG. 21, compare to DMSO treated control cells), and it was reduced only about 0-10% with 15 μM of the indicated test compound (FIG. 21). In contrast, when cells received both compounds, their growth was significantly reduced in a dose-dependent manner with cisplatin concentration at a 15 μM concentration of test compound (FIG. 21).

### 108. Characterization of Biological Activity (II)

The biological activity of a representative disclosed compound, (S)-T2AA ((S)-4-(4-(2-amino-3-hydroxypropyl)-2,6-diiodophenoxy)phenol), was assessed in various biological assays. Some of the experiments described herein are discussed in the section entitled “Characterization of Biological Activity (I),” and represent independent experimental repeats of the data therein.

a. METHODS

(1) Antibodies, Plasmids, Cell Culture, Oligonucleotides, and Peptides

The following antibodies were used per the manufacturers' recommendation: anti-PCNA PC10 mouse mAb (Cell Signaling 2586), anti-Polδ3 rabbit (Sigma Prestige Antibodies HPA039627), anti-BrdU B44 mouse mAb (BD Biosciences Immunocytometry Systems 347580), anti-phospho(Ser33)RPA32 (replication protein A 32-kDa subunit) rabbit (Bethyl Laboratories A300-246A), antiphospho(Ser³⁴⁵)Chk1 (checkpoint kinase 1) 133D3 rabbit (Cell Signaling 2348), Anti-phospho(Ser¹³⁹)histone H2AX JBW301 mouse (Millipore 05-636), IgG-HRP-conjugated secondary antibodies (Cell Signaling), and Alexa Fluor-conjugated secondary antibodies (Invitrogen). U2OS and HeLa cells were obtained from American Type Culture Collection (ATCC, Manassas, Va.) and cultured in Dulbecco's modified Eagle's medium (DMEM) containing 10% FBS. XP2OS(SV40) cells were cultured in RPMI1640 medium containing 10% FBS. All cells were maintained at 37° C. in a humidified, 5% carbon dioxide incubator. Peptides were synthesized by standard Fmoc chemistry on resin and purified by reverse phase HPLC. Oligonucleotides were custom ordered.

(2) Plasmids

The following plasmids were obtained from each respective investigator or source indicated: T7-PCNA (Toshiki Tsurimoto, Kyusyu University, Japan; Ohta, S., et al. (2002). J. Biol. Chem. 277, 40362-40367), pET8c-p21His6 (Richard Kriwacki, St. Jude Children's Research Hospital) (Kriwacki, R. W., et al. (1996). Proc. Natl. Acad. Sci. USA. 93, 11504-11509), CMV-TRβ and DR4-TRE-firefly luciferase reporter (Kip Guy, St. Jude Children's Research Hospital; Arnold, L. A., et al. (2006). Sci. STKE. 2006, p13), and TK-Renilla luciferase control reporter plasmid (Promega).

(3) Recombinant Protein Expression and Purification

The full length human PCNA (Hishiki, A., et al. (2008). Acta Crystallogr. Sect. F Struct. Biol. Cryst. Commun. 64, 819-821) was cloned into expression vector pGEMEX-1 and expressed in Escherichia coli strain BL21(DE3) (Novagen) in LB broth (Novagen) with ampicillin (100 μg/mL). PCNA was induced with 1 mM IPTG at OD₆₀₀=0.6 and growth continued for 15 hours at 30° C. All subsequent procedures were performed at 4° C. Bacterial cells were harvested by centrifugation for 20 min at 4,000×g in a Beckman JLA8.1 rotor and the pellets were suspended in lysis buffer (25 mM Tris-HCl pH 8.5, 300 mM NaCl, 1 mM DTT, 1 mM PMSF, 1 mM EDTA, 10% glycerol). Cells were lysed by microfluidizer and lysates were clarified by centrifugation at 40,800×g for 1 hour. The supernatant was loaded onto a 5 mL HiTrap Q HP column (GE Healthcare) and eluted using a linear gradient of NaCl (0.3-1.0 M). Fractions containing PCNA, as identified by SDS-PAGE, were pooled, brought slowly to a final ammonium sulfate concentration of 1.8 M and gently stirred for 1 hour. The protein solution was clarified by centrifugation at 34,800×g for 20 minutes, and the supernatant was loaded onto a 5 mL HiTrap Phenyl HP column (GE Healthcare) pre-equilibrated in 1.8 M ammonium sulfate, 1 mM EDTA, 10% glycerol, 25 mM Tris-HCl pH 8.0. PCNA was eluted with a linear gradient of 1.8 to 0 M ammonium sulfate in the same buffer. Fractions containing PCNA were pooled and the protein solution dialyzed in 10 mM HEPES-NaOH pH 7.4, 100 mM NaCl. The purified PCNA was a single band as judged by SDS-PAGE with Brilliant Blue G250 stain (BioRad).

The full length human p21 was cloned into expression vector pET24 and expressed in Escherichia coli strain BL21(DE3) in Turbo Prime broth (AthenaES) with kanamycin (30 μg/mL). p21 expression was induced with 1 mM IPTG at OD₆₀₀=7.2 and growth continued for 6 hours at 37° C. Bacterial cells were harvested by centrifugation for 20 minutes at 4,000×g in a Beckman JLA8.1 rotor and the pellets were suspended in lysis buffer (50 mM Tris-HCl pH 7.6, 200 mM NaCl, 0.05% Triton X-100, 10% glycerol). Cells were lysed by microfluidizer and the insoluble fraction containing p21 was collected by centrifugation at 38,000×g for 1 hour. The pellets were washed with lysis buffer plus 0.5 M urea and then dissolved in 100 mM Tris-HCl, pH 8.0, 100 mM NaH₂PO₄, 8 M urea. The protein solution was clarified by centrifugation at 48,000×g for 30 minutes. 20 mL of charged and pre-equilibrated Ni-NTA agarose (Qiagen) was added to the supernatant and mixed at room temperature for 2 hours. The beads were then washed with 100 mM Tris-HCl pH 6.3, 100 mM NaH₂PO₄, 8 M urea, and the p21 was eluted with 100 mM Tris-HCl pH 4.5, 100 mM NaH₂PO₄, 8 M urea. The purified human p21 was analyzed by SDS-PAGE and found to be essentially pure.

(4) Crystallization and Structure Determination of PCNA in Complex with T3

Human PCNA was overexpressed and purified by a procedure similar to that previously reported (Hishiki, A., et al. (2008) Acta Crystallogr. Sect. F Struct. Biol. Cryst. Commun. 64, 819-821.). Crystals of PCNA-T3 complex were obtained by sitting-drop vapor diffusion methods by mixing of equal volumes of protein solution (9 mg/ml PCNA, 1 mM T3, 9 mM HEPES-NaOH, pH 7.4, 90 mM NaCl, and 10% DMSO) and a reservoir solution (100 mM sodium cacodylate, pH 6.5, 200 mM NaCl, and 2.0 M ammonium sulfate). Cubic crystals were obtained in several days at 293 K. Prior to x-ray diffraction experiments, crystals were transferred to the reservoir solution containing 20% ethylene glycol for cryoprotection. X-ray diffraction data were collected under N₂ gas steam (100 K) at Photon Factory beamline BL-5A. Diffraction data were processed using the program HKL2000. The structure of the PCNA-T3 complex was solved by the molecular replacement method with the program MOLREP. Model building was performed with the program COOT. Structure refinement was performed with the programs CNS and REFMAC. The geometries of the final structure were validated with the program PROCHECK. Data collection and refinement statistics are given in Table 3. Values in parentheses are for the highest resolution shell. Ramachandran statistics indicate the fraction of residues in the most favored/allowed/disallowed regions, respectively.

Diffraction data were processed using the program HKL2000 (Otwinowski, Z., and Minor, W. (1997). Methods Enzymol. 276, 307-326). The structure of PCNA-T3 complex was solved by the molecular replacement method with the program MOLREP (Vagin, A., and Teplyakov, A. (1997). J. Appl. Crystallogr. 30, 1022-1025). Model building was performed with the program COOT (Emsley, P., and Cowtan, K. (2004). Acta Crystallogr. D Biol. Crystallogr. 60, 2126-2132). Structure refinement was performed with the programs CNS (Brunger, A. T., et al. (1998). Acta Crystallogr. D Biol. Crystallogr. 54, 905-921) and REFMAC (Murshudov, G. N., Vagin, A. A., and Dodson, E. J. (1997). Acta Crystallogr. D Biol. Crystallogr. 53, 240-255). The geometries of the final structure were validated with the program PROCHECK (Laskowski, R. A., et al. (1993). J. Appl. Crystallogr. 26, 283-291). Data collection and refinement statistics are given in Table 3. In Table 3, values in parentheses are for the highest resolution shell and Ramachandran statistics indicate the fraction of residues in the most favored/allowed/disallowed regions, respectively.

(5) TR Reporter Assay

HepG2 cells were cultured at 5×10⁶ cells in 100 mm culture dishes in 10 mL of DMEM/F-12 medium (1:1 mixture, Hyclone Laboratories) containing 2.5 mM 1-glutamine and 10% heat-inactivated charcoal-stripped serum (Hyclone Laboratories) for 6 h, and transfection mixture (CMV-TRβ plasmid 5 μg, DR4-TRE-firefly luciferase reporter plasmid 15 μg, TK-Renilla luciferase reporter plasmid 1.25 μg, FuGENE 6 (Roche Applied Science) 64 μL, Opti-MEM (Invitrogen) 460 μL) was added. The cells were incubated overnight, trypsin-lifted and transferred to 96-well plates (Corning) at 2×10⁴ cells/well in 75 μL of medium without phenol red. Six hours after plating, serially diluted compounds in 25 μL of the medium were added in triplicate. After incubation for 18 hours, luciferase activity was measured by using Dual-Glo (Promega) per manufacturer's recommendation in an EnVision plate reader. TR activity was reported by normalizing the Firefly luciferase activity with the Renilla luciferase activity for each well.

(6) Pull-Down Assay

A H is protein interaction pull-down kit (Thermo Scientific) was used, in which a 4:1 mixture of TBS and lysis buffer provided in the kit was used as the buffer throughout. For immobilizing p21, 100 μg of p21His₆ was incubated overnight at 4° C. with 25 μl (50% suspension) of prewashed cobalt chelate resin in 500 μl of the buffer and washed with the buffer four times. Separately, PCNA (500 nM) in 500 μl of the buffer was mixed with the indicated final concentration of T2AA and transferred to the p21-immobilized resin. After the mixture was incubated overnight at 4° C., the resin was washed with buffer four times, and the bound proteins were eluted with 100 μl of the buffer containing 400 mM imidazole. Each 10 μl of the eluted samples was analyzed by SDS-PAGE with Oriole fluorescent gel stain (BioRad) and a Fluor Chem imager (Alpha Innotech).

(7) Immunoblotting

The cells treated as indicated were washed twice with ice-cold PBS, collected in a spin tube, and lysed with radioimmune precipitation assay buffer supplemented with Halt protease inhibitor mixture and Halt phosphatase inhibitor mixture (Thermo Scientific) using approximately twice the volume of the cell pellet on ice for 0.5 h. Protein concentration was determined by a BCA assay (Thermo Scientific) according to the manufacturer's recommendation. Normalized amounts of samples were loaded on SDS-PAGE as indicated and electrotransferred to a PVDF membrane. The membrane was blocked with SuperBlock buffer (Thermo Scientific) and incubated with the indicated primary antibodies in SuperBlock at 4° C. overnight, rinsed with TBS, 0.05% Tween 20 three times for 10 min, incubated with the corresponding secondary IgG-HRP conjugate in SuperBlock at room temperature for 1 h, and rinsed with TBS, 0.05% Tween 20 for 15 min. Proteins probed on the membrane were visualized by chemiluminescence using WestPico reagent (Thermo Scientific) and developed on BioMax MR film (Eastman Kodak Co.).

(8) Cell-Growth Assay

The cells were treated with drugs as indicated in a black (transparent flat bottom) 384-well or 96-well plate. Cellular viability was determined by using Alamar Blue reagent (Invitrogen) on an EnVision plate reader according to the manufacturer's recommendations and normalized with signal from wells of DMSO treatment=100% and of no cells=0%.

(9) Chromatin Immunostaining

Cells grown on a coverslip were incubated with the indicated concentrations of T2AA for 24 h at 37° C., rinsed once with ice-cold PBS, pre-extracted with ice-cold CSK buffer (100 mM sodium chloride, 3 mM magnesium chloride, 10 mM HEPES, pH 7.4, 300 mM sucrose) containing 0.3% Triton X-100, Halt protease inhibitor mixture and Halt phosphatase inhibitor mixture (Thermo Scientific) for min on ice, and rinsed once with the CSK buffer to remove Triton X-100 from the specimens. The cells were fixed with 4% paraformaldehyde in PBS for 20 min at room temperature, rinsed three times with PBS for 5 min each, permeabilized with ice-cold methanol for 10 min at −20° C., and rinsed once with PBS for 5 min. The specimens were blocked with PBS containing 5% FBS and 0.03% Triton X-100 for 60 min, applied anti-PCNA antibody (1:3200) and anti-Polδ3 antibody (2.9 μg/ml) or anti-phospho(Ser³³)RPA32 antibody (4.0 μg/ml) in antibody buffer (PBS containing 1% FBS and 0.03% Triton X-100), incubated overnight in a humid chamber at 4° C., and rinsed with PBS three times for 5 min each at room temperature. The specimens were incubated with Alexa Fluor 555-conjugated goat anti-mouse IgG (Invitrogen) antibody (1:500) in the antibody buffer for 2 h in a humidified chamber at room temperature the dark, rinsed once with PBS for 5 min, incubated with Alexa Fluor 488-conjugated goat anti-rabbit IgG (Invitrogen) antibody (1:500) in the antibody buffer for 2 h in a humidified chamber at room temperature in the dark, and rinsed three times with PBS for 5 min. The coverslips were mounted VectaShield containing DAPI at 1 μg/ml (Vector Laboratories). Immunofluorescence imaging for chromatin was performed on a ClSi microscope (Nikon) with a Cascade 512B photomultiplier (Photometrics). Images were processed using EZC1 (Nikon) and Photoshop (Adobe) software. Phospho-Chk1 and γH2AX staining was performed in the same manner except using anti-phospho(Ser³⁴⁵)Chk1 antibody (1:50) or antiphospho(Ser¹³⁹)histone H2AX antibody (1:500) and omitting the pre-extraction and methanol treatment steps.

(10) DNA Replication Assay

DNA replication in U2OS and/or HeLa cells were analyzed by the rates of bromodeoxyuridine (BrdU) incorporation by a method reported previously (Taddei, A., et al. (1999) J. Cell Biol. 147, 1153-1166.). For immunofluorescence, cells were propagated at a subconfluent density onto a coverslip in a 6-well culture plate the day before the assay. Cells were treated with T2AA for 24 h at the indicated concentrations. During the last 15 min in the incubation period, BrdU was added at a concentration of 10 μM. Cells were washed with PBS and fixed with 4% paraformaldehyde in PBS for 15 min at room temperature. After permeabilization with 0.2% Triton X-100 in PBS for 15 min, cells were treated with 4N hydrochloric acid for 10 min and extensively rinsed with PBS. After blocking cells with 3% FBS in PBS for 20 min, anti-BrdU monoclonal antibody (1 μg/ml; clone 44, BD Pharmingen) was added to the cells and incubated at room temperature for 1 h. Cells were rinsed with PBS three times for 5 min each. Antimouse IgG conjugated with Alexa Fluor 555 (Invitrogen) was added and incubated at room temperature for 30 min. Cells were rinsed with PBS three times for 5 min each and mounted in VectaShield containing DAPI (1 μg/ml). Fluorescence microscopy was done on an E800 microscope (Nikon) with a DXM1120 digital camera (Nikon). Images were processed using Photoshop (Adobe). For quantitative analyses, BrdU incorporation was measured with the APC BrdU Flow Kit (BD Pharmingen) following the manufacturer's instructions. Cells were pulsed with 20 μM BrdU 15 min before harvesting. The populations of BrdU-positive cells (a) and mean BrdU intensity in S-phase cells (b) were recorded. Total BrdU incorporation was defined as a x b.

(11) Flow Cytometry

For cell cycle analysis, cells were cultured at 1×10⁶ cells/well of 6-well plates in 2 ml of medium, treated with drugs as indicated, trypsin-lifted, washed once with PBS, suspended in propidium iodide (PI) solution (0.05 mg/ml), and treated with ribonuclease A (2 μg/ml; Calbiochem) at room temperature for 30 min. For apoptosis analysis, cells were cultured at 1×10⁵ cells/well of 12-well plates in 1 ml of medium and treated with drugs as indicated. Dead cells detached in the medium were recovered by centrifuge, and live cells attached on the wells were lifted by trypsin. Both dead and live cells were combined and washed once with PBS, and the number of cells in each sample was adjusted to 1−3×10⁵. The cells were stained with Annexin-V-FITC reagent (Roche Applied Science) according to the manufacturer's recommendation, followed by staining with PI. For γH2AX staining, cells were cultured at 4×10⁵ cells/well of 12-well plates in 1 ml of medium, treated with drugs as indicated, trypsin-lifted and stained by using the FlowCellect H2A.X DNA Damage kit (FCCH025142, Millipore) according to the manufacturer's recommendations. All samples were filtered through 40-μm nylon mesh prior to running flow cytometry. The flow data were acquired using an LSRII flow cytometer equipped with 405-, 488-, 561-, and 640-nm lasers (BD Biosciences).

(12) TLS Assay in Mammalian Cultured Cells

The cisplatin-modified plasmid was constructed, and the TLS assay was performed as previously described (14) with some modifications. Briefly, two oligonucleotides, 5′-GGGAGATCTGGAAGGA-TCTG-3′ and 5′-AATTCAGATCCTTCCAGATCTCCC-3′, were annealed and inserted between the sites of EcoRV and EcoRI in pUCSV40H. The resultant plasmid was designated as pUCSV40H+ BglII. A 13-mer oligonucleotide modified with a 1,3-intrastrand d(GpTpG) platinum cross-link (Pt-GTG) was prepared as described previously (Shivji, M. K., Moggs, J. G., Kuraoka, I., and Wood, R. D. (2006) Assaying for the dual incisions of nucleotide excision repair using DNA with a lesion at a specific site. Methods Mol. Biol. 314, 435-456.). This oligonucleotide has the cross-link at the underlined site of 5′-CCTTCGTGCT-CCC-3′. An unmodified 13-mer oligonucleotide was used for the construction of control plasmid. These oligonucleotides were purified with HPLC and PAGE. To form a gapped duplex plasmid, pUCSV40H and pUCSV40H+ BglII were digested with EcoRV and ScaI, respectively, mixed, and incubated as described previously (Sawai, T., et al. (2009) Genes Environ. 31, 24-30). The gapped duplex plasmid was incubated with a 2-fold molar excess of the purified 13-mer oligonucleotide, which is complementary to the gap sequence except for a bulge in the middle in the presence of ATP (1 mM) and T4 DNA ligase (New England Biolabs, Ipswich, MA) at 16° C. for 6 min. The covalently closed circle plasmid was purified with equilibrium centrifugation on a CsCl gradient. The plasmids with and without the cisplatin adduct are hereafter referred to as Pt-GTG and Mock-GTG, respectively.

For cellular TLS assay, nucleotide excision repair (NER)-deficient XP20S(SV40) cells (Kawanishi, M., Enya, T., Suzuki, H., Takebe, H., Matsui, S., and Yagi, T. (1998) Mutagenic specificity of a derivative of 3-nitrobenzanthrone in the supF shuttle vector plasmids. Chem. Res. Toxicol. 11, 1468-1473) were used to avoid elimination of the cross-link by NER. The cells were transfected with Pt-GTG or Mock-GTG plasmid (150 ng) using a Qiagen Effectene Reagent kit (Qiagen GmbH, Hilden, Germany) as described previously (Sawai, T., et al. (2009) Genes Environ. 31, 24-30). Twenty-four hours after the transfection, the cells were exposed to 5 μM T2AA or 0.25% DMSO (as solvent control) for 48 h. The plasmids were extracted from the cells by using the QIAprep spin miniprep kit (Qiagen), digested with the restriction endonuclease DpnI (New England Biolabs) to remove the unreplicated plasmids, and introduced into Escherichia coli strain DH5α. The E. coli was plated onto LB agar plates containing ampicillin, X-gal, and isopropyl-β-D-thioga-lactopyranoside. In this system, if the cisplatin-modified strand was replicated as a template (i.e., TLS), the reading frame of the lacZ gene of progeny plasmids would be functional; hence, E. coli colonies became blue. If the opposite strand was replicated (i.e., damage-induced strand loss), progeny plasmids would have a dysfunctional reading frame of the lacZ gene; hence, E. coli colonies showed a white color. Therefore, TLS frequency (%) was defined as (number of blue colonies/number of total colonies)×100.

b. RESULTS

(1) T3 Binds to PCNA Cavity that Interacts with Pip-Box Sequences

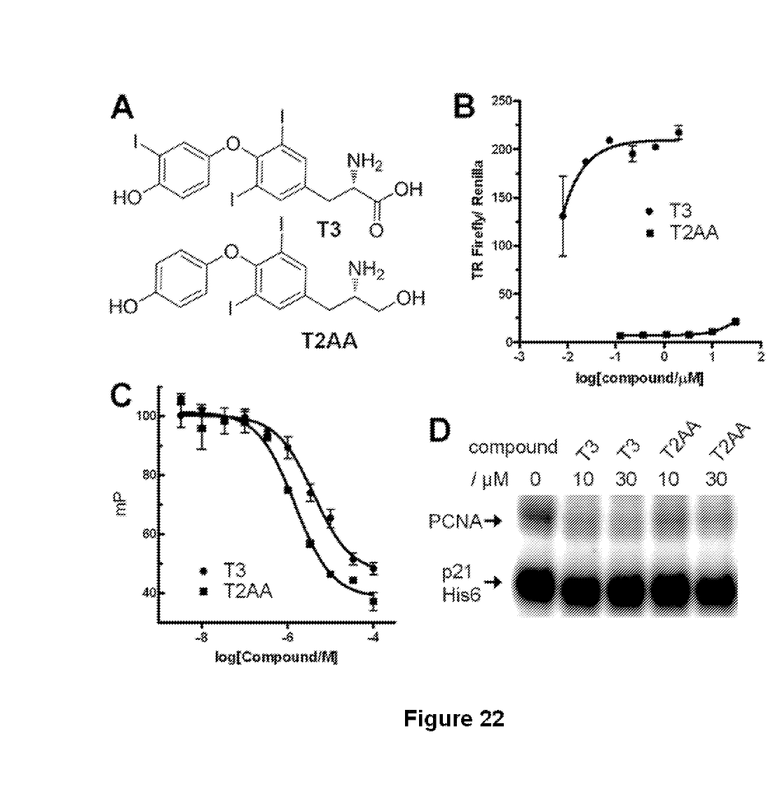

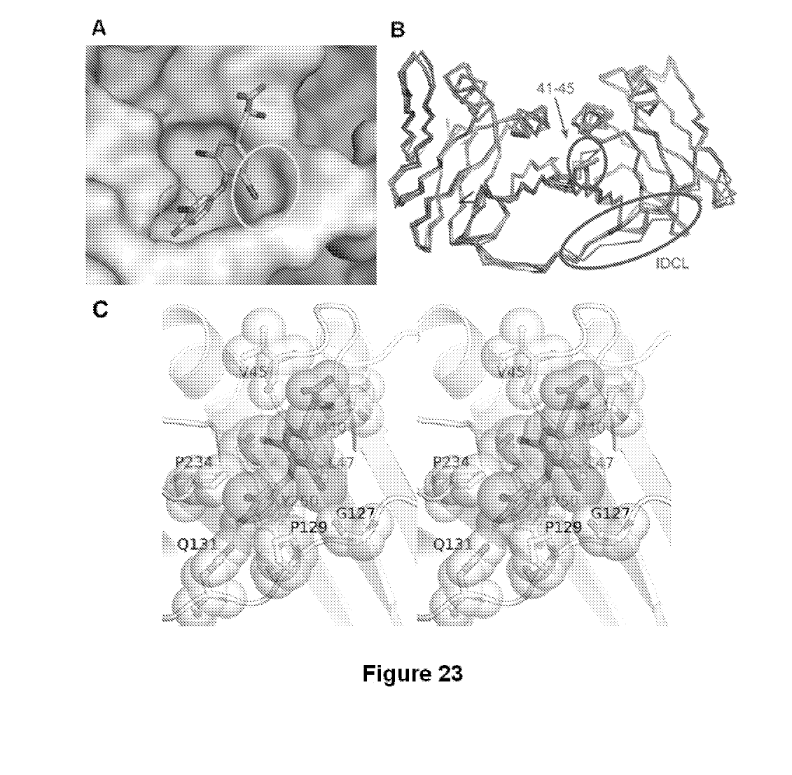

A high-throughput screening protocol to find agents that inhibit biochemical PCNA/PIP-box interactions was utilized. The method used conventional FP for screening chemical libraries by competing the binding of recombinant PCNA protein and fluorescently tagged PL-peptide that possesses a PIP-box sequence, which was previously optimized for high PCNA affinity (IC₅₀ of ˜100 nM in self-competition; data not shown) (Kontopidis, G., et al. (2005) Proc. Natl. Acad. Sci. U.S.A. 102, 1871-1876). The was used to screen ˜38,000 compounds, including FDA-approved drugs and those with known biological activity, and then carefully verified initial hits. After eliminating nonspecific compounds by establishing dose-response curves for the hit compounds with at least two independent experiments, it was determined that T3 (FIG. 22A) was a true inhibitor of PCNA/PL-peptide binding at IC₅₀ of ˜3 μM (FIG. 22C). To further determine if T3 is binding to the bona fide PIP-box interaction site of PCNA or an allosteric site to trigger conformational change that leads to abolishing PCNA/PL-peptide interaction, the co-crystal structure of the PCNA-T3 complex was solved (FIG. 23A; the detailed interactions of T3 with PCNA amino acids are depicted in FIG. 23C). Protein crystallography data collection and refinement statistics are shown below in Table 3.

TABLE 3

| | |
|---|---|
| Data collection | |
| Space group | I2₁3 |
| a = b = c(Å) | 143.1 |
| Resolution range (Å) | 50.0-2.10 (2.18-2.10) |
| Observed reflections | 164,205 |
| Unique reflections | 28,025 |
| R_{merge} | 0.066 (0.392) |
| Completeness (%) | 98.0 (92.9) |
| (I)/σ(I) | 11.0 (2.0) |
| Refinement | |
| Resolution range (Å) | 20.0-2.10 |
| R/R_{free} | 0.205/0.252 |
| Root mean square bond distances | 0.011/1.331 |
| (Å)/angles (degrees) | |
| Ramachandran statistics (%) | 95.1/4.9/0 |
| Protein Data Bank code | 3VKX |

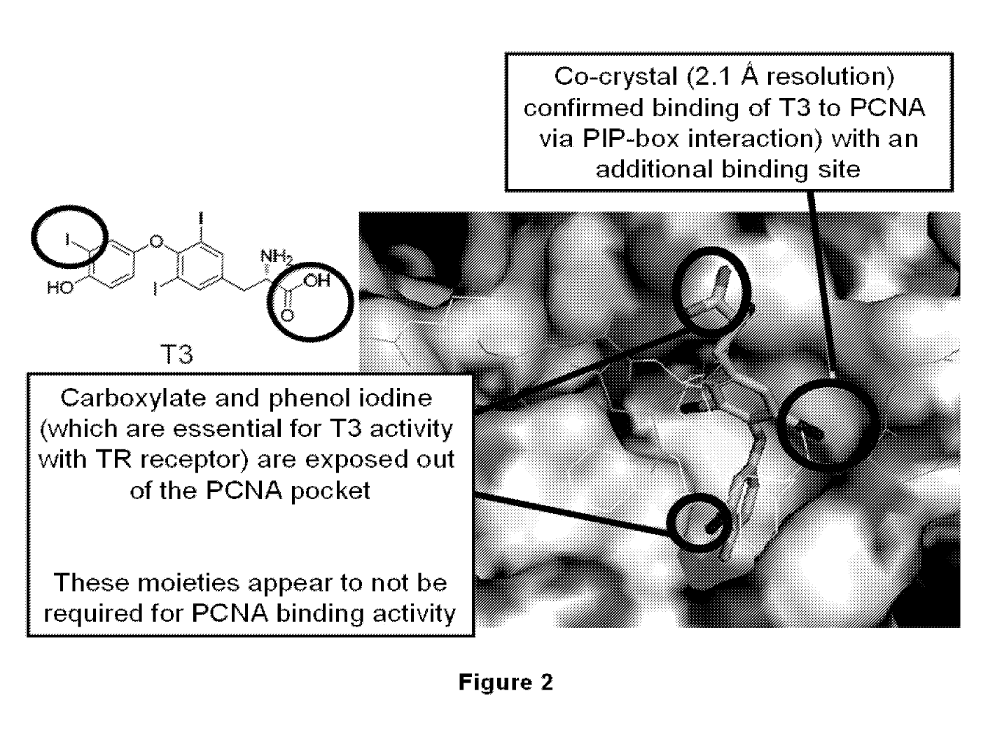

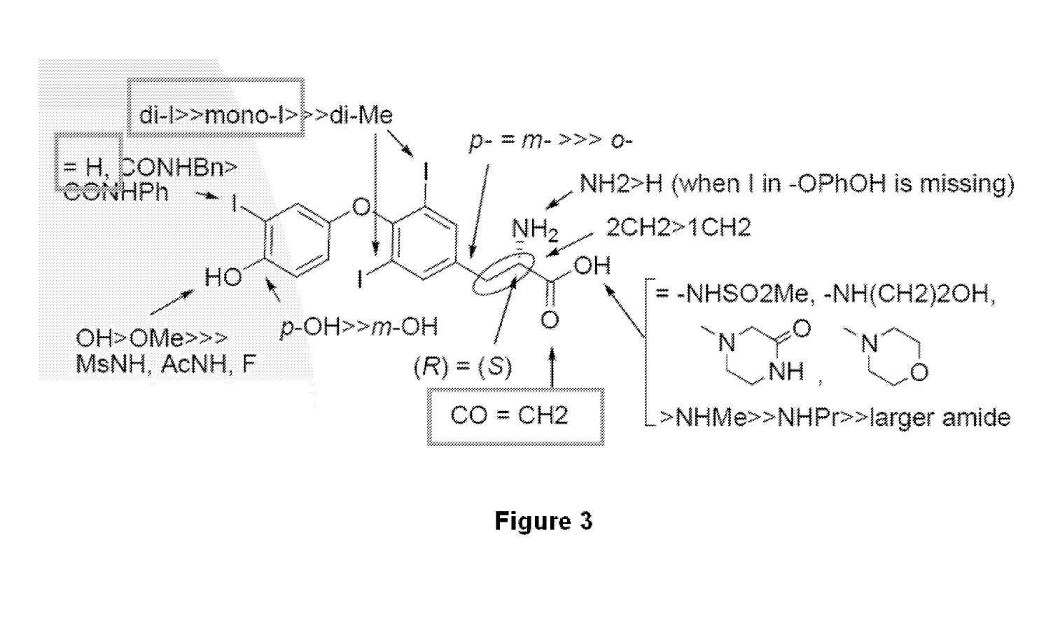

Without wishing to be bound by a particular theory, T3 can bind to the same PCNA cavity that PIP-box protein sequences tightly bind, by inducing an additional binding cavity with allosteric movement of the interdomain connecting loop (IDCL) of PCNA. The IDCL movement around the cavity is unique to the PCNA-T3 complex and is not observed in other PCNA complex structures reported to date (FIG. 2B). These data suggest that IDCL has a role in accommodating ligands with additional interactions. Indeed, 3,3′-diiodothyronine, a T3 derivative, showed remarkably lower affinity than that of T3 (data not shown). Without wishing to be bound by a particular theory, this is presumably because the 5-iodine atom is missing, and thus disables the induction of the IDCL movement.

Briefly, FIG. 22 shows the following: (Panel A) the chemical structure of T3 and T2AA. T2AA lacks 3′-iodine and carboxylic acid of T3 that are essential for the thyroid hormone activity of T3. (Panel B) thyroid hormone activity of T3 and T2AA determined in HepG2 cells were transiently transfected with an expression vector, CMV-TRβ, and a firefly luciferase reporter vector that contains thyroid hormone-responsive element, and were stimulated by titration of T3 (circle) or T2AA (square). T3 fully activated TRβ-TRE reporter at EC₅₀ 10 nM, and T2AA showed almost no activation. Error bars, S.E. (Panel C) inhibition of PCNA/PIP-box peptide interaction by T3 and T2AA. Fluorescent polarization value (in millipolarization units (mP)) was measured for titration of T3 (circle) or T2AA (square) in triplicate in a mixture of PCNA protein (100 nM) and fluorescein-tagged-SAVLQKKITDYFHPKK peptide (10 nM) that contains a PIP-box motif (underlined). Error bars, S.E. The inhibition curve was fitted by Prism (GraphPad) to determine IC₅₀ as 3 μM (T3) and 1 μM (T2AA). (Panel D) inhibition of PCNA/full-length p21 interaction by T3 and T2AA. p21-His6 protein was immobilized on cobalt beads and incubated overnight with PCNA (500 nM) containing the indicated concentration of T3 or T2AA. PCNA bound on the p21-immobilized beads was analyzed by SDS-PAGE/Oriole Orange staining.

(2) T2AA, T3 Derivative Lacking TR Activity, Inhibits PCNA Protein-Protein Interaction

T3 is a very potent thyroid hormone, and this property precludes its use as a PCNA inhibitor. The carboxylic acid and 3′-iodine of T3 is essential for its thyroid hormone activity (Leeson, P. D., et al. (1988) J. Med. Chem. 31, 37-54). In the PCNA-T3 crystal structure, the 3′-iodine atom of T3 is positioned out from the cavity (FIG. 2A), suggesting that it is dispensable for binding. Therefore, a T3 derivative compound was synthesized in which the carboxylic acid of T3 is replaced by an alcohol and the 3′-iodine is removed. The new compound T2AA (FIG. 22A) showed almost no thyroid hormone activity in a TR reporter assay (FIG. 22B) but inhibited the PCNA/PL-peptide binding at an IC50 value of ˜1 μM, a slightly better potency than that of T3 (FIG. 22C).

Next, it was determined if T2AA can inhibit PCNA interaction with a full-length protein. p21 is a protein containing a PIP-box with the highest affinity to PCNA known to date (5). Without wishing to be bound by a particular theory, T2AA inhibit inhibition of PCNA-p21 interaction can suggest that T2AA can perturb any PCNA/PIP-box protein interactions in cells. A pull-down assay was carried out by using recombinant p21 protein immobilized on cobalt beads. PCNAprotein was incubated with it in the presence of T3 or T2AA. They diminished the intensity of the PCNA band on the immobilized p21 dose-dependently (FIG. 22D). These data suggest that T2AA can inhibit any PCNA protein-protein interactions known to date once it reaches PCNA in the cells. It has been reported that PCNA-p21 interaction is mediated also at the IDCL region in addition to the PIP-box (Gulbis, J. M., et al. (1996) Cell 87, 297-306), thus, without wishing to be bound by a particular theory, the ability of T2AA to eliminate this interaction suggests that the PIP box interaction is the primary determinant for the PCNA-protein interaction.

Briefly, FIG. 23 shows the following: (Panel A) close-up view of PCNA-T3 interaction site (Protein Data Bank code 3VKX). PCNA is shown as a light gray surfaced model. T3 (FIG. 2A) is shown as a stick model, in which carbon, oxygen, nitrogen, and iodine are shown in dark gray, and hydrogen is omitted. Water molecules packed in the crystal were omitted. The 5-iodine interacts with the IDCL loop, inducing an extra cavity (gray oval, right side). The 3′-iodine (gray circle, left side) juts out from the PCNA interaction interface. (Panel B) superimposition of PCNAstructures bound to T3, p21 peptide (Protein Data Bank code 1AXC), and DNA Polδ peptide (Protein Data Bank code 2ZVK). The averaged root mean square deviation value is 0.84 Å for corresponding Cα atoms. All PCNA structures except that of the PCNA-T3 complex are structurally very similar, whereas the PCNA-T3 complex possesses significant perturbation of IDCL and the region of Asp41-Val45. (Panel C) stereo diagram of detailed interaction between T3 (darker gray) and PCNA (translucent white/light gray). T3 and residues of PCNA involved in the interaction with T3 are shown by sticks and transparent spheres.

(3) T2AA Inhibits PCNA-DNA Polymerases Interaction on Replication Foci in Cells but does not Remove Chromatin-Bound PCNA

The data showing that T2AA disrupts the PCNA-p21 interaction in vitro was extended to determine if T2AA inhibits PCNA interactions in cells, in the same way that p21 has been reported to control the cell cycle (Chen, J., et al. (1995) Nature 374, 386-388). It has been previously reported that DNA polymerase δ is recruited to the DNA replication fork by PCNA (Bravo, R., et al. (1987) Nature 326, 515-517) to synthesize the lagging strand, in which the subunit 3 (Polδ3) directly binds to PCNA with its PIP-box (Bruning, J. B., and Shamoo, Y. (2004) Structure. 12, 2209-2219). Affinity of the Polδ3 PIP-box to PCNA is much smaller than that of p21, which possibly allows dynamic replication regulation, such as polymerase switching and replication inhibition by p21 (Li, R., et al. (1994) Nature 371, 534-537). Therefore, theoretically T2AA should disrupt PCNA-Polδ3 interaction if enough concentration is achieved on the replication fork.

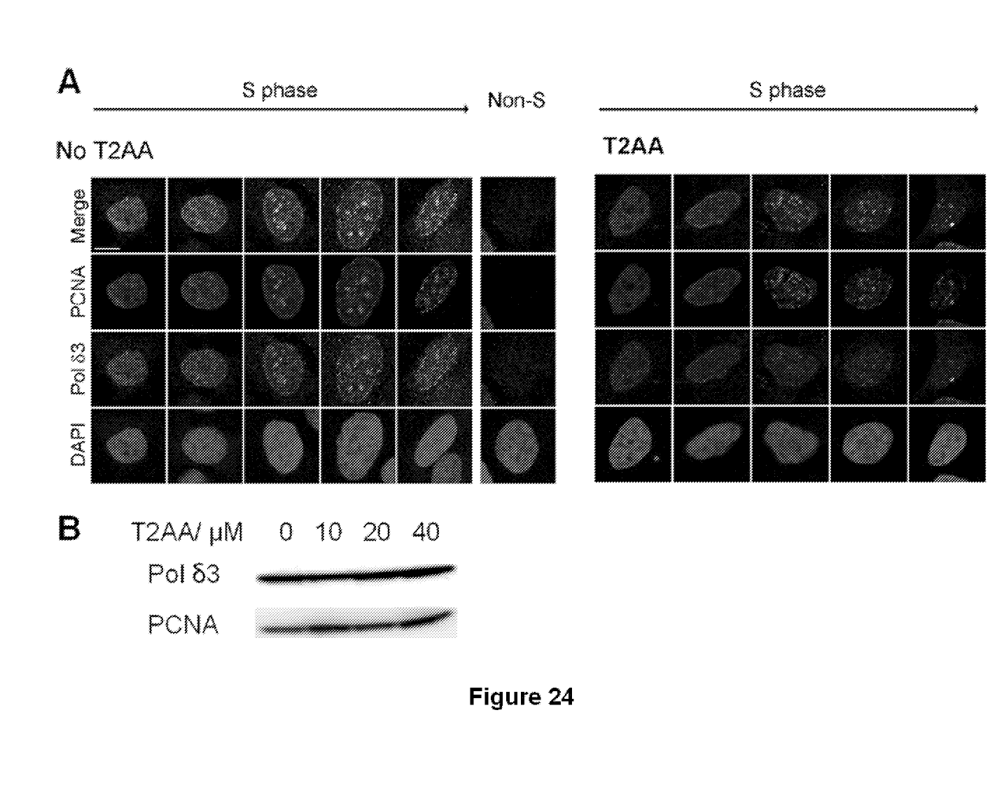

To verify this, the PCNA and Polδ3 in chromatin of S-phase cells upon treatment of T2AA was imaged. Cells were treated by T2AA, pre-extracted to remove proteins unbound to chromatin, and immunostained. PCNA and Polδ3 are clearly colocalized in the absence of T2AA. However, when cells were treated with T2AA, Polδ3 was exclusively washed out from the chromatin, but PCNA was not (FIG. 24A). Therefore, T2AA dissociated Polδ3 from PCNA but did not dissociate PCNA from the replication fork. On the other hand, expressions of Polδ3 protein in whole cell lysate were not reduced (FIG. 24B), showing that the elimination of chromatin Polδ3 by T2AA is not due to its down-regulation.

Briefly, FIG. 24 shows the following: (Panel A) U2OS cells were untreated or treated with T2AA (20 μM) for 24 h. Proteins unbound to chromatin were extracted away by CSK buffer/Triton X-100, and each indicated protein was immunostained.DNAPolδ3 is colocalized well with PCNA in untreated S-phase cells but was removed by the extraction. Without wishing to be bound by a particular theory, the data suggest that the PCNA-Polδ3 interaction on chromatin was disrupted by the T2AA treatment, but PCNA loading on replication forks was not. A non-5-phase cell is shown as a negative control for PCNA staining. DAPI served as nuclear stain. (Panel B) immunoblotting for Polδ3 in whole lysates of U2OS cells treated with T2AA showed no down-regulation. PCNA served as a loading control.

(4) T2AA Inhibits DNA Replication in Cancer Cells and their Proliferation

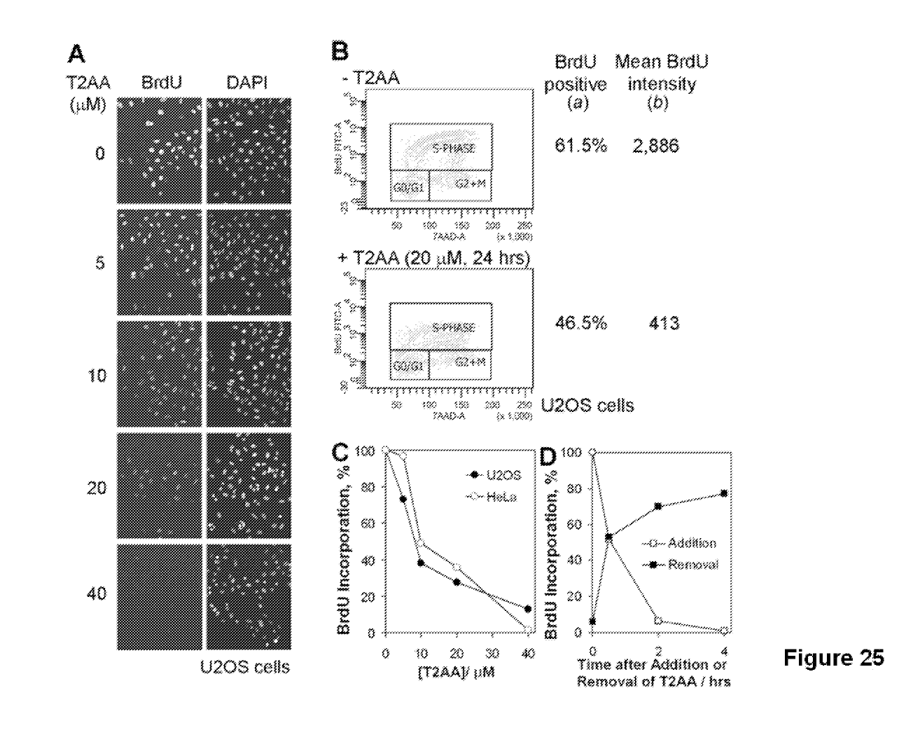

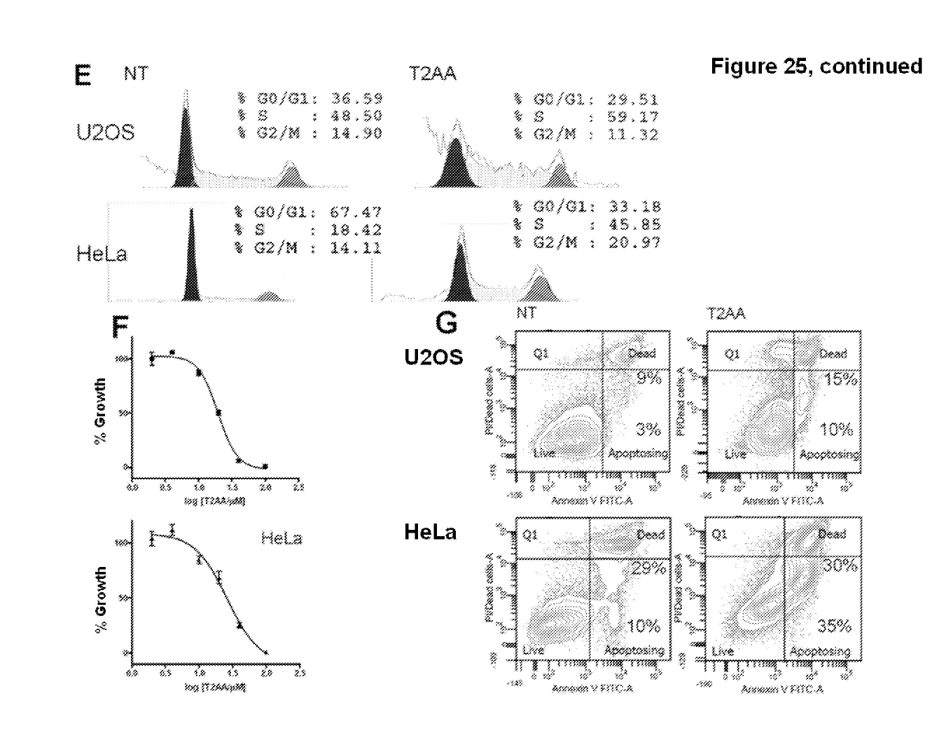

The indicated that T2AA disrupted PCNA-Polδ3 interaction on chromatin. Next, it was determined if T2AA could achieve relevant functional effects in cells. DNA polymerase δ synthesizes de novo DNA strands, which is measurable by nucleotide incorporation. Cells were treated with BrdU under titration of T2AA. The data show that T2AA inhibited the BrdU incorporation significantly in a dose-dependent manner (FIG. 25, A-C), which is consistent with the elimination of Polδ3 from chromatin. By performing the assays in a time-dependent manner after the addition or removal of T2AA, pharmacological action of T2AA appeared fairly rapid and was reversible. The BrdU incorporations were ˜90% inhibited within 2 h of T2AA treatment and ˜70% recovered after 2 h of T2AA release (FIG. 25D), which was ˜100% recovered after 18 h (data not shown). Parallel to the inhibition of DNA synthesis, T2AA arrested cells at S-phase (FIG. 25E), similarly to other agents that inhibit DNA replication/synthesis, such as aphidicolin and hydroxyurea. Further, when cells were cultured with T2AA, growth of both U2OS (p53 WT) and HeLa cells (p53 destroyed by E6) was inhibited with a similar efficacy (FIG. 25F). Without wishing to be bound by a particular theory, the data suggest that growth inhibition by T2AA is p53-independent. The growth inhibition was accompanied by increased cellular populations in early apoptosis but not necrosis (FIG. 25G). Without wishing to be bound by a particular theory, the data suggest that T2AA induced the growth inhibition by a specific mechanism and not by non-specific toxicity.

Briefly, FIG. 25 shows the following: (Panel A) T2AA inhibits DNA replication in cells. DNA replication was analyzed by BrdU incorporation after a 24-h treatment of U2OS cells with T2AA. Cells were treated with the drug at the indicated concentrations and labeled with 10 μM BrdU for 15 min. Incorporated BrdU was detected by immunostaining using anti-BrdU antibody. (Panels B and C) the BrdU incorporation and dose dependence of T2AA treatment were quantified by flow cytometry. The populations of cells positive for BrdU incorporation (S-phase) (a) and mean intensity of BrdU signal in the cells within the S-phase fraction (b) were determined. Total BrdU incorporation was defined as a x b. (Panel D) time dependence of the inhibition of DNA replication by T2AA was examined by flow cytometry. U2OScells were treated with 20 μM T2AA in an indicated period or released from a treatment with T2AA at 20 μM for 4 h. Cells were labeled with 20 μM BrdU for 15 min and analyzed by flow cytometry as in B and C. t_{1/2} of the response was read as ˜0.5 h in both the T2AA addition and release. (Panel E) T2AA arrested cells at S-phase. Cells were treated with T2AA (20 μM) for 24 h, stained by PI, and sorted by DNA contents. (Panel F) T2AA suppressed growth of cells. Cells were treated with the indicated titration of T2AA triplicate in 384-well plate for 3 days. Signals of each well were measured and normalized with that of well containing no cells (0%) and cells with no treatment (100%). Growth inhibition curve was fitted by Prism (GraphPad) to determine IC50 as 20 μM (U2OS) and 26 μM (HeLa). Error bars, S.E. G, T2AA induced early apoptosis. Cells were treated with T2AA (200M) for 5 days, stained, and sorted by Annexin-V and PI. The population percentages of early apoptosis (Apoptosing) and late apoptosis (Dead) cells are indicated. NT, no treatment.

(5) T2AA Induces DNA Replication Stress in Cells by Stalling Single-Stranded DNA

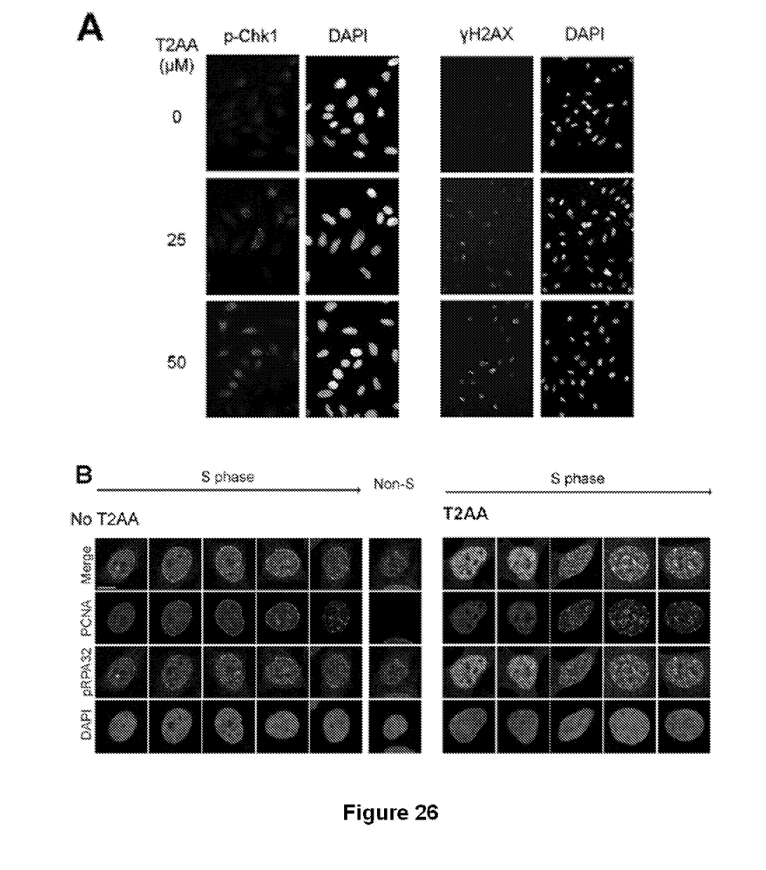

The data showed that T2AA inhibits DNA replication. Next, it was determined if T2AA induces DNA replication stress. When DNA replication is arrested in S-phase, cells contain unreplicated single-stranded DNA (ssDNA) in chromatin and subsequently activate the ATR-Chk1 pathway, leading to histone H2AX phosphorylation (Flynn, R. L. and Zou, L. (2011) Trends Biochem. Sci. 36, 133-140). Chk1 and H2AX were phosphorylated upon T2AA treatment (FIG. 26A). ATR phosphorylated RPA32 that was accumulated on ssDNA, and such RPA32 phosphorylation was observed in cells treated with aphidicolin or hydroxyurea (Vassin, V. M., et al. (2009) J. Cell Sci. 122, 4070-4080). To verify this for T2AA, the RPA32 phosphorylation in chromatin by immunostaining was examined. After 24 h of treatment, T2AA increased the immunofluorescence of phospho-RPA32 in nuclei (FIG. 26B). The phospho-RPA32 foci were not colocalized well with PCNA foci, which is consistent with a previous observation that phosphorylation of RPA32 prevents RPA association to replication forks (Vassin, V. M., Wold, M. S., and Borowiec, J. A. (2004) Mol. Cell. Biol. 24, 1930-1943) where PCNA should exist. Without wishing to be bound by a particular theory, these data suggest that T2AA-mediated DNA replication arrest activates the ATR-Chk1 pathway and accumulates stalled ssDNA.

Briefly, FIG. 26 shows the following: (Panel A) HeLa cells were treated with indicated concentrations of T2AA for 4 h. Immunostaining was performed for Ser³⁴⁵-phosphorylated Chk1 or Ser¹³⁹-phosphorylated histone H2AX. (Panel B) U2OS cells were untreated or treated with T2AA (40 μM) for 24 h. Proteins unbound to chromatin were extracted away by CSK buffer/Triton X-100, and each indicated protein was immunostained. RPA32 on the chromatins of S-phase cells was Ser³³-phosphorylated upon T2AA treatment. A non-5-phase cell is shown as a negative control for PCNA staining. DAPI served as nuclear staining agent.

(6) T2AA Inhibits TLS in Cells

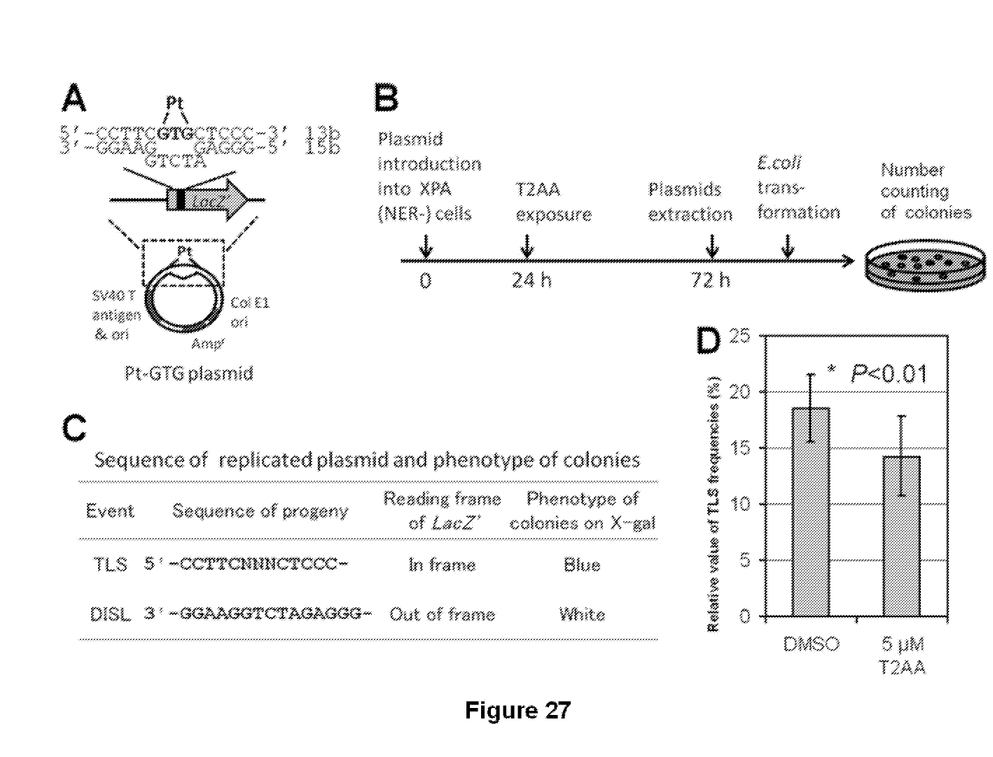

It has been reported that one of the important roles of PCNA is polymerase switching from regular replication polymerases (such as DNA Polδ) to translesion synthesis polymerases (such as DNA Polη) when the replication fork meets a damaged site on the DNA template (such as a cisplatin intrastrand cross-link, the major reaction product of DNA and cisplatin; Lehmann, A. R. (2006) Exp. Cell Res. 312, 2673-2676). The data showed that T2AA inhibited DNA synthesis in cells (FIG. 25A). Next, it was determined if T2AA can inhibit TLS that is PCNA-dependent (Hishiki, A., et al. (2009) J. Biol. Chem. 284, 10552-10560), using a cellular TLS assay was used wherein replications of a plasmid DNA containing an intrastrand cisplatin cross-link (Pt-GTG) in a coding region of the lacZ gene (FIG. 27A), in which another plasmid lacking the cross-link serves as a control for non-TLS plasmid replication (14). To avoid removal of the cross-link by NER, we used XP2OS(SV40), an NER-deficient XPA cell line (Kawanishi, M., et al. (1998) Chem. Res. Toxicol. 11, 1468-1473). The cells were transfected with the plasmid in the presence of T2AA. The replicated plasmids in the cells were recovered and analyzed for the TLS event by E. coli transformation/colony selection on X-gal plates (FIGS. 27, B and C). T2AA significantly reduced the occurrence of TLS compared with that of DMSO control (14.2 versus 18.5% (i.e. 23% reduction); FIG. 27D).

Briefly, FIG. 27 shows the following: (Panel A) shows the structure of the cisplatin-crosslinked Pt-GTG plasmid. (Panel B) shows the experimental protocol used in the study of FIG. 27. (Panel C) predicted sequences of replicated plasmid and phenotypes of colonies on the X-gal LB plates; DISL, damage induced strand loss. (Panel D) T2AA inhibited TLS across the cisplatin-cross-linked Pt-GTG adduct. Relative values of the TLS frequency (percentages) across Pt-GTG adduct in XPA cells treated with DMSO (18.5±3.0%) or T2AA (14.2±3.6%) are shown, which is the ratio of the TLS frequency of the modified plasmid to that of the non-modified plasmid (i.e. (TLS frequency of Pt-GTG plasmid/TLS frequency of Mock-GTG plasmid)×100). Data are means with S.D. values (error bars) from at least three independent experiments. The value significantly decreased (*, p<0.01) from that of the DMSO control. Statistical comparison was carried out using Student's t test for one-tailed comparison. Actual numbers of the colony counting are given in supplemental Table 4a and Table 4b below.

TABLE 4a
5 μM of T2AA

| | Mock GTG plasmid | | | Pt-GTG plasmid | | | |
|---|---|---|---|---|---|---|---|
| | Number of | | | Number of | | | Relative |
| | colonies | | Raito (%) | colonies | | Raito (%) | ratio |
| Exp. No. | blue | total | blue/total | blue | total | blue/total | (%) |
| 1 | 138 | 362 | 38.1 | 6 | 113 | 5.3 | 14.4 |
| | 117 | 342 | 34.2 | 4 | 115 | 3.5 | 9.4 |
| | | | | 45 | 681 | 6.6 | 17.9 |
| 2 | 215 | 598 | 36.0 | 5 | 81 | 6.2 | 16.7 |
| | 183 | 531 | 34.5 | 6 | 92 | 6.5 | 17.7 |
| | | | | 35 | 575 | 6.1 | 16.5 |
| 3 | 169 | 412 | 41.0 | 3 | 79 | 3.8 | 10.3 |
| | 219 | 578 | 37.9 | 4 | 116 | 3.4 | 9.3 |
| | | | | 43 | 728 | 5.9 | 16.0 |
| Total/ | 1041 | 2823 | 36.9 (100) | 151 | 2580 | 5.3 (14.2) | 14.2 ± |
| Average | | | | | | | 3.6 |
| (Relative | | | | | | | |
| value) | | | | | | | |

TABLE 4b
DMSO

| | Mock GTG plasmid | | | Pt-GTG plasmid | | | |
|---|---|---|---|---|---|---|---|
| | Number of | | | Number of | | | Relative |
| | colonies | | Ratio (%) | colonies | | Ratio (%) | ratio |
| Exp. No. | blue | total | blue/total | blue | total | blue/total | (%) |
| 1 | 188 | 429 | 43.8 | 9 | 118 | 7.6 | 18.2 |
| | 168 | 426 | 39.4 | 9 | 128 | 7.0 | 16.8 |
| | | | | 25 | 436 | 5.7 | 13.7 |
| 2 | 216 | 507 | 42.6 | 14 | 186 | 7.5 | 18.0 |
| | 251 | 590 | 42.5 | 55 | 717 | 7.7 | 18.3 |
| 3 | 142 | 331 | 42.9 | 10 | 102 | 9.8 | 23.4 |
| | 176 | 445 | 39.6 | 14 | 153 | 9.2 | 21.9 |
| | | | | 66 | 885 | 7.5 | 17.8 |
| Total/ | 1141 | 2728 | 41.8 (100) | 202 | 2725 | 7.8 (18.5) | 18.5 ± |
| Average | | | | | | | 3.0 |
| (Relative | | | | | | | |
| value) | | | | | | | |

(7) T2AA Increases Cellular DNA Damage Induced by Cisplatin

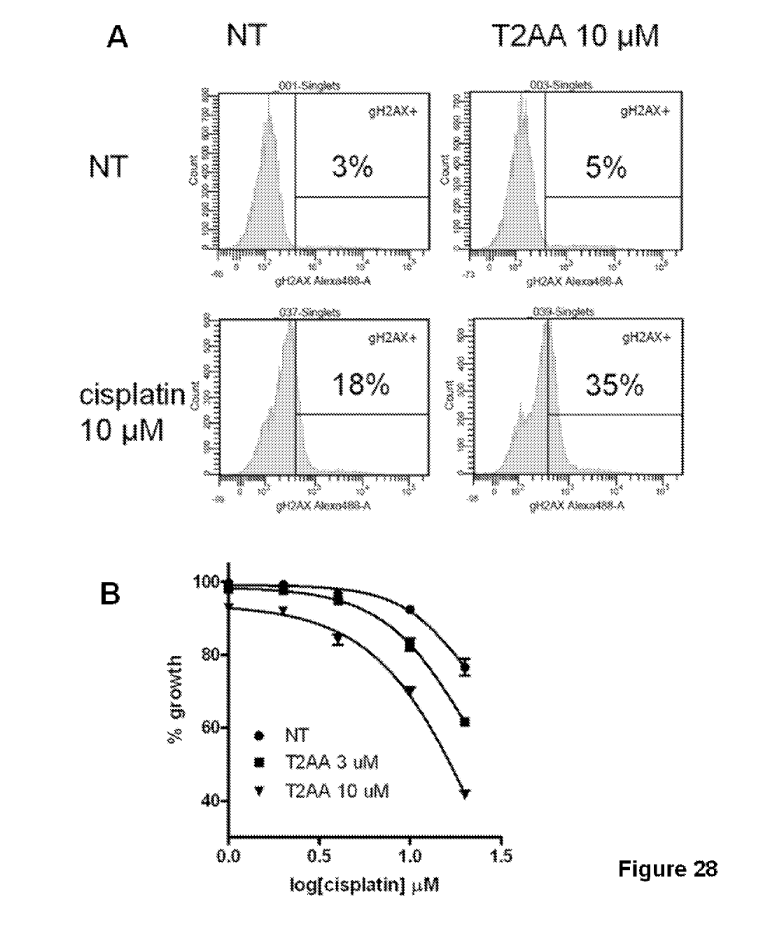

The data showed that T2AA inhibited the TLS in cells. Next, it was determined T2AA could increase cellular DNAdamage upon treatment with cisplatin. U2OS cells were treated with T2AA, cisplatin, or both, and then the DNA damage response was measured by γH2AX staining. The data show that cisplatin significantly induced γH2AX, whereas T2AA induced γH2AX only a little. In contrast, when both T2AA and cisplatin were added together, the γH2AX level was further increased much higher than the level combined from each single treatment (FIG. 28A). The influence of the cisplatin/T2AA-induced DNA damage on cell growth was assessed by removing the drugs to release the cells from S-phase arrest. The cells were cultured under drug-free conditions, and their viability was measured. U2OS cells are relatively resistant to cisplatin, and their growth is not significantly affected by the presence of up to 10 μM cisplatin, and it was reduced only less than 10% with 10 μM T2AA. In contrast, when cells received both compounds, their growth was significantly reduced (FIG. 28B).

Briefly, FIG. 28 shows the following: (Panel A) T2AA increased cisplatin-induced DNA damage response. U2OScells were treated with the indicated combination of T2AA and cisplatin for 18 h, stained for γH2AX, and analyzed by flow cytometry. (Panel B) growth of cells treated with cisplatin and T2AA. U2OS cells were treated with the indicated titration of cisplatin with T2AA for 18 h, replated in fresh medium to remove the drugs, and cultured for 3 days. Viability of the cells was measured by Alamar Blue reagent, and signals were normalized with those of no cell well (0%) and cells with no drugs (100%). NT, no treatment. Error bars, S.D.

### 109. Prospective In Vivo Activity

Generally compounds that inhibit PCNA protein activity in preclinical animal models of tumor growth and pathology. In vivo effects of the compounds described in the preceding examples are expected to be show activity in various models cancer biology known to the skilled person, such as a mouse subcutaneous xenograft model or, alternatively, the mouse orthotopic xenograft model. These models are typically conducted in an immunocompromised mouse, e.g. athymic nude mice, severely compromised immunodeficient (SCID) mice, or other immunocompromised mice (see reference (40) above), but may be conducted in other animal species as is convenient to the study goals. Alternatively, a genetically engineered mouse (GEM) model can be used to assess the efficacy of the disclosed compounds on inhibiting tumor growth. The genetic profile of GEM mice is altered such that one or several genes thought to be involved in transformation or malignancy are mutated, deleted or overexpressed; subsequently, the effect of altering these genes is studied over time and therapeutic responses to these tumors may be followed in vivo.

The subcutaneous xenograft model is frequently used by one skilled in the art to assess anti-cancer activity. Briefly, the cell-line of choice, e.g. MDA-MB-231, Panc-1, DU 145, or NIT3T3/v-Src cells, are grown in vitro in culture flasks, and then collected with using trypsin (if adherent) or by simple centrifugation (if suspension cultures), and then suspended in PBS at about 6×10 7 cells/mL. In one experimental approach, about 10⁵ cells are injected in mice subcutaneously on Day 0, and the tumors allowed to develop to about 10⁶ cells (about 7-10 days). Suitable mice strains to use in this include nu/nu nude mice or CAnN.Cg-Foxn1nu/CrlCrlj(nu/nu), and are readily available from suitable commercial sources (e.g. Harlan or Charles River). Drug is administered by a suitable route of administration, e.g. intravenous or intraperitoneally, on a dosing schedule suitable for the compound, e.g. daily for a period of five days or every third day for a period of two weeks or other schedule as determined from in vitro and in vivo data on potency, pharmacokinetics, and metabolism. The vehicle choice is determined based on the physical-chemical properties of the test compound. Exemplary vehicles include mixtures comprising DMSO, Cremaphor, and vegetable oils, e.g. 12.5% DMSO, 5% Cremaphor and 82.5% peanut oil; polysorbate:ethanol, e.g. 80:13; cremaphor:ethanol; and normal saline, e.g. phosphate-buffered saline. Body weight and tumor diameter are measured on a suitable schedule, e.g. every 3-4 days using calipers, and tumor volume determined by calculating the volume of an ellipsoid using the formula: length×width ²×0.5. Antitumor activities can be expressed as percent inhibition of tumor growth and percent regression of the tumor.

For example, compounds having a structure represented by a formula:

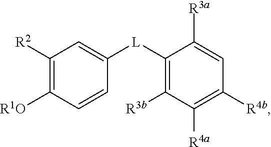

wherein L is selected from O, CH₂, CHOH, and C═O; wherein R¹ is selected from hydrogen and C1-C3 alkyl; wherein R² is selected from hydrogen, C1-C6 alkyl, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein one of R^{4a} and R^{4b} is selected from hydrogen, halo, C1-C3 alkyl, C1-C3 haloalkyl, and C1-C3 polyhaloalkyl, and the other is selected from:

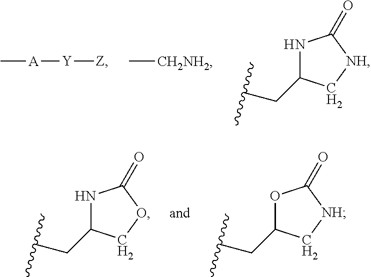

wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C₁-C₃ haloalkyl, and C1-C3 polyhaloalkyl; wherein Z is selected from (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹; wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C₁-C₃ alkoxy, C1-C3 haloalkyl, C1-C3 polyhaloalkyl, and heterocycloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof, are expected to show such in vivo effects.

For example, compounds having a structure represented by a formula:

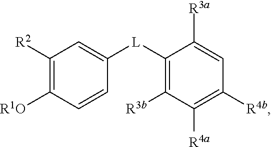

wherein L is selected from O, CH₂, CHOH, and C═O; wherein R¹ is selected from hydrogen and C1-C3 alkyl; wherein R² is selected from hydrogen, halo, (C═O)NR⁵SO₂R⁶, and (C═O)NR⁵R⁶; wherein R⁵ is selected from hydrogen and C1-C3 alkyl; wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein one of R^{4a} and R^{4b} is selected from hydrogen, halo, C1-C3 alkyl, C1-C3 haloalkyl, and C1-C3 polyhaloalkyl, and the other is -A-Y—Z; wherein A is optionally present, and when present is selected from O and CH₂; wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—; wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein Z is selected from (C═O)NR⁹R¹⁰, (C═O)NHSO₂R⁹, CH₂OH, CH₂NH₂, CH₂NH(C═O)R⁹, and CH₂NHSO₂R⁹; wherein R⁹ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl; wherein R¹⁰ is selected from hydrogen and C1-C6 alkyl; or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and wherein R¹¹ is selected from hydrogen and C1-C6 alkyl; or a pharmaceutically acceptable salt, hydrate, solvate, or polymorph thereof, are expected to show such in vivo effects.

For example, compounds having a structure represented by a formula:

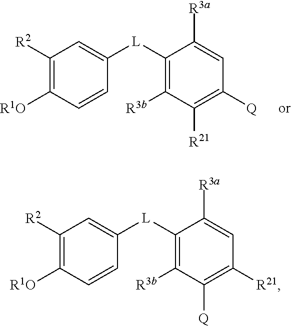

wherein L is selected from O, CH₂, CHOH, and C═O; wherein Q is selected from a structure represented by a formula:

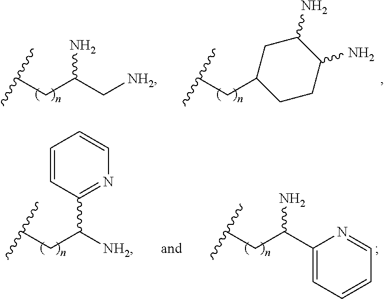

wherein n is an integer selected from 0, 1, 2, 3, 4, 5, and 6; wherein R¹ is selected from hydrogen and C1-C3 alkyl; wherein R² is selected from hydrogen, halo, (C═O)NR²²R²³, and (C═O)NSO₂R²³; wherein R²² is selected from hydrogen and C1-C3 alkyl; wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl; wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen; wherein R²¹ is selected from hydrogen, halo, C1-C3 alkyl, C1-C3 haloalkyl, and C1-C3 polyhaloalkyl; or a pharmaceutically acceptable salt, hydrate, solvate, or polymorph thereof, are expected to show such in vivo effects.

Moreover, compounds prepared using the disclosed synthetic methods are also expected to show such in vivo effects.

### 110. Prophetic Pharmaceutical Composition Examples

“Active ingredient” as used throughout these examples relates to one or more disclosed compounds, or a pharmaceutically acceptable salt, solvate, polymorph, hydrate and the stereochemically isomeric form thereof. The following examples of the formulation of the compounds of the present invention in tablets, suspension, injectables and ointments are prophetic.

Typical examples of recipes for the formulation of the invention are as given below. Various other dosage forms can be applied herein such as a filled gelatin capsule, liquid emulsion/suspension, ointments, suppositories or chewable tablet form employing the disclosed compounds in desired dosage amounts in accordance with the present invention. Various conventional techniques for preparing suitable dosage forms can be used to prepare the prophetic pharmaceutical compositions, such as those disclosed herein and in standard reference texts, for example the British and US Pharmacopoeias, Remington's Pharmaceutical Sciences (Mack Publishing Co.) and Martindale The Extra Pharmacopoeia (London The Pharmaceutical Press). The disclosure of this reference is hereby incorporated herein by reference.

a. Pharmaceutical Composition for Oral Administration

A tablet can be prepared as follows:

| Component | Amount | |
|---|---|---|
| Active ingredient | 10 to 500 | mg |
| Lactose | 100 | mg |
| Crystalline cellulose | 60 | mg |
| Magnesium stearate | 5 | |
| Starch (e.g. potato starch) | Amount necessary to yield total | |
| | weight indicated below | |
| Total (per capsule) | 1000 | mg |

Alternatively, about 100 mg of a disclosed compound, 50 mg of lactose (monohydrate), 50 mg of maize starch (native), 10 mg of polyvinylpyrrolidone (PVP 25) (e.g. from BASF, Ludwigshafen, Germany) and 2 mg of magnesium stearate are used per tablet. The mixture of active component, lactose and starch is granulated with a 5% solution (m/m) of the PVP in water. After drying, the granules are mixed with magnesium stearate for 5 min. This mixture is moulded using a customary tablet press (e.g. tablet format: diameter 8 mm, curvature radius 12 mm). The moulding force applied is typically about 15 kN.

Alternatively, a disclosed compound can be administered in a suspension formulated for oral use. For example, about 100-5000 mg of the desired disclosed compound, 1000 mg of ethanol (96%), 400 mg of xanthan gum, and 99 g of water are combined with stirring. A single dose of about 10-500 mg of the desired disclosed compound according can be provided by 10 ml of oral suspension.

In these Examples, active ingredient can be replaced with the same amount of any of the compounds according to the present invention, in particular by the same amount of any of the exemplified compounds. In some circumstances it may be desirable to use a capsule, e.g. a filled gelatin capsule, instead of a tablet form. The choice of tablet or capsule will depend, in part, upon physicochemical characteristics of the particular disclosed compound used.

Examples of alternative useful carriers for making oral preparations are lactose, sucrose, starch, talc, magnesium stearate, crystalline cellulose, methyl cellulose, hydroxypropyl cellulose, hydroxypropylmethyl cellulose, carboxymethyl cellulose, glycerin, sodium alginate, gum arabic, etc. These alternative carriers can be substituted for those given above as required for desired dissolution, absorption, and manufacturing characteristics.

The amount of a disclosed compound per tablet for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

b. Pharmaceutical Composition for Injectable Use

A parenteral composition can be prepared as follows:

| Component | Amount | |
|---|---|---|
| Active ingredient | 10 to 500 | mg |
| Sodium carbonate | 560 | mg* |
| Sodium hydroxide | 80 | mg* |
| Distilled, sterile water | Quantity sufficient to prepare | |
| | total volumen indicated below. | |
| Total (per capsule) | 10 | ml per ampule |

*Amount adjusted as required to maintain physiological pH in the context of the amount of active ingredient, and form of active ingredient, e.g. a particular salt form of the active ingredient.

Alternatively, a pharmaceutical composition for intravenous injection can be used, with composition comprising about 100-5000 mg of a disclosed compound, 15 g polyethyleneglycol 400 and 250 g water in saline with optionally up to about 15% Cremophor EL, and optionally up to 15% ethyl alcohol, and optionally up to 2 equivalents of a pharmaceutically suitable acid such as citric acid or hydrochloric acid are used. The preparation of such an injectable composition can be accomplished as follows: The disclosed compound and the polyethyleneglycol 400 are dissolved in the water with stirring. The solution is sterile filtered (pore size 0.22 μm) and filled into heat sterilized infusion bottles under aseptic conditions. The infusion bottles are sealed with rubber seals.

In a further example, a pharmaceutical composition for intravenous injection can be used, with composition comprising about 10-500 mg of a disclosed compound, standard saline solution, optionally with up to 15% by weight of Cremophor EL, and optionally up to 15% by weight of ethyl alcohol, and optionally up to 2 equivalents of a pharmaceutically suitable acid such as citric acid or hydrochloric acid. Preparation can be accomplished as follows: a desired disclosed compound is dissolved in the saline solution with stirring. Optionally Cremophor EL, ethyl alcohol or acid are added. The solution is sterile filtered (pore size 0.22 μm) and filled into heat sterilized infusion bottles under aseptic conditions. The infusion bottles are sealed with rubber seals.

In this Example, active ingredient can be replaced with the same amount of any of the compounds according to the present invention, in particular by the same amount of any of the exemplified compounds.

The amount of a disclosed compound per ampule for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

Carriers suitable for parenteral preparations are, for example, water, physiological saline solution, etc. which can be used with tris(hydroxymethyl)aminomethane, sodium carbonate, sodium hydroxide or the like serving as a solubilizer or pH adjusting agent. The parenteral preparations contain preferably 50 to 1000 mg of a disclosed compound per dosage unit.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 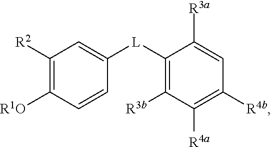
wherein L is selected from O, CH₂, CHOH, and C═O;
wherein R¹ is selected from hydrogen and C1-C3 alkyl;
wherein R² is selected from hydrogen, C1-C6 alkyl, halo, (C═O)NR⁵SO₂R⁶, and (C═O)NR⁵R⁶;
wherein R⁵ is selected from hydrogen and C1-C3 alkyl;
wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl;
wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen;
wherein one of R^{4a} and R^{4b} is selected from hydrogen, halo, C1-C3 alkyl, C1-C3 haloalkyl, and C1-C3 polyhaloalkyl, and the other is selected from: 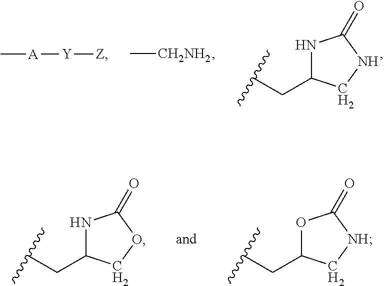
wherein A is optionally present, and when present is selected from O and CH₂;
wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—;
wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl;
wherein Z is selected from (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹;
wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl;
wherein R¹⁰ is selected from hydrogen and C1-C6 alkyl;
or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and
wherein R¹¹ is selected from hydrogen and C1-C6 alkyl;
or a pharmaceutically acceptable salt, hydrate, solvate, or polymorph thereof.

2. The compound of claim 1, wherein:
R² is selected from hydrogen, halo, (C═O)NR⁵SO₂R⁶, and (C═O)NR⁵R⁶;
one of R^{4a} and R^{4b} is selected from hydrogen, halo, C1-C3 alkyl, C1-C3 haloalkyl, and C1-C3 polyhaloalkyl, and the other is -A-Y—Z;
Z is selected from (C═O)NR⁹R¹⁰, (C═O)NHSO₂R⁹, CH₂OH, CH₂NH₂, CH₂NH(C═O)R⁹, and CH₂NHSO₂R⁹; and
R⁹ is selected from C1-C6 alkyl, C3-C6 cycloalkyl, C1-C6 heterocycloalkyl, aryl, heteroaryl, and is substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl.

3. The compound of claim 1, having a structure represented by a formula:

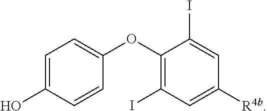

4. The compound of claim 1, having a structure represented by a formula:

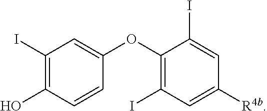

5. The compound of claim 1, having a structure represented by a formula: 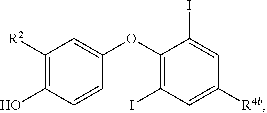
wherein R² is C1-C6 alkyl.

6. A compound having a structure represented by a formula: 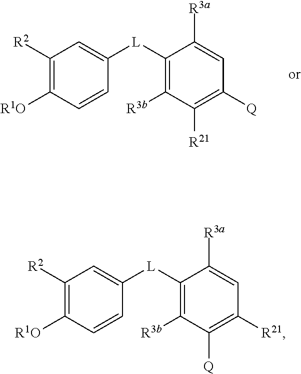
wherein L is selected from O, CH₂, CHOH, and C═O;
wherein Q is selected from a structure represented by a formula: 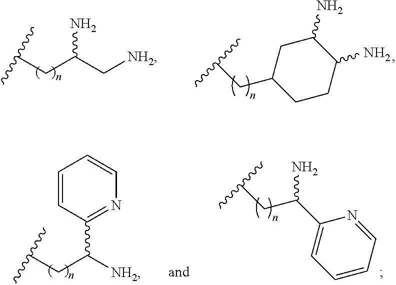
wherein n is an integer selected from 0, 1, 2, 3, 4, 5, and 6;
wherein R¹ is selected from hydrogen and C1-C3 alkyl;
wherein R² is selected from hydrogen, halo, (C═O)NR²²R²³, and (C═O)NSO₂R²³;
wherein R²² is selected from hydrogen and C1-C3 alkyl;
wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl;
wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen;
wherein R²¹ is selected from hydrogen, halo, C1-C3 alkyl, C1-C3 haloalkyl, and C1-C3 polyhaloalkyl;
or a pharmaceutically acceptable salt, hydrate, solvate, or polymorph thereof.

7. The compound of claim 1, wherein both of R^{3a} and R^{3b} are iodo.

8. A compound of claim 6, wherein the compound further comprises Pt(Z)₂; wherein each Z is selected from a halo; and wherein Q is selected from a structure represented by a formula:

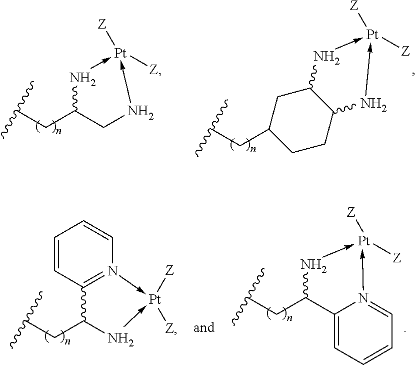

9. A compound of claim 6, wherein the compound further comprises platinum (II) oxalate; and wherein Q is selected from a structure represented by a formula:

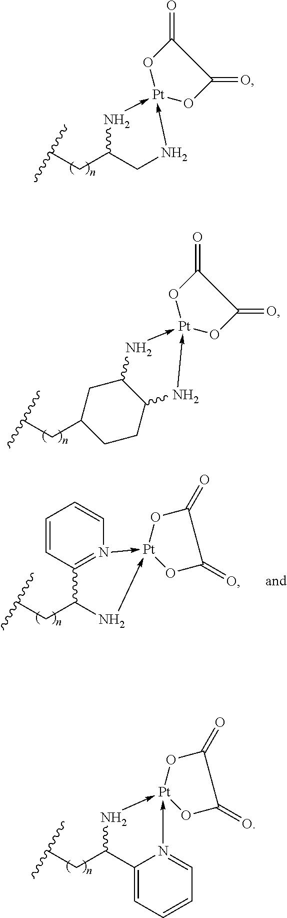

10. A compound of claim 6, wherein the compound further comprises platinum (II) 1,1-cyclobutanedicarboxylate; and wherein Q is selected from a structure represented by a formula:

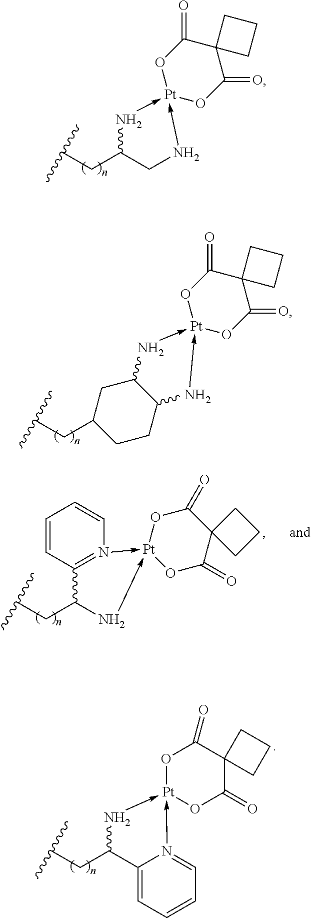

11. A pharmaceutical composition comprising a therapeutically effective amount of a compound of claim 1 and a pharmaceutically acceptable carrier.

12. A method for treatment of a proliferative disorder in a mammal comprising the step of administering to the mammal a therapeutically effective amount of at least one compound having a structure represented by formula: 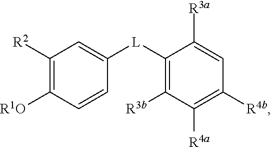
wherein L is selected from O, CH₂, CHOH, and C═O;
wherein R¹ is selected from hydrogen and C1-C3 alkyl;
wherein R² is selected from hydrogen, C1-C6 alkyl, halo, (C═O)NR⁵SO₂R⁶, and (C═O)NR⁵R⁶;
wherein R⁵ is selected from hydrogen and C1-C3 alkyl;
wherein R⁶ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl;
wherein each of R^{3a} and R^{3b} is independently selected from hydrogen, halo, cyano, C1-C3 alkyl, C1-C3 haloalkyl, and C1-C3 polyhaloalkyl;
wherein one of R^{4a} and R^{4b} is selected from hydrogen, halo, C1-C3 alkyl, C1-C3 haloalkyl, and C1-C3 polyhaloalkyl, and the other is selected from: 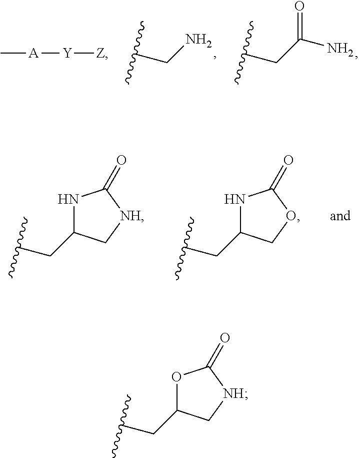
wherein A is optionally present, and when present is selected from O and CH₂;
wherein Y is selected from —CH₂—, —CH₂CH₂—, —CHNH₂—, —CHNH(C═O)R⁸—, —CHNH(C═O)OR⁸—, —CHNH(C═O)NHR⁸—, and —CHNHSO₂R⁸—;
wherein R⁸ is selected from C1-C6 alkyl, C3-C6 cycloalkyl, C1-C6 heterocycloalkyl, aryl, and heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl;
wherein Z is selected from CO₂H, (C═O)NR⁹R¹⁰, (C═O)NHSO₂R⁹, CH₂OH, CH₂NH₂, CH₂NH(C═O)R⁹, CH₂NH(C═O)NHR⁹, CH₂NH(C═O)NR⁹R¹⁰, and CH₂NHSO₂R⁹;
wherein R⁹ is selected from C1-C6 alkyl, C3-C6 cycloalkyl, C1-C6 heterocycloalkyl, aryl, heteroaryl, and benzyl, and is substituted with 0-5 groups independently selected from halo, hydroxyl, cyano, amino, SO₂R¹¹, C1-C3 alkyl, C1-C3 alkoxy, C1-C3 haloalkyl, C1-C3 polyhaloalkyl, and heterocycloalkyl;
wherein R¹⁰ is selected from hydrogen and C1-C6 alkyl;
or wherein R⁹ and R¹⁰ are optionally covalently bonded and, together with the intermediate carbons, comprise a 3- to 7-membered heterocycloalkyl substituted with 0-5 groups independently selected from hydroxyl, amino, (C═O), SO₂R¹¹, C1-C3 alkyl, C1-C3 alkoxy, C1-C3 haloalkyl, and C1-C3 polyhaloalkyl; and
wherein R¹¹ is selected from hydrogen and C1-C6 alkyl;
or a pharmaceutically acceptable salt, hydrate, solvate, or polymorph thereof.

13. The method of claim 12, wherein:
R² is selected from hydrogen, halo, (C═O)NR⁵SO₂R⁶, and (C═O)NR⁵R⁶;
one of R^{4a} and R^{4b} is selected from hydrogen, halo, C1-C3 alkyl, C1-C3 haloalkyl, and C1-C3 polyhaloalkyl, and the other is -A-Y—Z;
Z is selected from CO₂H, (C═O)NR⁹R¹⁰, (C═O)NHSO₂R⁹, CH₂OH, CH₂NH₂, CH₂NH(C═O)R⁹, and CH₂NHSO₂R⁹;
R⁹ is selected from C1-C6 alkyl, C3-C6 cycloalkyl, C1-C6 heterocycloalkyl, aryl, heteroaryl, and is substituted with 0-5 groups independently selected from halo, hydroxyl, cyano, amino, SO₂R¹¹, C1-C3 alkyl, C1-C3 alkoxy, C1-C3 haloalkyl, and C1-C3 polyhaloalkyl.

14. A method for treatment of a proliferative disorder in a mammal comprising the step of administering to the mammal a therapeutically effective amount of at least one compound having a structure represented by formula: 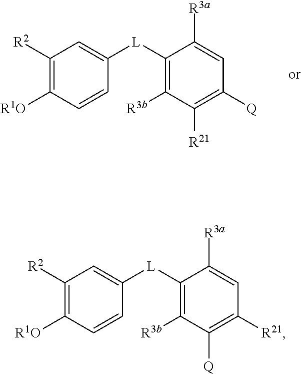
wherein L is selected from O, CH₂, CHOH, and C═O;
wherein Q is selected from a structure represented by a formula: 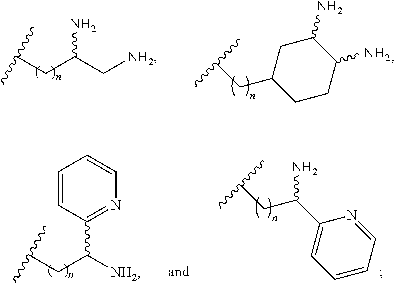
wherein n is an integer selected from 0, 1, 2, 3, 4, 5, and 6;
wherein R¹ is selected from hydrogen and C1-C3 alkyl;
wherein R² is selected from hydrogen, halo, (C═O)NR²²R²³, and (C═O)NSO₂R²³;
wherein R²² is selected from hydrogen and C1-C3 alkyl;
wherein R²³ is selected from C1-C6 alkyl, C3-C6 cycloalkyl, C1-C6 heterocycloalkyl, monocyclic aryl, and monocyclic heteroaryl, and substituted with 0-5 groups independently selected from halo, hydroxyl, cyano, amino, C1-C3 alkyl, C1-C3 alkoxy, C1-C3 haloalkyl, and C1-C3 polyhaloalkyl;
wherein each of R^{3a} and R^{3b} is independently selected from hydrogen, halo, cyano, C1-C3 alkyl, C1-C3 haloalkyl, and C1-C3 polyhaloalkyl, provided that R^{3a} and R^{3b} are not both hydrogen;
wherein R²¹ is selected from hydrogen, halo, C1-C3 alkyl, C1-C3 haloalkyl, and C1-C3 polyhaloalkyl;
or a pharmaceutically acceptable salt, hydrate, solvate, or polymorph thereof.

15. The method of claim 12, wherein the proliferative disorder is a hyperproliferative disorder.

16. The method of claim 15, wherein the hyperproliferative disorder is selected from a malignant, pre-malignant or non-malignant neoplastic disorder, inflammation, an autoimmune disorder, a haematological disorder, a skin disorder, a virally-induced hyperproliferative disorder, a myelodyplastic disorder or a myeloproliferative disorder.

17. The method of claim 15, wherein the hyperproliferative disorder is selected from cancer, benign tumours, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasia, agranuloma, MGUS (Monoclonal Gammopathy of Unknown Significance, neoplastic meningitis, polycythemia vera, scleromyxedema, papular mucinosis, amyloidosis and Wegener's granulomatosis.

18. The method of claim 15, wherein the hyperproliferative disorder is a cancer.

19. The method of claim 12, wherein the proliferative disorder is a cancer, and the compound has a structure represented by a formula: 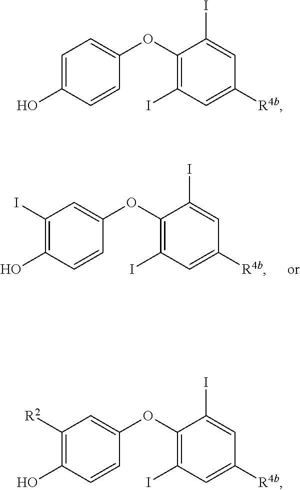
wherein R² is C1-C6 alkyl.

20. The method of claim 14, wherein the proliferative disorder is a cancer, and the compound has a structure represented by a formula: [chemical structure]
